(19) United States
(12) Patent Application Publication
(10) Pub. No.: US 2019/0204322 A1
(43) Pub. Date: Jul. 4, 2019

(54) MOLECULAR SUBTYPING, PROGNOSIS AND TREATMENT OF PROSTATE CANCER

(71) Applicant: GenomeDx Biosciences, Inc., Vancouver (CA)

(72) Inventors: Mohammed Alshalalfa, New Westminster (CA); Nicholas Erho, Vancouver (CA); Elai Davicioni, La Jolla, CA (US)

(21) Appl. No.: 15/758,308

(22) PCT Filed: Sep. 9, 2016
(86) PCT No.: PCT/IB2016/001344

§ 371 Date: Mar. 7, 2018

Related U.S. Application Data
(60) Provisional application No. 62/216,196, filed on Sep. 9, 2015.

(51) Int. Cl. G01N 33/574; C12Q 1/6886

(52) U.S. Cl. CPC G01N 33/57434; G01N 2800/56; C12Q 2600/112; C12Q 1/6886

(57) ABSTRACT

The present invention relates to methods, systems and kits for the diagnosis, prognosis and the determination of cancer progression of cancer in a subject. The invention also provides biomarkers that define subgroups of prostate cancer, clinically useful classifiers for distinguishing prostate cancer subtypes, bioinformatic methods for determining clinically useful classifiers, and methods of use of each of the foregoing. The methods, systems and kits can provide expression-based analysis of biomarkers for purposes of subtyping prostate cancer in a subject. Further disclosed herein, in certain instances, are probe sets for use in subtyping prostate cancer in a subject. Classifiers for subtyping a prostate cancer are provided. Methods of treating cancer based on molecular subtyping are also provided.

### RELATED APPLICATIONS

This application claims priority to U.S. Provisional Patent Application Ser. No. 62/216,196, filed on Sep. 9, 2015, which is hereby incorporated by reference herein in its entirety.

### FIELD OF THE INVENTION

The present invention relates to methods, systems and kits for the diagnosis, prognosis and the determination of cancer progression of cancer in a subject. The invention also provides biomarkers that define subgroups of prostate cancer, clinically useful classifiers for distinguishing prostate cancer subtypes, bioinformatic methods for determining clinically useful classifiers, and methods of use of each of the foregoing. The methods, systems and kits can provide expression-based analysis of biomarkers for purposes of subtyping prostate cancer in a subject. Further disclosed herein, in certain instances, are probe sets for use in subtyping prostate cancer in a subject. Classifiers for subtyping a prostate cancer are provided. Methods of treating cancer based on molecular subtyping are also provided.

### BACKGROUND OF THE INVENTION

Cancer is the uncontrolled growth of abnormal cells anywhere in a body. The abnormal cells are termed cancer cells, malignant cells, or tumor cells. Many cancers and the abnormal cells that compose the cancer tissue are further identified by the name of the tissue that the abnormal cells originated from (for example, prostate cancer). Cancer cells can proliferate uncontrollably and form a mass of cancer cells. Cancer cells can break away from this original mass of cells, travel through the blood and lymph systems, and lodge in other organs where they can again repeat the uncontrolled growth cycle. This process of cancer cells leaving an area and growing in another body area is often termed metastatic spread or metastatic disease. For example, if prostate cancer cells spread to a bone (or anywhere else), it can mean that the individual has metastatic prostate cancer.

Standard clinical parameters such as tumor size, grade, lymph node involvement and tumor-node-metastasis (TNM) staging (American Joint Committee on Cancer http://www.cancerstaging.org) may correlate with outcome and serve to stratify patients with respect to (neo)adjuvant chemotherapy, immunotherapy, antibody therapy and/or radiotherapy regimens. Incorporation of molecular markers in clinical practice may define tumor subtypes that are more likely to respond to targeted therapy. However, stage-matched tumors grouped by histological or molecular subtypes may respond differently to the same treatment regimen. Additional key genetic and epigenetic alterations may exist with important etiological contributions. A more detailed understanding of the molecular mechanisms and regulatory pathways at work in cancer cells and the tumor microenvironment (TME) could dramatically improve the design of novel anti-tumor drugs and inform the selection of optimal therapeutic strategies. The development and implementation of diagnostic, prognostic and therapeutic biomarkers to characterize the biology of each tumor may assist clinicians in making important decisions with regard to individual patient care and treatment. Thus, provided herein are methods, systems and kits for the diagnosis, prognosis and the determination of cancer progression of cancer in a subject. The invention also provides biomarkers that define subgroups of prostate cancer, clinically useful classifiers for distinguishing prostate cancer subtypes, bioinformatic methods for determining clinically useful classifiers, and methods of use of each of the foregoing. The methods, systems and kits can provide expression-based analysis of biomarkers for purposes of subtyping prostate cancer in a subject. Further disclosed herein, in certain instances, are probe sets for use in subtyping prostate cancer in a subject. Classifiers for subtyping a prostate cancer are provided. Methods of treating cancer based on molecular subtyping are also provided.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

The present invention relates to methods, systems and kits for the diagnosis, prognosis and the determination of cancer progression of cancer in a subject. The invention also provides biomarkers that define subgroups of prostate cancer, clinically useful classifiers for distinguishing prostate cancer subtypes, bioinformatic methods for determining clinically useful classifiers, and methods of use of each of the foregoing. The methods, systems and kits can provide expression-based analysis of biomarkers for purposes of subtyping prostate cancer in a subject. Further disclosed herein, in certain instances, are probe sets for use in subtyping prostate cancer in a subject. Classifiers for subtyping a prostate cancer are provided. Methods of treating cancer based on molecular subtyping are also provided.

In some embodiments, the present invention provides a method comprising: providing a biological sample from a prostate cancer subject; detecting the presence or expression level of at least one or more targets selected from Table 1, Table 2, Table 6, Table 7, Table 15 or SEQ ID NOs: 1-3348; and administering a treatment to the subject, wherein the treatment is selected from the group consisting of surgery, chemotherapy, radiation therapy, immunotherapy/biological therapy, hormonal therapy, and photodynamic therapy. In certain embodiments, the at least one or more targets is selected from the group consisting of ERG, ETV1, ETV4, ETV5, FLI1, SPINK1 or a combination thereof. In some embodiments, the at least one or more targets is selected from the group consisting of TDRD1, CACNA1D, NCALD, HLA-DMB, FAM65B, AMACR, SLC61A1, FKBP10 or a combination thereof. In other embodiments, the at least one or more targets is selected from the group consisting of TDRD1, CACNA1D, NCALD, HLA-DMB, FAM65B, AMACR, SLC61A1, FKBP10, HPGD, FAM3B, MIPEP, NCAPD3, INPP4B, ANPEP, TFF3, ALOX15B, MON1B or a combination thereof. In yet other embodiments, the at least one or more targets is selected from the group consisting of MME, BANK1, LEPREL1, VGLL3, NPR3, OR4K7P, OR4K6P, POTEB2, RP11, TTN, FAP5, GPR116 or a combination thereof. In another embodiment, the at least one or more targets is selected from the group consisting of SPINK1, BANK1, LEPREL1, TTN, POTEB2, OR4K7P, OR4K6P, FAB5P7, NPR1, RP11-403B2 or a combination thereof. In certain embodiments, the at least one or more targets is selected from the group consisting of GPR116, GRM7 or a combination thereof.

In some embodiments, the present invention provides a method comprising: providing a biological sample from a prostate cancer subject; detecting the presence or expression level of at least one or more targets selected from Table 1, Table 2, Table 6, Table 7, Table 15 or SEQ ID NOs: 1-3348. In certain embodiments, the at least one or more targets is selected from the group consisting of ERG, ETV1, ETV4, ETV5, FLI1, SPINK1 or a combination thereof. In some embodiments, the at least one or more targets is selected from the group consisting of TDRD1, CACNA1D, NCALD, HLA-DMB, FAM65B, AMACR, SLC61A1, FKBP10 or a combination thereof. In other embodiments, the at least one or more targets is selected from the group consisting of TDRD1, CACNA1D, NCALD, HLA-DMB, FAM65B, AMACR, SLC61A1, FKBP10, HPGD, FAM3B, MIPEP, NCAPD3, INPP4B, ANPEP, TFF3, ALOX15B, MON1B or a combination thereof. In yet other embodiments, the at least one or more targets is selected from the group consisting of MME, BANK1, LEPREL1, VGLL3, NPR3, OR4K7P, OR4K6P, POTEB2, RP11, TTN, FAP5, GPR116 or a combination thereof. In another embodiment, the at least one or more targets is selected from the group consisting of SPINK1, BANK1, LEPREL1, TTN, POTEB2, OR4K7P, OR4K6P, FAB5P7, NPR1, RP11-403B2 or a combination thereof. In certain embodiments, the at least one or more targets is selected from the group consisting of GPR116, GRM7 or a combination thereof.

In some embodiments, the present invention provides a method of subtyping prostate cancer in a subject, comprising: providing a biological sample comprising prostate cancer cells from the subject, and determining the level of expression or amplification of at least one or more targets selected from Table 1, Table 2, Table 6, Table 7, Table 15 or SEQ ID NOs: 1-3348 using at least one reagent that specifically binds to said targets; wherein the alteration of said expression level provides an indication of the prostate cancer subtype. In some embodiments, the alteration in the expression level of said target is reduced expression of said target. In other embodiments, the alteration in the expression level of said target is increased expression of said target. In yet other embodiments, the level of expression of said target is determined by using a method selected from the group consisting of in situ hybridization, a PCR-based method, an array-based method, an immunohistochemical method, an RNA assay method and an immunoassay method. In other embodiments, the reagent is selected from the group consisting of a nucleic acid probe, one or more nucleic acid primers, and an antibody. In still other embodiments, the target comprises a nucleic acid sequence. In certain embodiments, the at least one or more targets is selected from the group consisting of ERG, ETV1, ETV4, ETV5, FLI1, SPINK1 or a combination thereof. In some embodiments, the at least one or more targets is selected from the group consisting of TDRD1, CACNA1D, NCALD, HLA-DMB, FAM65B, AMACR, SLC61A1, FKBP10 or a combination thereof. In other embodiments, the at least one or more targets is selected from the group consisting of TDRD1, CACNA1D, NCALD, HLA-DMB, FAM65B, AMACR, SLC61A1, FKBP10, HPGD, FAM3B, MIPEP, NCAPD3, INPP4B, ANPEP, TFF3, ALOX15B, MON1B or a combination thereof. In yet other embodiments, the at least one or more targets is selected from the group consisting of MME, BANK1, LEPREL1, VGLL3, NPR3, OR4K7P, OR4K6P, POTEB2, RP11, TTN, FAP5, GPR116 or a combination thereof. In another embodiment, the at least one or more targets is selected from the group consisting of SPINK1, BANK1, LEPREL1, TTN, POTEB2, OR4K7P, OR4K6P, FAB5P7, NPR1, RP11-403B2 or a combination thereof. In certain embodiments, the at least one or more targets is selected from the group consisting of GPR116, GRM7 or a combination thereof.

In some embodiments the present invention provides methods of determining whether a subject has an ERG, ETS, SPINK1 positive prostate cancer or a triple negative cancer, comprising detecting the presence or expression level of at least one or more targets selected from TDRD1, CACNA1D, NCALD, HLA-DMB, FAM65B, AMACR, SLC61A1, GPR116, GRM7 and FKBP10, wherein an increase in TDRD1, CACNA1D, NCALD, GPR116, GRM7 and/or HLA-DM is indicative of ERG positive prostate cancer, an increase in FAM65B, AMACR, SLC61A1 and/or FKBP10 is indicative of ETS positive prostate cancer, an increase in HPGD, FAM3B, MIPEP, NCAPD3, INPP4B and/or ANPEP is indicative of SPINK-1 positive prostate cancer and an increase in TFF3, ALOX15B and/or MON1B is indicative of triple negative prostate cancer.

In some embodiments, the present invention also provides a method of diagnosing, prognosing, assessing the risk of recurrence or predicting benefit from therapy in a subject with prostate cancer, comprising: providing a biological sample comprising prostate cancer cells from the subject; assaying an expression level in the biological sample from the subject for a plurality of targets using at least one reagent that specifically binds to said targets, wherein the plurality of targets comprises one or more targets selected from Table 1, Table 2, Table 6, Table 7, Table 15 or SEQ ID NOs: 1-3348; and diagnosing, prognosing, assessing the risk of recurrence or predicting benefit from therapy in the subject based on the expression levels of the plurality of targets. In some embodiments, the expression level of the target is reduced expression of the target. In other embodiments, the expression level of said target is increased expression of said target. In yet other embodiments, the level of expression of said target is determined by using a method selected from the group consisting of in situ hybridization, a PCR-based method, an array-based method, an immunohistochemical method, an RNA assay method and an immunoassay method. In other embodiments, the reagent is selected from the group consisting of a nucleic acid probe, one or more nucleic acid primers, and an antibody. In other embodiments, the target comprises a nucleic acid sequence. In certain embodiments, the at least one or more targets is selected from the group consisting of ERG, ETV1, ETV4, ETV5, FLI1, SPINK1 or a combination thereof. In some embodiments, the at least one or more targets is selected from the group consisting of TDRD1, CACNA1D, NCALD, HLA-DMB, FAM65B, AMACR, SLC61A1, FKBP10 or a combination thereof. In other embodiments, the at least one or more targets is selected from the group consisting of TDRD1, CACNA1D, NCALD, HLA-DMB, FAM65B, AMACR, SLC61A1, FKBP10, HPGD, FAM3B, MIPEP, NCAPD3, INPP4B, ANPEP, TFF3, ALOX15B, MON1B or a combination thereof. In yet other embodiments, the at least one or more targets is selected from the group consisting of MME, BANK1, LEPREL1, VGLL3, NPR3, OR4K7P, OR4K6P, POTEB2, RP11, TTN, FAP5, GPR116 or a combination thereof. In another embodiment, the at least one or more targets is selected from the group consisting of SPINK1, BANK1, LEPREL1, TTN, POTEB2, OR4K7P, OR4K6P, FAB5P7, NPR1, RP11-403B2 or a combination thereof. In certain embodiments, the at least one or more targets is selected from the group consisting of GPR116, GRM7 or a combination thereof.

In some embodiments, the present invention provides a system for analyzing a cancer, comprising, a probe set comprising a plurality of target sequences, wherein the plurality of target sequences hybridizes to one or more targets selected from Table 1, Table 2, Table 6, Table 7, Table 15 or SEQ ID NOs: 1-3348; or the plurality of target sequences comprises one or more targets selected from Table 1, Table 2, Table 6, Table 7, Table 15 or SEQ ID NOs: 1-3348; and a computer model or algorithm for analyzing an expression level and/or expression profile of the target hybridized to the probe in a sample from a subject suffering from prostate cancer. In some embodiments, the method further comprises a label that specifically binds to the target, the probe, or a combination thereof. In certain embodiments, the at least one or more targets is selected from the group consisting of ERG, ETV1, ETV4, ETV5, FLI1, SPINK1 or a combination thereof. In some embodiments, the at least one or more targets is selected from the group consisting of TDRD1, CACNA1D, NCALD, HLA-DMB, FAM65B, AMACR, SLC61A1, FKBP10 or a combination thereof. In other embodiments, the at least one or more targets is selected from the group consisting of TDRD1, CACNA1D, NCALD, HLA-DMB, FAM65B, AMACR, SLC61A1, FKBP10, HPGD, FAM3B, MIPEP, NCAPD3, INPP4B, ANPEP, TFF3, ALOX15B, MON1B or a combination thereof. In yet other embodiments, the at least one or more targets is selected from the group consisting of MME, BANK1, LEPREL1, VGLL3, NPR3, OR4K7P, OR4K6P, POTEB2, RP11, TTN, FAP5, GPR116 or a combination thereof. In another embodiment, the at least one or more targets is selected from the group consisting of SPINK1, BANK1, LEPREL1, TTN, POTEB2, OR4K7P, OR4K6P, FAB5P7, NPR1, RP11-403B2 or a combination thereof. In certain embodiments, the at least one or more targets is selected from the group consisting of GPR116, GRM7 or a combination thereof.

In some embodiments, the present invention provides a method comprising: (a) providing a biological sample from a subject with prostate cancer; (b) detecting the presence or expression level in the biological sample for a plurality of targets, wherein the plurality of targets comprises one or more targets selected from Table 1, Table 2, Table 6, Table 7, Table 15 or SEQ ID NOs: 1-3348; (c) subtyping the prostate cancer in the subject based on the presence or expression levels of the plurality of targets; and (d) administering a treatment to the subject, wherein the treatment is selected from the group consisting of surgery, chemotherapy, radiation therapy, immunotherapy/biological therapy, hormonal therapy, and photodynamic therapy. In certain embodiments, the at least one or more targets is selected from the group consisting of ERG, ETV1, ETV4, ETV5, FLI1, SPINK1 or a combination thereof. In some embodiments, the at least one or more targets is selected from the group consisting of TDRD1, CACNA1D, NCALD, HLA-DMB, FAM65B, AMACR, SLC61A1, FKBP10 or a combination thereof. In other embodiments, the at least one or more targets is selected from the group consisting of TDRD1, CACNA1D, NCALD, HLA-DMB, FAM65B, AMACR, SLC61A1, FKBP10, HPGD, FAM3B, MIPEP, NCAPD3, INPP4B, ANPEP, TFF3, ALOX15B, MON1B or a combination thereof. In yet other embodiments, the at least one or more targets is selected from the group consisting of MME, BANK1, LEPREL1, VGLL3, NPR3, OR4K7P, OR4K6P, POTEB2, RP11, TTN, FAP5, GPR116 or a combination thereof. In another embodiment, the at least one or more targets is selected from the group consisting of SPINK1, BANK1, LEPREL1, TTN, POTEB2, OR4K7P, OR4K6P, FAB5P7, NPR1, RP11-403B2 or a combination thereof. In certain embodiments, the at least one or more targets is selected from the group consisting of GPR116, GRM7 or a combination thereof.

In some embodiments, the present invention provides a method comprising: (a) providing a biological sample from a subject with prostate cancer; (b) detecting the presence or expression level in the biological sample for a plurality of targets, wherein the plurality of targets comprises one or more targets selected from Table 1, Table 2, Table 6, Table 7, Table 15 or SEQ ID NOs: 1-3348; and (c) subtyping the prostate cancer in the subject based on the presence or expression levels of the plurality of targets. In certain embodiments, the at least one or more targets is selected from the group consisting of ERG, ETV1, ETV4, ETV5, FLI1, SPINK1 or a combination thereof. In some embodiments, the at least one or more targets is selected from the group consisting of TDRD1, CACNA1D, NCALD, HLA-DMB, FAM65B, AMACR, SLC61A1, FKBP10 or a combination thereof. In other embodiments, the at least one or more targets is selected from the group consisting of TDRD1, CACNA1D, NCALD, HLA-DMB, FAM65B, AMACR, SLC61A1, FKBP10, HPGD, FAM3B, MIPEP, NCAPD3, INPP4B, ANPEP, TFF3, ALOX15B, MON1B or a combination thereof. In yet other embodiments, the at least one or more targets is selected from the group consisting of MME, BANK1, LEPREL1, VGLL3, NPR3, OR4K7P, OR4K6P, POTEB2, RP11, TTN, FAP5, GPR116 or a combination thereof. In another embodiment, the at least one or more targets is selected from the group consisting of SPINK1, BANK1, LEPREL1, TTN, POTEB2, OR4K7P, OR4K6P, FAB5P7, NPR1, RP11-403B2 or a combination thereof. In certain embodiments, the at least one or more targets is selected from the group consisting of GPR116, GRM7 or a combination thereof.

In some embodiments, the present invention provides a method of treating a subject with prostate cancer, comprising: providing a biological sample comprising prostate cancer cells from the subject; determining the level of expression or amplification of at least one or more targets selected from Table 1, Table 2, Table 6, Table 7, Table 15 or SEQ ID NOs: 1-3348 using at least one reagent that specifically binds to said targets; subtyping the prostate cancer based on the level of expression or amplification of the at least one or more targets; and prescribing a treatment regimen based on the prostate cancer subtype. In some embodiments, the prostate cancer subtype is selected from the group consisting of ERG+, ETS+, SPINK1+, and Triple-Negative. In other embodiments the prostate cancer subtype is selected from the group consisting of MME+, Hetero, VGLL3+ or NOD. In certain embodiments, the at least one or more targets is selected from the group consisting of ERG, ETV1, ETV4, ETV5, FLI1, SPINK1 or a combination thereof. In some embodiments, the at least one or more targets is selected from the group consisting of TDRD1, CACNA1D, NCALD, HLA-DMB, FAM65B, AMACR, SLC61A1, FKBP10 or a combination thereof. In other embodiments, the at least one or more targets is selected from the group consisting of TDRD1, CACNA1D, NCALD, HLA-DMB, FAM65B, AMACR, SLC61A1, FKBP10, HPGD, FAM3B, MIPEP, NCAPD3, INPP4B, ANPEP, TFF3, ALOX15B, MON1B or a combination thereof. In yet other embodiments, the at least one or more targets is selected from the group consisting of MME, BANK1, LEPREL1, VGLL3, NPR3, OR4K7P, OR4K6P, POTEB2, RP11, TTN, FAP5, GPR116 or a combination thereof. In another embodiment, the at least one or more targets is selected from the group consisting of SPINK1, BANK1, LEPREL1, TTN, POTEB2, OR4K7P, OR4K6P, FAB5P7, NPR1, RP11-403B2 or a combination thereof. In certain embodiments, the at least one or more targets is selected from the group consisting of GPR116, GRM7 or a combination thereof.

In some embodiments, the present invention provides a kit for analyzing a prostate cancer, comprising, a probe set comprising a plurality of target sequences, wherein the plurality of target sequences comprises at least one target sequence listed in Table 1, Table 2, Table 6, Table 7, Table 15 or SEQ ID NOs: 1-3348; and a computer model or algorithm for analyzing an expression level and/or expression profile of the target sequences in a sample. In certain embodiments, the at least one or more targets is selected from the group consisting of ERG, ETV1, ETV4, ETV5, FLI1, SPINK1 or a combination thereof. In some embodiments, the at least one or more targets is selected from the group consisting of TDRD1, CACNA1D, NCALD, HLA-DMB, FAM65B, AMACR, SLC61A1, FKBP10 or a combination thereof. In other embodiments, the at least one or more targets is selected from the group consisting of TDRD1, CACNA1D, NCALD, HLA-DMB, FAM65B, AMACR, SLC61A1, FKBP10, HPGD, FAM3B, MIPEP, NCAPD3, INPP4B, ANPEP, TFF3, ALOX15B, MON1B or a combination thereof. In yet other embodiments, the at least one or more targets is selected from the group consisting of MME, BANK1, LEPREL1, VGLL3, NPR3, OR4K7P, OR4K6P, POTEB2, RP11, TTN, FAP5, GPR116 or a combination thereof. In another embodiment, the at least one or more targets is selected from the group consisting of SPINK1, BANK1, LEPREL1, TTN, POTEB2, OR4K7P, OR4K6P, FAB5P7, NPR1, RP11-403B2 or a combination thereof. In certain embodiments, the at least one or more targets is selected from the group consisting of GPR116, GRM7 or a combination thereof. In some embodiments, the method further comprises a computer model or algorithm for correlating the expression level or expression profile with disease state or outcome. In other embodiments, the method further comprises a computer model or algorithm for designating a treatment modality for the individual. In yet other embodiments, the method further comprises a computer model or algorithm for normalizing expression level or expression profile of the target sequences. In some embodiments, the method further comprises sequencing the plurality of targets. In some embodiments, the method further comprises hybridizing the plurality of targets to a solid support. In some embodiments, the solid support is a bead or array. In some embodiments, assaying the expression level of a plurality of targets may comprise the use of a probe set. In some embodiments, assaying the expression level may comprise the use of a classifier. The classifier may comprise a probe selection region (PSR). In some embodiments, the classifier may comprise the use of an algorithm. The algorithm may comprise a machine learning algorithm. In some embodiments, assaying the expression level may also comprise sequencing the plurality of targets.

Further disclosed herein methods for molecular subtyping of prostate cancer, wherein the subtypes have an AUC value of at least about 0.40 to predict patient outcomes. In some embodiments, patient outcomes are selected from the group consisting of biochemical recurrence (BCR), metastasis (MET) and prostate cancer death (PCSM) after radical prostatectomy. The AUC of the subtype may be at least about 0.40, 0.45, 0.50, 0.55, 0.60, 0.61, 0.62, 0.63, 0.64, 0.65, 0.66, 0.67, 0.68, 0.69, 0.70 or more.

Further disclosed herein is a method for subtyping a prostate cancer, comprising determining the level of expression or amplification of at least one or more targets of the present invention, wherein the significance of the expression level of the one or more targets is based on one or more metrics selected from the group comprising T-test, P-value, KS (Kolmogorov Smirnov) P-value, accuracy, accuracy P-value, positive predictive value (PPV), negative predictive value (NPV), sensitivity, specificity, AUC, AUC P-value (Auc.pvalue), Wilcoxon Test P-value, Median Fold Difference (MFD), Kaplan Meier (KM) curves, survival AUC (survAUC), Kaplan Meier P-value (KM P-value), Univariable Analysis Odds Ratio P-value (uvaORPval), multivariable analysis Odds Ratio P-value (mvaORPval), Univariable Analysis Hazard Ratio P-value (uvaHRPval) and Multivariable Analysis Hazard Ratio P-value (mvaHRPval). The significance of the expression level of the one or more targets may be based on two or more metrics selected from the group comprising AUC, AUC P-value (Auc.pvalue), Wilcoxon Test P-value, Median Fold Difference (MFD), Kaplan Meier (KM) curves, survival AUC (survAUC), Univariable Analysis Odds Ratio P-value (uvaORPval), multivariable analysis Odds Ratio P-value (mvaORPval), Kaplan Meier P-value (KM P-value), Univariable Analysis Hazard Ratio P-value (uvaHRPval) and Multivariable Analysis Hazard Ratio P-value (mvaHRPval). The molecular subtypes of the present invention are useful for predicting clinical characteristics of subjects with prostate cancer. In some embodiments, the clinical characteristics are selected from the group consisting of seminal vesical invasion (SVI), lymph node invasion (LNI), prostate-specific antigen (PSA), and gleason score (GS).

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference.

### BRIEF DESCRIPTION OF THE DRAWINGS

FIG. 1 sets forth data showing microarray expression data for molecular subtyping.

FIG. 2 sets forth data showing probe set expression across the ERG locus.

FIG. 3 sets forth data showing m-ERG scores plotted with stratification by F-ERG status.

FIG. 4 sets forth data showing m-ERG model scores in normal and tumor tissue.

FIG. 5 sets forth data showing m-ERG scores and technical replicates from 30 cohort samples.

FIGS. 6A-D set forth gene expression data for various molecular subtypes.

FIG. 7 sets forth data showing Beeswarm plots for core-level expression of ETV1, ETV4, ETV5, FLI1 and SPINK1.

FIG. 8 sets forth data showing m-ERG⁺ and TripleNeg expression centroids.

FIG. 9 sets forth data showing microarray expression data useful for molecular subtyping.

FIG. 10 sets forth data showing performance of a multigene PCa prognostic predictor (Decipher) is similar across molecular subtypes.

FIGS. 11A-C set forth data showing performance assessment of multiple prognostic signatures from genome-wide expression profiling data stratified by molecular subtypes.

FIGS. 12A-C show Kaplan Meier analysis that demonstrates similar PCa outcome measures across molecular subtypes.

FIG. 13 sets forth data showing Beeswarm plots for core-level expression of MME, BANK1, LEPREL1, VGLL3, NPR3, TTN, OR4K6P, OR4K7P, POTEB2, RP11.403 B1, FABP5P7 and GPR116 in prostate cancer samples.

FIGS. 14A-B set forth data showing molecular characterization of the heterogeneity of PCa.

FIG. 15 shows Kaplan Meier analysis with prognosis of various molecular subtypes.

FIGS. 16A-B set forth data showing use of outliers to subtype the four subtypes (ERG, ETS, SPINK, TripleNeg).

FIGS. 17A-C show Kaplan Meier analysis of subtypes in TripleNeg/SPINK subgroup.

FIG. 18 shows Kaplan Meier analysis of GPR116 in ERG+.

FIGS. 19 A-D show Kaplan Meier analysis of GPR116 in ERG+ patients.

FIGS. 20A-B set forth data showing that GPR116 is a predictive biomarker of ADT resistance in ERG+ patients

FIGS. 21A-C set forth data showing core-level expression of GPR116 and GRM7 in prostate cancer samples.

### DETAILED DESCRIPTION OF THE INVENTION

The present invention discloses systems and methods for diagnosing, predicting, and/or monitoring the status or outcome of a prostate cancer in a subject using expression-based analysis of a plurality of targets. Generally, the method comprises (a) optionally providing a sample from a subject; (b) assaying the expression level for a plurality of targets in the sample; and (c) diagnosing, predicting and/or monitoring the status or outcome of a prostate cancer based on the expression level of the plurality of targets.

Assaying the expression level for a plurality of targets in the sample may comprise applying the sample to a microarray. In some instances, assaying the expression level may comprise the use of an algorithm. The algorithm may be used to produce a classifier. Alternatively, the classifier may comprise a probe selection region. In some instances, assaying the expression level for a plurality of targets comprises detecting and/or quantifying the plurality of targets. In some embodiments, assaying the expression level for a plurality of targets comprises sequencing the plurality of targets. In some embodiments, assaying the expression level for a plurality of targets comprises amplifying the plurality of targets. In some embodiments, assaying the expression level for a plurality of targets comprises quantifying the plurality of targets. In some embodiments, assaying the expression level for a plurality of targets comprises conducting a multiplexed reaction on the plurality of targets.

In some instances, the plurality of targets comprises one or more targets selected from Table 1, Table 2, Table 6, Table 7, Table 15 or SEQ ID NOs: 1-3348. In some instances, the plurality of targets comprises at least about 2, at least about 3, at least about 4, at least about 5, at least about 6, at least about 7, at least about 8, at least about 9, or at least about 10 targets selected from Table 1, Table 2, Table 6, Table 7, Table 15 or SEQ ID NOs: 1-3348. In certain instances, the one or more targets is selected from ERG, ETV1, ETV4, ETV5, FLI1, and/or SPINK1; TDRD1, CACNA1D, NCALD, HLA-DMB, FAM65B, AMACR, SLC61A1, and/or FKBP10; TDRD1, CACNA1D, NCALD, HLA-DMB, FAM65B, AMACR, SLC61A1, FKBP10, HPGD, FAM3B, MIPEP, NCAPD3, INPP4B, ANPEP, TFF3, ALOX15B, and/or MON1B; MME, BANK1, LEPREL1, VGLL3, NPR3, OR4K7P, OR4K6P, POTEB2, RP11, TTN, FAP5, and/or GPR116; SPINK1, BANK1, LEPREL1, TTN, POTEB2, OR4K7P, OR4K6P, FAB5P7, NPR1, and/or RP11-403B2; GPR116, GRM7; or a combination thereof.

Further disclosed herein are methods for subtyping prostate cancer. Generally, the method comprises: (a) providing a sample comprising prostate cancer cells from a subject; (b) assaying the expression level for a plurality of targets in the sample; and (c) subtyping the cancer based on the expression level of the plurality of targets. In some instances, the plurality of targets comprises one or more targets selected from Table 1, Table 2, Table 6, Table 7, Table 15 or SEQ ID NOs: 1-3348. In some instances, the plurality of targets comprises at least about 2, at least about 3, at least about 4, at least about 5, at least about 6, at least about 7, at least about 8, at least about 9, or at least about 10 targets selected from Table 1, Table 2, Table 6, Table 7, Table 15 or SEQ ID NOs: 1-3348. In certain instances, the one or more targets is selected from ERG, ETV1, ETV4, ETV5, FLI1, and/or SPINK1; TDRD1, CACNA1D, NCALD, HLA-DMB, FAM65B, AMACR, SLC61A1, and/or FKBP10; TDRD1, CACNA1D, NCALD, HLA-DMB, FAM65B, AMACR, SLC61A1, FKBP10, HPGD, FAM3B, MIPEP, NCAPD3, INPP4B, ANPEP, TFF3, ALOX15B, and/or MON1B; MME, BANK1, LEPREL1, VGLL3, NPR3, OR4K7P, OR4K6P, POTEB2, RP11, TTN, FAP5, and/or GPR116; SPINK1, BANK1, LEPREL1, TTN, POTEB2, OR4K7P, OR4K6P, FAB5P7, NPR1, and/or RP11-403B2; GPR116, GRM7; or a combination thereof.

In some instances, subtyping the prostate cancer comprises determining whether the cancer would respond to an anti-cancer therapy. Alternatively, subtyping the prostate cancer comprises identifying the cancer as non-responsive to an anti-cancer therapy. Optionally, subtyping the prostate cancer comprises identifying the cancer as responsive to an anti-cancer therapy.

Before the present invention is described in further detail, it is to be understood that this invention is not limited to the particular methodology, compositions, articles or machines described, as such methods, compositions, articles or machines can, of course, vary. It is also to be understood that the terminology used herein is for the purpose of describing particular embodiments only, and is not intended to limit the scope of the present invention.

### Targets

The methods disclosed herein often comprise assaying the expression level of a plurality of targets. The plurality of targets may comprise coding targets and/or non-coding targets of a protein-coding gene or a non protein-coding gene. A protein-coding gene structure may comprise an exon and an intron. The exon may further comprise a coding sequence (CDS) and an untranslated region (UTR). The protein-coding gene may be transcribed to produce a pre-mRNA and the pre-mRNA may be processed to produce a mature mRNA. The mature mRNA may be translated to produce a protein.

A non protein-coding gene structure may comprise an exon and intron. Usually, the exon region of a non protein-coding gene primarily contains a UTR. The non protein-coding gene may be transcribed to produce a pre-mRNA and the pre-mRNA may be processed to produce a non-coding RNA (ncRNA).

A coding target may comprise a coding sequence of an exon. A non-coding target may comprise a UTR sequence of an exon, intron sequence, intergenic sequence, promoter sequence, non-coding transcript, CDS antisense, intronic antisense, UTR antisense, or non-coding transcript antisense. A non-coding transcript may comprise a non-coding RNA (ncRNA).

In some instances, the plurality of targets may be differentially expressed. In some instances, a plurality of probe selection regions (PSRs) is differentially expressed.

In some instances, the plurality of targets comprises one or more targets selected from Table 1, Table 2, Table 6, Table 7, Table 15 or SEQ ID NOs: 1-3348. In some instances, the plurality of targets comprises at least about 2, at least about 3, at least about 4, at least about 5, at least about 6, at least about 7, at least about 8, at least about 9, or at least about 10 targets selected from Table 1, Table 2, Table 6, Table 7, Table 15 or SEQ ID NOs: 1-3348. In certain instances, the plurality targets are selected from ERG, ETV1, ETV4, ETV5, FLI1, and/or SPINK1; TDRD1, CACNA1D, NCALD, HLA-DMB, FAM65B, AMACR, SLC61A1, and/or FKBP10; TDRD1, CACNA1D, NCALD, HLA-DMB, FAM65B, AMACR, SLC61A1, FKBP10, HPGD, FAM3B, MIPEP, NCAPD3, INPP4B, ANPEP, TFF3, ALOX15B, and/or MON1B; MME, BANK1, LEPREL1, VGLL3, NPR3, OR4K7P, OR4K6P, POTEB2, RP11, TTN, FAP5, and/or GPR116; SPINK1, BANK1, LEPREL1, TTN, POTEB2, OR4K7P, OR4K6P, FAB5P7, NPR1, and/or RP11-403B2; GPR116, GRM7; or a combination thereof.

In some instances, the plurality of targets comprises a coding target, non-coding target, or any combination thereof. In some instances, the coding target comprises an exonic sequence. In other instances, the non-coding target comprises a non-exonic or exonic sequence. Alternatively, a non-coding target comprises a UTR sequence, an intronic sequence, antisense, or a non-coding RNA transcript. In some instances, a non-coding target comprises sequences which partially overlap with a UTR sequence or an intronic sequence. A non-coding target also includes non-exonic and/or exonic transcripts. Exonic sequences may comprise regions on a protein-coding gene, such as an exon, UTR, or a portion thereof. Non-exonic sequences may comprise regions on a protein-coding, non protein-coding gene, or a portion thereof. For example, non-exonic sequences may comprise intronic regions, promoter regions, intergenic regions, a non-coding transcript, an exon anti-sense region, an intronic anti-sense region, UTR anti-sense region, non-coding transcript anti-sense region, or a portion thereof. In other instances, the plurality of targets comprises a non-coding RNA transcript.

The plurality of targets may comprise one or more targets selected from a classifier disclosed herein. The classifier may be generated from one or more models or algorithms. The one or more models or algorithms may be Naïve Bayes (NB), recursive Partitioning (Rpart), random forest (RF), support vector machine (SVM), k-nearest neighbor (KNN), high dimensional discriminate analysis (HDDA), or a combination thereof. The classifier may have an AUC of equal to or greater than 0.60. The classifier may have an AUC of equal to or greater than 0.61. The classifier may have an AUC of equal to or greater than 0.62. The classifier may have an AUC of equal to or greater than 0.63. The classifier may have an AUC of equal to or greater than 0.64. The classifier may have an AUC of equal to or greater than 0.65. The classifier may have an AUC of equal to or greater than 0.66. The classifier may have an AUC of equal to or greater than 0.67. The classifier may have an AUC of equal to or greater than 0.68. The classifier may have an AUC of equal to or greater than 0.69. The classifier may have an AUC of equal to or greater than 0.70. The classifier may have an AUC of equal to or greater than 0.75. The classifier may have an AUC of equal to or greater than 0.77. The classifier may have an AUC of equal to or greater than 0.78. The classifier may have an AUC of equal to or greater than 0.79. The classifier may have an AUC of equal to or greater than 0.80. The AUC may be clinically significant based on its 95% confidence interval (CI). The accuracy of the classifier may be at least about 70%. The accuracy of the classifier may be at least about 73%. The accuracy of the classifier may be at least about 75%. The accuracy of the classifier may be at least about 77%. The accuracy of the classifier may be at least about 80%. The accuracy of the classifier may be at least about 83%. The accuracy of the classifier may be at least about 84%. The accuracy of the classifier may be at least about 86%. The accuracy of the classifier may be at least about 88%. The accuracy of the classifier may be at least about 90%. The p-value of the classifier may be less than or equal to 0.05. The p-value of the classifier may be less than or equal to 0.04. The p-value of the classifier may be less than or equal to 0.03. The p-value of the classifier may be less than or equal to 0.02. The p-value of the classifier may be less than or equal to 0.01. The p-value of the classifier may be less than or equal to 0.008. The p-value of the classifier may be less than or equal to 0.006. The p-value of the classifier may be less than or equal to 0.004. The p-value of the classifier may be less than or equal to 0.002. The p-value of the classifier may be less than or equal to 0.001.

The plurality of targets may comprise one or more targets selected from a Random Forest (RF) classifier. The plurality of targets may comprise two or more targets selected from a Random Forest (RF) classifier. The plurality of targets may comprise three or more targets selected from a Random Forest (RF) classifier. The plurality of targets may comprise 2, 3, 4, 5, 6, 7, 8, 9, 10, 11, 12, 13, 14, 15 or more targets selected from a Random Forest (RF) classifier. The RF classifier may be an RF2, and RF3, or an RF4 classifier. The RF classifier may be an RF15 classifier (e.g., a Random Forest classifier with 15 targets).

A RF classifier of the present invention may comprise two or more targets comprising two or more targets selected from Table 1, Table 2, Table 6, Table 7, Table 15 or SEQ ID NOs: 1-3348. In certain instances, the two or more targets are selected from ERG, ETV1, ETV4, ETV5, FLI1, and/or SPINK1; TDRD1, CACNA1D, NCALD, HLA-DMB, FAM65B, AMACR, SLC61A1, and/or FKBP10; TDRD1, CACNA1D, NCALD, HLA-DMB, FAM65B, AMACR, SLC61A1, FKBP10, HPGD, FAM3B, MIPEP, NCAPD3, INPP4B, ANPEP, TFF3, ALOX15B, and/or MON1B; MME, BANK1, LEPREL1, VGLL3, NPR3, OR4K7P, OR4K6P, POTEB2, RP11, TTN, FAP5, and/or GPR116; SPINK1, BANK1, LEPREL1, TTN, POTEB2, OR4K7P, OR4K6P, FAB5P7, NPR1, and/or RP11-403B2; GPR116, GRM7; or a combination thereof.

The plurality of targets may comprise one or more targets selected from an SVM classifier. The plurality of targets may comprise 2, 3, 4, 5, 6, 7, 8, 9, 10 or more targets selected from an SVM classifier. The plurality of targets may comprise 12, 13, 14, 15, 17, 20, 22, 25, 27, 30 or more targets selected from an SVM classifier. The plurality of targets may comprise 32, 35, 37, 40, 43, 45, 47, 50, 53, 55, 57, 60 or more targets selected from an SVM classifier. The SVM classifier may be an SVM2 classifier.

A SVM classifier of the present invention may comprise two or more targets comprising two or more targets selected from Table 1, Table 2, Table 6, Table 7, Table 15 or SEQ ID NOs: 1-3348. In certain instances, the two or more targets are selected from ERG, ETV1, ETV4, ETV5, FLI1, and/or SPINK1; TDRD1, CACNA1D, NCALD, HLA-DMB, FAM65B, AMACR, SLC61A1, and/or FKBP10; TDRD1, CACNA1D, NCALD, HLA-DMB, FAM65B, AMACR, SLC61A1, FKBP10, HPGD, FAM3B, MIPEP, NCAPD3, INPP4B, ANPEP, TFF3, ALOX15B, and/or MON1B; MME, BANK1, LEPREL1, VGLL3, NPR3, OR4K7P, OR4K6P, POTEB2, RP11, TTN, FAP5, and/or GPR116; SPINK1, BANK1, LEPREL1, TTN, POTEB2, OR4K7P, OR4K6P, FAB5P7, NPR1, and/or RP11-403B2; GPR116, GRM7; or a combination thereof.

The plurality of targets may comprise one or more targets selected from a KNN classifier. The plurality of targets may comprise 2, 3, 4, 5, 6, 7, 8, 9, 10 or more targets selected from a KNN classifier. The plurality of targets may comprise 12, 13, 14, 15, 17, 20, 22, 25, 27, 30 or more targets selected from a KNN classifier. The plurality of targets may comprise 32, 35, 37, 40, 43, 45, 47, 50, 53, 55, 57, 60 or more targets selected from a KNN classifier. The plurality of targets may comprise 65, 70, 75, 80, 85, 90, 95, 100 or more targets selected from a KNN classifier.

The KNN classifier may be a KNN2 classifier. A KNN classifier of the present invention may comprise two or more targets comprising two or more targets selected from Table 1, Table 2, Table 6, Table 7, Table 15 or SEQ ID NOs: 1-3348. In certain instances, the two or more targets are selected from ERG, ETV1, ETV4, ETV5, FLI1, and/or SPINK1; TDRD1, CACNA1D, NCALD, HLA-DMB, FAM65B, AMACR, SLC61A1, and/or FKBP10; TDRD1, CACNA1D, NCALD, HLA-DMB, FAM65B, AMACR, SLC61A1, FKBP10, HPGD, FAM3B, MIPEP, NCAPD3, INPP4B, ANPEP, TFF3, ALOX15B, and/or MON1B; MME, BANK1, LEPREL1, VGLL3, NPR3, OR4K7P, OR4K6P, POTEB2, RP11, TTN, FAP5, and/or GPR116; SPINK1, BANK1, LEPREL1, TTN, POTEB2, OR4K7P, OR4K6P, FAB5P7, NPR1, and/or RP11-403B2; GPR116, GRM7; or a combination thereof.

The plurality of targets may comprise one or more targets selected from a Naïve Bayes (NB) classifier. The plurality of targets may comprise 2, 3, 4, 5, 6, 7, 8, 9, 10 or more targets selected from an NB classifier. The plurality of targets may comprise 12, 13, 14, 15, 17, 20, 22, 25, 27, 30 or more targets selected from an NB classifier. The plurality of targets may comprise 32, 35, 37, 40, 43, 45, 47, 50, 53, 55, 57, 60 or more targets selected from a NB classifier. The plurality of targets may comprise 65, 70, 75, 80, 85, 90, 95, 100 or more targets selected from a NB classifier.

The NB classifier may be a NB2 classifier. An NB classifier of the present invention may comprise two or more targets comprising two or more targets selected from Table 1, Table 2, Table 6, Table 7, Table 15 or SEQ ID NOs: 1-3348. In certain instances, the two or more targets are selected from ERG, ETV1, ETV4, ETV5, FLI1, and/or SPINK1; TDRD1, CACNA1D, NCALD, HLA-DMB, FAM65B, AMACR, SLC61A1, and/or FKBP10; TDRD1, CACNA1D, NCALD, HLA-DMB, FAM65B, AMACR, SLC61A1, FKBP10, HPGD, FAM3B, MIPEP, NCAPD3, INPP4B, ANPEP, TFF3, ALOX15B, and/or MON1B; MME, BANK1, LEPREL1, VGLL3, NPR3, OR4K7P, OR4K6P, POTEB2, RP11, TTN, FAP5, and/or GPR116; SPINK1, BANK1, LEPREL1, TTN, POTEB2, OR4K7P, OR4K6P, FAB5P7, NPR1, and/or RP11-403B2; GPR116, GRM7; or a combination thereof.

The plurality of targets may comprise one or more targets selected from a recursive Partitioning (Rpart) classifier. The plurality of targets may comprise 2, 3, 4, 5, 6, 7, 8, 9, 10 or more targets selected from an Rpart classifier. The plurality of targets may comprise 12, 13, 14, 15, 17, 20, 22, 25, 27, 30 or more targets selected from an Rpart classifier. The plurality of targets may comprise 32, 35, 37, 40, 43, 45, 47, 50, 53, 55, 57, 60 or more targets selected from an Rpart classifier. The plurality of targets may comprise 65, 70, 75, 80, 85, 90, 95, 100 or more targets selected from an Rpart classifier.

The Rpart classifier may be an Rpart2 classifier. An Rpart classifier of the present invention may comprise two or more targets comprising two or more targets selected from Table 1, Table 2, Table 6, Table 7, Table 15 or SEQ ID NOs: 1-3348. In certain instances, the two or more targets are selected from ERG, ETV1, ETV4, ETV5, FLI1, and/or SPINK1; TDRD1, CACNA1D, NCALD, HLA-DMB, FAM65B, AMACR, SLC61A1, and/or FKBP10; TDRD1, CACNA1D, NCALD, HLA-DMB, FAM65B, AMACR, SLC61A1, FKBP10, HPGD, FAM3B, MIPEP, NCAPD3, INPP4B, ANPEP, TFF3, ALOX15B, and/or MON1B; MME, BANK1, LEPREL1, VGLL3, NPR3, OR4K7P, OR4K6P, POTEB2, RP11, TTN, FAP5, and/or GPR116; SPINK1, BANK1, LEPREL1, TTN, POTEB2, OR4K7P, OR4K6P, FAB5P7, NPR1, and/or RP11-403B2; GPR116, GRM7; or a combination thereof.

The plurality of targets may comprise one or more targets selected from a high dimensional discriminate analysis (HDDA) classifier. The plurality of targets may comprise two or more targets selected from a high dimensional discriminate analysis (HDDA) classifier. The plurality of targets may comprise three or more targets selected from a high dimensional discriminate analysis (HDDA) classifier. The plurality of targets may comprise 5, 6, 7, 8, 9, 10, 11, 12, 13, 14, 15 or more targets selected from a high dimensional discriminate analysis (HDDA) classifier.

### Probes/Primers

The present invention provides for a probe set for diagnosing, monitoring and/or predicting a status or outcome of a prostate cancer in a subject comprising a plurality of probes, wherein (i) the probes in the set are capable of detecting an expression level of at least one target selected from; and (ii) the expression level determines the cancer status of the subject with at least about 40% specificity.

The probe set may comprise one or more polynucleotide probes. Individual polynucleotide probes comprise a nucleotide sequence derived from the nucleotide sequence of the target sequences or complementary sequences thereof. The nucleotide sequence of the polynucleotide probe is designed such that it corresponds to, or is complementary to the target sequences. The polynucleotide probe can specifically hybridize under either stringent or lowered stringency hybridization conditions to a region of the target sequences, to the complement thereof, or to a nucleic acid sequence (such as a cDNA) derived therefrom.

The selection of the polynucleotide probe sequences and determination of their uniqueness may be carried out in silico using techniques known in the art, for example, based on a BLASTN search of the polynucleotide sequence in question against gene sequence databases, such as the Human Genome Sequence, UniGene, dbEST or the non-redundant database at NCBI. In one embodiment of the invention, the polynucleotide probe is complementary to a region of a target mRNA derived from a target sequence in the probe set. Computer programs can also be employed to select probe sequences that may not cross hybridize or may not hybridize non-specifically.

In some instances, microarray hybridization of RNA, extracted from prostate cancer tissue samples and amplified, may yield a dataset that is then summarized and normalized by the fRMA technique. After removal (or filtration) of cross-hybridizing PSRs, and PSRs containing less than 4 probes, the remaining PSRs can be used in further analysis. Following fRMA and filtration, the data can be decomposed into its principal components and an analysis of variance model is used to determine the extent to which a batch effect remains present in the first 10 principal components.

These remaining PSRs can then be subjected to filtration by a T-test between CR (clinical recurrence) and non-CR samples. Using a p-value cut-off of 0.01, the remaining features (e.g., PSRs) can be further refined. Feature selection can be performed by regularized logistic regression using the elastic-net penalty. The regularized regression may be bootstrapped over 1000 times using all training data; with each iteration of bootstrapping, features that have non-zero co-efficient following 3-fold cross validation can be tabulated. In some instances, features that were selected in at least 25% of the total runs were used for model building.

The polynucleotide probes of the present invention may range in length from about 15 nucleotides to the full length of the coding target or non-coding target. In one embodiment of the invention, the polynucleotide probes are at least about 15 nucleotides in length. In another embodiment, the polynucleotide probes are at least about 20 nucleotides in length. In a further embodiment, the polynucleotide probes are at least about 25 nucleotides in length. In another embodiment, the polynucleotide probes are between about 15 nucleotides and about 500 nucleotides in length. In other embodiments, the polynucleotide probes are between about 15 nucleotides and about 450 nucleotides, about 15 nucleotides and about 400 nucleotides, about 15 nucleotides and about 350 nucleotides, about 15 nucleotides and about 300 nucleotides, about 15 nucleotides and about 250 nucleotides, about 15 nucleotides and about 200 nucleotides in length. In some embodiments, the probes are at least 15 nucleotides in length. In some embodiments, the probes are at least 15 nucleotides in length. In some embodiments, the probes are at least 20 nucleotides, at least 25 nucleotides, at least 50 nucleotides, at least 75 nucleotides, at least 100 nucleotides, at least 125 nucleotides, at least 150 nucleotides, at least 200 nucleotides, at least 225 nucleotides, at least 250 nucleotides, at least 275 nucleotides, at least 300 nucleotides, at least 325 nucleotides, at least 350 nucleotides, at least 375 nucleotides in length.

The polynucleotide probes of a probe set can comprise RNA, DNA, RNA or DNA mimetics, or combinations thereof, and can be single-stranded or double-stranded. Thus the polynucleotide probes can be composed of naturally-occurring nucleobases, sugars and covalent internucleoside (backbone) linkages as well as polynucleotide probes having non-naturally-occurring portions which function similarly. Such modified or substituted polynucleotide probes may provide desirable properties such as, for example, enhanced affinity for a target gene and increased stability. The probe set may comprise a coding target and/or a non-coding target. Preferably, the probe set comprises a combination of a coding target and non-coding target.

In some embodiments, the probe set comprise a plurality of target sequences that hybridize to at least about 5 coding targets and/or non-coding targets selected from Table 1, Table 2, Table 6, Table 7, Table 15 or SEQ ID NOs: 1-3348. Alternatively, the probe set comprise a plurality of target sequences that hybridize to at least about 10 coding targets and/or non-coding targets selected from Table 1, Table 2, Table 6, Table 7, Table 15 or SEQ ID NOs: 1-3348. In some embodiments, the probe set comprise a plurality of target sequences that hybridize to at least about 15 coding targets and/or non-coding targets selected from Table 1, Table 2, Table 6, Table 7, Table 15 or SEQ ID NOs: 1-3348. In some embodiments, the probe set comprise a plurality of target sequences that hybridize to at least about 20 coding targets and/or non-coding targets selected from Table 1, Table 2, Table 6, Table 7, Table 15 or SEQ ID NOs: 1-3348. In some embodiments, the probe set comprise a plurality of target sequences that hybridize to at least about 30 coding targets and/or non-coding targets selected from Table 1, Table 2, Table 6, Table 7, Table 15 or SEQ ID NOs: 1-3348. In certain instances, the plurality of targets are selected from ERG, ETV1, ETV4, ETV5, FLI1, and/or SPINK1; TDRD1, CACNA1D, NCALD, HLA-DMB, FAM65B, AMACR, SLC61A1, and/or FKBP10; TDRD1, CACNA1D, NCALD, HLA-DMB, FAM65B, AMACR, SLC61A1, FKBP10, HPGD, FAM3B, MIPEP, NCAPD3, INPP4B, ANPEP, TFF3, ALOX15B, and/or MON1B; MME, BANK1, LEPREL1, VGLL3, NPR3, OR4K7P, OR4K6P, POTEB2, RP11, TTN, FAP5, and/or GPR116; SPINK1, BANK1, LEPREL1, TTN, POTEB2, OR4K7P, OR4K6P, FAB5P7, NPR1, and/or RP11-403B2; GPR116, GRM7; or a combination thereof.

The system of the present invention further provides for primers and primer pairs capable of amplifying target sequences defined by the probe set, or fragments or subsequences or complements thereof. The nucleotide sequences of the probe set may be provided in computer-readable media for in silico applications and as a basis for the design of appropriate primers for amplification of one or more target sequences of the probe set.

Primers based on the nucleotide sequences of target sequences can be designed for use in amplification of the target sequences. For use in amplification reactions such as PCR, a pair of primers can be used. The exact composition of the primer sequences is not critical to the invention, but for most applications the primers may hybridize to specific sequences of the probe set under stringent conditions, particularly under conditions of high stringency, as known in the art. The pairs of primers are usually chosen so as to generate an amplification product of at least about 50 nucleotides, more usually at least about 100 nucleotides. Algorithms for the selection of primer sequences are generally known, and are available in commercial software packages. These primers may be used in standard quantitative or qualitative PCR-based assays to assess transcript expression levels of RNAs defined by the probe set. Alternatively, these primers may be used in combination with probes, such as molecular beacons in amplifications using real-time PCR.

In one embodiment, the primers or primer pairs, when used in an amplification reaction, specifically amplify at least a portion of a nucleic acid sequence of a target selected from Table 1, Table 2, Table 6, Table 7, Table 15 or SEQ ID NOs: 1-3348 (or subgroups thereof as set forth herein), an RNA form thereof, or a complement to either thereof. In certain instances, the nucleic acid sequence is selected from ERG, ETV1, ETV4, ETV5, FLI1, and/or SPINK1; TDRD1, CACNA1D, NCALD, HLA-DMB, FAM65B, AMACR, SLC61A1, and/or FKBP10; TDRD1, CACNA1D, NCALD, HLA-DMB, FAM65B, AMACR, SLC61A1, FKBP10, HPGD, FAM3B, MIPEP, NCAPD3, INPP4B, ANPEP, TFF3, ALOX15B, and/or MON1B; MME, BANK1, LEPREL1, VGLL3, NPR3, OR4K7P, OR4K6P, POTEB2, RP11, TTN, FAP5, and/or GPR116; SPINK1, BANK1, LEPREL1, TTN, POTEB2, OR4K7P, OR4K6P, FAB5P7, NPR1, and/or RP11-403B2; GPR116, GRM7; or a combination thereof.

A label can optionally be attached to or incorporated into a probe or primer polynucleotide to allow detection and/or quantitation of a target polynucleotide representing the target sequence of interest. The target polynucleotide may be the expressed target sequence RNA itself, a cDNA copy thereof, or an amplification product derived therefrom, and may be the positive or negative strand, so long as it can be specifically detected in the assay being used. Similarly, an antibody may be labeled.

In certain multiplex formats, labels used for detecting different targets may be distinguishable. The label can be attached directly (e.g., via covalent linkage) or indirectly, e.g., via a bridging molecule or series of molecules (e.g., a molecule or complex that can bind to an assay component, or via members of a binding pair that can be incorporated into assay components, e.g. biotin-avidin or streptavidin). Many labels are commercially available in activated forms which can readily be used for such conjugation (for example through amine acylation), or labels may be attached through known or determinable conjugation schemes, many of which are known in the art.

Labels useful in the invention described herein include any substance which can be detected when bound to or incorporated into the biomolecule of interest. Any effective detection method can be used, including optical, spectroscopic, electrical, piezoelectrical, magnetic, Raman scattering, surface plasmon resonance, colorimetric, calorimetric, etc. A label is typically selected from a chromophore, a lumiphore, a fluorophore, one member of a quenching system, a chromogen, a hapten, an antigen, a magnetic particle, a material exhibiting nonlinear optics, a semiconductor nanocrystal, a metal nanoparticle, an enzyme, an antibody or binding portion or equivalent thereof, an aptamer, and one member of a binding pair, and combinations thereof. Quenching schemes may be used, wherein a quencher and a fluorophore as members of a quenching pair may be used on a probe, such that a change in optical parameters occurs upon binding to the target introduce or quench the signal from the fluorophore. One example of such a system is a molecular beacon. Suitable quencher/fluorophore systems are known in the art. The label may be bound through a variety of intermediate linkages. For example, a polynucleotide may comprise a biotin-binding species, and an optically detectable label may be conjugated to biotin and then bound to the labeled polynucleotide. Similarly, a polynucleotide sensor may comprise an immunological species such as an antibody or fragment, and a secondary antibody containing an optically detectable label may be added.

Chromophores useful in the methods described herein include any substance which can absorb energy and emit light. For multiplexed assays, a plurality of different signaling chromophores can be used with detectably different emission spectra. The chromophore can be a lumophore or a fluorophore. Typical fluorophores include fluorescent dyes, semiconductor nanocrystals, lanthanide chelates, polynucleotide-specific dyes and green fluorescent protein.

In some embodiments, polynucleotides of the invention comprise at least 20 consecutive bases of the nucleic acid sequence of a target selected from Table 1, Table 2, Table 6, Table 7, Table 15 or SEQ ID NOs: 1-3348 or a complement thereto. The polynucleotides may comprise at least 21, 22, 23, 24, 25, 27, 30, 32, 35 or more consecutive bases of the nucleic acids sequence of a target selected from Table 1, Table 2, Table 6, Table 7, Table 15 or SEQ ID NOs: 1-3348, as applicable. In certain instances, the target is selected from ERG, ETV1, ETV4, ETV5, FLI1, and/or SPINK1; TDRD1, CACNA1D, NCALD, HLA-DMB, FAM65B, AMACR, SLC61A1, and/or FKBP10; TDRD1, CACNA1D, NCALD, HLA-DMB, FAM65B, AMACR, SLC61A1, FKBP10, HPGD, FAM3B, MIPEP, NCAPD3, INPP4B, ANPEP, TFF3, ALOX15B, and/or MON1B; MME, BANK1, LEPREL1, VGLL3, NPR3, OR4K7P, OR4K6P, POTEB2, RP11, TTN, FAP5, and/or GPR116; SPINK1, BANK1, LEPREL1, TTN, POTEB2, OR4K7P, OR4K6P, FAB5P7, NPR1, and/or RP11-403B2; GPR116, GRM7; or a combination thereof.

The polynucleotides may be provided in a variety of formats, including as solids, in solution, or in an array. The polynucleotides may optionally comprise one or more labels, which may be chemically and/or enzymatically incorporated into the polynucleotide.

In some embodiments, one or more polynucleotides provided herein can be provided on a substrate. The substrate can comprise a wide range of material, either biological, nonbiological, organic, inorganic, or a combination of any of these. For example, the substrate may be a polymerized Langmuir Blodgett film, functionalized glass, Si, Ge, GaAs, GaP, SiO₂, SiN₄, modified silicon, or any one of a wide variety of gels or polymers such as (poly)tetrafluoroethylene, (poly)vinylidenedifluoride, polystyrene, cross-linked polystyrene, polyacrylic, polylactic acid, polyglycolic acid, poly(lactide coglycolide), polyanhydrides, poly(methyl methacrylate), poly(ethylene-co-vinyl acetate), polysiloxanes, polymeric silica, latexes, dextran polymers, epoxies, polycarbonates, or combinations thereof. Conducting polymers and photoconductive materials can be used.

The substrate can take the form of an array, a photodiode, an optoelectronic sensor such as an optoelectronic semiconductor chip or optoelectronic thin-film semiconductor, or a biochip. The location(s) of probe(s) on the substrate can be addressable; this can be done in highly dense formats, and the location(s) can be microaddressable or nanoaddressable.

### Diagnostic Samples

Diagnostic samples for use with the systems and in the methods of the present invention comprise nucleic acids suitable for providing RNAs expression information. In principle, the biological sample from which the expressed RNA is obtained and analyzed for target sequence expression can be any material suspected of comprising prostate cancer tissue or cells. The diagnostic sample can be a biological sample used directly in a method of the invention. Alternatively, the diagnostic sample can be a sample prepared from a biological sample.

In one embodiment, the sample or portion of the sample comprising or suspected of comprising cancer tissue or cells can be any source of biological material, including cells, tissue or fluid, including bodily fluids. Non-limiting examples of the source of the sample include an aspirate, a needle biopsy, a cytology pellet, a bulk tissue preparation or a section thereof obtained for example by surgery or autopsy, lymph fluid, blood, plasma, serum, tumors, and organs. In some embodiments, the sample is from urine. Alternatively, the sample is from blood, plasma or serum. In some embodiments, the sample is from saliva.

The samples may be archival samples, having a known and documented medical outcome, or may be samples from current patients whose ultimate medical outcome is not yet known.

In some embodiments, the sample may be dissected prior to molecular analysis. The sample may be prepared via macrodissection of a bulk tumor specimen or portion thereof, or may be treated via microdissection, for example via Laser Capture Microdissection (LCM).

The sample may initially be provided in a variety of states, as fresh tissue, fresh frozen tissue, fine needle aspirates, and may be fixed or unfixed. Frequently, medical laboratories routinely prepare medical samples in a fixed state, which facilitates tissue storage. A variety of fixatives can be used to fix tissue to stabilize the morphology of cells, and may be used alone or in combination with other agents. Exemplary fixatives include crosslinking agents, alcohols, acetone, Bouin's solution, Zenker solution, Helv solution, osmic acid solution and Carnoy solution.

Crosslinking fixatives can comprise any agent suitable for forming two or more covalent bonds, for example an aldehyde. Sources of aldehydes typically used for fixation include formaldehyde, paraformaldehyde, glutaraldehyde or formalin. Preferably, the crosslinking agent comprises formaldehyde, which may be included in its native form or in the form of paraformaldehyde or formalin. One of skill in the art would appreciate that for samples in which crosslinking fixatives have been used special preparatory steps may be necessary including for example heating steps and proteinase-k digestion; see methods.

One or more alcohols may be used to fix tissue, alone or in combination with other fixatives. Exemplary alcohols used for fixation include methanol, ethanol and isopropanol.

Formalin fixation is frequently used in medical laboratories. Formalin comprises both an alcohol, typically methanol, and formaldehyde, both of which can act to fix a biological sample.

Whether fixed or unfixed, the biological sample may optionally be embedded in an embedding medium. Exemplary embedding media used in histology including paraffin, Tissue-Tek® V.I.P.™, Paramat, Paramat Extra, Paraplast, Paraplast X-tra, Paraplast Plus, Peel Away Paraffin Embedding Wax, Polyester Wax, Carbowax Polyethylene Glycol, Polyfin™, Tissue Freezing Medium TFMFM, Cryo-Gef™, and OCT Compound (Electron Microscopy Sciences, Hatfield, Pa.). Prior to molecular analysis, the embedding material may be removed via any suitable techniques, as known in the art. For example, where the sample is embedded in wax, the embedding material may be removed by extraction with organic solvent(s), for example xylenes. Kits are commercially available for removing embedding media from tissues. Samples or sections thereof may be subjected to further processing steps as needed, for example serial hydration or dehydration steps.

In some embodiments, the sample is a fixed, wax-embedded biological sample. Frequently, samples from medical laboratories are provided as fixed, wax-embedded samples, most commonly as formalin-fixed, paraffin embedded (FFPE) tissues.

Whatever the source of the biological sample, the target polynucleotide that is ultimately assayed can be prepared synthetically (in the case of control sequences), but typically is purified from the biological source and subjected to one or more preparative steps. The RNA may be purified to remove or diminish one or more undesired components from the biological sample or to concentrate it. Conversely, where the RNA is too concentrated for the particular assay, it may be diluted.

### RNA Extraction

RNA can be extracted and purified from biological samples using any suitable technique. A number of techniques are known in the art, and several are commercially available (e.g., FormaPure nucleic acid extraction kit, Agencourt Biosciences, Beverly Mass., High Pure FFPE RNA Micro Kit, Roche Applied Science, Indianapolis, Ind.). RNA can be extracted from frozen tissue sections using TRIzol (Invitrogen, Carlsbad, Calif.) and purified using RNeasy Protect kit (Qiagen, Valencia, Calif.). RNA can be further purified using DNAse I treatment (Ambion, Austin, Tex.) to eliminate any contaminating DNA. RNA concentrations can be made using a Nanodrop ND-1000 spectrophotometer (Nanodrop Technologies, Rockland, Del.). RNA can be further purified to eliminate contaminants that interfere with cDNA synthesis by cold sodium acetate precipitation. RNA integrity can be evaluated by running electropherograms, and RNA integrity number (RIN, a correlative measure that indicates intactness of mRNA) can be determined using the RNA 6000 PicoAssay for the Bioanalyzer 2100 (Agilent Technologies, Santa Clara, Calif.).

### Kits

Kits for performing the desired method(s) are also provided, and comprise a container or housing for holding the components of the kit, one or more vessels containing one or more nucleic acid(s), and optionally one or more vessels containing one or more reagents. The reagents include those described in the composition of matter section above, and those reagents useful for performing the methods described, including amplification reagents, and may include one or more probes, primers or primer pairs, enzymes (including polymerases and ligases), intercalating dyes, labeled probes, and labels that can be incorporated into amplification products.

In some embodiments, the kit comprises primers or primer pairs specific for those subsets and combinations of target sequences described herein. The primers or pairs of primers suitable for selectively amplifying the target sequences. The kit may comprise at least two, three, four or five primers or pairs of primers suitable for selectively amplifying one or more targets. The kit may comprise at least 5, 10, 15, 20, 30, 40, 50, 60, 70, 80, 90, 100 or more primers or pairs of primers suitable for selectively amplifying one or more targets.

In some embodiments, the primers or primer pairs of the kit, when used in an amplification reaction, specifically amplify a non-coding target, coding target, exonic, or non-exonic target described herein, a nucleic acid sequence corresponding to a target selected from Table 1, Table 2, Table 6, Table 7, Table 15 or SEQ ID NOs: 1-3348, an RNA form thereof, or a complement to either thereof. The kit may include a plurality of such primers or primer pairs which can specifically amplify a corresponding plurality of different amplify a non-coding target, coding target, exonic, or non-exonic transcript described herein, a nucleic acid sequence corresponding to a target selected from Table 1, Table 2, Table 6, Table 7, Table 15 or SEQ ID NOs: 1-3348, RNA forms thereof, or complements thereto. At least two, three, four or five primers or pairs of primers suitable for selectively amplifying the one or more targets can be provided in kit form. In some embodiments, the kit comprises from five to fifty primers or pairs of primers suitable for amplifying the one or more targets. In certain instances, the target is selected from ERG, ETV1, ETV4, ETV5, FLI1, and/or SPINK1; TDRD1, CACNA1D, NCALD, HLA-DMB, FAM65B, AMACR, SLC61A1, and/or FKBP10; TDRD1, CACNA1D, NCALD, HLA-DMB, FAM65B, AMACR, SLC61A1, FKBP10, HPGD, FAM3B, MIPEP, NCAPD3, INPP4B, ANPEP, TFF3, ALOX15B, and/or MON1B; MME, BANK1, LEPREL1, VGLL3, NPR3, OR4K7P, OR4K6P, POTEB2, RP11, TTN, FAP5, and/or GPR116; SPINK1, BANK1, LEPREL1, TTN, POTEB2, OR4K7P, OR4K6P, FAB5P7, NPR1, and/or RP11-403B2; GPR116, GRM7; or a combination thereof.

The reagents may independently be in liquid or solid form. The reagents may be provided in mixtures. Control samples and/or nucleic acids may optionally be provided in the kit. Control samples may include tissue and/or nucleic acids obtained from or representative of tumor samples from patients showing no evidence of disease, as well as tissue and/or nucleic acids obtained from or representative of tumor samples from patients that develop systemic cancer.

The nucleic acids may be provided in an array format, and thus an array or microarray may be included in the kit. The kit optionally may be certified by a government agency for use in prognosing the disease outcome of cancer patients and/or for designating a treatment modality.

Instructions for using the kit to perform one or more methods of the invention can be provided with the container, and can be provided in any fixed medium. The instructions may be located inside or outside the container or housing, and/or may be printed on the interior or exterior of any surface thereof. A kit may be in multiplex form for concurrently detecting and/or quantitating one or more different target polynucleotides representing the expressed target sequences.

### Amplification and Hybridization

Following sample collection and nucleic acid extraction, the nucleic acid portion of the sample comprising RNA that is or can be used to prepare the target polynucleotide(s) of interest can be subjected to one or more preparative reactions. These preparative reactions can include in vitro transcription (IVT), labeling, fragmentation, amplification and other reactions. mRNA can first be treated with reverse transcriptase and a primer to create cDNA prior to detection, quantitation and/or amplification; this can be done in vitro with purified mRNA or in situ, e.g., in cells or tissues affixed to a slide.

By “amplification” is meant any process of producing at least one copy of a nucleic acid, in this case an expressed RNA, and in many cases produces multiple copies. An amplification product can be RNA or DNA, and may include a complementary strand to the expressed target sequence. DNA amplification products can be produced initially through reverse translation and then optionally from further amplification reactions. The amplification product may include all or a portion of a target sequence, and may optionally be labeled. A variety of amplification methods are suitable for use, including polymerase-based methods and ligation-based methods. Exemplary amplification techniques include the polymerase chain reaction method (PCR), the lipase chain reaction (LCR), ribozyme-based methods, self-sustained sequence replication (3 SR), nucleic acid sequence-based amplification (NASBA), the use of Q Beta replicase, reverse transcription, nick translation, and the like.

Asymmetric amplification reactions may be used to preferentially amplify one strand representing the target sequence that is used for detection as the target polynucleotide. In some cases, the presence and/or amount of the amplification product itself may be used to determine the expression level of a given target sequence. In other instances, the amplification product may be used to hybridize to an array or other substrate comprising sensor polynucleotides which are used to detect and/or quantitate target sequence expression.

The first cycle of amplification in polymerase-based methods typically forms a primer extension product complementary to the template strand. If the template is single-stranded RNA, a polymerase with reverse transcriptase activity is used in the first amplification to reverse transcribe the RNA to DNA, and additional amplification cycles can be performed to copy the primer extension products. The primers for a PCR must, of course, be designed to hybridize to regions in their corresponding template that can produce an amplifiable segment; thus, each primer must hybridize so that its 3′ nucleotide is paired to a nucleotide in its complementary template strand that is located 3′ from the 3′ nucleotide of the primer used to replicate that complementary template strand in the PCR.

The target polynucleotide can be amplified by contacting one or more strands of the target polynucleotide with a primer and a polymerase having suitable activity to extend the primer and copy the target polynucleotide to produce a full-length complementary polynucleotide or a smaller portion thereof. Any enzyme having a polymerase activity that can copy the target polynucleotide can be used, including DNA polymerases, RNA polymerases, reverse transcriptases, enzymes having more than one type of polymerase or enzyme activity. The enzyme can be thermolabile or thermostable. Mixtures of enzymes can also be used. Exemplary enzymes include: DNA polymerases such as DNA Polymerase I (“Pol I”), the Klenow fragment of Pol I, T4, T7, Sequenase® T7, Sequenase® Version 2.0 T7, Tub, Taq, Tth, Pfic, Pfu, Tsp, Tfl, Tli and Pyrococcus sp GB-D DNA polymerases; RNA polymerases such as E. coli, SP6, T3 and T7 RNA polymerases; and reverse transcriptases such as AMV, M-MuLV, MMLV, RNAse H MMLV (SuperScript®), SuperScript® II, ThermoScript®, HIV-1, and RAV2 reverse transcriptases. All of these enzymes are commercially available. Exemplary polymerases with multiple specificities include RAV2 and Tli (exo-) polymerases. Exemplary thermostable polymerases include Tub, Taq, Tth, Pfic, Pfu, Tsp, Tfl, Tli and Pyrococcus sp. GB-D DNA polymerases.

Suitable reaction conditions are chosen to permit amplification of the target polynucleotide, including pH, buffer, ionic strength, presence and concentration of one or more salts, presence and concentration of reactants and cofactors such as nucleotides and magnesium and/or other metal ions (e.g., manganese), optional cosolvents, temperature, thermal cycling profile for amplification schemes comprising a polymerase chain reaction, and may depend in part on the polymerase being used as well as the nature of the sample. Cosolvents include formamide (typically at from about 2 to about 10%), glycerol (typically at from about 5 to about 10%), and DMSO (typically at from about 0.9 to about 10%). Techniques may be used in the amplification scheme in order to minimize the production of false positives or artifacts produced during amplification. These include “touchdown” PCR, hot-start techniques, use of nested primers, or designing PCR primers so that they form stem-loop structures in the event of primer-dimer formation and thus are not amplified. Techniques to accelerate PCR can be used, for example centrifugal PCR, which allows for greater convection within the sample, and comprising infrared heating steps for rapid heating and cooling of the sample. One or more cycles of amplification can be performed. An excess of one primer can be used to produce an excess of one primer extension product during PCR; preferably, the primer extension product produced in excess is the amplification product to be detected. A plurality of different primers may be used to amplify different target polynucleotides or different regions of a particular target polynucleotide within the sample.

An amplification reaction can be performed under conditions which allow an optionally labeled sensor polynucleotide to hybridize to the amplification product during at least part of an amplification cycle. When the assay is performed in this manner, real-time detection of this hybridization event can take place by monitoring for light emission or fluorescence during amplification, as known in the art.

Where the amplification product is to be used for hybridization to an array or microarray, a number of suitable commercially available amplification products are available. These include amplification kits available from NuGEN, Inc. (San Carlos, Calif.), including the WT-Ovation™ System, WT-Ovation™ System v2, WT-Ovation™ Pico System, WT-Ovation™ FFPE Exon Module, WT-Ovation™ FFPE Exon Module RiboAmp and RiboAmp^{Plus} RNA Amplification Kits (MDS Analytical Technologies (formerly Arcturus) (Mountain View, Calif.), Genisphere, Inc. (Hatfield, Pa.), including the RampUp Plus™ and SenseAmp™ RNA Amplification kits, alone or in combination. Amplified nucleic acids may be subjected to one or more purification reactions after amplification and labeling, for example using magnetic beads (e.g., RNAClean magnetic beads, Agencourt Biosciences).

Multiple RNA biomarkers can be analyzed using real-time quantitative multiplex RT-PCR platforms and other multiplexing technologies such as GenomeLab GeXP Genetic Analysis System (Beckman Coulter, Foster City, Calif.), SmartCycler® 9600 or GeneXpert® Systems (Cepheid, Sunnyvale, Calif.), ABI 7900 HT Fast Real Time PCR system (Applied Biosystems, Foster City, Calif.), LightCycler® 480 System (Roche Molecular Systems, Pleasanton, Calif.), xMAP 100 System (Luminex, Austin, Tex.) Solexa Genome Analysis System (Illumina, Hayward, Calif.), OpenArray Real Time qPCR (BioTrove, Woburn, Mass.) and BeadXpress System (Illumina, Hayward, Calif.).

Detection and/or Quantification of Target Sequences

Any method of detecting and/or quantitating the expression of the encoded target sequences can in principle be used in the invention. The expressed target sequences can be directly detected and/or quantitated, or may be copied and/or amplified to allow detection of amplified copies of the expressed target sequences or its complement.

Methods for detecting and/or quantifying a target can include Northern blotting, sequencing, array or microarray hybridization, by enzymatic cleavage of specific structures (e.g., an Invader® assay, Third Wave Technologies, e.g. as described in U.S. Pat. Nos. 5,846,717, 6,090,543; 6,001,567; 5,985,557; and 5,994,069) and amplification methods, e.g. RT-PCR, including in a TaqMan® assay (PE Biosystems, Foster City, Calif., e.g. as described in U.S. Pat. Nos. 5,962,233 and 5,538,848), and may be quantitative or semi-quantitative, and may vary depending on the origin, amount and condition of the available biological sample. Combinations of these methods may also be used. For example, nucleic acids may be amplified, labeled and subjected to microarray analysis.

In some instances, target sequences may be detected by sequencing. Sequencing methods may comprise whole genome sequencing or exome sequencing. Sequencing methods such as Maxim-Gilbert, chain-termination, or high-throughput systems may also be used. Additional, suitable sequencing techniques include classic dideoxy sequencing reactions (Sanger method) using labeled terminators or primers and gel separation in slab or capillary, sequencing by synthesis using reversibly terminated labeled nucleotides, pyrosequencing, 454 sequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, and SOLiD sequencing.

Additional methods for detecting and/or quantifying a target include single-molecule sequencing (e.g., Helicos, PacBio), sequencing by synthesis (e.g., Illumina, Ion Torrent), sequencing by ligation (e.g., ABI SOLID), sequencing by hybridization (e.g., Complete Genomics), in situ hybridization, bead-array technologies (e.g., Luminex xMAP, Illumina BeadChips), branched DNA technology (e.g., Panomics, Genisphere). Sequencing methods may use fluorescent (e.g., Illumina) or electronic (e.g., Ion Torrent, Oxford Nanopore) methods of detecting nucleotides.

### Reverse Transcription for QRT-PCR Analysis

Reverse transcription can be performed by any method known in the art. For example, reverse transcription may be performed using the Omniscript kit (Qiagen, Valencia, Calif.), Superscript III kit (Invitrogen, Carlsbad, Calif.), for RT-PCR. Target-specific priming can be performed in order to increase the sensitivity of detection of target sequences and generate target-specific cDNA.

### TaqMan® Gene Expression Analysis

TaqMan® RT-PCR can be performed using Applied Biosystems Prism (ABI) 7900 HT instruments in a 5 1.11 volume with target sequence-specific cDNA equivalent to 1 ng total RNA.

Primers and probes concentrations for TaqMan analysis are added to amplify fluorescent amplicons using PCR cycling conditions such as 95° C. for 10 minutes for one cycle, 95° C. for 20 seconds, and 60° C. for 45 seconds for 40 cycles. A reference sample can be assayed to ensure reagent and process stability. Negative controls (e.g., no template) should be assayed to monitor any exogenous nucleic acid contamination.

### Classification Arrays

The present invention contemplates that a probe set or probes derived therefrom may be provided in an array format. In the context of the present invention, an “array” is a spatially or logically organized collection of polynucleotide probes. An array comprising probes specific for a coding target, non-coding target, or a combination thereof may be used. Alternatively, an array comprising probes specific for two or more of transcripts of a target selected from Table 1, Table 2, Table 6, Table 7, or Table 15 or a product derived thereof can be used. Desirably, an array may be specific for 5, 10, 15, 20, 25, 30 or more of transcripts of a target selected from Table 1, Table 2, Table 6, Table 7, or Table 15. Expression of these sequences may be detected alone or in combination with other transcripts. In some embodiments, an array is used which comprises a wide range of sensor probes for prostate-specific expression products, along with appropriate control sequences. In some instances, the array may comprise the Human Exon 1.0 ST Array (HuEx 1.0 ST, Affymetrix, Inc., Santa Clara, Calif.).

Typically the polynucleotide probes are attached to a solid substrate and are ordered so that the location (on the substrate) and the identity of each are known. The polynucleotide probes can be attached to one of a variety of solid substrates capable of withstanding the reagents and conditions necessary for use of the array. Examples include, but are not limited to, polymers, such as (poly)tetrafluoroethylene, (poly)vinylidenedifluoride, polystyrene, polycarbonate, polypropylene and polystyrene; ceramic; silicon; silicon dioxide; modified silicon; (fused) silica, quartz or glass; functionalized glass; paper, such as filter paper; diazotized cellulose; nitrocellulose filter; nylon membrane; and polyacrylamide gel pad. Substrates that are transparent to light are useful for arrays that may be used in an assay that involves optical detection.

Examples of array formats include membrane or filter arrays (for example, nitrocellulose, nylon arrays), plate arrays (for example, multiwell, such as a 24-, 96-, 256-, 384-, 864- or 1536-well, microtitre plate arrays), pin arrays, and bead arrays (for example, in a liquid “slurry”). Arrays on substrates such as glass or ceramic slides are often referred to as chip arrays or “chips.” Such arrays are well known in the art. In one embodiment of the present invention, the Cancer Prognosticarray is a chip.

### Data Analysis

In some embodiments, one or more pattern recognition methods can be used in analyzing the expression level of target sequences. The pattern recognition method can comprise a linear combination of expression levels, or a nonlinear combination of expression levels. In some embodiments, expression measurements for RNA transcripts or combinations of RNA transcript levels are formulated into linear or non-linear models or algorithms (e.g., an ‘expression signature’) and converted into a likelihood score. This likelihood score indicates the probability that a biological sample is from a patient who may exhibit no evidence of disease, who may exhibit systemic cancer, or who may exhibit biochemical recurrence. The likelihood score can be used to distinguish these disease states. The models and/or algorithms can be provided in machine readable format, and may be used to correlate expression levels or an expression profile with a disease state, and/or to designate a treatment modality for a patient or class of patients.

Assaying the expression level for a plurality of targets may comprise the use of an algorithm or classifier. Array data can be managed, classified, and analyzed using techniques known in the art. Assaying the expression level for a plurality of targets may comprise probe set modeling and data pre-processing. Probe set modeling and data pre-processing can be derived using the Robust Multi-Array (RMA) algorithm or variants GC-RMA, JRMA, Probe Logarithmic Intensity Error (PLIER) algorithm or variant iterPLIER. Variance or intensity filters can be applied to pre-process data using the RMA algorithm, for example by removing target sequences with a standard deviation of <10 or a mean intensity of <100 intensity units of a normalized data range, respectively.

Alternatively, assaying the expression level for a plurality of targets may comprise the use of a machine learning algorithm. The machine learning algorithm may comprise a supervised learning algorithm. Examples of supervised learning algorithms may include Average One-Dependence Estimators (AODE), Artificial neural network (e.g., Backpropagation), Bayesian statistics (e.g., Naive Bayes classifier, Bayesian network, Bayesian knowledge base), Case-based reasoning, Decision trees, Inductive logic programming, Gaussian process regression, Group method of data handling (GMDH), Learning Automata, Learning Vector Quantization, Minimum message length (decision trees, decision graphs, etc.), Lazy learning, Instance-based learning Nearest Neighbor Algorithm, Analogical modeling, Probably approximately correct learning (PAC) learning, Ripple down rules, a knowledge acquisition methodology, Symbolic machine learning algorithms, Subsymbolic machine learning algorithms, Support vector machines, Random Forests, Ensembles of classifiers, Bootstrap aggregating (bagging), and Boosting. Supervised learning may comprise ordinal classification such as regression analysis and Information fuzzy networks (IFN). Alternatively, supervised learning methods may comprise statistical classification, such as AODE, Linear classifiers (e.g., Fisher's linear discriminant, Logistic regression, Naive Bayes classifier, Perceptron, and Support vector machine), quadratic classifiers, k-nearest neighbor, Boosting, Decision trees (e.g., C4.5, Random forests), Bayesian networks, and Hidden Markov models.

The machine learning algorithms may also comprise an unsupervised learning algorithm. Examples of unsupervised learning algorithms may include artificial neural network, Data clustering, Expectation-maximization algorithm, Self-organizing map, Radial basis function network, Vector Quantization, Generative topographic map, Information bottleneck method, and IBSEAD. Unsupervised learning may also comprise association rule learning algorithms such as Apriori algorithm, Eclat algorithm and FP-growth algorithm. Hierarchical clustering, such as Single-linkage clustering and Conceptual clustering, may also be used. Alternatively, unsupervised learning may comprise partitional clustering such as K-means algorithm and Fuzzy clustering.

In some instances, the machine learning algorithms comprise a reinforcement learning algorithm. Examples of reinforcement learning algorithms include, but are not limited to, temporal difference learning, Q-learning and Learning Automata. Alternatively, the machine learning algorithm may comprise Data Pre-processing.

Preferably, the machine learning algorithms may include, but are not limited to, Average One-Dependence Estimators (AODE), Fisher's linear discriminant, Logistic regression, Perceptron, Multilayer Perceptron, Artificial Neural Networks, Support vector machines, Quadratic classifiers, Boosting, Decision trees, C4.5, Bayesian networks, Hidden Markov models, High-Dimensional Discriminant Analysis, and Gaussian Mixture Models. The machine learning algorithm may comprise support vector machines, Naïve Bayes classifier, k-nearest neighbor, high-dimensional discriminant analysis, or Gaussian mixture models. In some instances, the machine learning algorithm comprises Random Forests.

### Cancer

The systems, compositions and methods disclosed herein may be used to diagnosis, monitor and/or predict the status or outcome of a cancer. Generally, a cancer is characterized by the uncontrolled growth of abnormal cells anywhere in a body. The abnormal cells may be termed cancer cells, malignant cells, or tumor cells. Cancer is not confined to humans; animals and other living organisms can get cancer.

In some instances, the cancer may be malignant. Alternatively, the cancer may be benign. The cancer may be a recurrent and/or refractory cancer. Most cancers can be classified as a carcinoma, sarcoma, leukemia, lymphoma, myeloma, or a central nervous system cancer.

The cancer may be a sarcoma. Sarcomas are cancers of the bone, cartilage, fat, muscle, blood vessels, or other connective or supportive tissue. Sarcomas include, but are not limited to, bone cancer, fibrosarcoma, chondrosarcoma, Ewing's sarcoma, malignant hemangioendothelioma, malignant schwannoma, bilateral vestibular schwannoma, osteosarcoma, soft tissue sarcomas (e.g. alveolar soft part sarcoma, angiosarcoma, cystosarcoma phylloides, dermatofibrosarcoma, desmoid tumor, epithelioid sarcoma, extraskeletal osteosarcoma, fibrosarcoma, hemangiopericytoma, hemangiosarcoma, Kaposi's sarcoma, leiomyosarcoma, liposarcoma, lymphangiosarcoma, lymphosarcoma, malignant fibrous histiocytoma, neurofibrosarcoma, rhabdomyosarcoma, and synovial sarcoma).

Alternatively, the cancer may be a carcinoma. Carcinomas are cancers that begin in the epithelial cells, which are cells that cover the surface of the body, produce hormones, and make up glands. By way of non-limiting example, carcinomas include breast cancer, pancreatic cancer, lung cancer, colon cancer, colorectal cancer, rectal cancer, kidney cancer, bladder cancer, stomach cancer, prostate cancer, liver cancer, ovarian cancer, brain cancer, vaginal cancer, vulvar cancer, uterine cancer, oral cancer, penic cancer, testicular cancer, esophageal cancer, skin cancer, cancer of the fallopian tubes, head and neck cancer, gastrointestinal stromal cancer, adenocarcinoma, cutaneous or intraocular melanoma, cancer of the anal region, cancer of the small intestine, cancer of the endocrine system, cancer of the thyroid gland, cancer of the parathyroid gland, cancer of the adrenal gland, cancer of the urethra, cancer of the renal pelvis, cancer of the ureter, cancer of the endometrium, cancer of the cervix, cancer of the pituitary gland, neoplasms of the central nervous system (CNS), primary CNS lymphoma, brain stem glioma, and spinal axis tumors. In some instances, the cancer is a skin cancer, such as a basal cell carcinoma, squamous, melanoma, nonmelanoma, or actinic (solar) keratosis. Preferably, the cancer is a prostate cancer. Alternatively, the cancer may be a thyroid cancer, bladder cancer, or pancreatic cancer.

In some instances, the cancer is a lung cancer. Lung cancer can start in the airways that branch off the trachea to supply the lungs (bronchi) or the small air sacs of the lung (the alveoli). Lung cancers include non-small cell lung carcinoma (NSCLC), small cell lung carcinoma, and mesotheliomia. Examples of NSCLC include squamous cell carcinoma, adenocarcinoma, and large cell carcinoma. The mesothelioma may be a cancerous tumor of the lining of the lung and chest cavity (pleura) or lining of the abdomen (peritoneum). The mesothelioma may be due to asbestos exposure. The cancer may be a brain cancer, such as a glioblastoma.

Alternatively, the cancer may be a central nervous system (CNS) tumor. CNS tumors may be classified as gliomas or nongliomas. The glioma may be malignant glioma, high grade glioma, diffuse intrinsic pontine glioma. Examples of gliomas include astrocytomas, oligodendrogliomas (or mixtures of oligodendroglioma and astocytoma elements), and ependymomas. Astrocytomas include, but are not limited to, low-grade astrocytomas, anaplastic astrocytomas, glioblastoma multiforme, pilocytic astrocytoma, pleomorphic xanthoastrocytoma, and subependymal giant cell astrocytoma. Oligodendrogliomas include low-grade oligodendrogliomas (or oligoastrocytomas) and anaplastic oligodendriogliomas. Nongliomas include meningiomas, pituitary adenomas, primary CNS lymphomas, and medulloblastomas. In some instances, the cancer is a meningioma.

The cancer may be a leukemia. The leukemia may be an acute lymphocytic leukemia, acute myelocytic leukemia, chronic lymphocytic leukemia, or chronic myelocytic leukemia. Additional types of leukemias include hairy cell leukemia, chronic myelomonocytic leukemia, and juvenile myelomonocytic-leukemia.

In some instances, the cancer is a lymphoma. Lymphomas are cancers of the lymphocytes and may develop from either B or T lymphocytes. The two major types of lymphoma are Hodgkin's lymphoma, previously known as Hodgkin's disease, and non-Hodgkin's lymphoma. Hodgkin's lymphoma is marked by the presence of the Reed-Sternberg cell. Non-Hodgkin's lymphomas are all lymphomas which are not Hodgkin's lymphoma. Non-Hodgkin lymphomas may be indolent lymphomas and aggressive lymphomas. Non-Hodgkin's lymphomas include, but are not limited to, diffuse large B cell lymphoma, follicular lymphoma, mucosa-associated lymphatic tissue lymphoma (MALT), small cell lymphocytic lymphoma, mantle cell lymphoma, Burkitt's lymphoma, mediastinal large B cell lymphoma, Waldenström macroglobulinemia, nodal marginal zone B cell lymphoma (NMZL), splenic marginal zone lymphoma (SMZL), extranodal marginal zone B cell lymphoma, intravascular large B cell lymphoma, primary effusion lymphoma, and lymphomatoid granulomatosis.

### Cancer Staging

Diagnosing, predicting, or monitoring a status or outcome of a cancer may comprise determining the stage of the cancer. Generally, the stage of a cancer is a description (usually numbers I to IV with IV having more progression) of the extent the cancer has spread. The stage often takes into account the size of a tumor, how deeply it has penetrated, whether it has invaded adjacent organs, how many lymph nodes it has metastasized to (if any), and whether it has spread to distant organs. Staging of cancer can be used as a predictor of survival, and cancer treatment may be determined by staging. Determining the stage of the cancer may occur before, during, or after treatment. The stage of the cancer may also be determined at the time of diagnosis.

Cancer staging can be divided into a clinical stage and a pathologic stage. Cancer staging may comprise the TNM classification. Generally, the TNM Classification of Malignant Tumours (TNM) is a cancer staging system that describes the extent of cancer in a patient's body. T may describe the size of the tumor and whether it has invaded nearby tissue, N may describe regional lymph nodes that are involved, and M may describe distant metastasis (spread of cancer from one body part to another). In the TNM (Tumor, Node, Metastasis) system, clinical stage and pathologic stage are denoted by a small “c” or “p” before the stage (e.g., cT3N1M0 or pT2N0).

Often, clinical stage and pathologic stage may differ. Clinical stage may be based on all of the available information obtained before a surgery to remove the tumor. Thus, it may include information about the tumor obtained by physical examination, radiologic examination, and endoscopy. Pathologic stage can add additional information gained by examination of the tumor microscopically by a pathologist. Pathologic staging can allow direct examination of the tumor and its spread, contrasted with clinical staging which may be limited by the fact that the information is obtained by making indirect observations at a tumor which is still in the body. The TNM staging system can be used for most forms of cancer.

Alternatively, staging may comprise Ann Arbor staging. Generally, Ann Arbor staging is the staging system for lymphomas, both in Hodgkin's lymphoma (previously called Hodgkin's disease) and Non-Hodgkin lymphoma (abbreviated NHL). The stage may depend on both the place where the malignant tissue is located (as located with biopsy, CT scanning and increasingly positron emission tomography) and on systemic symptoms due to the lymphoma (“B symptoms”: night sweats, weight loss of >10% or fevers). The principal stage may be determined by location of the tumor. Stage I may indicate that the cancer is located in a single region, usually one lymph node and the surrounding area. Stage I often may not have outward symptoms. Stage II can indicate that the cancer is located in two separate regions, an affected lymph node or organ and a second affected area, and that both affected areas are confined to one side of the diaphragm—that is, both are above the diaphragm, or both are below the diaphragm. Stage III often indicates that the cancer has spread to both sides of the diaphragm, including one organ or area near the lymph nodes or the spleen. Stage IV may indicate diffuse or disseminated involvement of one or more extralymphatic organs, including any involvement of the liver, bone marrow, or nodular involvement of the lungs.

Modifiers may also be appended to some stages. For example, the letters A, B, E, X, or S can be appended to some stages. Generally, A or B may indicate the absence of constitutional (B-type) symptoms is denoted by adding an “A” to the stage; the presence is denoted by adding a “B” to the stage. E can be used if the disease is “extranodal” (not in the lymph nodes) or has spread from lymph nodes to adjacent tissue. X is often used if the largest deposit is >10 cm large (“bulky disease”), or whether the mediastinum is wider than ⅓ of the chest on a chest X-ray. S may be used if the disease has spread to the spleen.

The nature of the staging may be expressed with CS or PS. CS may denote that the clinical stage as obtained by doctor's examinations and tests. PS may denote that the pathological stage as obtained by exploratory laparotomy (surgery performed through an abdominal incision) with splenectomy (surgical removal of the spleen).

### Therapeutic Regimens

Diagnosing, predicting, or monitoring a status or outcome of a cancer may comprise treating a cancer or preventing a cancer progression. In addition, diagnosing, predicting, or monitoring a status or outcome of a cancer may comprise identifying or predicting responders to an anti-cancer therapy. In some instances, diagnosing, predicting, or monitoring may comprise determining a therapeutic regimen. Determining a therapeutic regimen may comprise administering an anti-cancer therapy. Alternatively, determining a therapeutic regimen may comprise modifying, recommending, continuing or discontinuing an anti-cancer regimen. In some instances, if the sample expression patterns are consistent with the expression pattern for a known disease or disease outcome, the expression patterns can be used to designate one or more treatment modalities (e.g., therapeutic regimens, anti-cancer regimen). An anti-cancer regimen may comprise one or more anti-cancer therapies. Examples of anti-cancer therapies include surgery, chemotherapy, radiation therapy, immunotherapy/biological therapy, photodynamic therapy.

Surgical oncology uses surgical methods to diagnose, stage, and treat cancer, and to relieve certain cancer-related symptoms. Surgery may be used to remove the tumor (e.g., excisions, resections, debulking surgery), reconstruct a part of the body (e.g., restorative surgery), and/or to relieve symptoms such as pain (e.g., palliative surgery). Surgery may also include cryosurgery. Cryosurgery (also called cryotherapy) may use extreme cold produced by liquid nitrogen (or argon gas) to destroy abnormal tissue. Cryosurgery can be used to treat external tumors, such as those on the skin. For external tumors, liquid nitrogen can be applied directly to the cancer cells with a cotton swab or spraying device. Cryosurgery may also be used to treat tumors inside the body (internal tumors and tumors in the bone). For internal tumors, liquid nitrogen or argon gas may be circulated through a hollow instrument called a cryoprobe, which is placed in contact with the tumor. An ultrasound or Mill may be used to guide the cryoprobe and monitor the freezing of the cells, thus limiting damage to nearby healthy tissue. A ball of ice crystals may form around the probe, freezing nearby cells. Sometimes more than one probe is used to deliver the liquid nitrogen to various parts of the tumor. The probes may be put into the tumor during surgery or through the skin (percutaneously). After cryosurgery, the frozen tissue thaws and may be naturally absorbed by the body (for internal tumors), or may dissolve and form a scab (for external tumors).

Chemotherapeutic agents may also be used for the treatment of cancer. Examples of chemotherapeutic agents include alkylating agents, anti-metabolites, plant alkaloids and terpenoids, vinca alkaloids, podophyllotoxin, taxanes, topoisomerase inhibitors, and cytotoxic antibiotics. Cisplatin, carboplatin, and oxaliplatin are examples of alkylating agents. Other alkylating agents include mechlorethamine, cyclophosphamide, chlorambucil, ifosfamide. Alkylating agents may impair cell function by forming covalent bonds with the amino, carboxyl, sulfhydryl, and phosphate groups in biologically important molecules. Alternatively, alkylating agents may chemically modify a cell's DNA.

Anti-metabolites are another example of chemotherapeutic agents. Anti-metabolites may masquerade as purines or pyrimidines and may prevent purines and pyrimidines from becoming incorporated in to DNA during the “S” phase (of the cell cycle), thereby stopping normal development and division. Antimetabolites may also affect RNA synthesis. Examples of metabolites include azathioprine and mercaptopurine.

Alkaloids may be derived from plants and block cell division may also be used for the treatment of cancer. Alkyloids may prevent microtubule function. Examples of alkaloids are vinca alkaloids and taxanes. Vinca alkaloids may bind to specific sites on tubulin and inhibit the assembly of tubulin into microtubules (M phase of the cell cycle). The vinca alkaloids may be derived from the Madagascar periwinkle, Catharanthus roseus (formerly known as Vinca rosea). Examples of vinca alkaloids include, but are not limited to, vincristine, vinblastine, vinorelbine, or vindesine. Taxanes are diterpenes produced by the plants of the genus Taxus (yews). Taxanes may be derived from natural sources or synthesized artificially. Taxanes include paclitaxel (Taxol) and docetaxel (Taxotere). Taxanes may disrupt microtubule function. Microtubules are essential to cell division, and taxanes may stabilize GDP-bound tubulin in the microtubule, thereby inhibiting the process of cell division. Thus, in essence, taxanes may be mitotic inhibitors. Taxanes may also be radiosensitizing and often contain numerous chiral centers.

Alternative chemotherapeutic agents include podophyllotoxin. Podophyllotoxin is a plant-derived compound that may help with digestion and may be used to produce cytostatic drugs such as etoposide and teniposide. They may prevent the cell from entering the G1 phase (the start of DNA replication) and the replication of DNA (the S phase).

Topoisomerases are essential enzymes that maintain the topology of DNA. Inhibition of type I or type II topoisomerases may interfere with both transcription and replication of DNA by upsetting proper DNA supercoiling. Some chemotherapeutic agents may inhibit topoisomerases. For example, some type I topoisomerase inhibitors include camptothecins: irinotecan and topotecan. Examples of type II inhibitors include amsacrine, etoposide, etoposide phosphate, and teniposide.

Another example of chemotherapeutic agents is cytotoxic antibiotics. Cytotoxic antibiotics are a group of antibiotics that are used for the treatment of cancer because they may interfere with DNA replication and/or protein synthesis. Cytotoxic antiobiotics include, but are not limited to, actinomycin, anthracyclines, doxorubicin, daunorubicin, valrubicin, idarubicin, epirubicin, bleomycin, plicamycin, and mitomycin.

In some instances, the anti-cancer treatment may comprise radiation therapy. Radiation can come from a machine outside the body (external-beam radiation therapy) or from radioactive material placed in the body near cancer cells (internal radiation therapy, more commonly called brachytherapy). Systemic radiation therapy uses a radioactive substance, given by mouth or into a vein that travels in the blood to tissues throughout the body.

External-beam radiation therapy may be delivered in the form of photon beams (either x-rays or gamma rays). A photon is the basic unit of light and other forms of electromagnetic radiation. An example of external-beam radiation therapy is called 3-dimensional conformal radiation therapy (3D-CRT). 3D-CRT may use computer software and advanced treatment machines to deliver radiation to very precisely shaped target areas. Many other methods of external-beam radiation therapy are currently being tested and used in cancer treatment. These methods include, but are not limited to, intensity-modulated radiation therapy (IMRT), image-guided radiation therapy (IGRT), Stereotactic radiosurgery (SRS), Stereotactic body radiation therapy (SBRT), and proton therapy.

Intensity-modulated radiation therapy (IMRT) is an example of external-beam radiation and may use hundreds of tiny radiation beam-shaping devices, called collimators, to deliver a single dose of radiation. The collimators can be stationary or can move during treatment, allowing the intensity of the radiation beams to change during treatment sessions. This kind of dose modulation allows different areas of a tumor or nearby tissues to receive different doses of radiation. IMRT is planned in reverse (called inverse treatment planning). In inverse treatment planning, the radiation doses to different areas of the tumor and surrounding tissue are planned in advance, and then a high-powered computer program calculates the required number of beams and angles of the radiation treatment. In contrast, during traditional (forward) treatment planning, the number and angles of the radiation beams are chosen in advance and computers calculate how much dose may be delivered from each of the planned beams. The goal of IMRT is to increase the radiation dose to the areas that need it and reduce radiation exposure to specific sensitive areas of surrounding normal tissue.

Another example of external-beam radiation is image-guided radiation therapy (IGRT). In IGRT, repeated imaging scans (CT, MRI, or PET) may be performed during treatment. These imaging scans may be processed by computers to identify changes in a tumor's size and location due to treatment and to allow the position of the patient or the planned radiation dose to be adjusted during treatment as needed. Repeated imaging can increase the accuracy of radiation treatment and may allow reductions in the planned volume of tissue to be treated, thereby decreasing the total radiation dose to normal tissue.

Tomotherapy is a type of image-guided IMRT. A tomotherapy machine is a hybrid between a CT imaging scanner and an external-beam radiation therapy machine. The part of the tomotherapy machine that delivers radiation for both imaging and treatment can rotate completely around the patient in the same manner as a normal CT scanner. Tomotherapy machines can capture CT images of the patient's tumor immediately before treatment sessions, to allow for very precise tumor targeting and sparing of normal tissue.

Stereotactic radiosurgery (SRS) can deliver one or more high doses of radiation to a small tumor. SRS uses extremely accurate image-guided tumor targeting and patient positioning. Therefore, a high dose of radiation can be given without excess damage to normal tissue. SRS can be used to treat small tumors with well-defined edges. It is most commonly used in the treatment of brain or spinal tumors and brain metastases from other cancer types. For the treatment of some brain metastases, patients may receive radiation therapy to the entire brain (called whole-brain radiation therapy) in addition to SRS. SRS requires the use of a head frame or other device to immobilize the patient during treatment to ensure that the high dose of radiation is delivered accurately.

Stereotactic body radiation therapy (SBRT) delivers radiation therapy in fewer sessions, using smaller radiation fields and higher doses than 3D-CRT in most cases. SBRT may treat tumors that lie outside the brain and spinal cord. Because these tumors are more likely to move with the normal motion of the body, and therefore cannot be targeted as accurately as tumors within the brain or spine, SBRT is usually given in more than one dose. SBRT can be used to treat small, isolated tumors, including cancers in the lung and liver. SBRT systems may be known by their brand names, such as the CyberKnife®.

In proton therapy, external-beam radiation therapy may be delivered by proton. Protons are a type of charged particle. Proton beams differ from photon beams mainly in the way they deposit energy in living tissue. Whereas photons deposit energy in small packets all along their path through tissue, protons deposit much of their energy at the end of their path (called the Bragg peak) and deposit less energy along the way. Use of protons may reduce the exposure of normal tissue to radiation, possibly allowing the delivery of higher doses of radiation to a tumor.

Other charged particle beams such as electron beams may be used to irradiate superficial tumors, such as skin cancer or tumors near the surface of the body, but they cannot travel very far through tissue.

Internal radiation therapy (brachytherapy) is radiation delivered from radiation sources (radioactive materials) placed inside or on the body. Several brachytherapy techniques are used in cancer treatment. Interstitial brachytherapy may use a radiation source placed within tumor tissue, such as within a prostate tumor. Intracavitary brachytherapy may use a source placed within a surgical cavity or a body cavity, such as the chest cavity, near a tumor. Episcleral brachytherapy, which may be used to treat melanoma inside the eye, may use a source that is attached to the eye. In brachytherapy, radioactive isotopes can be sealed in tiny pellets or “seeds.” These seeds may be placed in patients using delivery devices, such as needles, catheters, or some other type of carrier. As the isotopes decay naturally, they give off radiation that may damage nearby cancer cells. Brachytherapy may be able to deliver higher doses of radiation to some cancers than external-beam radiation therapy while causing less damage to normal tissue.

Brachytherapy can be given as a low-dose-rate or a high-dose-rate treatment. In low-dose-rate treatment, cancer cells receive continuous low-dose radiation from the source over a period of several days. In high-dose-rate treatment, a robotic machine attached to delivery tubes placed inside the body may guide one or more radioactive sources into or near a tumor, and then removes the sources at the end of each treatment session. High-dose-rate treatment can be given in one or more treatment sessions. An example of a high-dose-rate treatment is the MammoSite® system. Bracytherapy may be used to treat patients with breast cancer who have undergone breast-conserving surgery.

The placement of brachytherapy sources can be temporary or permanent. For permanent brachytherapy, the sources may be surgically sealed within the body and left there, even after all of the radiation has been given off. In some instances, the remaining material (in which the radioactive isotopes were sealed) does not cause any discomfort or harm to the patient. Permanent brachytherapy is a type of low-dose-rate brachytherapy. For temporary brachytherapy, tubes (catheters) or other carriers are used to deliver the radiation sources, and both the carriers and the radiation sources are removed after treatment. Temporary brachytherapy can be either low-dose-rate or high-dose-rate treatment. Brachytherapy may be used alone or in addition to external-beam radiation therapy to provide a “boost” of radiation to a tumor while sparing surrounding normal tissue.

In systemic radiation therapy, a patient may swallow or receive an injection of a radioactive substance, such as radioactive iodine or a radioactive substance bound to a monoclonal antibody. Radioactive iodine (131I) is a type of systemic radiation therapy commonly used to help treat cancer, such as thyroid cancer. Thyroid cells naturally take up radioactive iodine. For systemic radiation therapy for some other types of cancer, a monoclonal antibody may help target the radioactive substance to the right place. The antibody joined to the radioactive substance travels through the blood, locating and killing tumor cells. For example, the drug ibritumomab tiuxetan (Zevalin®) may be used for the treatment of certain types of B-cell non-Hodgkin lymphoma (NHL). The antibody part of this drug recognizes and binds to a protein found on the surface of B lymphocytes. The combination drug regimen of tositumomab and iodine I 131 tositumomab (Bexxar®) may be used for the treatment of certain types of cancer, such as NHL. In this regimen, nonradioactive tositumomab antibodies may be given to patients first, followed by treatment with tositumomab antibodies that have 131I attached. Tositumomab may recognize and bind to the same protein on B lymphocytes as ibritumomab. The nonradioactive form of the antibody may help protect normal B lymphocytes from being damaged by radiation from 131I.

Some systemic radiation therapy drugs relieve pain from cancer that has spread to the bone (bone metastases). This is a type of palliative radiation therapy. The radioactive drugs samarium-153-lexidronam (Quadramet®) and strontium-89 chloride (Metastron®) are examples of radiopharmaceuticals may be used to treat pain from bone metastases.

Biological therapy (sometimes called immunotherapy, biotherapy, or biological response modifier (BRM) therapy) uses the body's immune system, either directly or indirectly, to fight cancer or to lessen the side effects that may be caused by some cancer treatments. Biological therapies include interferons, interleukins, colony-stimulating factors, monoclonal antibodies, vaccines, gene therapy, and nonspecific immunomodulating agents.

Interferons (IFNs) are types of cytokines that occur naturally in the body. Interferon alpha, interferon beta, and interferon gamma are examples of interferons that may be used in cancer treatment.

Like interferons, interleukins (ILs) are cytokines that occur naturally in the body and can be made in the laboratory. Many interleukins have been identified for the treatment of cancer. For example, interleukin-2 (IL-2 or aldesleukin), interleukin 7, and interleukin 12 have may be used as an anti-cancer treatment. IL-2 may stimulate the growth and activity of many immune cells, such as lymphocytes, that can destroy cancer cells. Interleukins may be used to treat a number of cancers, including leukemia, lymphoma, and brain, colorectal, ovarian, breast, kidney and prostate cancers.

Colony-stimulating factors (CSFs) (sometimes called hematopoietic growth factors) may also be used for the treatment of cancer. Some examples of CSFs include, but are not limited to, G-CSF (filgrastim) and GM-CSF (sargramostim). CSFs may promote the division of bone marrow stem cells and their development into white blood cells, platelets, and red blood cells. Bone marrow is critical to the body's immune system because it is the source of all blood cells. Because anticancer drugs can damage the body's ability to make white blood cells, red blood cells, and platelets, stimulation of the immune system by CSFs may benefit patients undergoing other anti-cancer treatment, thus CSFs may be combined with other anti-cancer therapies, such as chemotherapy. CSFs may be used to treat a large variety of cancers, including lymphoma, leukemia, multiple myeloma, melanoma, and cancers of the brain, lung, esophagus, breast, uterus, ovary, prostate, kidney, colon, and rectum.

Another type of biological therapy includes monoclonal antibodies (MOABs or MoABs). These antibodies may be produced by a single type of cell and may be specific for a particular antigen. To create MOABs, a human cancer cells may be injected into mice. In response, the mouse immune system can make antibodies against these cancer cells. The mouse plasma cells that produce antibodies may be isolated and fused with laboratory-grown cells to create “hybrid” cells called hybridomas. Hybridomas can indefinitely produce large quantities of these pure antibodies, or MOABs. MOABs may be used in cancer treatment in a number of ways. For instance, MOABs that react with specific types of cancer may enhance a patient's immune response to the cancer. MOABs can be programmed to act against cell growth factors, thus interfering with the growth of cancer cells.

MOABs may be linked to other anti-cancer therapies such as chemotherapeutics, radioisotopes (radioactive substances), other biological therapies, or other toxins. When the antibodies latch onto cancer cells, they deliver these anti-cancer therapies directly to the tumor, helping to destroy it. MOABs carrying radioisotopes may also prove useful in diagnosing certain cancers, such as colorectal, ovarian, and prostate.

Rituxan® (rituximab) and Herceptin® (trastuzumab) are examples of MOABs that may be used as a biological therapy. Rituxan may be used for the treatment of non-Hodgkin lymphoma. Herceptin can be used to treat metastatic breast cancer in patients with tumors that produce excess amounts of a protein called HER2. Alternatively, MOABs may be used to treat lymphoma, leukemia, melanoma, and cancers of the brain, breast, lung, kidney, colon, rectum, ovary, prostate, and other areas.

Cancer vaccines are another form of biological therapy. Cancer vaccines may be designed to encourage the patient's immune system to recognize cancer cells. Cancer vaccines may be designed to treat existing cancers (therapeutic vaccines) or to prevent the development of cancer (prophylactic vaccines). Therapeutic vaccines may be injected in a person after cancer is diagnosed. These vaccines may stop the growth of existing tumors, prevent cancer from recurring, or eliminate cancer cells not killed by prior treatments. Cancer vaccines given when the tumor is small may be able to eradicate the cancer. On the other hand, prophylactic vaccines are given to healthy individuals before cancer develops. These vaccines are designed to stimulate the immune system to attack viruses that can cause cancer. By targeting these cancer-causing viruses, development of certain cancers may be prevented. For example, cervarix and gardasil are vaccines to treat human papilloma virus and may prevent cervical cancer. Therapeutic vaccines may be used to treat melanoma, lymphoma, leukemia, and cancers of the brain, breast, lung, kidney, ovary, prostate, pancreas, colon, and rectum. Cancer vaccines can be used in combination with other anti-cancer therapies.

Gene therapy is another example of a biological therapy. Gene therapy may involve introducing genetic material into a person's cells to fight disease. Gene therapy methods may improve a patient's immune response to cancer. For example, a gene may be inserted into an immune cell to enhance its ability to recognize and attack cancer cells. In another approach, cancer cells may be injected with genes that cause the cancer cells to produce cytokines and stimulate the immune system.

In some instances, biological therapy includes nonspecific immunomodulating agents. Nonspecific immunomodulating agents are substances that stimulate or indirectly augment the immune system. Often, these agents target key immune system cells and may cause secondary responses such as increased production of cytokines and immunoglobulins. Two nonspecific immunomodulating agents used in cancer treatment are bacillus Calmette-Guerin (BCG) and levamisole. BCG may be used in the treatment of superficial bladder cancer following surgery. BCG may work by stimulating an inflammatory, and possibly an immune, response. A solution of BCG may be instilled in the bladder. Levamisole is sometimes used along with fluorouracil (5-FU) chemotherapy in the treatment of stage III (Dukes' C) colon cancer following surgery. Levamisole may act to restore depressed immune function.

Photodynamic therapy (PDT) is an anti-cancer treatment that may use a drug, called a photosensitizer or photosensitizing agent, and a particular type of light. When photosensitizers are exposed to a specific wavelength of light, they may produce a form of oxygen that kills nearby cells. A photosensitizer may be activated by light of a specific wavelength. This wavelength determines how far the light can travel into the body. Thus, photosensitizers and wavelengths of light may be used to treat different areas of the body with PDT.

In the first step of PDT for cancer treatment, a photosensitizing agent may be injected into the bloodstream. The agent may be absorbed by cells all over the body but may stay in cancer cells longer than it does in normal cells. Approximately 24 to 72 hours after injection, when most of the agent has left normal cells but remains in cancer cells, the tumor can be exposed to light. The photosensitizer in the tumor can absorb the light and produces an active form of oxygen that destroys nearby cancer cells. In addition to directly killing cancer cells, PDT may shrink or destroy tumors in two other ways. The photosensitizer can damage blood vessels in the tumor, thereby preventing the cancer from receiving necessary nutrients. PDT may also activate the immune system to attack the tumor cells.

The light used for PDT can come from a laser or other sources. Laser light can be directed through fiber optic cables (thin fibers that transmit light) to deliver light to areas inside the body. For example, a fiber optic cable can be inserted through an endoscope (a thin, lighted tube used to look at tissues inside the body) into the lungs or esophagus to treat cancer in these organs. Other light sources include light-emitting diodes (LEDs), which may be used for surface tumors, such as skin cancer. PDT is usually performed as an outpatient procedure. PDT may also be repeated and may be used with other therapies, such as surgery, radiation, or chemotherapy.

Extracorporeal photopheresis (ECP) is a type of PDT in which a machine may be used to collect the patient's blood cells. The patient's blood cells may be treated outside the body with a photosensitizing agent, exposed to light, and then returned to the patient. ECP may be used to help lessen the severity of skin symptoms of cutaneous T-cell lymphoma that has not responded to other therapies. ECP may be used to treat other blood cancers, and may also help reduce rejection after transplants.

Additionally, photosensitizing agent, such as porfimer sodium or Photofrin®, may be used in PDT to treat or relieve the symptoms of esophageal cancer and non-small cell lung cancer. Porfimer sodium may relieve symptoms of esophageal cancer when the cancer obstructs the esophagus or when the cancer cannot be satisfactorily treated with laser therapy alone. Porfimer sodium may be used to treat non-small cell lung cancer in patients for whom the usual treatments are not appropriate, and to relieve symptoms in patients with non-small cell lung cancer that obstructs the airways. Porfimer sodium may also be used for the treatment of precancerous lesions in patients with Barrett esophagus, a condition that can lead to esophageal cancer.

Laser therapy may use high-intensity light to treat cancer and other illnesses. Lasers can be used to shrink or destroy tumors or precancerous growths. Lasers are most commonly used to treat superficial cancers (cancers on the surface of the body or the lining of internal organs) such as basal cell skin cancer and the very early stages of some cancers, such as cervical, penile, vaginal, vulvar, and non-small cell lung cancer.

Lasers may also be used to relieve certain symptoms of cancer, such as bleeding or obstruction. For example, lasers can be used to shrink or destroy a tumor that is blocking a patient's trachea (windpipe) or esophagus. Lasers also can be used to remove colon polyps or tumors that are blocking the colon or stomach.

Laser therapy is often given through a flexible endoscope (a thin, lighted tube used to look at tissues inside the body). The endoscope is fitted with optical fibers (thin fibers that transmit light). It is inserted through an opening in the body, such as the mouth, nose, anus, or vagina. Laser light is then precisely aimed to cut or destroy a tumor.

Laser-induced interstitial thermotherapy (LITT), or interstitial laser photocoagulation, also uses lasers to treat some cancers. LITT is similar to a cancer treatment called hyperthermia, which uses heat to shrink tumors by damaging or killing cancer cells. During LITT, an optical fiber is inserted into a tumor. Laser light at the tip of the fiber raises the temperature of the tumor cells and damages or destroys them. LITT is sometimes used to shrink tumors in the liver.

Laser therapy can be used alone, but most often it is combined with other treatments, such as surgery, chemotherapy, or radiation therapy. In addition, lasers can seal nerve endings to reduce pain after surgery and seal lymph vessels to reduce swelling and limit the spread of tumor cells.

Lasers used to treat cancer may include carbon dioxide (CO2) lasers, argon lasers, and neodymium:yttrium-aluminum-garnet (Nd:YAG) lasers. Each of these can shrink or destroy tumors and can be used with endoscopes. CO2 and argon lasers can cut the skin's surface without going into deeper layers. Thus, they can be used to remove superficial cancers, such as skin cancer. In contrast, the Nd:YAG laser is more commonly applied through an endoscope to treat internal organs, such as the uterus, esophagus, and colon. Nd:YAG laser light can also travel through optical fibers into specific areas of the body during LITT. Argon lasers are often used to activate the drugs used in PDT.

For patients with high test scores consistent with systemic disease outcome after prostatectomy, additional treatment modalities such as adjuvant chemotherapy (e.g., docetaxel, mitoxantrone and prednisone), systemic radiation therapy (e.g., samarium or strontium) and/or anti-androgen therapy (e.g., surgical castration, finasteride, dutasteride) can be designated. Such patients would likely be treated immediately with anti-androgen therapy alone or in combination with radiation therapy in order to eliminate presumed micro-metastatic disease, which cannot be detected clinically but can be revealed by the target sequence expression signature.

Such patients can also be more closely monitored for signs of disease progression. For patients with intermediate test scores consistent with biochemical recurrence only (BCR-only or elevated PSA that does not rapidly become manifested as systemic disease only localized adjuvant therapy (e.g., radiation therapy of the prostate bed) or short course of anti-androgen therapy would likely be administered. For patients with low scores or scores consistent with no evidence of disease (NED) adjuvant therapy would not likely be recommended by their physicians in order to avoid treatment-related side effects such as metabolic syndrome (e.g., hypertension, diabetes and/or weight gain), osteoporosis, proctitis, incontinence or impotence. Patients with samples consistent with NED could be designated for watchful waiting, or for no treatment. Patients with test scores that do not correlate with systemic disease but who have successive PSA increases could be designated for watchful waiting, increased monitoring, or lower dose or shorter duration anti-androgen therapy.

Target sequences can be grouped so that information obtained about the set of target sequences in the group can be used to make or assist in making a clinically relevant judgment such as a diagnosis, prognosis, or treatment choice.

A patient report is also provided comprising a representation of measured expression levels of a plurality of target sequences in a biological sample from the patient, wherein the representation comprises expression levels of target sequences corresponding to any one, two, three, four, five, six, eight, ten, twenty, thirty or more of the target sequences corresponding to a target selected from Table 1, Table 2, Table 6, Table 7, or Table 15, the subsets described herein, or a combination thereof. In some embodiments, the representation of the measured expression level(s) may take the form of a linear or nonlinear combination of expression levels of the target sequences of interest. The patient report may be provided in a machine (e.g., a computer) readable format and/or in a hard (paper) copy. The report can also include standard measurements of expression levels of said plurality of target sequences from one or more sets of patients with known disease status and/or outcome. The report can be used to inform the patient and/or treating physician of the expression levels of the expressed target sequences, the likely medical diagnosis and/or implications, and optionally may recommend a treatment modality for the patient.

Also provided are representations of the gene expression profiles useful for treating, diagnosing, prognosticating, and otherwise assessing disease. In some embodiments, these profile representations are reduced to a medium that can be automatically read by a machine such as computer readable media (magnetic, optical, and the like). The articles can also include instructions for assessing the gene expression profiles in such media. For example, the articles may comprise a readable storage form having computer instructions for comparing gene expression profiles of the portfolios of genes described above. The articles may also have gene expression profiles digitally recorded therein so that they may be compared with gene expression data from patient samples. Alternatively, the profiles can be recorded in different representational format. A graphical recordation is one such format. Clustering algorithms can assist in the visualization of such data.

### Subtyping

The inventors of the present invention discovered multiple subtypes of prostate cancer, including, for example, ERG+; ETS+; SPINK1+; and triple-negative. Additional subtypes of prostate cancer that are useful in the methods of the present invention, include, ERG+GPR116+, ERG+GRM7+, ERG+GRM7+GPR116+, ERG+GPR116-, ETS+, MME+, VGLL3+, hetero, and NOD. Molecular subtyping is a method of classifying prostate cancers into one of multiple genetically-distinct categories, or subtypes. Each subtype responds differently to different kinds of treatments, and some subtypes indicate a higher risk of recurrence. As described herein, each subtype has a unique molecular and clinical fingerprint.

Differential expression analysis one or more of the targets listed in Table 1, Table 2, Table 6, Table 7, or Table 15 allow for the identification of the molecular subtype of a prostate cancer.

In some instances, the molecular subtyping methods of the present invention are used in combination with other biomarkers, like tumor grade and hormone levels, for analyzing the prostate cancer.

### Clinical Associations and Patient Outcomes

Molecular subtypes of the present invention have distinct clinical associations. Clinical associations that correlate to molecular subtypes include, for example, preoperative serum PSA, Gleason score (GS), extraprostatic extension (EPE), surgical margin status (SM), lymph node involvement (LNI), and seminal vesicle invasion (SVI).

In some embodiments, molecular subtypes of the present invention are used to predict patient outcomes such as biochemical recurrence (BCR), metastasis (MET) and prostate cancer death (PCSM) after radical prostatectomy.

### Treatment Response Prediction

In some embodiment, the molecular subtypes of the present invention are useful for predicting response to Androgen Deprivation Therapy (ADT) following radical prostatectomy.

In other embodiments, the molecular subtypes of the present invention are useful for predicting response to Radiation Therapy (RT) following radical prostatectomy.

### EXAMPLES

### Example 1: Development and Validation of a Genomic Classifier to Predict ERG Status in Prostate Cancer Tissue

A genomic classifier to predict ERG status in prostate cancer tissue was developed as follows. Prostate tumor tissue specimens were obtained from 252 patients who underwent radical prostatectomy for prostate cancer (252 training samples). Total RNA was extracted from the prostate cancer tissue samples. The extracted RNA was amplified, labeled and hybridized to Human Exon 1.0 ST microarrays (Affymetrix, Santa Clara, Calif.) covering 1.4 million probesets that were summarized to ˜22,000 core-level gene expression profiles. The SCAN algorithm was used for individual patient profile pre-processing and normalization.

A Random Forrest (RF) supervised model (m-ERG) to predict ERG rearrangement status as assessed by fluorescence in situ hybridization (FISH-ERG) was developed using the gene expression profiles obtained above. The m-ERG model generated scores ranging from 0 to 1, with higher scores indicating increased likelihood of ERG rearrangement presence. Based on cut-off optimization methods, a m-ERG score above 0.6 was used to define m-ERG+ profiles.

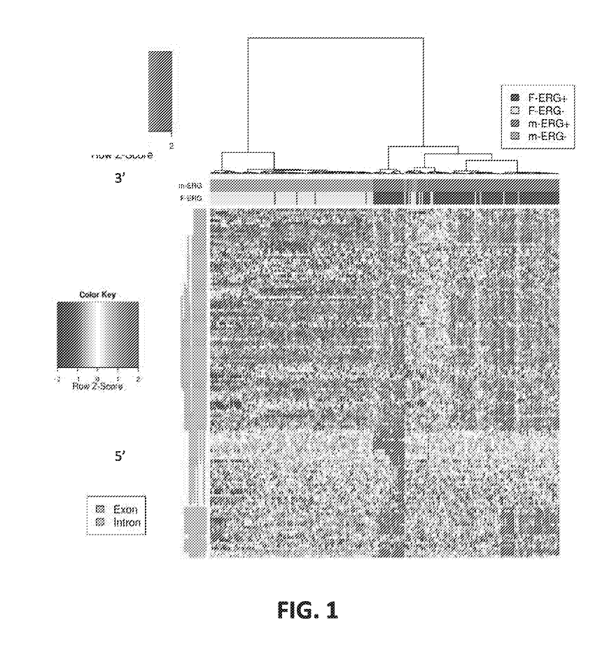

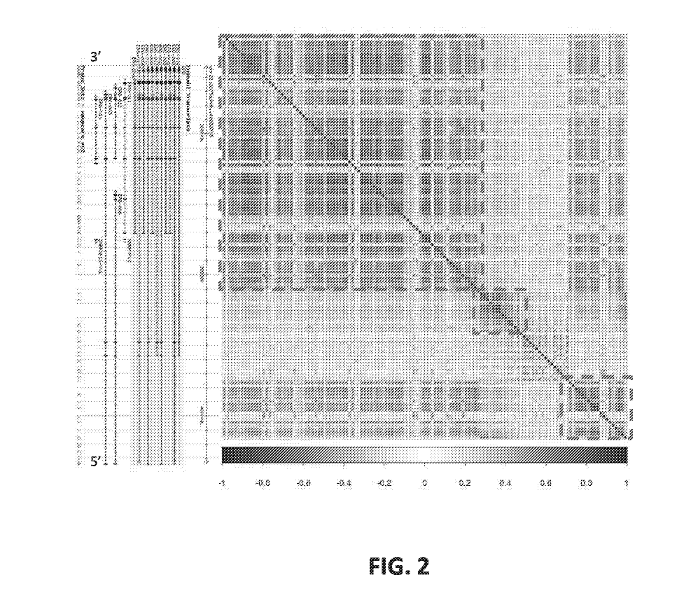

Informative probesets on the microarray for the m-ERG predictor were identified through a multi-step procedure. As shown in FIG. 1, clustering analysis of expression the 132 probesets mapping to the ERG locus demonstrated that they are highly informative of FISH-ERG status and probesets were highly correlated (see FIG. 2). These 132 probesets were filtered by removing redundant and non-informative features (e.g., not expressed above background) and then used to train a random forests (RF) classifier for predicting FISH-ERG status. The final model used the expression values of 3 ERG locus and 2 low expressing probesets predicting ERG rearrangement and predicted FISH-ERG status with an AUC of 0.98 in the training set.

These results showed that a genomic classifier of the present invention could be utilized to predict ERG status in prostate cancer subjects. These results suggested that the methods and markers of the present invention would be useful for diagnosing, prognosing, determining the progression of cancer, or predicting benefit from therapy in a subject having prostate cancer.

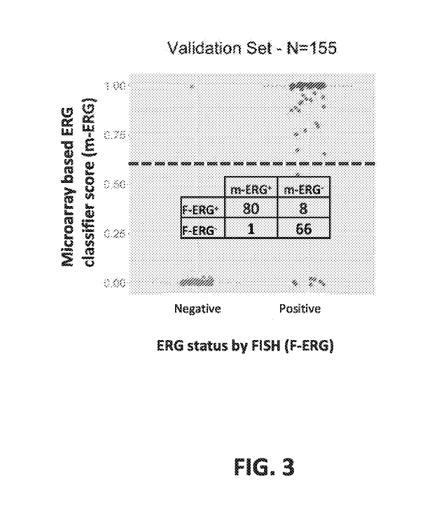

Another series of experiments were performed to validate the ERG status genomic classifier developed above. Total RNA was extracted from 155 prostate cancer tissue samples with known FISH-ERG information (155 validation samples) and gene expression profiles were obtained as described above. In the validation samples (n=155 profiles, not used for training m-ERG), the m-ERG model had an AUC of 0.94 and an overall accuracy of 95% (FIG. 3).

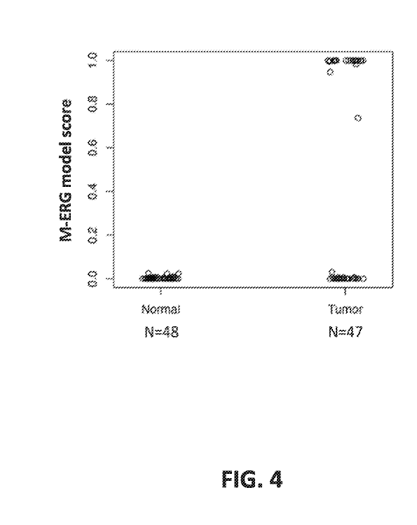

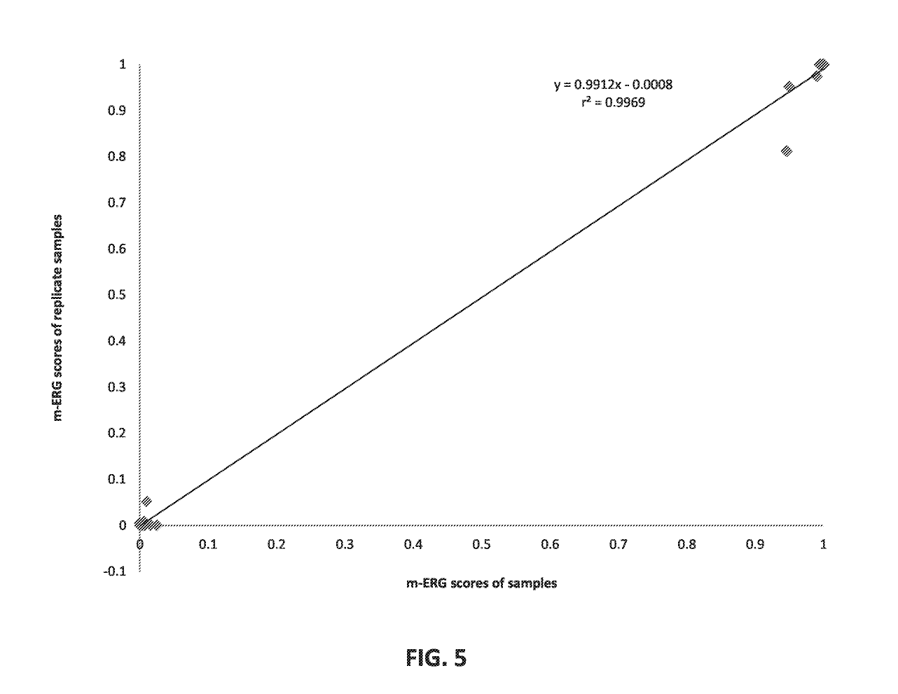

Next, the m-ERG genomic classifier was tested in another cohort where matched prostate cancer (PCa) and non-neoplastic radical prostatectomy (RP) specimen profiles were available for 48 patients. This analysis demonstrated the specificity of the m-ERG for PCa, with none of the non-neoplastic specimens being classified as m-ERG+(see FIG. 4). Technical replicates from 30 patients from a different cohort demonstrated near perfect correlation (R²=0.99), demonstrating the reproducibility of the model (FIG. 5).

The m-ERG genomic classifier was also evaluated in replicate assays from a panel of four commonly used prostate cancer cell lines profiled in the MSKCC study. VCAP cells, which endogenously over-express ERG due to TMPRSS2:ERG fusion, were classified as m-ERG+, while PC3, LNCaP and DU145 cells (known ERG rearrangement negative cells) were classified as m-ERG− (data not shown).

These results showed that a genomic classifier of the present invention could be utilized to predict ERG status in prostate cancer subjects. These results suggested that the methods and markers of the present invention would be useful for diagnosing, prognosing, determining the progression of cancer, or predicting benefit from therapy in a subject having prostate cancer.

### Example 2: Development of ETV1, ETV4, ETV5, FLI1 and SPINK1 Microarray-Based Classification Models in Prostate Cancer Patients

Microarray-based genomic classifiers for ETV1, ETV4, ETV5, FLI1 and SPINK1 status for prostate cancer tissue was developed as follows. To classify patient samples using the microarray-based expression of ETV1, ETV4, ETV5, FLI1 and SPINK1 genes, unsupervised gene outlier analysis method was applied to the core probesets expression for each gene. The outlier analysis method was applied on the entire discovery cohort in Example 1 to define expression threshold to classify each sample as an outlier (or not) for each gene, and then use the defined threshold to classify the remaining samples from the evaluation cohorts. Patients with outlier profiles were annotated as m-ETS+(m-ETV1+, m-ETV4+, m-ETV5+ or m-FLI1+) or m-SPINK1+.

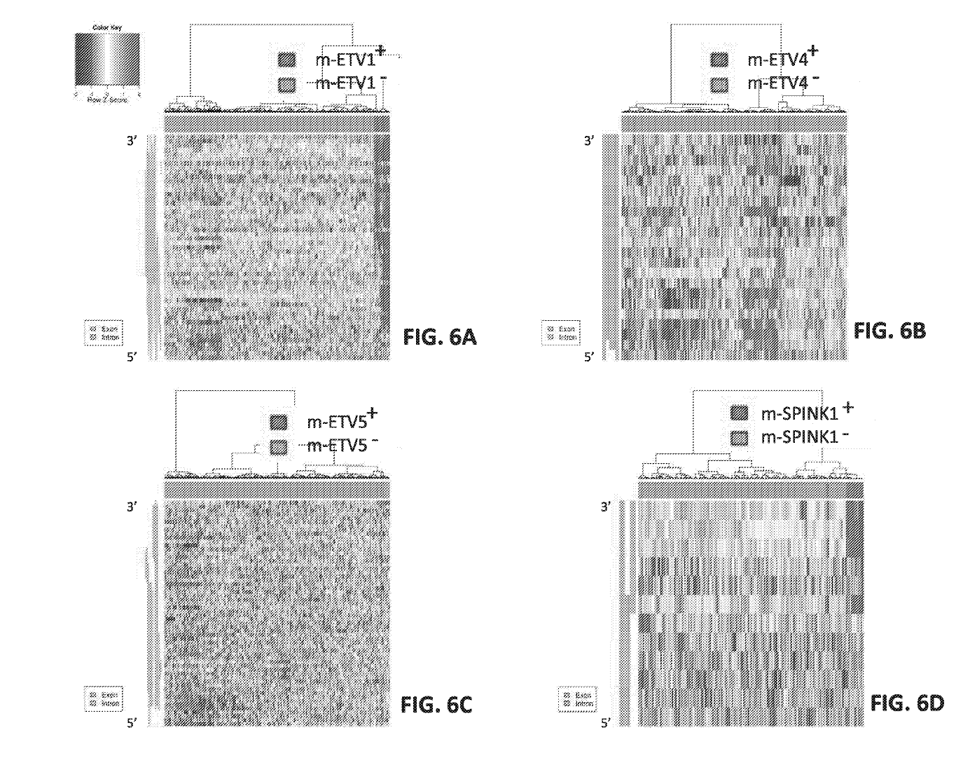

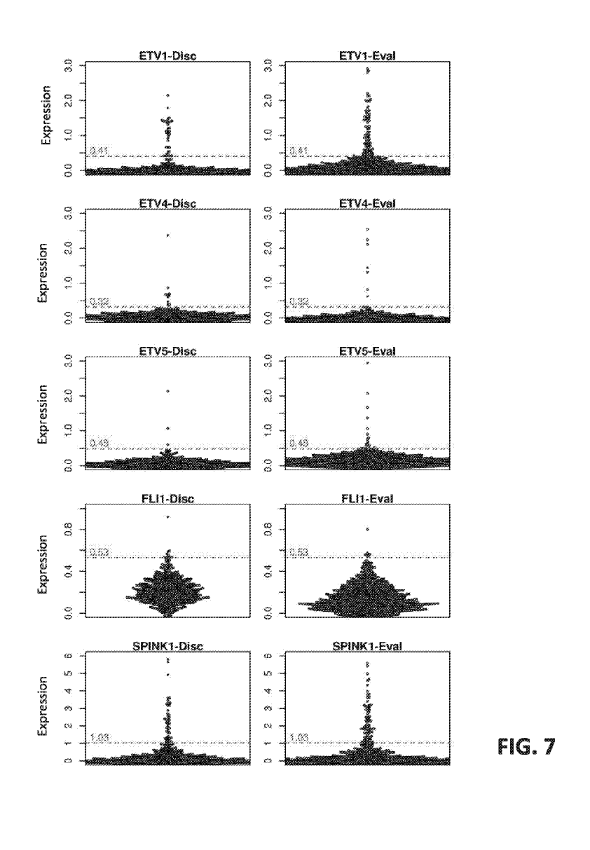

Heatmaps of ETV1 (FIG. 6A), ETV4 (FIG. 6B), ETV5 (FIG. 6C) and SPINK1 (FIG. 6D) exon/intron expression showed that a subset of patients have overexpression of some exons from each gene. Outlier analysis was first performed for a single cohort (Discovery samples) to define outlier thresholds or cut-point expression level for each gene, which was then applied to classify the patients in the remaining evaluation cohorts (FIG. 7). As shown in Table 1 below, for the Discovery samples, microarray outlier analysis classified 5% (n=31), 1.7% (n=10), 0.5% (n=3), 1% (n=5) and 7.7% (n=45) as m-ETV1+, m-ETV4+, m-ETV5+, m-FLI+ and m-SP1NK1+.

TABLE 1
Distribution of assigned molecular PCa subtype across the discovery
(n = 580) and evaluation (N = 997) samples.

| | Discovery | Evaluation |
|---|---|---|
| | (n samples) | (n samples) |
| Subtype | | |
| m-ERG⁺ | 268 | 430 |
| m-ETS⁺ | 49 | 99 |
| m-ETV1⁺ | 31 | 71 |
| m-ETV4⁺ | 10 | 7 |
| m-ETV5⁺ | 3 | 20 |
| m-FLI1⁺ | 5 | 1 |
| m-SPINK1⁺ | 45 | 74 |
| TripleNeg | 214 | 361 |
| Conflict cases | | |
| m-ERG⁺/m-ETV1⁺ | 4 | 21 |
| m-ERG⁺/m-ETV4⁺ | 1 | 1 |
| m-ERG⁺/m-ETV5⁺ | 0 | 5 |
| m-ERG⁺/m-FLI1⁺ | 0 | 4 |
| m-ERG⁺/m-SPINK1⁺ | 3 | 7 |

These results showed that a genomic classifier of the present invention could be utilized to predict ERGm ETV1, ETV4, ETV5, FLI1 and SPINK1 status in prostate cancer subjects. These results suggested that the methods and markers of the present invention would be useful for diagnosing, prognosing, determining the progression of cancer, or predicting benefit from therapy in a subject having cancer.

### Example 3: Molecular Subtyping of Prostate Cancer Patients Using Genomic Classifiers

The microarray-based classifiers for ERG, ETS (ETV1, ETV4, ETV5 and FLI1) and SPINK1 were used to subtype 1,577 prostate cancer patients as follows. Tumor profiles with high m-ERG score (m-ERG+) and m-ETV1−, m-ETV4−, m-ETV5−, m-FLI1− and m-SPINK1− were classified as m-ERG+ subtype. Profiles that were m-ETV1+, m-ETV4+, m-ETV5+ or m-FLI1+ and m-ERG− were classified as m-ETS+ subtype, and those that were m-SPINK1+ and m-ERG− were classified as m-SPINK1+ subtype. Finally, patient profiles that are m-ERG−, m-ETV1−, m-ETV4−, m-ETV5−, m-FLI1− and m-SPINK1− were classified as the ‘triple negative’ subtype. The four subtypes from this step were used to characterize the clinical and molecular characteristics of each subtype.

Overall, microarray outlier analysis classified 46% (n=738), 8% (n=102), 1% (n=17), 1.6% (n=23), 0.6% (n=6) and 8.4% (n=119) as m-ERG+, m-ETV1+, m-ETV4+, m-ETV5+, m-FLI+ and m-SPINK1+, respectively; 36.5% (n=575) lacked any outlier expression and were considered TripleNeg. Additionally, 3% (n=46) of patient profiles had outlier expression for two or more markers, which were defined as conflict cases. To focus on cases with clearly defined subtypes, the conflict cases were removed and the four ETS family members were collapsed into one group, generating four molecular subtypes with an overall prevalence of 45%, 9%, 8% and 38% for m-ERG+, m-ETS+, m-SPINK1+ and TripleNeg, respectively.

These results showed that a genomic classifier of the present invention could be utilized to predict ERG, ETV1, ETV4, ETV5, FLI1 and SPINK1 status in prostate cancer subjects. These results suggested that the methods and markers of the present invention would be useful for diagnosing, prognosing, determining the progression of cancer, or predicting benefit from therapy in a subject having cancer.

### Example 4: Clustering of Prostate Cancer Molecular Subtypes

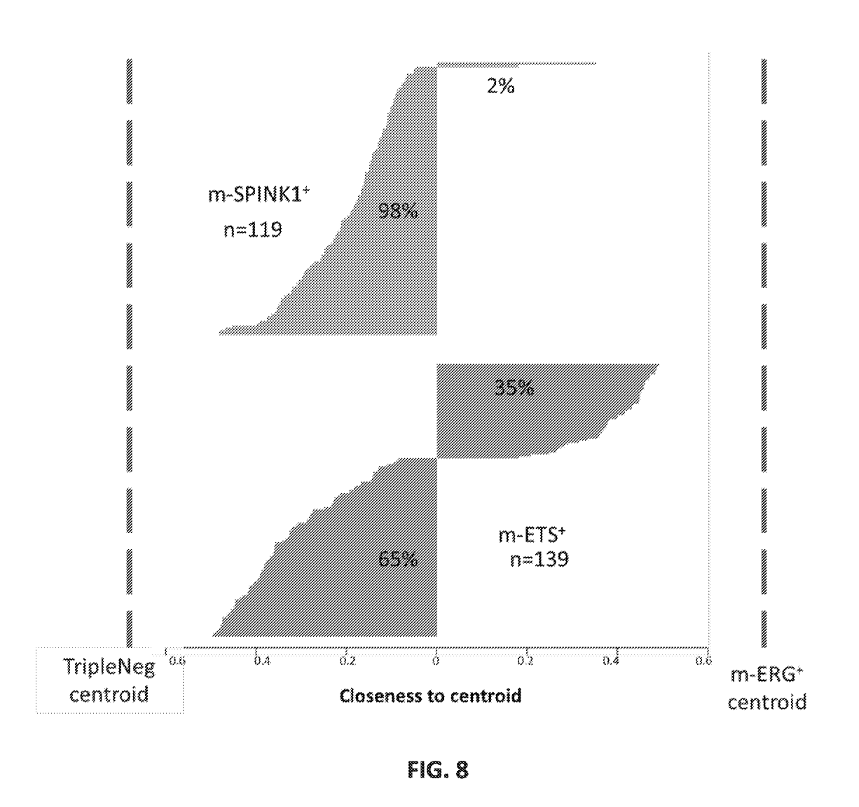

The following study was carried out to determine if m-ETS+ and m-SPINK1+ subtypes represent distinct molecular entities or are best classified as m-ERG+ and TripleNeg. Transcriptome-wide differential expression analysis was performed to identify 360 probesets with AUC>0.7 for discriminating m-ERG+ and TripleNeg. Using these 360 probesets to cluster all the patients (n=1531 excluding conflict cases) using fuzzy c-means clustering technique, with a c value=2 (number of clusters), all the m-SPINK1+ samples clustered with TripleNeg, whereas m-ETS+ samples clustered with both m-ERG+ and TripleNeg. When the number of clusters was varied from c=3-5, m-SPINK1+ tumors consistently clustered with TripleNeg tumors. In contrast, m-ETS+ tumors were distributed across clusters that had both m-ERG+ and TripleNeg tumors. To quantify how similar or different m-SPINK1+ and m-ETS+ subtypes are to m-ERG+ and TripleNeg, the distance between each m-SPINK1+ or m-ETS+ sample and the centroids of m-ERG+ and TripleNeg subtypes were calculated (based on the expression profile of the 360 top discriminatory probesets). These results showed that 98% (117/119) of m-SPINK1+ tumors had cluster distances closer to the TripleNeg centroid. In contrast, 35% of m-ETS+ tumors (48/139) had cluster distances closer to the m-ERG+ centroid, while 65% of m-ETS+ tumors were closer to the TripleNeg centroid (FIG. 8).

Results revealed that most m-SPINK1+PCa cluster with TripleNeg based on global and supervised gene expression, unlike m-ETS+PCa, which shared molecular overlap with both TripleNeg and m-ERG+ subtypes. These findings highlight important clinical differences between m-ERG+ and other m-ETS+PCa, as well as overall similarity between m-SPINK1+ and TripleNeg PCa. These results suggest at least three general molecular subtypes for prostate cancer: m-ERG+; m-ETS+; and m-SPINK1+/TripleNeg.

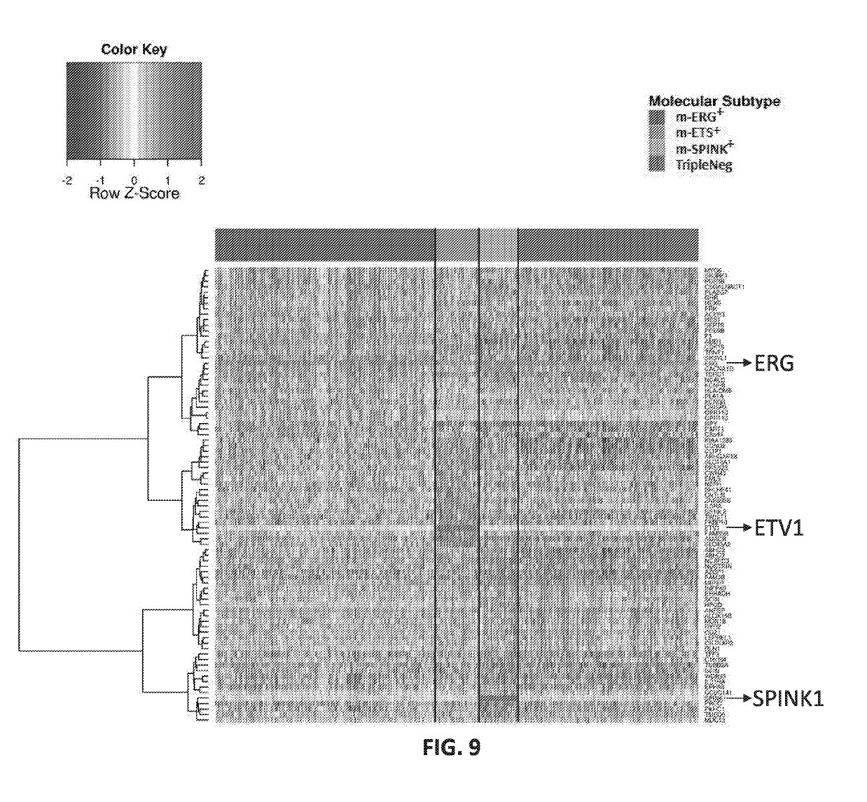

The most predictive genes for each subtype were defined based on AUC for discrimination of each subtype from the others. As shown in Table 2 below, 76, 15, 14 and 3 genes had an AUC>0.7 for m-ERG+, m-ETS+, m-SP1NK1+, and TripleNeg, respectively. Heatmap of these discriminatory genes across all samples demonstrated two main dendrogram branches corresponding to m-ERG+ and Triple Negative predictive genes. m-ETS+ tumors shared expression pattern of m-ERG+ predictive genes but also uniquely expressed a subset of genes, while the m-SPINK1+ tumors share a highly similar expression pattern with TripleNeg PCa (FIG. 9).

Further analysis identified TDRD1, CACNA1D, NCALD and HLA-DMB as the most specific m-ERG+ genes (AUCs=0.83-0.90). FAM65B and AMACR are the most predictive genes of m-ETS+ subtype with AUC of 0.76 and 0.74 respectively. Other genes that are specific for m-ETS+ subtype include SLC61A1 and FKBP10.

These results showed that the methods and markers of the present invention are useful for predicting ERG, ETV1, ETV4, ETV5, FLI1 and SPINK1 status in prostate cancer subjects. These results suggested that the methods and markers of the present invention would be useful for diagnosing, prognosing, determining the progression of cancer, or predicting benefit from therapy in a subject having cancer.

TABLE 2

| m-ERG⁺ | | m-ETS⁺ | | m-SPINK1⁺ | | TripleNeg | |
|---|---|---|---|---|---|---|---|
| Gene | AUC | Gene | AUC | Gene | AUC | Gene | AUC |
| TDRD1 | 0.91 | FAM65B | 0.76 | HPGD | 0.83 | TFF3 | 0.71 |
| CACNA1D | 0.89 | AMACR | 0.75 | FAM3B | 0.76 | ALOX15B | 0.70 |
| NCALD | 0.84 | ZNF385B | 0.74 | MIPEP | 0.73 | MON1B | 0.70 |
| HLA-DMB | 0.83 | CDK19 | 0.73 | NCAPD3 | 0.73 | | |
| KCNH8 | 0.82 | ARHGAP18 | 0.73 | INPP4B | 0.73 | | |
| PDE3B | 0.81 | IL5RA | 0.73 | ANPEP | 0.73 | | |
| PLA2G7 | 0.79 | SLC16A1 | 0.73 | TFF3 | 0.71 | | |
| CSGALNACT1 | 0.79 | CNTLN | 0.72 | IL31RA | 0.71 | | |
| PART1 | 0.78 | FKBP10 | 0.72 | EHHADH | 0.71 | | |
| HES1 | 0.78 | SLC45A2 | 0.71 | RP11-45B20.2 | 0.71 | | |
| F3 | 0.78 | CLIP1 | 0.70 | CCDC141 | 0.71 | | |
| GPR110 | 0.77 | HEXB | 0.70 | RLN1 | 0.71 | | |
| SH3RF1 | 0.77 | NEFH | 0.70 | ABHD2 | 0.70 | | |
| PDE8B | 0.77 | ODZ1 | 0.70 | SCIN | 0.70 | | |
| SEPT9 | 0.76 | SS18L2 | 0.70 | | | | |
| CRISP3 | 0.76 | | | | | | |
| AMD1 | 0.76 | | | | | | |
| KCNG3 | 0.76 | | | | | | |
| PLA1A | 0.76 | | | | | | |
| MYO6 | 0.76 | | | | | | |
| FRK | 0.76 | | | | | | |
| GPR110 | 0.76 | | | | | | |
| SH3YL1 | 0.76 | | | | | | |
| ACER3 | 0.75 | | | | | | |
| C8orf4 | 0.75 | | | | | | |
| GHR | 0.75 | | | | | | |
| ITPR1 | 0.74 | | | | | | |
| KHDRBS3 | 0.74 | | | | | | |
| NPY | 0.74 | | | | | | |
| GUCY1A3 | 0.74 | | | | | | |
| ARHGDIB | 0.74 | | | | | | |
| LAMC2 | 0.73 | | | | | | |
| VWA2 | 0.73 | | | | | | |
| ZNF432 | 0.73 | | | | | | |
| MORN1 | 0.73 | | | | | | |
| CYorf15B | 0.73 | | | | | | |
| AMPD3 | 0.72 | | | | | | |
| QDPR | 0.72 | | | | | | |
| HDAC1 | 0.72 | | | | | | |
| KIF16B | 0.72 | | | | | | |
| GJB1 | 0.72 | | | | | | |
| ITPR3 | 0.72 | | | | | | |
| ZNF615 | 0.72 | | | | | | |
| ANKRD6 | 0.72 | | | | | | |
| APOD | 0.72 | | | | | | |
| STEAP4 | 0.72 | | | | | | |
| RGS17 | 0.72 | | | | | | |
| MAP7 | 0.72 | | | | | | |
| C22orf36 | 0.72 | | | | | | |
| NKAIN1 | 0.71 | | | | | | |
| CHN2 | 0.71 | | | | | | |
| LRRFIP1 | 0.71 | | | | | | |
| SERGEF | 0.71 | | | | | | |
| ATP8A2 | 0.71 | | | | | | |
| NDRG1 | 0.71 | | | | | | |
| CDC42SE1 | 0.71 | | | | | | |
| LUZP2 | 0.71 | | | | | | |
| HNF1B | 0.71 | | | | | | |
| TFAP2A | 0.71 | | | | | | |
| ANKRD34B | 0.71 | | | | | | |
| SLC12A2 | 0.71 | | | | | | |
| PRAC | 0.71 | | | | | | |
| SLC5A4 | 0.71 | | | | | | |
| ACSL3 | 0.71 | | | | | | |
| CD24P4 | 0.71 | | | | | | |
| DNASE2B | 0.71 | | | | | | |
| SLC22A3 | 0.71 | | | | | | |
| ODC1 | 0.71 | | | | | | |
| SMOC2 | 0.71 | | | | | | |
| UGDH | 0.70 | | | | | | |
| DSC2 | 0.70 | | | | | | |
| WNK2 | 0.70 | | | | | | |
| RAB3B | 0.70 | | | | | | |
| FAM198B | 0.70 | | | | | | |
| KCNC2 | 0.70 | | | | | | |
| SNAP91 | 0.70 | | | | | | |

### Example 5: Clinical Associations of Prostate Cancer Molecular Subtypes

Clinical associations of prostate cancer molecular subtypes of the present invention were determined. On univariable analysis, race, preoperative PSA, Gleason score (GS), extraprostatic extension (EPE) and seminal vesicle invasion (SVI) status were non-uniformly distributed across microarray defined subtypes (Table 3). Multinomial multivariable analysis was used to compare subtypes to each other on the basis of clinical and pathological characteristics (Table 4). Compared to TripleNeg, m-ERG+PCa was associated with lower pre-operative PSA (OR=0.47, p<0.001) and lower Gleason score (OR=0.43, p<0.001), but nearly twice as likely to have EPE (OR=1.80, p<0.001) and nearly five times more likely to occur in men of European ancestry (p<0.001) (Table 4). The m-ETS+ subtype was more likely to have SVI compared to both TripleNeg (OR=2.27, p=0.004) or m-ERG+PCa (OR=1.96, p=0.01) (Table 4). Both TripleNeg and m-SPINK1+ tumors had significantly higher preoperative PSA (OR=2.12, p<0.001 and OR=1.73, p=0.05, respectively) and higher Gleason scores (OR=2.3, p<0.001 and OR=3.0, p<0.001, respectively), and were more common in African American patients (OR=5.44, p=0.002 and OR=16.87, p<0.001, respectively) compared to m-ERG+ tumors. Interestingly, m-SPINK1+ is significantly associated with lack of SMS compared to m-ERG+(OR=0.58, p=0.006). These clinicopathologic associations are consistent with the genomic analysis above that demonstrates that m-SPINK1+ and TripleNeg are highly similar, while m-ERG+ and m-ETS+ share distinct features.

These results showed that the molecular subtypes of the present invention have distinct clinical associations. These results suggested that the methods and markers of the present invention would be useful for diagnosing, prognosing, determining the progression of cancer, or predicting benefit from therapy in a subject having cancer.

TABLE 3

| | m- | m- | m- | | |
|---|---|---|---|---|---|
| Parameter | ERG⁺ | ETS⁺ | SPINK1⁺ | TripleNeg | p-value |
| Race | | | | | |
| Caucasian | 46% | 9% | 8% | 38% | 0.005** |
| Black/ | 22% | 13% | 15% | 50% | |
| African | | | | | |
| American | | | | | |
| Others | 40% | 7% | 13% | 40% | |
| Patient age | 63[37-79] | 62[43-78] | 65[47-76] | 64[40-78] | |
| (yrs)^{#} | | | | | |
| Pre-Op PSA | | | | | |
| <10 ng/mL | 49% | 8% | 8% | 36% | 0.003* |
| 10-20 ng/mL | 46% | 10% | 9% | 35% | |
| >20 ng/mL | 34% | 10% | 8% | 49% | |
| Path GS | | | | | |
| <=6 | 47% | 8% | 7% | 38% | <0.001* |
| 7 | 51% | 9% | 7% | 33% | |
| 8 | 34% | 11% | 10% | 46% | |
| >=9 | 34% | 11% | 9% | 46% | |
| EPE | | | | | |
| positive | 49% | 10% | 6% | 35% | <0.001** |
| negative | 39% | 8% | 9% | 43% | |
| SVI | | | | | |
| positive | 45% | 14% | 6% | 36% | 0.001** |
| negative | 45% | 7% | 9% | 40% | |
| SM | | | | | |
| positive | 46% | 10% | 7% | 39% | 0.24** |
| negative | 44% | 9% | 9% | 39% | |
| LNI | | | | | |
| positive | 43% | 13% | 6% | 37% | 0.28** |
| negative | 45% | 9% | 8% | 39% | |

^{#}except for median and range for age.
Pre-OP PSA = pre-operative serum PSA; Path GS = pathologic Gleason score at prostatectomy; EPE = extraprostatic extension; SVI = seminal vesicle invasion; SM = surgical margin status; LNI = lymph node involvement.
*Results from Chi-squared text.
**Results from Fisher's exact text.

TABLE 4

| | | m-ERG⁺ OR | | MVA | m-ETS⁺ OR | | MVA | m-SPINK⁺ OR | | MVA | ANOVA |
|---|---|---|---|---|---|---|---|---|---|---|---|
| | Variable | Estimate (95% CI) | | pvalue | Estimate (95% CI) | | pvalue | Estimate (95% CI) | | pvalue | p - value |
| Reference: | Pre-Op PSA | 0.47 | (0.33-0.68) | <0.001 | 0.48 | (0.26-0.88) | 0.021 | 0.81 | (0.44-1.51) | 0.42 | <0.001 |
| TripleNeg | Race(Black/ | 0.18 | (0.07-0.52) | 0.002 | 0.21 | (0.03-1.6) | 0.12 | 3.10 | (1.23-7.82) | 0.02 | <0.001 |
| | African American) | | | | | | | | | | |
| | EPE | 1.80 | (1.34-2.41) | <0.001 | 1.23 | (0.75-2.01) | 0.34 | 0.76 | (0.46-1.26) | 0.37 | <0.001 |
| | SVI | 1.16 | (0.83-1.62) | 0.24 | 2.27 | (1.35-3.82) | 0.004 | 0.84 | (0.47-1.53) | 0.51 | 0.01 |
| | PathGS < 7 | 0.96 | (0.61-1.51) | 0.93 | 0.75 | (0.31-1.81) | 0.58 | 0.89 | (0.39-2.04) | 0.79 | <0.001 |
| | PathGS > 7 | 0.43 | (0.32-0.6) | <0.001 | 0.75 | (0.45-1.26) | 0.46 | 1.31 | (0.78-2.21) | 0.39 | <0.001 |
| | SMS | 1.18 | (0.89-1.56) | 0.29 | 1.27 | (0.79-2.04) | 0.53 | 0.69 | (0.42-1.12) | 0.13 | 0.13 |
| | Age | 1 | (0.98-1.02) | 0.71 | 0.97 | (0.94-1) | 0.045 | 1.01 | (0.97-1.05) | 0.62 | 0.24 |
| | LNI | 1.27 | (0.77-2.11) | 0.42 | 1.6 | (0.78-3.27) | 0.22 | 1.16 | (0.49-2.76) | 0.73 | 0.60 |
| | | m-ETS⁺ OR | | MVA | m-SPINK⁺ OR | | MVA | TripleNeg OR | | MVA | ANOVA |
| | Variable | Estimate (95% CI) | | pvalue | Estimate (95% CI) | | pvalue | Estimate (95% CI) | | pvalue | p - value |
| Reference: | Pre-Op PSA | 1.01 | (0.54-1.88) | 0.92 | 1.73 | (0.91-3.27) | 0.05 | 2.12 | (1.47-3.06) | <0.001 | <0.001 |
| m-ERG⁺ | Race(Black/ | 1.12 | (0.13-9.88) | 0.61 | 16.87 | (5.13-55.48) | <0.001 | 5.44 | (1.94-15.29) | 0.002 | <0.001 |
| | African American) | | | | | | | | | | |
| | EPE | 0.68 | (0.42-1.11) | 0.13 | 0.42 | (0.25-0.7) | 0.001 | 0.56 | (0.41-0.75) | <0.001 | <0.001 |
| | SVI | 1.96 | (1.18-3.24) | 0.01 | 0.73 | (0.4-1.32) | 0.37 | 0.86 | (0.62-1.2) | 0.24 | 0.014 |
| | PathGS < 7 | 0.78 | (0.33-1.85) | 0.5 | 0.93 | (0.41-2.13) | 0.96 | 1.04 | (0.66-1.64) | 0.93 | <0.001 |
| | PathGS > 7 | 1.74 | (1.05-2.88) | 0.05 | 3.01 | (1.77-5.13) | <0.001 | 2.30 | (1.68-3.15) | <0.001 | <0.001 |
| | SMS | 1.08 | (0.68-1.72) | 0.91 | 0.58 | (0.36-0.95) | 0.006 | 0.85 | (0.64-1.12) | 0.29 | 0.12 |
| | Age | 0.97 | (0.94-1) | 0.08 | 1.01 | (0.98-1.05) | 0.71 | 1 | (0.98-1.02) | 0.71 | 0.23 |
| | LNI | 1.25 | (0.62-2.53) | 0.59 | 0.91 | (0.38-2.17) | 0.57 | 0.78 | (0.47-1.3) | 0.42 | 0.60 |

OR = odds ratio; CI = confidence interval; Pre-OP PSA = pre-operative serum PSA (reference: <20 ng/mL); Race (reference: Caucasian); EPE = extraprostatic extension; SVI = seminal vesicle invasion; PathGS = pathologic Gleason score at prostatectomy (reference: Gleason score 7); SMS = surgical margin status; LNI = lymph node involvement.

### Example 6: Impact of Prostate Cancer Molecular Subtyping on Prognosis

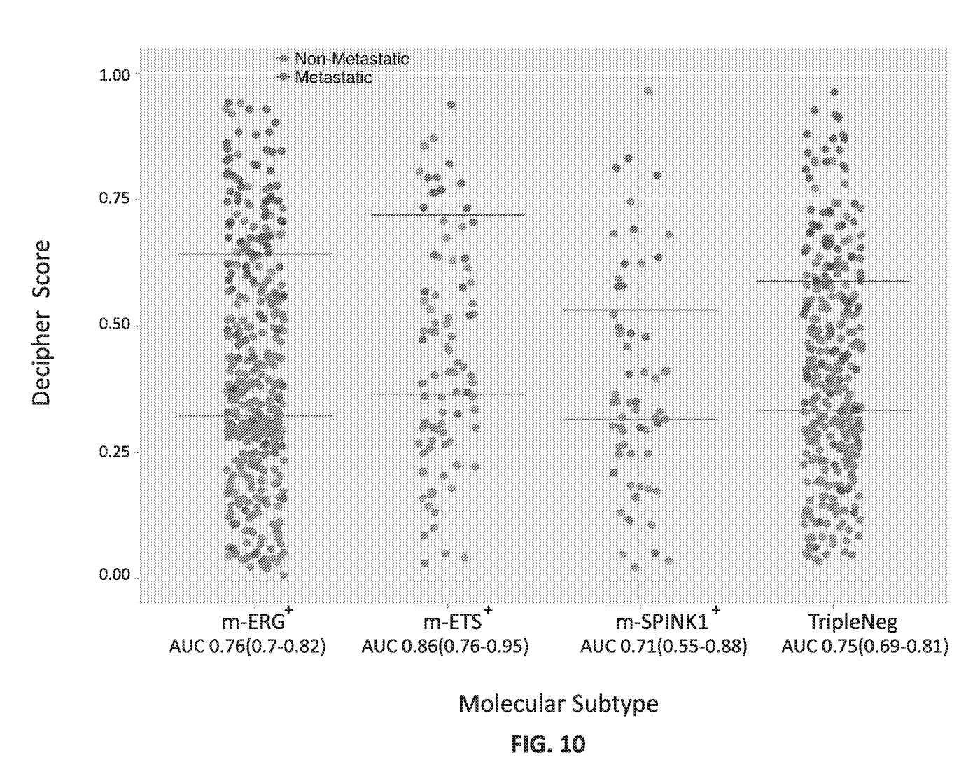

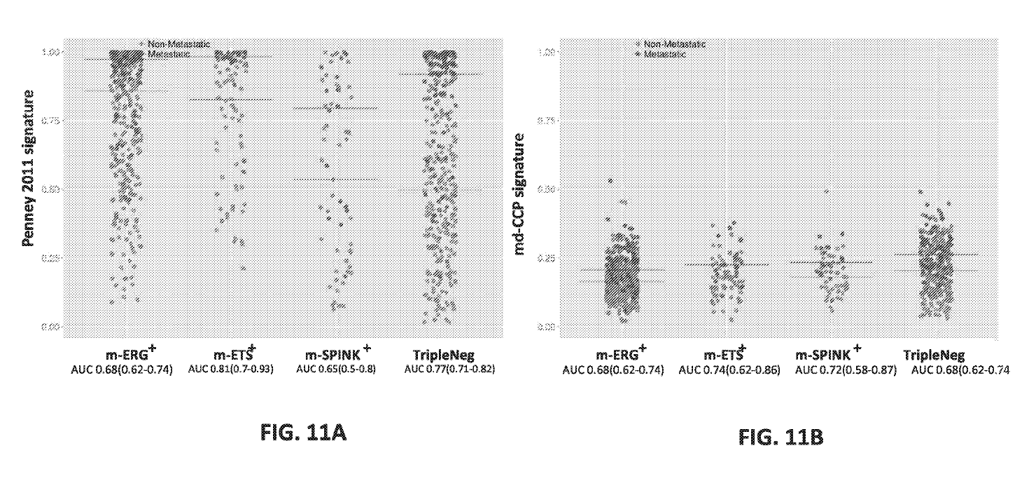

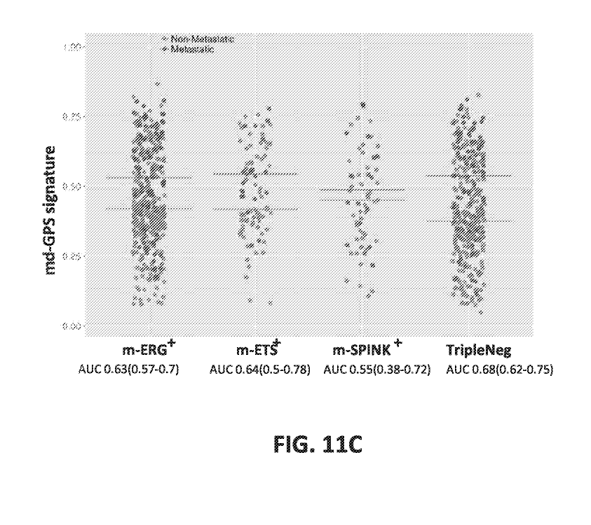

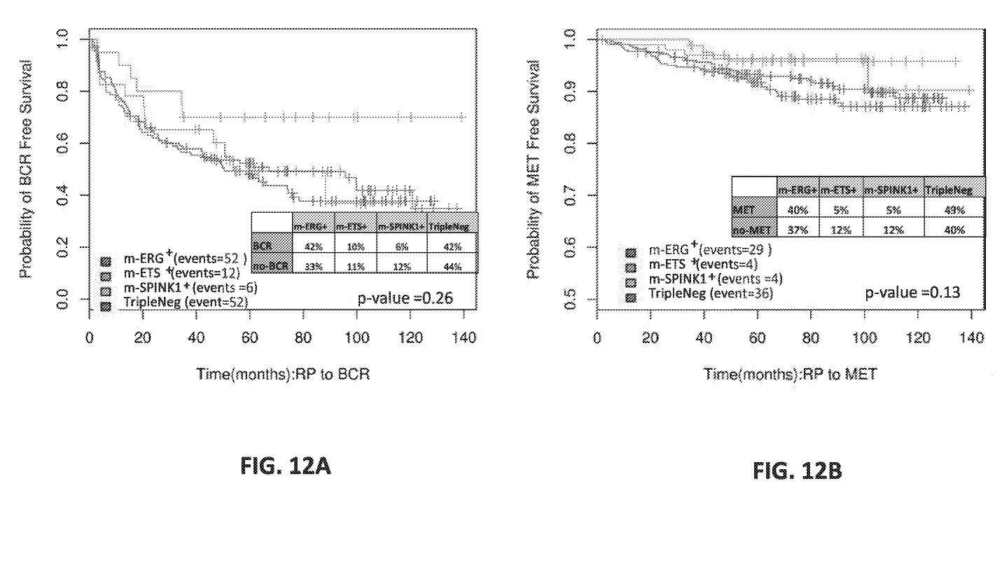

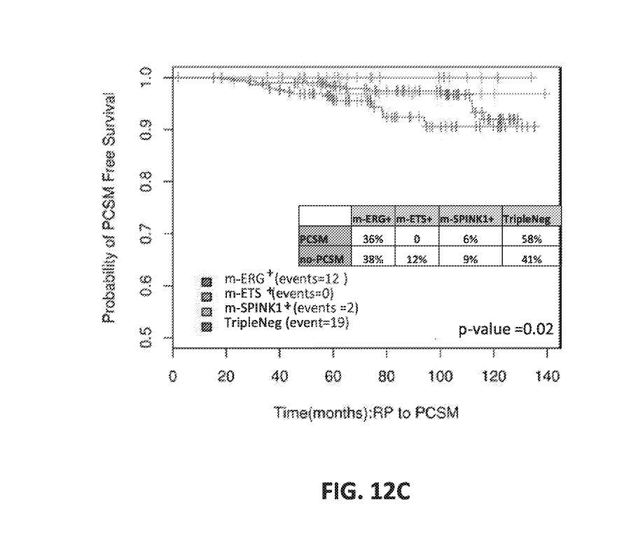

To determine the impact of molecular subtyping on prognosis, the ability of the subtypes to predict patient outcomes such as biochemical recurrence (BCR), metastasis (MET) and prostate cancer death (PCSM) after radical prostatectomy was assessed (see Table 5). ROC analysis showed that the subtypes discriminate for survival endpoints (AUC˜0.5). Likewise, the prognostic biomarker panel Decipher shows similar discrimination (as measured by AUC metric) for metastasis in all four subtypes (FIG. 10). Other prognostic signatures such as CCP, GPS and the Penney et al. signature which can be derived from global gene expression data, showed similar discrimination for metastasis in all subtypes except GPS, which was not discriminative in the m-SPINK+ subtype (FIGS. 11A-C). Kaplan-Meier analyses failed to show significant differences in time to events for BCR (FIG. 12A) and metastasis (FIG. 12B) endpoints between the subtypes. However, a trend toward significance was observed with the Triple Negative subtype patients having worse PCSM than the other subtypes (FIG. 12C).

These results showed that the molecular subtypes of the present invention are useful for prognosing prostate cancer and they set up the basis for further subtyping of prostate cancer. These results suggested that the methods and markers of the present invention would be useful for diagnosing, prognosing, determining the progression of cancer, or predicting benefit from therapy in a subject having cancer.

TABLE 5

| Parameter | AUC for BCR | AUC for MET | AUC for PCSM |
|---|---|---|---|
| m-ERG⁺ | 0.49 | 0.48 | 0.46 |
| m-ETS⁺ | 0.5 | 0.5 | 0.51 |
| m-SPINK1⁺ | 0.49 | 0.5 | 0.51 |
| TripleNeg | 0.5 | 0.5 | 0.52 |
| Path GS | 0.66 | 0.73 | 0.74 |
| Pre-PSA | 0.62 | 0.59 | 0.58 |

### Example 7: Development of Microarray-Based Classifiers for MME (CD10), BANK1, LEPREL1 (P3112), VGLL3, NPR3, TTN, OR4K7P, OR4K6P, POTEB2, RP11-403B2.10, and FABP5P7 in Prostate Cancer Patients

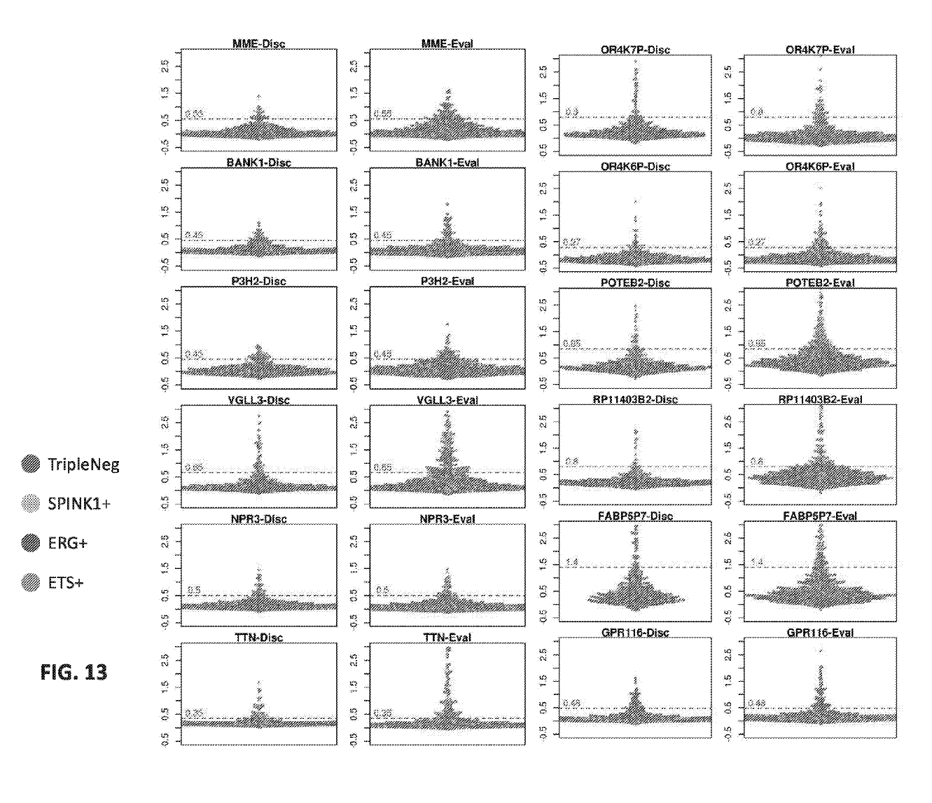

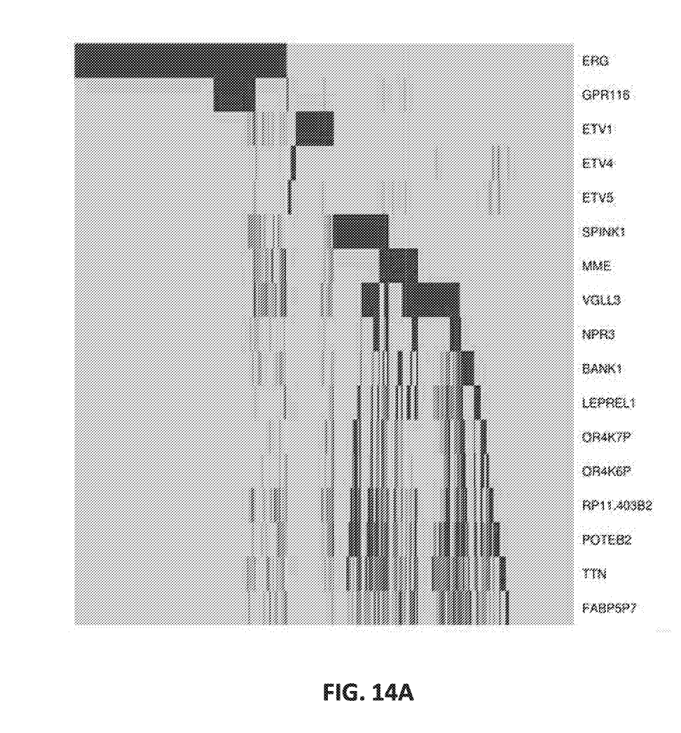

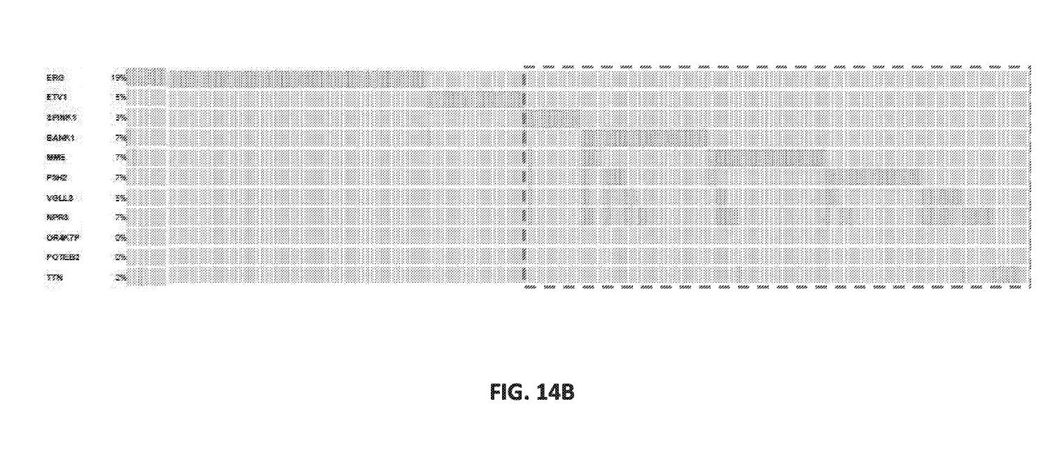

Microarray-based genomic classifiers for MME (CD10), BANK1, LEPREL1 (P3H2), VGLL3, NPR3, TTN, OR4K7P, OR4K6P, POTEB2, RP11-403B2.10, and FABP5P7 status for prostate cancer tissue was developed as follows. An outlier analysis method was applied on the entire discovery cohort as described in Examples 1 and 2. This allowed for the identification of outlier genes expressed in the TripleNeg or m-SPINK+ subtypes but not expressed in the m-ERG+ or m-ETS+ subtypes. Defined expression thresholds were used to classify each sample as an outlier (or not) for each gene. The defined thresholds were also used to classify the remaining samples from the evaluation cohorts (n=1305 pooled from 7 cohorts). Based on this method, we identified 11 genes with outlier profiles in the TripleNeg or m-SPINK+ subtypes. Beeswarm plots (FIG. 13) show the overexpression of the 11 genes in TripleNeg (green) and m-SPINK1+(cyan) subtype patients. The percentage of the 11 outliers ranged from 6% up to 18% across all patients (see Table 6). Between the TripleNeg and m-SPINK+ subgroups, around 70% were assigned to a subgroup.

TABLE 6

| | Percent (%) of outliers | Percent (%) of outliers in |
|---|---|---|
| | in Discovery (n = 545) | Evaluation (n = 1305) |
| MME | 5.50 | 11.34 |
| BANK1 | 6.24 | 7.20 |
| LEPREL1 | 6.79 | 8.74 |
| VGLL3 | 8.26 | 21.69 |
| NPR3 | 6.61 | 5.36 |
| OR4K7P | 5.32 | 7.13 |
| OR4K6P | 8.81 | 5.52 |
| POTEB2 | 4.77 | 15.63 |
| RP11 | 2.57 | 11.65 |
| TTN | 6.61 | 10.96 |
| FAP5 | 7.89 | 9.27 |
| GPR116 | 8.81 | 8.43 |

Percent of samples with outlier profile for each gene in the discovery and evaluation set.

These results showed that a genomic classifier of the present invention could be utilized to predict MME (CD10), BANK1, LEPREL1 (P3H2), VGLL3, NPR3, TTN, OR4K7P, OR4K6P, POTEB2, RP11-403B2.10, and FABP5P7 status in prostate cancer subjects. These results suggested that the methods and markers of the present invention would be useful for diagnosing, prognosing, determining the progression of cancer, or predicting benefit from therapy in a subject having cancer.

### Example 8: Development of GPR116 Microarray-Based Classifier in Prostate Cancer Patients

A microarray-based genomic classifier for GPR116 status for prostate cancer tissue was developed as follows. The outlier analysis method was applied on the entire discovery cohort as described in Examples 1 and 2. Outlier genes expressed in the m-ERG+ subset were identified. A threshold was defined to classify patients as an outlier (or not) and then the defined threshold was used to classify the remaining samples from the evaluation cohorts (n=1305 pooled from 7 cohorts).

One gene (GPR116) was identified as an outlier profile in the m-ERG+ subgroup. Beeswarm plots (see FIG. 13) showing the overexpression of the GPR116 in m-ERG+(red) patients. Out of the 1,850 prostate cancer patients, 8.5% were GPR116+, making up to 20% of the m-ERG+ subgroup.

These results showed that a genomic classifier of the present invention could be utilized to predict GPR116 status in ERG+ prostate cancer subjects. These results suggested that the methods and markers of the present invention would be useful for diagnosing, prognosing, determining the progression of cancer, or predicting benefit from therapy in a subject having cancer.

### Example 9: Outlier Genes are ERG-Negative Specific and are not Mutually Exclusive

The outlier expression of the 11 genes in Example 7 is nearly mutually exclusive as between ERG and ETS. However, they are not mutually exclusive with each other based on expression data from HuEx array (see FIG. 14A). OR4K7P and OR4K6P were highly correlated and patients with OR4K7P outlier expression were also OR4K6P outlier. Similarly, POTEB2 and RP11-403B2 were highly correlated and are located close to each other on Ch15 q11.

Similar results from RNAseq (see FIG. 14B) data obtained from TCGA data using cbioportal online tools showed that overexpression of MME, BANK1, LEPREL1, VGLL3, NPR3, TTN were mutually exclusive with ERG and ETV1 overexpression supporting that results from the Human exon platform.

These results suggested that the methods and markers of the present invention would be useful for diagnosing, prognosing, determining the progression of cancer, or predicting benefit from therapy in a subject having cancer.

### Example 10: Prognostic Impact of Individual Gene Outliers

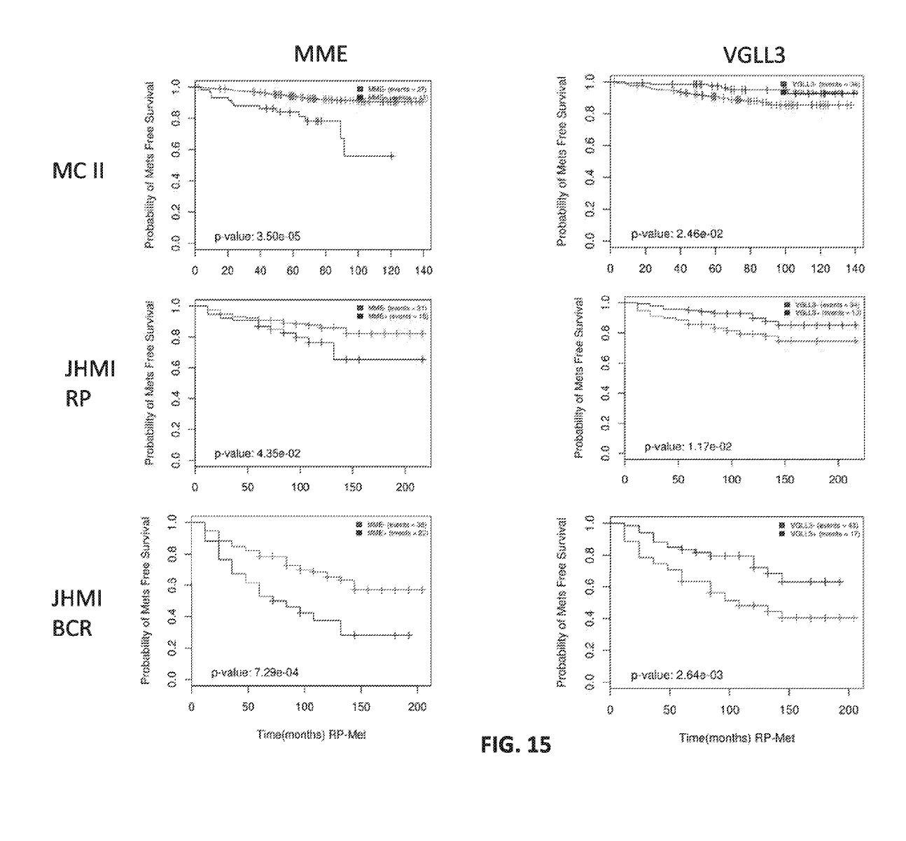

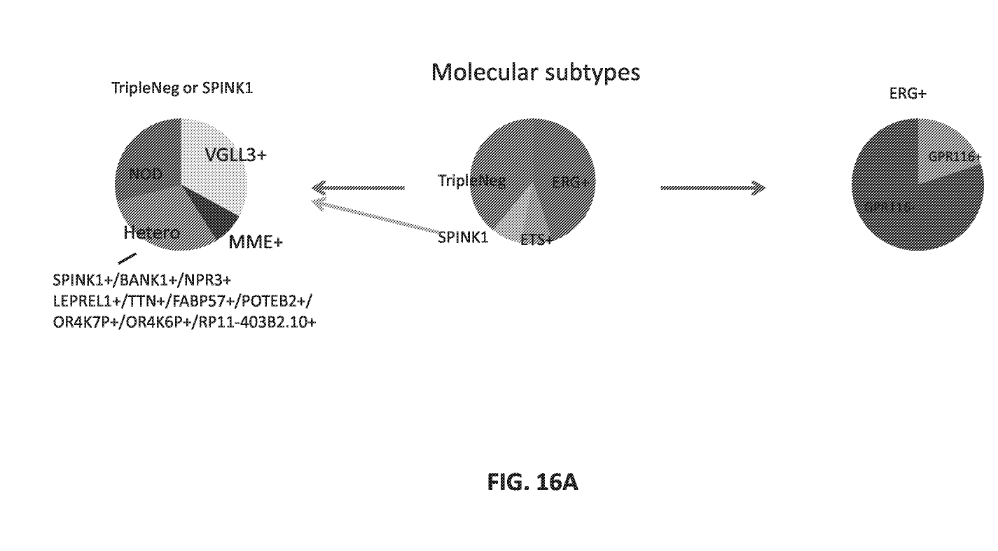

To characterize the clinical utility of the gene outliers, survival analysis using Kaplan-Meiers and logrank test in three case-cohorts (MC II, n=232), (JHMI-RP, n=262) and (JHMI-BCR, n=213) was performed. Table 7 shows logrank p-values of the 12 gene outliers in the three cohorts. MME outliers (overexpression) showed to be associated with worse prognosis of metastasis after radical prostatectomy (RP) in the cohorts. VGLL3 outliers were significantly associated with better prognosis (FIG. 15).

TABLE 7

| | | MC II | JHMI- | JHMI- |
|---|---|---|---|---|
| | | (n = 232) | RP(n = 262) | BCR(n = 213) |
| TripleNeg | MME | 0.00003 | 0.04 | 0.0007 |
| | BANK1 | 0.44 | 0.87 | 0.04 |
| | LEPREL1 | 0.99 | 0.74 | 0.97 |
| | VGLL3 | 0.024 | 0.011 | 0.0026 |
| | NPR3 | 0.36 | 0.016 | 0.045 |
| | OR4K6P | 0.16 | 0.54 | 0.25 |
| | OR4K7P | 0.37 | 0.96 | 0.033 |
| | POTEB2 | 0.6 | 0.25 | 0.54 |
| | RP11.403 | 0.85 | 0.033 | 0.82 |
| | TTN | 0.22 | 0.37 | 0.1 |
| | FABP5P7 | 0.86 | 0.2 | 0.57 |
| ERG+ | GPR116 | 0.00082 | 0.047 | 0.18 |

Prognostic values of the 12 outlier genes across all patients in each cohort.

These results suggested that the methods and markers of the present invention would be useful for diagnosing, prognosing, determining the progression of cancer, or predicting benefit from therapy in a subject having cancer.

### Example 11: Subgroups Based on Outliers in the SPINK1 and TripleNeg Subtypes

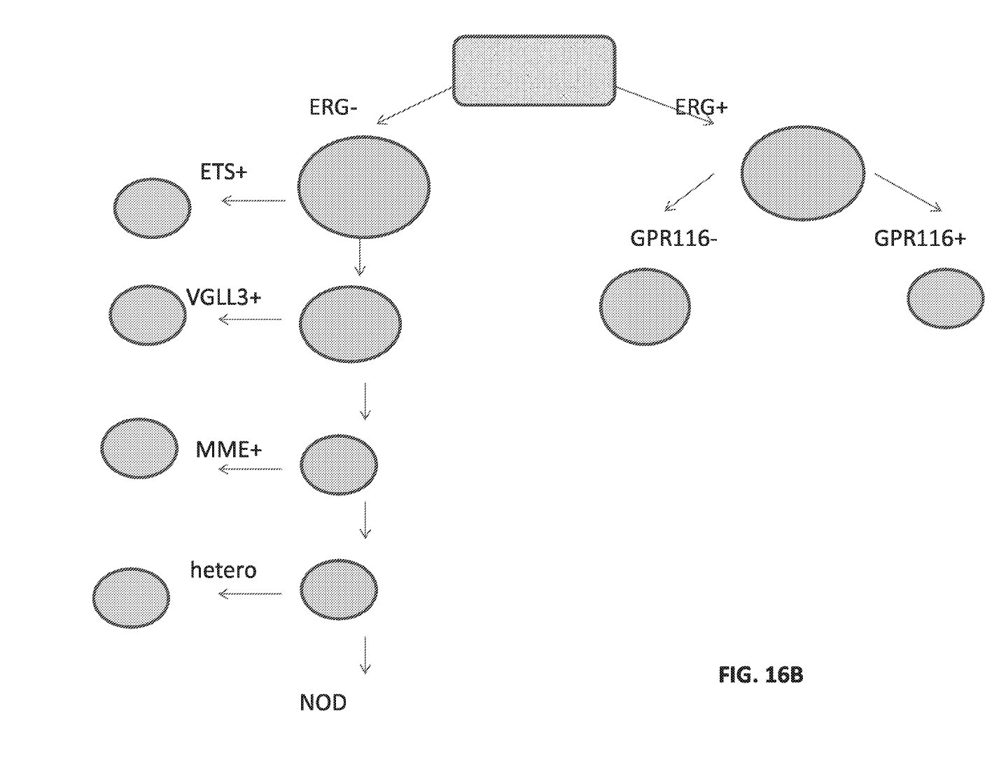

Additional subgroups were identified for the four molecular subtypes identified in Examples 1 and 2 above. FIG. 16A shows subgrouping for m-ERG+ based on GPR116 expression. The m-SPINK1+ and TripleNeg subtypes were sub-grouped into four groups: VGLL3+; MME+; hetero (SPINK1+, BANK1+, LEPREL1+, TTN+, POTEB2+, OR4K7P+, OR4K6P+, FABP5P7+, NPR1+, RP11-403B2+); and NOD (no outlier detected). TripleNeg and m-SPINK+ were combined as they were shown to be molecularly and clinically similar (see Examples 4 and 5). Genes (MME, VGLL3) were used to group the patients into four groups. FIG. 16B shows a flowchart for subgrouping prostate cancer patients into seven clinically distinct subgroups (ERG+GPR116+, ERG+GPR116-, ERG-ETS+, ERG-VGLL3+, ERG-MME+, ERG− hetero, and NOD).

These results suggested that the methods and markers of the present invention would be useful for diagnosing, prognosing, determining the progression of cancer, or predicting benefit from therapy in a subject having cancer.

### Example 12: VGLL3+ Group is Associated with Favorable Outcome

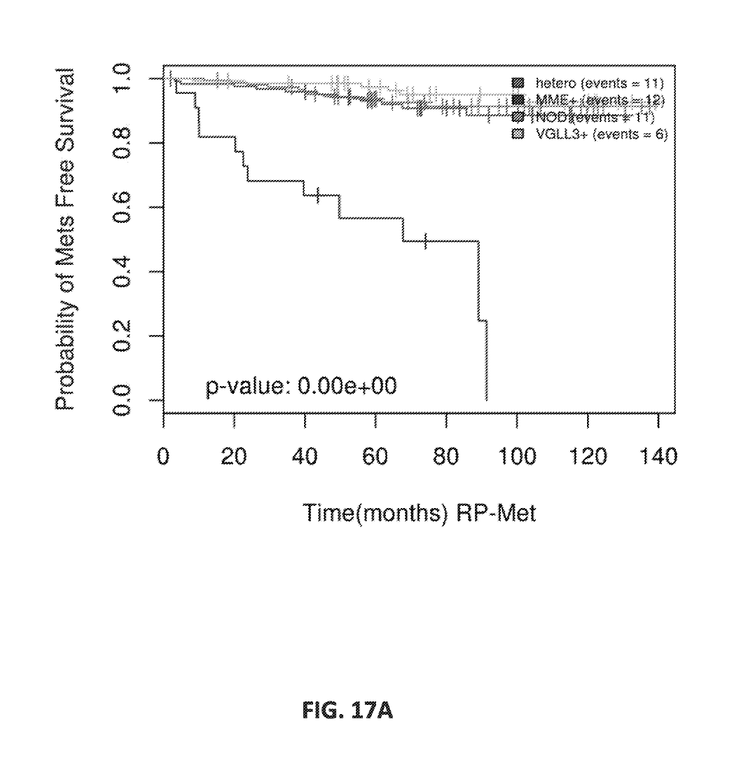

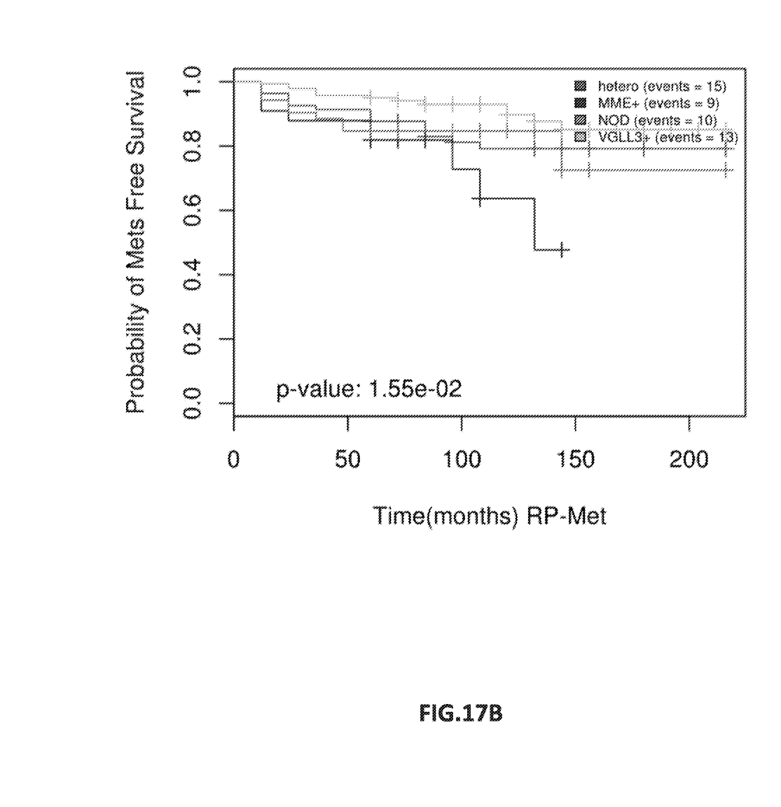

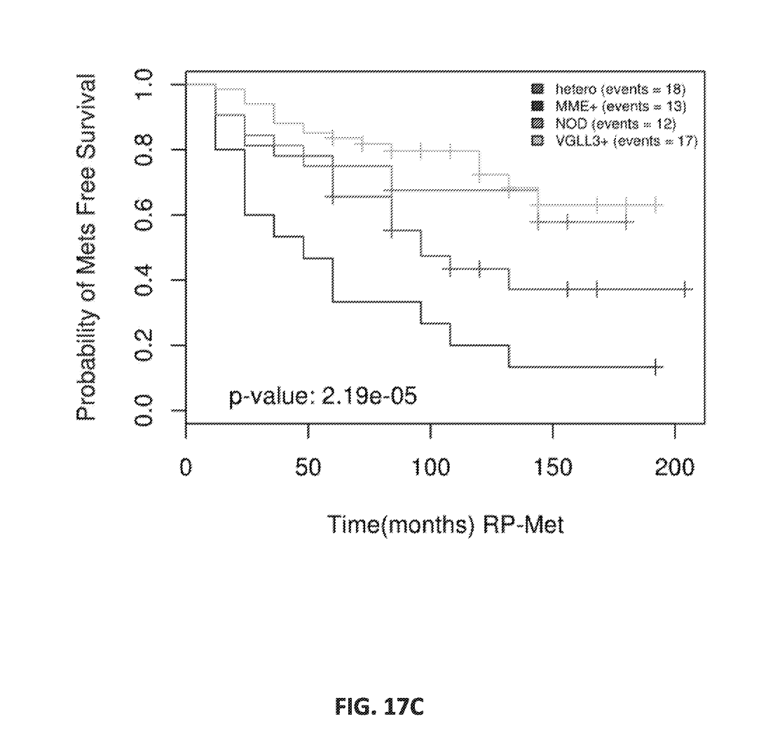

Based on survival analysis in the TripleNeg/m-SPINK+ subgroups in three case cohorts, VGLL3+ was associated with better outcome whereas NOD and hetero group showed no improvement (FIG. 17). These results suggest that VGLL3+ have a protective role in patients lacking the ERG, ETV1, ETV4 and ETV5 fusions. In univariable analysis, VGLL3+ was shown to be an independent prognostic biomarker of favorable outcome in the TripleNeg/SPINK+ subgroup (OR:0.5, p=0.049) (see Table 8). Additional clinical associations with the VGLL3+ subgroup demonstrated that VGLL3+ is associated with lack of SVI (OR:0.4, p=0.005) with reference to NOD and associated with lower pre-PSA (OR: 0.48, p=0.005) and lower path GS (OR: 0.43, p<0.001) with reference to hetero group (see Table 9).

TABLE 8

| | | 95% | |
|---|---|---|---|
| | | Confidence | p- |
| Variable | Estimate | Interval | value |
| hetero (Ref: NOD) | 0.825 | 0.443-1.536 | 0.543 |
| MME+(Ref: NOD) | 2.978 | 1.123-7.898 | 0.028 |
| VGLL3+(Ref: NOD) | 0.508 | 0.258-0.998 | 0.049 |
| LNI | 1.592 | 0.72-3.523 | 0.251 |
| SVI | 2.242 | 1.222-4.113 | 0.009 |
| EPE | 1.231 | 0.706-2.148 | 0.464 |
| SMS | 1.118 | 0.664-1.883 | 0.675 |
| Pre-Op PSA (Ref <20) | 1.058 | 0.579-1.936 | 0.854 |
| PathGS 4 + 3 (Ref: 6 or 3 + 4) | 2.22 | 0.906-5.442 | 0.081 |
| PathGS >7 (Ref: 6 or 3 + 4) | 5.091 | 2.754-9.411 | <0.001 |

MVA of clinical variables and subtypes in the TripleNeg/SPINK+ subgroup.

TABLE 9

| | Reference (NOD) | | | Reference (Hetero) | | |
|---|---|---|---|---|---|---|
| | | 95% Confidence | p- | | 95% Confidence | p- |
| Variable | Estimate | Interval | value | Estimate | Interval | value |
| LNI | 0.55 | 0.27-1.14 | 0.108 | 0.59 | 0.29-1.2 | 0.142 |
| SVI | 0.5 | 0.31-0.81 | 0.005 | 0.66 | 0.41-1.08 | 0.101 |
| EPE | 0.95 | 0.64-1.43 | 0.82 | 0.85 | 0.57-1.26 | 0.406 |
| SMS | 0.88 | 0.6-1.31 | 0.537 | 0.84 | 0.57-1.23 | 0.367 |
| Pre-Op PSA (Ref < 20) | 1.18 | 0.68-2.04 | 0.567 | 0.48 | 0.29-0.8 | 0.005 |
| PathGS 4 + 3 (Ref: 6 or | 1.06 | 0.62-1.83 | 0.823 | 0.78 | 0.45-1.35 | 0.371 |
| 3 + 4) | | | | | | |
| PathGS > 7 (Ref: 6 or | 1.04 | 0.65-1.68 | 0.866 | 0.43 | 0.27-0.69 | <0.001 |
| 3 + 4) | | | | | | |
| Age | 1.01 | 0.98-1.04 | 0.446 | 0.98 | 0.95-1.01 | 0.268 |
| Race (Ref: Caucasian) | 1.2 | 0.7-2.08 | 0.507 | 0.62 | 0.35-1.1 | 0.099 |

UVA of clinical associations with VGLL3+ subgroup with reference to NOD and hetero.

These results suggested that the methods and markers of the present invention would be useful for diagnosing, prognosing, determining the progression of cancer, or predicting benefit from therapy in a subject having cancer.

### Example 13: MME+ Subgroup is Associated with Unfavorable Outcome

Patients with MME+ were significantly associated with metastasis outcome (FIG. 17). MME+ defined a very aggressive subset of patients lacking the ERG and ETS gene fusions. MME+ was an independent prognostic marker in the TripleNeg/SPINK subset (OR:2.9, p=0.03) (see Table 8) suggesting that incorporating MME+ with ERG-based classifiers would define a very aggressive subtype of patients that require immediate post-operative therapy. UVA of clinical association with MME+ in the TripleNeg/SPINK1 (Table 10) showed that MME+ is associated with high path GS (OR:7.5, p<0.001) with reference to NOD, and associated with SVI (OR:1.9, p=0.04) and lower pre-PSA (OR:0.05, p=0.05), higher path GS (OR:3.15, p=0.003) and SVI (OR:2.5, p=0.003) with reference to hetero. These results suggest that MME+ and VGLL3+ defined subtypes, within the TripleNeg/SPINK subgroups, that are clinically and molecularly distinct.

TABLE 10

| | Reference (NOD) | | | Reference (Hetero) | | |
|---|---|---|---|---|---|---|
| | | 95% Confidence | p- | | 95% Confidence | p- |
| Variable | Estimate | Interval | value | Estimate | Interval | value |
| LNI | 1.15 | 0.5-2.65 | 0.747 | 1.31 | 0.57-3.02 | 0.519 |
| SVI | 1.88 | 1.03-3.43 | 0.038 | 2.48 | 1.36-4.51 | 0.003 |
| EPE | 1.42 | 0.77-2.6 | 0.261 | 1.17 | 0.65-2.09 | 0.598 |
| SMS | 0.75 | 0.42-1.33 | 0.32 | 0.72 | 0.41-1.25 | 0.241 |
| Pre-Op PSA (Ref < 20) | 1.02 | 0.47-2.22 | 0.961 | 0.47 | 0.22-1 | 0.051 |
| PathGS 4 + 3 (Ref: 6 or | 1.61 | 0.56-4.66 | 0.378 | 1.16 | 0.4-3.31 | 0.786 |
| 3 + 4) | | | | | | |
| PathGS > 7 (Ref: 6 or | 7.52 | 3.41-16.63 | <0.001 | 3.15 | 1.47-6.73 | 0.003 |
| 3 + 4) | | | | | | |
| Age | 0.98 | 0.94-1.02 | 0.348 | 0.95 | 0.91-0.99 | 0.028 |
| Race (Ref: Caucasian) | 2.31 | 0.96-5.56 | 0.063 | 1.09 | 0.54-2.2 | 0.801 |

UVA of clinical associations with MME+ subgroup with reference to NOD and hetero.

These results suggested that the methods and markers of the present invention would be useful for diagnosing, prognosing, determining the progression of cancer, or predicting benefit from therapy in a subject having cancer.

### Example 14: Hetero Group is Associated with Unfavorable Clinical Variables

Based on univariate analysis in the TripleNeg/SPINK1+ subgroup (Table 11), the hetero subgroup was associated with lack of SVI (OR:0.67, p=0.059), higher pre-PSA (OR:2.2, p=0.001) and higher gleason grade (OR:2.16, p=0.001). These results suggest that the hetero group is associated with unfavorable clinical variables confirming that it is clinical distinct from the NOD group.

TABLE 11

| | | 95% | |
|---|---|---|---|
| | | Confidence | p- |
| Variable | Estimate | Interval | value |
| LNI | 0.97 | 0.54-1.76 | 0.923 |
| SVI | 0.67 | 0.44-1.02 | 0.059 |
| EPE | 1.23 | 0.84-1.8 | 0.293 |
| SMS | 1.04 | 0.73-1.5 | 0.815 |
| Pre-Op PSA (Ref <20) | 2.2 | 1.37-3.52 | 0.001 |
| PathGS 4 + 3 (Ref: 6 or 3 + 4) | 1.36 | 0.76-2.42 | 0.297 |
| PathGS >7 (Ref: 6 or 3 + 4) | 2.16 | 1.39-3.35 | 0.001 |
| Age | 1.02 | 0.99-1.05 | 0.232 |
| Race (Ref: Caucasian) | 1.79 | 1.01-3.18 | 0.046 |

UVA of clinical associations with hetero subgroup with reference to NOD

These results suggested that the methods and markers of the present invention would be useful for diagnosing, prognosing, determining the progression of cancer, or predicting benefit from therapy in a subject having cancer.

### Example 15: GPR116 Defines an Aggressive Subset of ERG+ Patients

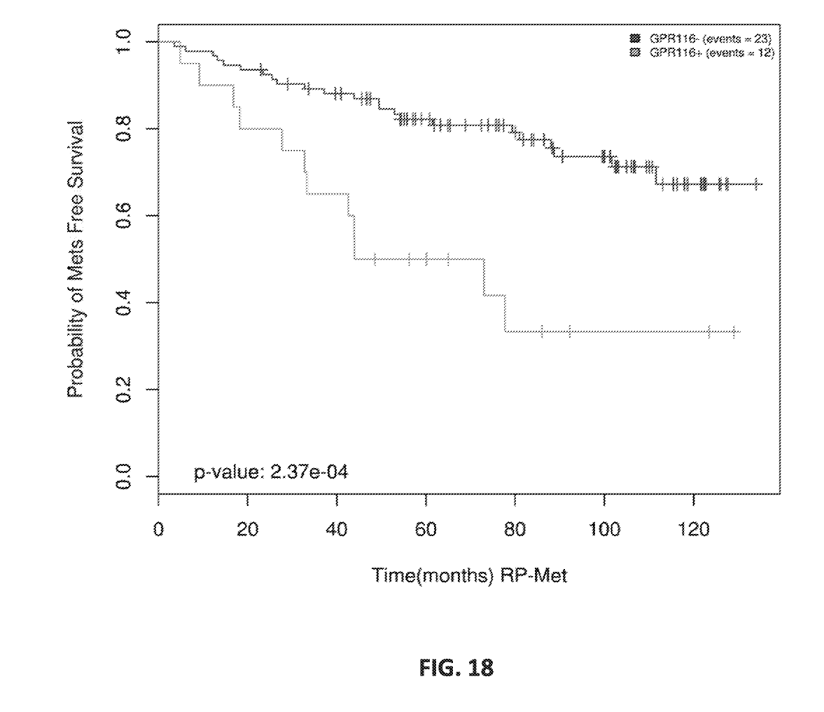

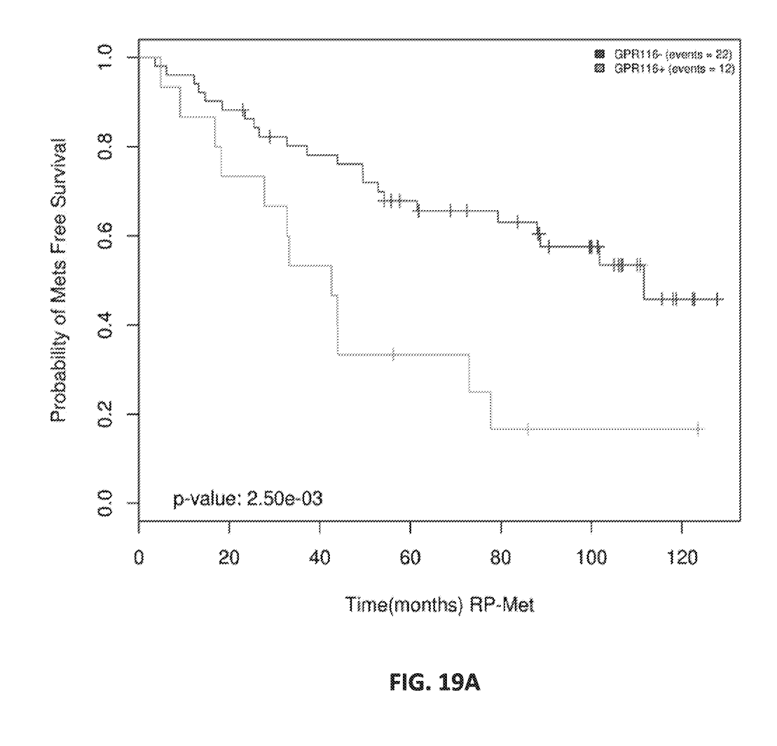

Patients with high GPR116 are a subset of ERG+ patients. We clinically characterized the association between GPR116 expression and metastasis in ERG+ subgroup. GPR116 positive prostate cancer samples were highly associated with metastasis in MC II (FIG. 18A) and GPR116 status was an independent prognostic biomarker (OR:1.7, p=0.11) (Table 12). UVA of clinical variables associated with GPR116 in ERG+ showed that GPR116+ is associated with EPE (OR:1.8, p=0.008) and higher Gleason Score (GS) (OR:1.57, p=0.05) (Table 13).

TABLE 12

| | | 95% | |
|---|---|---|---|
| | | Confidence | p- |
| Variable | Estimate | Interval | value |
| GPR116+ (Ref: GPR116−) | 1.742 | 0.877-3.459 | 0.113 |
| LNI | 2.352 | 0.987-5.604 | 0.054 |
| SVI | 1.39 | 0.721-2.681 | 0.325 |
| EPE | 1.056 | 0.535-2.085 | 0.875 |
| SMS | 0.824 | 0.444-1.529 | 0.54 |
| Pre-Op PSA (Ref <20) | 1.325 | 0.546-3.212 | 0.534 |
| PathGS 4 + 3 (Ref: 6 or 3 + 4) | 5.582 | 2.335-13.347 | <0.001 |
| PathGS >7 (Ref: 6 or 3 + 4) | 13.011 | 5.907-28.658 | <0.001 |

MVA of GPR116 and clinical variables in ERG+ after adjusting for treatment

TABLE 13

| | | 95% | |
|---|---|---|---|
| | | Confidence | p- |
| Variable | Estimate | Interval | value |
| LNI | 0.72 | 0.37-1.41 | 0.338 |
| SVI | 0.83 | 0.53-1.3 | 0.414 |
| EPE | 1.79 | 1.16-2.76 | 0.008 |
| SMS | 0.77 | 0.53-1.12 | 0.177 |
| Pre-Op PSA (Ref <20) | 0.98 | 0.54-1.79 | 0.951 |
| PathGS 4 + 3 (Ref: 6 or 3 + 4) | 1.22 | 0.68-2.18 | 0.501 |
| PathGS >7 (Ref: 6 or 3 + 4) | 1.57 | 1-2.47 | 0.053 |
| Age | 0.99 | 0.97-1.02 | 0.708 |
| Race (Ref: Caucasian) | 0.11 | 0.02-0.83 | 0.032 |

UVA of clinical variables associated with GPR116 in the ERG+ subset

### Example 16: GPR116 is a Predictive Biomarker of ADT Failure in ERG+ Patients

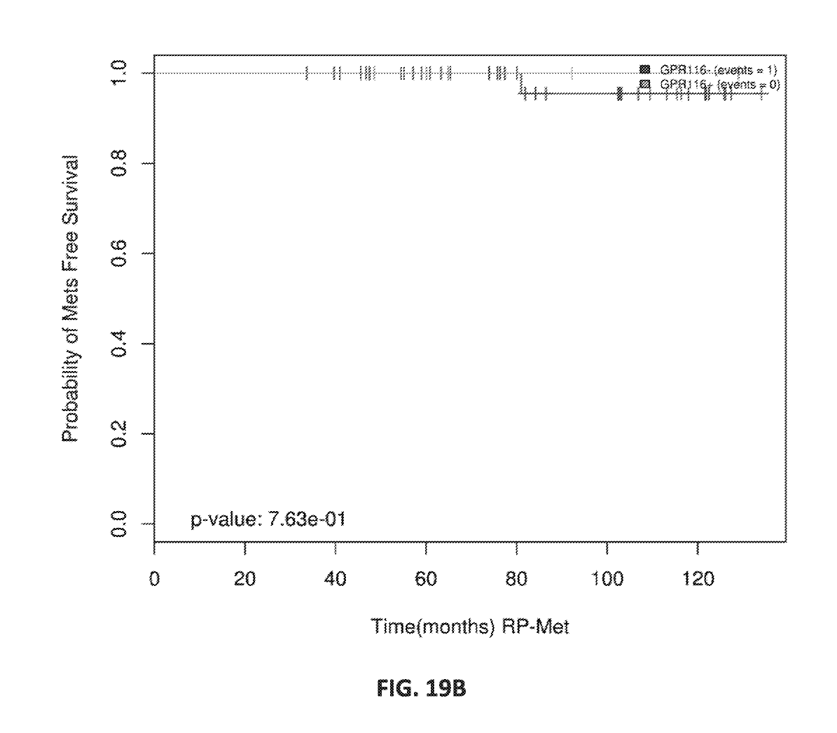

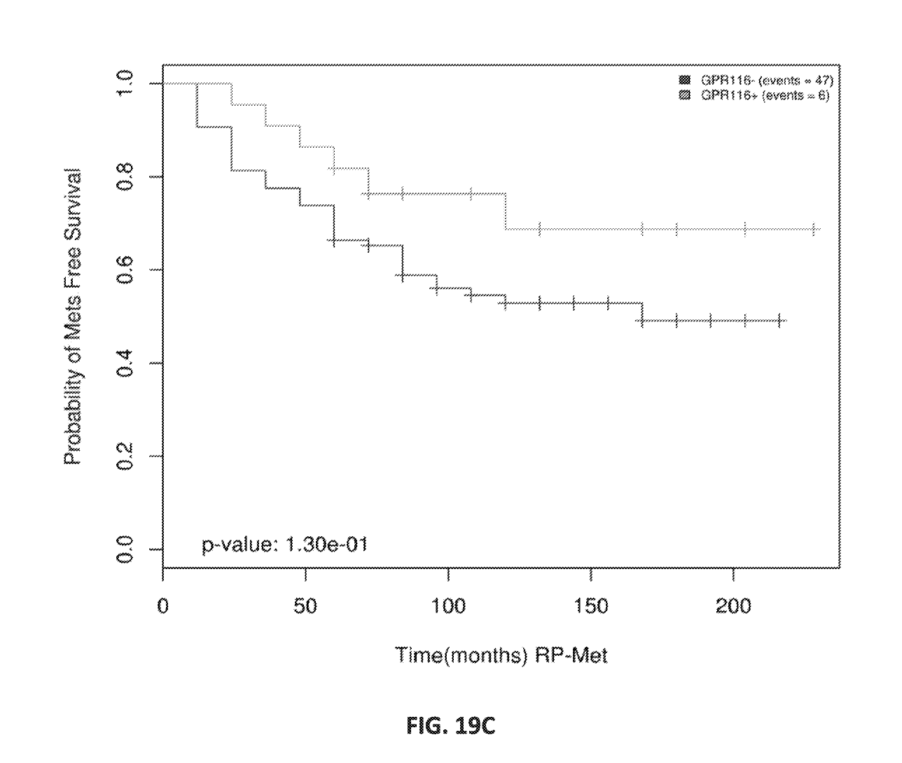

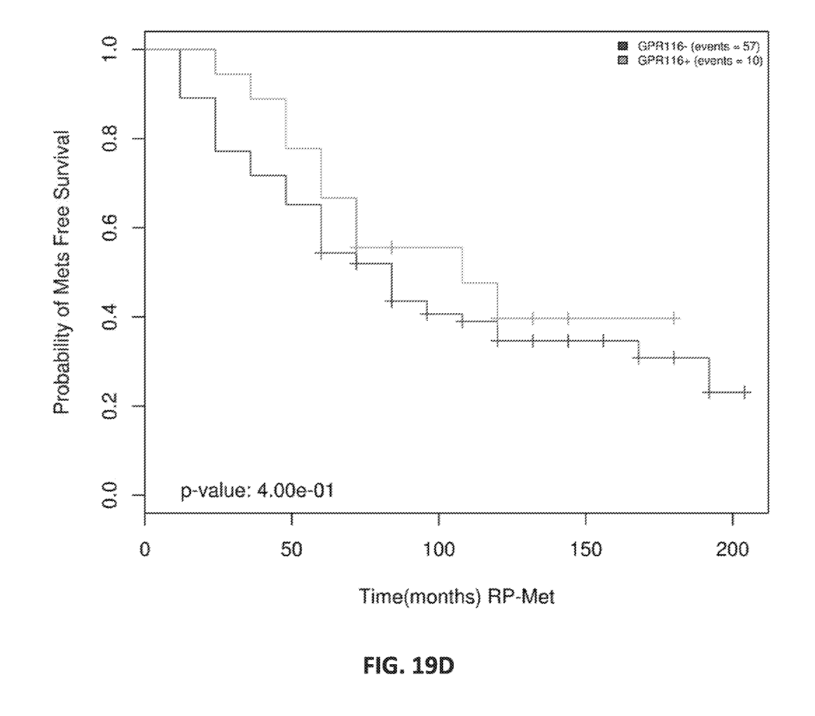

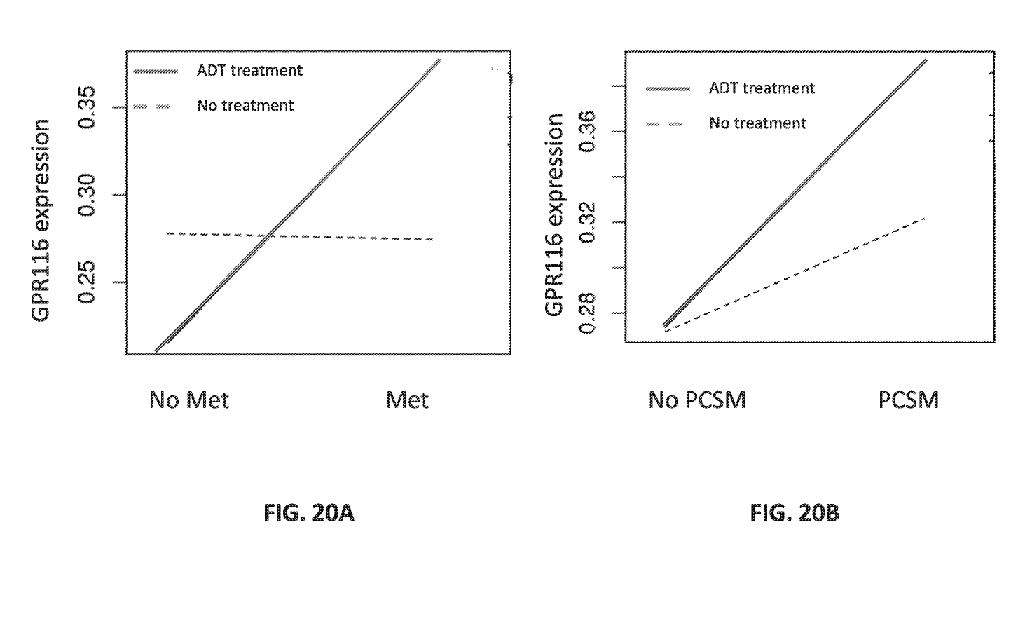

Evaluation of the prognosis of GPR116+ in ERG+ subset with hormonal (ADT) treatment in MCII dataset showed that patients with GPR116+ developed metastasis unlike GPR116− (see FIG. 19A). However, in patients with ERG+ that did not receive hormonal therapy from the same cohorts, GPR116+ was not associated with metastasis (FIG. 19B). To further confirm this observation, we evaluated the survival analysis of GPR116 in ERG+ in natural history cohorts with no treatment till the time of metastasis and found that GPR116 is not associated with metastasis (JHMI-RP: FIG. 19C & JHMI-BCR: FIG. 19D). Additionally, we found that GPR116+ is an independent prognostic biomarker in ERG+ with hormonal treatment (OR:5.1, p=0.02) (Table 14). When we evaluated the interaction between treatment and GPR116 in ERG+ after adjusting for clinical variables and found that the interaction is very significant (OR: 40, p=0.005) (FIGS. 20A and 20B). These results suggest that GPR116 is a predictive biomarker of ADT failure in the ERG+ subgroup and it adds independent prognostic information for metastasis in the ERG+ patients treated with ADT.

TABLE 14

| | | 95% | |
|---|---|---|---|
| | | Confidence | p- |
| Variable | Estimate | Interval | value |
| GPR116+ (Ref: GPR116−) | 5.136 | 1.285-20.525 | 0.021 |
| LNI | 2.075 | 0.646-6.66 | 0.22 |
| SVI | 0.438 | 0.141-1.361 | 0.153 |
| EPE | 0.649 | 0.201-2.098 | 0.47 |
| SMS | 0.794 | 0.236-2.668 | 0.709 |
| Pre-Op PSA (Ref <20) | 1.075 | 0.29-3.981 | 0.914 |
| PathGS 4 + 3 (Ref: 6 or 3 + 4) | 1.167 | 0.201-6.78 | 0.863 |
| PathGS >7 (Ref: 6 or 3 + 4) | 10.976 | 1.926-62.549 | 0.007 |

MVA of GPR116 and clinical variables in ERG+ treated with hormonal therapy

### Example 17: GPR116 and GRM7 are Overexpressed in ERG+ Prostate Cancers

GPR116 and GRM7 status for subtyping prostate cancer tissue was assessed as follows. The outlier analysis method was applied on a single cohort of 2,293 prostate cancer samples as described in Examples 1 and 2. Outlier genes expressed in the ERG+ subset were identified. A threshold was defined to classify patients as an outlier (or not) and then the defined threshold was used to classify the remaining samples from the cohort (n=2,293).

Two genes (GPR116 and GRM7) were identified as an outlier profile in the ERG+ subgroup (Table 15). Out of the 2,293 prostate cancer patients, 42% were ERG+. Beeswarm plots (FIGS. 21A and 21B) show the overexpression of GPR116 and GRM7 in ERG+ patients. From these, 22% showed high-expression of GPR116+ and 21% showed high expression of GRM7+, and 8% of ERG+ samples showed increased expression of GRM7 and GPR116. GPR116 and GRM7 defined a subgroup of 35% of the ERG+ samples.

TABLE 15

| Subtype | Gene |
|---|---|
| ERG+ | GPR116 |
| ERG+ | GRM7 |

Two outlier genes in ERG+ subgroup.

These results showed that GPR116 and GRM7 status could be used to identify ERG+ prostate cancer subjects. These results further showed that methods and markers of the present invention could be used to subtype prostate cancer. These results suggested that the methods and markers of the present invention would be useful for diagnosing, prognosing, determining the progression of cancer, or predicting benefit from therapy in a subject having cancer.

### Example 18: Listing of Targets

Table 16 is a listing of the sequences for the targets in Table 1, Table 2, Table 6, Table 7 and Table 15 and for targets having a sequence of SEQ ID NOs: 1-3348.

TABLE 16

| SEQ ID | | | |
|---|---|---|---|
| NO. | Gene | Probeset | Sequence |
| 1 | BANK1 | 2737595 | CCCTAGGAGTATGTGTAAAACTTGT |
| 2 | BANK1 | 2737595 | GTTAACTAGAGTCCTACACCCTAGG |
| 3 | BANK1 | 2737595 | CCGTAGTGATGAAGGTTAACTAGAG |
| 4 | BANK1 | 2737595 | GAGTCCTACACCCTAGGAGTATGTG |
| 5 | BANK1 | 2737594 | TGTTTGGTGAATGAGAACTTAAGAG |
| 6 | BANK1 | 2737594 | GTGAATGAGAACTTAAGAGAAGATC |
| 7 | BANK1 | 2737594 | ATTGTTTGGTGAATGAGAACTTAAG |
| 8 | BANK1 | 2737594 | TCTTAATTGTTTGGTGAATGAGAAC |
| 9 | BANK1 | 2737670 | CTTTGACGGGTGTTACTTTTATTCA |
| 10 | BANK1 | 2737670 | CCTTTGACGGGTGTTACTTTTATTC |
| 11 | BANK1 | 2737621 | GTCCGTTCCCCGAACCCTCGGGCCT |
| 12 | BANK1 | 2737621 | GGTCGTCGCGGTCCGTTCCCCGAAC |
| 13 | BANK1 | 2737621 | GTCGCGGTCCGTTCCCCGAACCCTC |
| 14 | BANK1 | 2737621 | TACGACGGTCGTCGCGGTCCGTTCC |
| 15 | BANK1 | 2737619 | ACTCGGGGCCCCGACGCAAAGGACT |
| 16 | BANK1 | 2737619 | CTCGGGGCCCCGACGCAAAGGACTC |
| 17 | BANK1 | 2737619 | TACTCGGGGCCCCGACGCAAAGGAC |
| 18 | BANK1 | 2737613 | GAAAGACGACTAAATCAATGGGTAC |
| 19 | BANK1 | 2737613 | AAAGACGACTAAATCAATGGGTACC |
| 20 | BANK1 | 2737613 | GTGAAAGACGACTAAATCAATGGGT |
| 21 | BANK1 | 2737613 | AGAGACGAATAGGTCGAAGTGAAAG |
| 22 | BANK1 | 2737649 | ATACTTCTCCTATAACGGAGTAAAA |
| 23 | BANK1 | 2737649 | AATACTTCTCCTATAACGGAGTAAA |
| 24 | BANK1 | 2737649 | TACTTCTCCTATAACGGAGTAAAAG |
| 25 | BANK1 | 2737636 | ACGAAGTTACAAGTCCTCGTTGGAC |
| 26 | BANK1 | 2737636 | TAACGACTTTCCGTACCAGTGTTTC |
| 27 | BANK1 | 2737636 | GGGCGTGTATAACGACTTTCCGTAC |
| 28 | BANK1 | 2737636 | AGAGGTGACACGTCGTTTTAAACCG |
| 29 | BANK1 | 2737652 | GCGGCGCTGGACATCGATTACGGAA |
| 30 | BANK1 | 2737652 | CCTTTCTGGAGTGAAGTGGAATGGT |
| 31 | BANK1 | 2737652 | GGTCATACTACTGAACATACACAAG |
| 32 | BANK1 | 2737652 | ACAAGTAAGGACCACGACTAGGTCT |
| 33 | BANK1 | 2737668 | TGAGTGGTAACACGTGGTAGGTCCA |
| 34 | BANK1 | 2737668 | TATTTGAGTGGTAACACGTGGTAGG |
| 35 | BANK1 | 2737668 | ATTTGAGTGGTAACACGTGGTAGGT |
| 36 | BANK1 | 2737620 | AGGTCCATCGCGAGCCGCCCGTCGT |
| 37 | BANK1 | 2737620 | AGAGACCGGCCCTCTCAGGTCCATC |
| 38 | BANK1 | 2737620 | ACGCGTCCGGGGAGCCGAAGTTGGC |
| 39 | BANK1 | 2737620 | TCTTTTAGCGCCCCTCAGAGACCGG |
| 40 | BANK1 | 2737650 | TTCTGTATGCCCGTCTCACGTCTAC |
| 41 | BANK1 | 2737650 | GTAGTACTTTCGTCCTTCTGTATGC |
| 42 | BANK1 | 2737650 | CTTTCGTCCTTCTGTATGCCCGTCT |
| 43 | BANK1 | 2737650 | GTCCTTCTGTATGCCCGTCTCACGT |
| 44 | BANK1 | 2737661 | TGAGAGTCCCCGACAGATTGACTAC |
| 45 | BANK1 | 2737661 | CCCGACAGATTGACTACCAGTCCTT |
| 46 | BANK1 | 2737661 | GACAGATTGACTACCAGTCCTTCTT |
| 47 | BANK1 | 2737661 | GAGTCCCCGACAGATTGACTACCAG |
| 48 | BANK1 | 2737628 | TAATCTTGTGCCGGTCGGGAAACCT |
| 49 | BANK1 | 2737628 | ATGTAGTTCATTATTCGCGTAATCT |
| 50 | BANK1 | 2737628 | TCTCTACTTCATTAACCACTATGAC |
| 51 | BANK1 | 2737628 | CATTATTCGCGTAATCTTGTGCCGG |
| 52 | BANK1 | 2737624 | TCTTTAAGTCGGATAAGAAACAAAA |
| 53 | BANK1 | 2737624 | AACGACCAAAACGAACGTAGACTAC |
| 54 | BANK1 | 2737624 | CGAACGTAGACTACTCATCTTTAAG |
| 55 | BANK1 | 2737624 | CCTGATGAACGAACGACCAAAACGA |
| 56 | BANK1 | 2737651 | TTTGTGTCGGGTGATCTCCAACCGT |
| 57 | BANK1 | 2737612 | GGTCTCGATACCACGTTTTCCGCCC |
| 58 | BANK1 | 2737612 | TCGATACCACGTTTTCCGCCCCAGC |
| 59 | BANK1 | 2737612 | TTCCGCCCCAGCGATCCCGGTGAGT |
| 60 | BANK1 | 2737612 | GAGGGTCTCGATACCACGTTTTCCG |
| 61 | BANK1 | 2737674 | GTCTCAAGGTCAGTAATAACAATGT |
| 62 | BANK1 | 2737674 | AAATTCATCGACCAAGTAAAAGACT |
| 63 | BANK1 | 2737674 | CCCGTGATTGGAGTTGTCTAATAAG |
| 64 | BANK1 | 2737674 | GTCTCTTCAATTTACGCCACATCGT |
| 65 | BANK1 | 2737656 | GACCCTCAGCCAGAAAGTAATATTT |
| 66 | BANK1 | 2737656 | GTACTATAACCGGTTAGACTCATAT |
| 67 | BANK1 | 2737656 | ATGAAAACGACTCTAACTACTGTCA |
| 68 | BANK1 | 2737656 | CTCCTTTGATGTGGAATGTATCGAG |
| 69 | BANK1 | 2737672 | ACTTTGAGTGCTTAGATGCCTGTAA |
| 70 | BANK1 | 2737672 | AATATTACTTTGAGTGCTTAGATGC |
| 71 | BANK1 | 2737672 | GTGCTTAGATGCCTGTAAAACGAAA |
| 72 | BANK1 | 2737672 | GATGCCTGTAAAACGAAAGTCCCAC |
| 73 | BANK1 | 2737675 | CATTTAAATATTCTTAATCGGTTAT |
| 74 | BANK1 | 2737675 | ATTTAAATATTCTTAATCGGTTATT |
| 75 | BANK1 | 2737675 | GGTTATTTTAACGAAGAGCCGGAAA |
| 76 | BANK1 | 2737675 | ATCGGTTATTTTAACGAAGAGCCGG |
| 77 | BANK1 | 2737627 | AGTTGTAAGGTTGTCTGGATGCTCG |
| 78 | BANK1 | 2737627 | ACCACGAAGGGTGACTTTAAGGTAC |
| 79 | BANK1 | 2737627 | GCTCGTTTTGTAAGACCCCTTTATT |
| 80 | BANK1 | 2737627 | TCTTTGTGGTATGGTGATCGTCACC |
| 81 | BANK1 | 2737655 | TCTTCAGTTTTGACCCCAGTAGGAC |
| 82 | BANK1 | 2737655 | CCCAGTAGGACCACAATCTGTTCTT |
| 83 | BANK1 | 2737655 | CCAGTAGGACCACAATCTGTTCTTT |
| 84 | BANK1 | 2737630 | CTACCTTAGCAATTTCGATGTTGGT |
| 85 | BANK1 | 2737630 | GTTACAGATGACACTACCTTAGCAA |
| 86 | BANK1 | 2737630 | GTTTCCTTACGGATAAGTCTTACCG |
| 87 | BANK1 | 2737630 | CGGATAAGTCTTACCGTCTAAGTCC |
| 88 | BANK1 | 2737671 | CTTAAACCAAAGACAACGTTCTTTC |
| 89 | BANK1 | 2737671 | ACGAGCTGGGGTTCAACTTTTCCTT |
| 90 | BANK1 | 2737671 | GGACGAGCTGGGGTTCAACTTTTCC |
| 91 | BANK1 | 2737671 | CAAAGACAACGTTCTTTCTAGTAAT |
| 92 | BANK1 | 2737673 | TCGCTTAAGTATGATACTGTCGTCT |
| 93 | BANK1 | 2737673 | GGACGAAGTATACCCATATAATGAT |
| 94 | BANK1 | 2737673 | ACGAGAGAAATTTCGCTTAAGTATG |
| 95 | BANK1 | 2737673 | TTCGAACTTAAACCTAACGGACGAG |
| 96 | BANK1 | 2737625 | CTACCCTCTAGAGTTGACTTGTCCT |
| 97 | BANK1 | 2737625 | CTCAACGACTTGAATTGCAGAATGT |
| 98 | BANK1 | 2737625 | GGACAATATAGCGAACCTCTTAAAG |
| 99 | BANK1 | 2737625 | GAGAAAAGCCGTAAACCTCAACGAC |
| 100 | BANK1 | 2737634 | GAACTACCACAGGAATGTAGGTATA |
| 101 | BANK1 | 2737634 | AAGTTTGTACTCTATGGTATAATAC |
| 102 | BANK1 | 2737634 | TAATACTCAAGGTCAGAGAAGTTTG |
| 103 | BANK1 | 2737634 | CAGGAATGTAGGTATAAGTTTGTAC |
| 104 | BANK1 | 2737658 | TACTGTTCAAGACACCAGAAGGATT |
| 105 | BANK1 | 2737658 | CTGTCGGTCTTCTGTTAGACTACTA |
| 106 | BANK1 | 2737663 | CTTAGTACATCTTAGGGACCGCAAC |
| 107 | BANK1 | 2737663 | TATGTGTACACAAATCCACGTCTGG |
| 108 | BANK1 | 2737663 | CCGTGGTGACCATCCTCTCTAGACA |
| 109 | BANK1 | 2737663 | CCGTCGTCGTTTGGTAGTGATACAT |
| 110 | BANK1 | 2737611 | ACACAGGTAGCGAGAGTCTCGTCGA |
| 111 | BANK1 | 2737664 | TGTTGATGCTCTGACGTAATAACCC |
| 112 | BANK1 | 2737664 | TAATGCTGTTGATGCTCTGACGTAA |
| 113 | BANK1 | 2737664 | GATGCTCTGACGTAATAACCCTTTT |
| 114 | BANK1 | 2737664 | CTCTTTAATGCTGTTGATGCTCTGA |
| 115 | ERG | 3931789 | TCGACCCCAACAGTAACTCTTTAAG |
| 116 | ERG | 3931789 | ATCAAGTCGTGGACCAGTGTTTAGT |
| 117 | ERG | 3931789 | ACTCTTTAAGATCAAGTCGTGGACC |
| 118 | ERG | 3931789 | TCGGAGGTATAAATACGGACCTTAC |
| 119 | ERG | 3931794 | AAAACGACGGGGTTTGGGTATGACC |
| 120 | ERG | 3931794 | ACGACGGGGTTTGGGTATGACCTTA |
| 121 | ERG | 3931794 | ACGGGGTTTGGGTATGACCTTAAGT |
| 122 | ERG | 3931794 | GGGTTTGGGTATGACCTTAAGTGGT |
| 123 | ERG | 3931859 | GTCTGAAAACTAGAATTACCAGTTC |
| 124 | ERG | 3931859 | GAGTCTGAAAACTAGAATTACCAGT |
| 125 | ERG | 3931859 | TATCTACACTGAAACTGAGTACAAG |
| 126 | ERG | 3931859 | TGAAAACTAGAATTACCAGTTCACG |
| 127 | ERG | 3931791 | GGAAATGTCATAATGGCCCTGATAC |
| 128 | ERG | 3931791 | AAGGCAAACTACCTGTCGACAGTCG |
| 129 | ERG | 3931791 | CTGTGCTCTCTCTGACACCGGGTAG |
| 130 | ERG | 3931791 | ACAGTCGAAAGAGTTTGACACTTCT |
| 131 | ERG | 3931797 | TACTGCCTAGGGCTGCTCCACCGGG |
| 132 | ERG | 3931797 | ACTGCCTAGGGCTGCTCCACCGGGC |
| 133 | ERG | 3931797 | GCCTAGGGCTGCTCCACCGGGCCGC |
| 134 | ERG | 3931797 | GCGGGAGGCAATGATGATACTGTTC |
| 135 | ERG | 3931785 | GGTCAGGTCCAATAATCGTTCAGAA |
| 136 | ERG | 3931785 | TGAACCTATTAGTGAGTCAAGAGAG |
| 137 | ERG | 3931785 | CAAGAGAGAAGTTCTGACAGAGTAC |
| 138 | ERG | 3931785 | ATTGTACTATTATGACTCAAGGAAG |
| 139 | ERG | 3931832 | CTACTTGATGCCGTCGATGTACCTC |
| 140 | ERG | 3931832 | ACCCGTCGGGTCTGTGGCAACCCTA |
| 141 | ERG | 3931832 | GCTCGCGTCTCAATAGCACGGTCGT |
| 142 | ERG | 3931832 | GGTTTGTACTGGTGCTTGCTCGCGT |
| 143 | ERG | 3931783 | GTCCACGTCGTCTCTACCGATGTCG |
| 144 | ERG | 3931783 | TCCACGTCGTCTCTACCGATGTCGA |
| 145 | ERG | 3931790 | AAAGGAAACTCAGCGCTTGCGACAC |
| 146 | ERG | 3931790 | ATGCTCAACTAGAGCCGGTCGGTTT |
| 147 | ERG | 3931790 | TTAGTGCGTCCGTAAAACCCATCCG |
| 148 | ERG | 3931790 | CGAACCGGATCGTACCGTTTAGTCT |
| 149 | ERG | 3931798 | GACCTCGAGGACAGCCTGTCGAGGT |
| 150 | ERG | 3931798 | ACAGCCTGTCGAGGTTGAGGTCGAC |
| 151 | ERG | 3931798 | CAGCCTGTCGAGGTTGAGGTCGACG |
| 152 | ERG | 3931798 | TCGAGGACAGCCTGTCGAGGTTGAG |
| 153 | ERG | 3931782 | AGCACTCCACTGATTAATCTCTTAT |
| 154 | ERG | 3931782 | TTATTTCAGCACTCCACTGATTAAT |
| 155 | ERG | 3931782 | ACGTCGCGGGGTTTCACTGGGTAAC |
| 156 | ERG | 3931782 | TTCGATCAAATAAATCGAAGAGTAA |
| 157 | ERG | 3931824 | ACCGGAAGGTCTGCAGTTGTAGAAC |
| 158 | ERG | 3931824 | AATAAGGTCTTGTAGCTACCCTTCC |
| 159 | ERG | 3931824 | TCATGTCTGGTACACGCCGTCACCG |
| 160 | ERG | 3931824 | CACGTTCTACTGGTTCCTGCTGAAG |
| 161 | ERG | 3931793 | GAAGAGTAGACCCGTGAATGATGAT |
| 162 | ERG | 3931793 | GGAAGAGTAGACCCGTGAATGATGA |
| 163 | ERG | 3931787 | AGATCTCAGTCAAAGGGACCCGTAG |
| 164 | ERG | 3931787 | GGACTACAACGACCGATAGGGAACT |
| 165 | ERG | 3931787 | ACGCTTCCGCGATCGGCTTTGTAGA |
| 166 | ERG | 3931787 | GTCCTCGAGAGTGATCCATCTGTCG |
| 167 | ERG | 3931812 | CTCTAGTCGGACCTGGCCAGTGCCG |
| 168 | ERG | 3931812 | GTGCCGGTGGGGTGCGGGGTCAGCT |
| 169 | ERG | 3931812 | TAGTCGGACCTGGCCAGTGCCGGTG |
| 170 | ERG | 3931812 | AAATGGTATACTCGGGGGGTCCTCT |
| 171 | ERG | 3931792 | TCCCTCAATGACTTCAGAATGATGT |
| 172 | ERG | 3931792 | CTCCGAAAAGGGTAGTCGCACGTAA |
| 173 | ERG | 3931792 | ACCTGTATAGTAGACACCTGACTGG |
| 174 | ERG | 3931792 | GGTAGCGGTGTTTGAGATAGCCTCT |
| 175 | ERG | 3931819 | ACGGGATTCAGTGCACTATGTTTCT |
| 176 | ERG | 3931819 | GACGGAGACAACTAAACCTCTGATT |
| 177 | ERG | 3931819 | ACAGGACGACTCTAGGCACGGGATT |
| 178 | ERG | 3931819 | CTTCGGTCAGGGTCTGTCAGAATAA |
| 179 | ERG | 3931809 | TGTTCATCGGCGGAACGTTTAGGTC |
| 180 | ERG | 3931809 | CTGGTTGTTCATCGGCGGAACGTTT |
| 181 | ERG | 3931809 | TAAGAACCTGGTTGTTCATCGGCGG |
| 182 | ERG | 3931809 | GAATAGTCTAAGAACCTGGTTGTTC |
| 183 | ERG | 3931796 | CAAGCTGAAGGTGCCCTAGCGGGTC |
| 184 | ERG | 3931796 | GATGTTCAAGCTGAAGGTGCCCTAG |
| 185 | ERG | 3931796 | GGTACCCTTCGCGATGCGGATGTTC |
| 186 | ERG | 3931796 | CGATGCGGATGTTCAAGCTGAAGGT |
| 187 | ERG | 3931786 | CACTTCAACGGTTTGGAGACACGAC |
| 188 | ERG | 3931786 | ACGGGCATAGAGGAATCCCTTTTAT |
| 189 | ERG | 3931786 | GAAACTTCAGCCGTCCTGTGCTAAT |
| 190 | ERG | 3931786 | GCCTCGGGTTGGTAGGTAGTAAAAC |
| 191 | ERG | 3931810 | GTTTTGACTTCTGGTCGCAGGAGTC |
| 192 | ERG | 3931810 | ACGGGTTTTGACTTCTGGTCGCAGG |
| 193 | ERG | 3931810 | GGTTTTGACTTCTGGTCGCAGGAGT |
| 194 | ERG | 3931810 | CGGGTTTTGACTTCTGGTCGCAGGA |
| 195 | ERG | 3931849 | AACAAACTCACACGGATGCCTTGCG |
| 196 | ERG | 3931849 | CAAACTCACACGGATGCCTTGCGGT |
| 197 | ERG | 3931849 | AACACTCACTCCTGGTCAGCAACAA |
| 198 | ERG | 3931849 | TCACTCCTGGTCAGCAACAAACTCA |
| 199 | ERG | 3931850 | ATATGTACGATTGATTCCGTCGACG |
| 200 | ERG | 3931850 | GTACGATTGATTCCGTCGACGGATG |
| 201 | ERG | 3931850 | GGATGGAACCGGCCGTCCATCCGTC |
| 202 | ERG | 3931850 | TTGTTAGATATGTACGATTGATTCC |
| 203 | ERG | 3931820 | AAAAGGGTTTATGAAGTCATATAGG |
| 204 | ERG | 3931820 | TGCGTTTCTTAATGTTGATCCGGTC |
| 205 | ERG | 3931820 | GACTTCGATGCGTTTCTTAATGTTG |
| 206 | ERG | 3931820 | TAGGACTTCGATGCGTTTCTTAATG |
| 207 | ERG | 3931788 | CCGTTTATTTCGCAGTACCTATCGA |
| 208 | ERG | 3931788 | ACCGTTTATTTCGCAGTACCTATCG |
| 209 | ERG | 3931848 | GAGTCGTCCTAACCGACAGAGTTGG |
| 210 | ERG | 3931848 | TCTGAAGGTTCTACTCGGGTGCGCA |
| 211 | ERG | 3931848 | AGGAGGTCGCTGATACCTGTCTGAA |
| 212 | ERG | 3931848 | ACCTTACATTGGGATCGGTCCACTT |
| 213 | ERG | 3931833 | TTGAGAGGACTACTTACGTCACACC |
| 214 | ERG | 3931833 | GAGGACTACTTACGTCACACCGGTT |
| 215 | ERG | 3931833 | TACGTCACACCGGTTTCCGCCCTTC |
| 216 | ERG | 3931833 | GACTACTTACGTCACACCGGTTTCC |
| 217 | ERG | 3931822 | AATGTTTTGAGAGGTGCCAATTACG |
| 218 | ERG | 3931822 | GAGAAGGTGTAAACTGAAGTCTACT |
| 219 | ERG | 3931822 | ACTACAACTATTTCGGAATGTTTTG |
| 220 | ERG | 3931822 | GCCAATTACGTACGATCTTTGTGTC |
| 221 | ERG | 3931784 | AGAACCGAACGGGACTACATATGAG |
| 222 | ERG | 3931784 | GAACAGAAGTTAACCGAAAGCCCGG |
| 223 | ERG | 3931784 | AAGCCCGGAACATACACCATTTTAG |
| 224 | ERG | 3931784 | CATGTTAGAATGAGGACGACCGTTC |
| 225 | ERG | 3931860 | CAACTGTTCTTAACGGGGAGGTTCT |
| 226 | ERG | 3931860 | TACGTGTCAACTGTTCTTAACGGGG |
| 227 | ERG | 3931860 | AACGGGGAGGTTCTAGAGTAACGAC |
| 228 | ERG | 3931860 | CTTAACGGGGAGGTTCTAGAGTAAC |
| 229 | ERG | 3931795 | TGTCTTCTACTTGAAACACCGCGGG |
| 230 | ERG | 3931795 | TCTGGAGGGCATGTACCCGAGGATA |
| 231 | ERG | 3931795 | AGGATAGTGCGGGTGGGTGTCTTCT |
| 232 | ERG | 3931795 | CTACTTGAAACACCGCGGGGTGGGA |
| 233 | ERG | 3931865 | TTTTGATGAAAGACCAGTCTCTCTT |
| 234 | ERG | 3931865 | TAGAGTAGGCGAGATTTGTTGGAGT |
| 235 | ERG | 3931865 | AATTGCTAGTTATTTGAACTAGCGT |
| 236 | ERG | 3931865 | ATGAAAGACCAGTCTCTCTTCGTTA |
| 237 | ERG | 3931877 | AGGGCCTGGGTCGTCGAGTATAGTT |
| 238 | ERG | 3931877 | CAGGGCCTGGGTCGTCGAGTATAGT |
| 239 | ERG | 3931877 | TCTGACAGGGCCTGGGTCGTCGAGT |
| 240 | ERG | 3931877 | GACAGGGCCTGGGTCGTCGAGTATA |
| 241 | ERG | 3931878 | CTGGGCTCCTTTCGGCACAACTGGT |
| 242 | ERG | 3931878 | CCTAGAAACCTCTGGGCTCCTTTCG |
| 243 | ERG | 3931878 | TCCTTTCGGCACAACTGGTTTTCGT |
| 244 | ERG | 3931878 | GAAACCTCTGGGCTCCTTTCGGCAC |
| 245 | ERG | 3931893 | TACTCTCTTCTCCTCGCCGCGAGTC |
| 246 | ERG | 3931894 | GCGACGCCCTGTCCAAGGATCTCTA |
| 247 | ERG | 3931894 | TTACCCCTCTCACACGTTCTCTAGC |
| 248 | ERG | 3931894 | GTTCTCTAGCGACGCCCTGTCCAAG |
| 249 | ERG | 3931894 | GATCTCTAGCGAGGCCCTGCCAGCA |
| 250 | ETV1 | 3039189 | GGTCCGTCAAAATACTACTGTGGAC |
| 251 | ETV1 | 3039189 | ACATACAAACTTTTCCCGGGGTCCG |
| 252 | ETV1 | 3039189 | ATACTACTGTGGACACAACAGGGTC |
| 253 | ETV1 | 3039189 | ACACAACAGGGTCTTTTTAAGCTAC |
| 254 | ETV1 | 3039191 | CGGTGAGGTAAATATACTCCGTTCT |
| 255 | ETV1 | 3039191 | GAGGTAAATATACTCCGTTCTTCCG |
| 256 | ETV1 | 3039191 | TGAGGTAAATATACTCCGTTCTTCC |
| 257 | ETV1 | 3039191 | GGTGAGGTAAATATACTCCGTTCTT |
| 258 | ETV1 | 3039211 | GTTCTAGATTCAGTTAATGTCCTTT |
| 259 | ETV1 | 3039211 | AGTTCTAGATTCAGTTAATGTCCTT |
| 260 | ETV1 | 3039211 | TTCTAGATTCAGTTAATGTCCTTTG |
| 261 | ETV1 | 3039200 | TGAGTATGTGGCTTTGGACTGGCCC |
| 262 | ETV1 | 3039200 | GACTGGCCCGGAAGGGTCGAGTGGA |
| 263 | ETV1 | 3039200 | GGTGTGGTAGGTCGTGCGGTCACAG |
| 264 | ETV1 | 3039200 | AGGGAGGTAGCGTCAGGTATGGTCT |
| 265 | ETV1 | 3039217 | GTCACTAAACCTATTCCGTATCAAA |
| 266 | ETV1 | 3039217 | AGTCACTAAACCTATTCCGTATCAA |
| 267 | ETV1 | 3039217 | AAGTCACTAAACCTATTCCGTATCA |
| 268 | ETV1 | 3039222 | AACACTTTCTCTGCGCCTCGGTTAC |
| 269 | ETV1 | 3039222 | CCGTCGCTAGGTAGTCAAACCTAAC |
| 270 | ETV1 | 3039222 | TAACTGTCGGGCTTTAGACTAGAAC |
| 271 | ETV1 | 3039222 | CGTCGTTCGGCGGACTAACTGTCGG |
| 272 | ETV1 | 3039213 | ACACACTAGACTCCAAATGTAAGAA |
| 273 | ETV1 | 3039213 | TAGACTCCAAATGTAAGAAAATTTC |
| 274 | ETV1 | 3039213 | CACTAGACTCCAAATGTAAGAAAAT |
| 275 | ETV1 | 3039213 | CACACTAGACTCCAAATGTAAGAAA |
| 276 | ETV1 | 3039178 | CAGACCATTAGTGTAGTTCGGAAAT |
| 277 | ETV1 | 3039178 | GTTAGAGACGAAGGTACCAGTGTAT |
| 278 | ETV1 | 3039178 | GATCAAAGGGCATCTACGACATTGG |
| 279 | ETV1 | 3039178 | CAATAAGTCTTGTGGCGTGCCTCCT |
| 280 | ETV1 | 3039187 | CATATACAGAGAGATGACTGGTATC |
| 281 | ETV1 | 3039187 | CTCCATATACAGAGAGATGACTGGT |
| 282 | ETV1 | 3039187 | AATTCTCCATATACAGAGAGATGAC |
| 283 | ETV1 | 3039187 | TAAAATTCTCCATATACAGAGAGAT |
| 284 | ETV1 | 3039179 | AACAATACAGGTACTTTTCACGAAG |
| 285 | ETV1 | 3039179 | AGAGTAACTTAACCGATGAGTTTGT |
| 286 | ETV1 | 3039179 | ACTACTGTACAATTATGGGTTATCT |
| 287 | ETV1 | 3039179 | CGACAAACGACAGAGAACTACTACT |
| 288 | ETV1 | 3039184 | TACTATTTGAATCGGCAAGTGAGGC |
| 289 | ETV1 | 3039184 | ACCGGGCTGCAACCCCGTAAGTCTT |
| 290 | ETV1 | 3039184 | ATAATGATACTCTTTCCTTAATACG |
| 291 | ETV1 | 3039184 | AATCGGCAAGTGAGGCGATAATGAT |
| 292 | ETV1 | 3039212 | CTTTTAAGTAATTGTCTCTAGACCG |
| 293 | ETV1 | 3039212 | TTAAGTAATTGTCTCTAGACCGAGT |
| 294 | ETV1 | 3039212 | AGTAATTGTCTCTAGACCGAGTACT |
| 295 | ETV1 | 3039212 | ATTGTCTCTAGACCGAGTACTAAGT |
| 296 | ETV1 | 3039182 | TGTACCTTGCAGTGTAGTTGCTCCT |
| 297 | ETV1 | 3039182 | GGGATGTTGCTTCCGATGCACATAA |
| 298 | ETV1 | 3039182 | AGAGGTACCGGAAAGGTCTATTAGT |
| 299 | ETV1 | 3039182 | GAAACTACTCTCGTACCGGATGTAC |
| 300 | ETV1 | 3039199 | CAGGATACATGGTTGCGGTCTACAG |
| 301 | ETV1 | 3039199 | ATTCGTCCTCATGGTGCTGGGTCAC |
| 302 | ETV1 | 3039199 | CGGTCGAAAGACTTGGGACATTGAG |
| 303 | ETV1 | 3039199 | AGGAGGAAACGGCTGCTACGGTTCC |
| 304 | ETV1 | 3039176 | TGTTCACATATATGGCACCGATAAC |
| 305 | ETV1 | 3039176 | ACGTATATCTGAGGTCATAATCAAT |
| 306 | ETV1 | 3039176 | TCCATCCCAGAAAAAACGTATATCT |
| 307 | ETV1 | 3039176 | GTCTCGAGTTGATCATGAAAATCCT |
| 308 | ETV1 | 3039185 | TAAATTTGACTAACTCGGACTTCTC |
| 309 | ETV1 | 3039185 | CGAGAAGACCTACTGGGAAGTTTAA |
| 310 | ETV1 | 3039185 | GACCAGCTCCGTACCTTAAATTTGA |
| 311 | ETV1 | 3039185 | ATGGTTGCCGCTCCTAGTGAAGTCG |
| 312 | ETV1 | 3039204 | TGTCTTACTCTTTAGATGAGTTACT |
| 313 | ETV1 | 3039204 | ACTTCGGACTGAATGTTGTCTTACT |
| 314 | ETV1 | 3039204 | TTCGGACTGAATGTTGTCTTACTCT |
| 315 | ETV1 | 3039204 | CTTCGGACTGAATGTTGTCTTACTC |
| 316 | ETV1 | 3039223 | CGACTCCTGGGTCGCGGATGGCCGG |
| 317 | ETV1 | 3039223 | TCCTGGGTCGCGGATGGCCGGCTCG |
| 318 | ETV1 | 3039223 | TGGGTCGCGGATGGCCGGCTCGTGG |
| 319 | ETV1 | 3039223 | GCGGATGGCCGGCTCGTGGGGGATC |
| 320 | ETV1 | 3039221 | TTCACGACCCGATATTAATTACAAA |
| 321 | ETV1 | 3039221 | TCTCCGCGAAAGCCGAAGGTTCCCC |
| 322 | ETV1 | 3039221 | CCTTCACGACCCGATATTAATTACA |
| 323 | ETV1 | 3039221 | CGGAAAGCGGATCGCACCGGAAGTC |
| 324 | ETV1 | 3039220 | TGTAGCGGAGAACAAGCCTAAAAAC |
| 325 | ETV1 | 3039220 | GTTCAGAGCAACTAGCGGTAACGAT |
| 326 | ETV1 | 3039220 | GTGTGCAAACGCTTAGTCTCGACGG |
| 327 | ETV1 | 3039220 | GCGCGTCCCTTTGTAGCTCTCACAT |
| 328 | ETV1 | 3039218 | CGTTCACGGAATGTACCAGTGGTTA |
| 329 | ETV1 | 3039181 | GAGACCGCGGTTTGACTCAGTATCC |
| 330 | ETV1 | 3039181 | GTTCGTCCCGCAAAAACGCGAAAAG |
| 331 | ETV1 | 3039181 | CGGTACCTGACACGTGAAATAAACT |
| 332 | ETV1 | 3039181 | AATGCACATAGACCACGGTGGAACG |
| 333 | ETV1 | 3039207 | CAAACATGGTCTGATAGTCCGACTT |
| 334 | ETV1 | 3039207 | AACATGGTCTGATAGTCCGACTTTC |
| 335 | ETV1 | 3039207 | CATGGTCTGATAGTCCGACTTTCAA |
| 336 | ETV1 | 3039207 | TCAAACATGGTCTGATAGTCCGACT |
| 337 | ETV1 | 3039210 | CCGTTCTTGATTACGTGGTTCTGAA |
| 338 | ETV1 | 3039210 | AGTGTAGACGAAAACCGTTCTTGAT |
| 339 | ETV1 | 3039210 | TGTTAGTGTAGACGAAAACCGTTCT |
| 340 | ETV1 | 3039210 | TTACGTGGTTCTGAAGTTCAAGATT |
| 341 | ETV1 | 3039183 | CGAGAACCAAAACCATAATGTTCGG |
| 342 | ETV1 | 3039183 | ATTTGCGGTGTATAGTAACGTAACG |
| 343 | ETV1 | 3039183 | GTAACGACTTCGCTCAAAAAGTGAG |
| 344 | ETV1 | 3039183 | TCCGAAAGACCTTCCAGGATGAAAC |
| 345 | ETV1 | 3039209 | AGAAGAAAGGTGGAACAAGTGTTGT |
| 346 | ETV1 | 3039209 | TACGAAGTTCTAAATTCACGTTCAC |
| 347 | ETV1 | 3039209 | TAAATTCACGTTCACAGAAGAAAGG |
| 348 | ETV1 | 3039209 | CAGAAGAAAGGTGGAACAAGTGTTG |
| 349 | ETV1 | 3039202 | AAAAGTACCGGACGGTGACTTTTAG |
| 350 | ETV1 | 3039202 | AGTACCGGACGGTGACTTTTAGTTC |
| 351 | ETV1 | 3039202 | CGAAAAGTACCGGACGGTGACTTTT |
| 352 | ETV1 | 3039202 | AAGTACCGGACGGTGACTTTTAGTT |
| 353 | ETV1 | 3039214 | ACGATTCTAGCCGTGACCCTTCGTT |
| 354 | ETV1 | 3039214 | TGAGAGGGACGGACGAATTGTATTC |
| 355 | ETV1 | 3039214 | CTAGGTAATCTGAATACATACGTAC |
| 356 | ETV1 | 3039214 | CAACAAAACTACGACTCTATGGTAC |
| 357 | ETV1 | 3039201 | ACGTCAGTTCTTGTCGGGAAATTTA |
| 358 | ETV1 | 3039201 | CTCTTTTCACGGACATGTTACAGTC |
| 359 | ETV1 | 3039201 | TCTTGTCGGGAAATTTAAGTCGATA |
| 360 | ETV1 | 3039201 | AGACGGACGTCAGTTCTTGTCGGGA |
| 361 | ETV1 | 3039180 | TAACCCGGTACGATTGCAATAGTGT |
| 362 | ETV1 | 3039180 | GTCCATCTAATTATTTAGACCGTCG |
| 363 | ETV1 | 3039180 | CATCACTGAGTGACTTGATTTATGT |
| 364 | ETV1 | 3039180 | AAAACGACAAAATTGCATCACTGAG |
| 365 | ETV1 | 3039227 | CTCGAAGTGACAAGTCGGAGCCCCG |
| 366 | ETV1 | 3039227 | CGTCAAGGGCGAGTTTTACGAATAT |
| 367 | ETV1 | 3039227 | CCCGGGTCCGCGAAGGACCTTAGAG |
| 368 | ETV1 | 3039227 | GGAGTCTATCATGGGTACTCGAAGT |
| 369 | ETV1 | 3039226 | TCTCCTTCACTTTCGCAGTTCATGT |
| 370 | ETV1 | 3039226 | CGGGAGTGACGAATTGCAGGATCAA |
| 371 | ETV1 | 3039226 | ACTTTGGGCTCGGTAGAGTGGCGAG |
| 372 | ETV1 | 3039226 | GAATTGCAGGATCAATAACAGGAAC |
| 373 | ETV4 | 3758529 | GGCCGGCACGCCGGCCTCCCTCGCC |
| 374 | ETV4 | 3758529 | CCGGCACGCCGGCCTCCCTCGCCGG |
| 375 | ETV4 | 3758529 | GGGCCGGCACGCCGGCCTCCCTCGC |
| 376 | ETV4 | 3758529 | GGCACGCCGGCCTCCCTCGCCGGCC |
| 377 | ETV4 | 3758524 | ATGGTCTGTCACTACTCGTCAAACA |
| 378 | ETV4 | 3758524 | CTACTCGTCAAACAAGGACTAAAGG |
| 379 | ETV4 | 3758524 | CAAGGACTAAAGGTAAGTCTTTTGG |
| 380 | ETV4 | 3758524 | GTCTGTCACTACTCGTCAAACAAGG |
| 381 | ETV4 | 3758526 | TCGAACGCGCTTCGCGACTAGCCGG |
| 382 | ETV4 | 3758526 | TTTAGCGGGCCTTTACCCTCGAACG |
| 383 | ETV4 | 3758526 | GCTTCGCGACTAGCCGGGCGACCCC |
| 384 | ETV4 | 3758526 | CGGGCCTTTACCCTCGAACGCGCTT |
| 385 | ETV4 | 3758511 | GGGCCAAACAGTCAAGAACCACGAG |
| 386 | ETV4 | 3758511 | ACAACCCCTTTGGAAGTAGACTTTG |
| 387 | ETV4 | 3758511 | AGGGTGACGCCCCTCTGTCTTCGGA |
| 388 | ETV4 | 3758511 | TTCCGCGAAGGGTTGAAGTATGACC |
| 389 | ETV4 | 3758527 | GATTCGCGGAGTCCCACTGAGCGCC |
| 390 | ETV4 | 3758527 | GCGGAGTCCCACTGAGCGCCCGTAA |
| 391 | ETV4 | 3758527 | TGATTCGCGGAGTCCCACTGAGCGC |
| 392 | ETV4 | 3758527 | GTCCCACTGAGCGCCCGTAAGAGGG |
| 393 | ETV4 | 3758513 | CGAGGCTATGATAATACTCTTTCCG |
| 394 | ETV4 | 3758513 | AGCGAGGCTATGATAATACTCTTTC |
| 395 | ETV4 | 3758513 | GCTATGATAATACTCTTTCCGTAGT |
| 396 | ETV4 | 3758513 | GCGAGCGAGGCTATGATAATACTCT |
| 397 | ETV4 | 3758519 | ACATGGAGGTGTGTCTCCCGAAGAG |
| 398 | ETV4 | 3758519 | TACATGGAGGTGTGTCTCCCGAAGA |
| 399 | ETV4 | 3758516 | ACGTAAAGCTCTCCCCGGCGGGATG |
| 400 | ETV4 | 3758516 | GGTAAAGTAACGGACCTGCCCGGCC |
| 401 | ETV4 | 3758516 | CGGCCCCTTACCTCAAGTTCGAGTA |
| 402 | ETV4 | 3758516 | ACCTACTGGGTTGTTTACGGGTAAA |
| 403 | ETV4 | 3758521 | TGTCTGCCTGAAGCGGATGCTGAGT |
| 404 | ETV4 | 3758521 | CCCACCACTAGTTTGTCCTTGTCTG |
| 405 | ETV4 | 3758521 | AGTTACCCGTGTCCATGGGTCCCCG |
| 406 | ETV4 | 3758521 | GGACATACTTGTCCGCCCGGTCGGT |
| 407 | ETV4 | 3758532 | CGGGCTTTTTGTTCAGCCACGCGAC |
| 408 | ETV4 | 3758532 | CGACGCGGGCTTTTTGTTCAGCCAC |
| 409 | ETV4 | 3758532 | GGCTTTTTGTTCAGCCACGCGACCC |
| 410 | ETV4 | 3758532 | AGACGACGCGGGCTTTTTGTTCAGC |
| 411 | ETV4 | 3758531 | GTCTTTGCCGCTCGGGCCGAGGACC |
| 412 | ETV4 | 3758531 | GGGCCCATTTCGTCCCGACGTCTTT |
| 413 | ETV4 | 3758531 | CGTCTTTCGTCTTTGCCGCTCGGGC |
| 414 | ETV4 | 3758531 | GGCCCATTTCGTCCCGACGTCTTTC |
| 415 | ETV4 | 3758528 | CTATGAACCTGGTCGTTCACGGGAT |
| 416 | ETV4 | 3758528 | ACCTGGTCGTTCACGGGATGTGGAA |
| 417 | ETV4 | 3758528 | CGGCCTATGAACCTGGTCGTTCACG |
| 418 | ETV4 | 3758528 | TCGCCTCCTACTTTCGGCCTATGAA |
| 419 | ETV4 | 3758522 | ACCACGGGAACCTGTCAGCGGGGAT |
| 420 | ETV4 | 3758522 | CCTTAAAGGACTCTAGGAGACCGTG |
| 421 | ETV4 | 3758522 | CGGTACCCATGGAGCCCCTTGTATC |
| 422 | ETV4 | 3758522 | GGGCCCGTCTCGTTGCCTTAAAGGA |
| 423 | ETV4 | 3758530 | GGAGGGACCTGCCACACGCTTGCGT |
| 424 | ETV4 | 3758530 | GGACCTGCCACACGCTTGCGTCGGG |
| 425 | ETV4 | 3758530 | CCTGCCACACGCTTGCGTCGGGGGA |
| 426 | ETV4 | 3758530 | AGGGACCTGCCACACGCTTGCGTCG |
| 427 | ETV4 | 3758525 | AAGGTCCTCTGCACCGAGCGACTTC |
| 428 | ETV4 | 3758525 | AGATTCAGTGAAGGTCCTCTGCACC |
| 429 | ETV4 | 3758525 | CCTAGATTCAGTGAAGGTCCTCTGC |
| 430 | ETV4 | 3758525 | AGTGAAGGTCCTCTGCACCGAGCGA |
| 431 | ETV4 | 3758512 | CCGACTCAAACTGGCCGGACAGTCA |
| 432 | ETV4 | 3758512 | TGTCAGGGAAACAGGGTGAACCTAC |
| 433 | ETV4 | 3758512 | AAGAGAAACCGGAAGGGCCTGTTAG |
| 434 | ETV4 | 3758512 | CTGTTAGTCGCAGGTCGAGAGTTCC |
| 435 | ETV4 | 3758523 | TACCGCTCGTCACGGAAATGAGGTC |
| 436 | ETV4 | 3758523 | CGTCCTTCGGCGGTGAGGGGATGGT |
| 437 | ETV4 | 3758523 | GTGGTACCGCTCGTCACGGAAATGA |
| 438 | ETV4 | 3758523 | CGTGTCTGGGCCGGGACAGGACGTC |
| 439 | ETV4 | 3758536 | GGGTCACCCTCCGGACCCTGGGACT |
| 440 | ETV4 | 3758536 | TACCGGGTCACCCTCCGGACCCTGG |
| 441 | ETV4 | 3758536 | CCGGACCCTGGGACTTCTCTCGGGT |
| 442 | ETV4 | 3758536 | CGGACCCTGGGACTTCTCTCGGGTC |
| 443 | ETV5 | 2709148 | TTGATAACTGTCCTAACACAGGTGG |
| 444 | ETV5 | 2709148 | TGATAACTGTCCTAACACAGGTGGA |
| 445 | ETV5 | 2709169 | ACTCGTCGGGAAGTACTATTTTAGG |
| 446 | ETV5 | 2709169 | GGGAATAGGACTAACGCACTAGGTC |
| 447 | ETV5 | 2709169 | TAACGCACTAGGTCACACTCGTCGG |
| 448 | ETV5 | 2709169 | CACTCGTCGGGAAGTACTATTTTAG |
| 449 | ETV5 | 2709182 | ACCATAATCTCTGCGACTTTCGTGG |
| 450 | ETV5 | 2709182 | TTCACCATAATCTCTGCGACTTTCG |
| 451 | ETV5 | 2709182 | TTACGACTTTGGAGAGTTTCACCAT |
| 452 | ETV5 | 2709182 | ACGACTTTGGAGAGTTTCACCATAA |
| 453 | ETV5 | 2709181 | TGCCCAAAATACTAGTCGTTCAGGG |
| 454 | ETV5 | 2709181 | AAAATACTAGTCGTTCAGGGAAAAT |
| 455 | ETV5 | 2709181 | TACCTGCCCAAAATACTAGTCGTTC |
| 456 | ETV5 | 2709181 | CCAAAATACTAGTCGTTCAGGGAAA |
| 457 | ETV5 | 2709134 | GTGCCAACGTAAGGGTAACCTGAGT |
| 458 | ETV5 | 2709134 | ACCGGTACACTTTCGGGCGGAACAA |
| 459 | ETV5 | 2709134 | GACGGTTCGACGCAATATAAGACAT |
| 460 | ETV5 | 2709134 | TCCCGGCACGGTTGAATACTTCTGT |
| 461 | ETV5 | 2709175 | CAAGGACTACTACTTGTCAAACAGG |
| 462 | ETV5 | 2709175 | GACTACTACTTGTCAAACAGGGTCT |
| 463 | ETV5 | 2709175 | CTACTACTTGTCAAACAGGGTCTAA |
| 464 | ETV5 | 2709175 | AGGACTACTACTTGTCAAACAGGGT |
| 465 | ETV5 | 2709153 | TGAGGGTACTAGAGTAACACGGTGA |
| 466 | ETV5 | 2709153 | TCACCCCTGGTGGTTTAACAGATTC |
| 467 | ETV5 | 2709153 | TTTAACAGATTCGTCTCCACTCGAC |
| 468 | ETV5 | 2709153 | GTTTTGAGGGTACTAGAGTAACACG |
| 469 | ETV5 | 2709177 | AGTCCTAGAGTCAGTTGAAGTTCTC |
| 470 | ETV5 | 2709177 | TAGAGTCAGTTGAAGTTCTCCGAAC |
| 471 | ETV5 | 2709177 | GAGTCAGTTGAAGTTCTCCGAACCA |
| 472 | ETV5 | 2709177 | TCAGTTGAAGTTCTCCGAACCAATC |
| 473 | ETV5 | 2709135 | ACCGAAAGGGCCTATTGGTCGCAGG |
| 474 | ETV5 | 2709135 | TGGTCGCAGGCAAGGACTTCCGTCT |
| 475 | ETV5 | 2709135 | AATGGAGGACCTGTACCTGGCGACG |
| 476 | ETV5 | 2709135 | CCTCTCGCTATGCAGATGTTTAAAC |
| 477 | ETV5 | 2709144 | ACACAACACGGACTCTCTGACCTTC |
| 478 | ETV5 | 2709144 | GCTAATATGAAACTGCTGTGAACAC |
| 479 | ETV5 | 2709144 | TATGAAACTGCTGTGAACACAACAC |
| 480 | ETV5 | 2709144 | GGGCTAATATGAAACTGCTGTGAAC |
| 481 | ETV5 | 2709139 | GACTCGGCGAGAGAGGCGATAATGA |
| 482 | ETV5 | 2709139 | CTCGGCGAGAGAGGCGATAATGATA |
| 483 | ETV5 | 2709139 | GCGATAATGATACTTTTCCCGTAGT |
| 484 | ETV5 | 2709139 | GGCGATAATGATACTTTTCCCGTAG |
| 485 | ETV5 | 2709154 | ACTACGGACTTTTGGTCATAGGTAG |
| 486 | ETV5 | 2709154 | ACGGTTTCTACTACGGACTTTTGGT |
| 487 | ETV5 | 2709154 | GGTGTCGTCGTTTGTAAACGCCAGG |
| 488 | ETV5 | 2709154 | CCAGGGGGCTGGTGGTGTAGTCGGG |
| 489 | ETV5 | 2709149 | TGTGCCCAAGGTCAGTGGTTACCCT |
| 490 | ETV5 | 2709149 | GGGTCAATGGTAGCCGTTTACAGTC |
| 491 | ETV5 | 2709149 | TCGGAGCCCTAATGACGCAGCTAAG |
| 492 | ETV5 | 2709149 | TGAGATACTTGTACCCCAGGGCCCG |
| 493 | ETV5 | 2709157 | TCCGAGAACCACGATTGATACCTCT |
| 494 | ETV5 | 2709157 | CTCTTTTCACGGAGATGTTGATAAC |
| 495 | ETV5 | 2709157 | TCGACAGCAGAACATCGGTACTCGT |
| 496 | ETV5 | 2709157 | TCTAGTTTGCCCTCGACGTGTCGGG |
| 497 | ETV5 | 2709143 | TCAAGTTCGACTATCTTGGCCTTCT |
| 498 | ETV5 | 2709143 | ATGGTACATAGCTCTCCCCGGGGGA |
| 499 | ETV5 | 2709143 | GAATGGTCTCCGCTCCAAGGGAAGT |
| 500 | ETV5 | 2709143 | TGGGAAGAACTACTGGGTCGGTTAC |
| 501 | ETV5 | 2709187 | CCGGGTCGGAAAGCGGGTCCGCGGG |
| 502 | ETV5 | 2709187 | CACGCGCCTCGCCAAGTGGCAGAAG |
| 503 | ETV5 | 2709187 | TCGCCAAGTGGCAGAAGCCTCGCCA |
| 504 | ETV5 | 2709187 | GAAGCCTCGCCAAGCCGGGTCGGAA |
| 505 | ETV5 | 2709179 | CTCCCGCCGGACACTAACTGTCTTT |
| 506 | ETV5 | 2709179 | CTCCTTCAAAAACCTGTGTCTAGAC |
| 507 | ETV5 | 2709179 | TGTCTAGACCGAGTGCTAAGACTTC |
| 508 | ETV5 | 2709179 | ACCTGTGTCTAGACCGAGTGCTAAG |
| 509 | ETV5 | 2709146 | AAACGAACTGATTATGGGTTCGAAT |
| 510 | ETV5 | 2709146 | CGAACTGATTATGGGTTCGAATTTT |
| 511 | ETV5 | 2709146 | AAGAAACGAACTGATTATGGGTTCG |
| 512 | ETV5 | 2709146 | GAGAAAGAAACGAACTGATTATGGG |
| 513 | ETV5 | 2709147 | AGGATGTACTCTCCCCCAATAAAGA |
| 514 | ETV5 | 2709147 | ACGGTCAGTAGGATGTACTCTCCCC |
| 515 | ETV5 | 2709147 | CTCCCCCAATAAAGAGGTCGTCGGT |
| 516 | ETV5 | 2709147 | CACGGATTGACGGTCAGTAGGATGT |
| 517 | ETV5 | 2709168 | ATTCCAGTGTCATCTCCTTCGGCGG |
| 518 | ETV5 | 2709168 | GGACAGTAGAGATTACTCAACCCTC |
| 519 | ETV5 | 2709168 | CCTTCGGCGGGACAGTAGAGATTAC |
| 520 | ETV5 | 2709168 | CCAGTGTCATCTCCTTCGGCGGGAC |
| 521 | FABP5P7 | 3104939 | CGTCGACCTTCCTTCTACCGCGGAC |
| 522 | FABP5P7 | 3104939 | TCTACCGCGGACCACCTGTCGTTTC |
| 523 | FABP5P7 | 3104939 | TCGTTTCCGAAACTACTTATGTACT |
| 524 | FABP5P7 | 3104939 | AAACTACTTATGTACTTCCTCGATC |
| 525 | FABP5P7 | 3104943 | CACTACCATTTTTGGAGTGGTATTT |
| 526 | FABP5P7 | 3104943 | TAACATAGTAGTGAACACTACCATT |
| 527 | FABP5P7 | 3104943 | CCCGCGTTACCGGTTCGGTCTAACA |
| 528 | FABP5P7 | 3104943 | GGAGTGGTATTTTTGACTCTCGTGA |
| 529 | FABP5P7 | 3104944 | AAACTTCTTTGGTGTCGACTACCGT |
| 530 | FABP5P7 | 3104944 | TGGTGTCGACTACCGTCTTTTTGAG |
| 531 | FABP5P7 | 3104944 | TTGGTGTCGACTACCGTCTTTTTGA |
| 532 | FABP5P7 | 3104944 | CTTCTTTGGTGTCGACTACCGTCTT |
| 533 | FABP5P7 | 3104946 | TGTCTACCACGTAACCAAGTCGTAG |
| 534 | FABP5P7 | 3104946 | GACGTTGAAATGTCTACCACGTAAC |
| 535 | FABP5P7 | 3104946 | ACAGACGTTGAAATGTCTACCACGT |
| 536 | FABP5P7 | 3104946 | CGTTGAAATGTCTACCACGTAACCA |
| 537 | FABP5P7 | 3104948 | CTCACACAGTACTTGTTACAGTGGA |
| 538 | FABP5P7 | 3104948 | CACACAGTACTTGTTACAGTGGACA |
| 539 | FABP5P7 | 3104948 | TGGACATGAGCCTAGATACTTTTTC |
| 540 | FABP5P7 | 3104948 | ACAGTGGACATGAGCCTAGATACTT |
| 541 | FABP5P7 | 3374836 | GAACACTACCATTTTTGGAGTGGTA |
| 542 | FABP5P7 | 3374836 | AGACGTTGAAATGTCTACCACGTAA |
| 543 | FABP5P7 | 3374836 | CCCGCGTTACCGGTTCGGTCTAACA |
| 544 | FABP5P7 | 3374836 | ACCTCACACAGTACTTGTTACAGTG |
| 545 | FABP5P7 | 3374837 | CGTCGACCTTCCTTCTACCGCGGAC |
| 546 | FABP5P7 | 3374837 | TCGTTTCCGAAACTACTTATGTACT |
| 547 | FABP5P7 | 3374837 | ACTACTTATGTACTTCCTCGATCCT |
| 548 | FABP5P7 | 3374837 | TTCTACCGCGGACCACCTGTCGTTT |
| 549 | FABP5P7 | 3517698 | ACTACTTATGTACTTCCTCGATCCT |
| 550 | FABP5P7 | 3517698 | TCGTTTCCGAAACTACTTATGTACT |
| 551 | FABP5P7 | 3517698 | TTCTACCGCGGACCACCTGTCGTTT |
| 552 | FABP5P7 | 3517698 | CGTCGACCTTCCTTCTACCGCGGAC |
| 553 | FLI1 | 3355756 | ATGGGTGGGACTCGCCGTCGGCACC |
| 554 | FLI1 | 3355756 | GGCGTGCGTCCCGAACGCGACCGAC |
| 555 | FLI1 | 3355756 | GGTCGACGGAGTAATTTCTCGTCGG |
| 556 | FLI1 | 3355756 | GGACATGGGTGGGACTCGCCGTCGG |
| 557 | FLI1 | 3355736 | CGACATTGGCCCAGTTACACACCTT |
| 558 | FLI1 | 3355736 | GCTCCAGTCCGACATTGGCCCAGTT |
| 559 | FLI1 | 3355736 | GACATTGGCCCAGTTACACACCTTA |
| 560 | FLI1 | 3355736 | CAGTCCGACATTGGCCCAGTTACAC |
| 561 | FLI1 | 3355789 | AACGTCCATTAACAACTGAAAAAAT |
| 562 | FLI1 | 3355789 | CAATTCGACTGTTGACAGTTTCTTC |
| 563 | FLI1 | 3355789 | TCCCTAAAAGGACGAGATATATTCG |
| 564 | FLI1 | 3355789 | ACGAAACCTTTACGCACATTGTCAT |
| 565 | FLI1 | 3355735 | AGCGAGGCGATGTTGTTGTTTGCAC |
| 566 | FLI1 | 3355735 | AAAGTAGGCCAATTGACAGAGAAAG |
| 567 | FLI1 | 3355735 | TGTTGTTTGCACGTGTCCCCTCACT |
| 568 | FLI1 | 3355735 | GGGCTAAGCGTTTCACTTCAGTGAA |
| 569 | FLI1 | 3355788 | TCGTATTATACGGATATCGACTTTT |
| 570 | FLI1 | 3355788 | GTATTATACGGATATCGACTTTTCC |
| 571 | FLI1 | 3355788 | TCAGTGACTGAATACTCTTTCGTTT |
| 572 | FLI1 | 3355788 | TTTTCGTATTATACGGATATCGACT |
| 573 | FLI1 | 3355750 | GAAACTGAGTCGCATGCCTCGCCGT |
| 574 | FLI1 | 3355750 | TGTACTGACGGAGCCCCTCAGGACT |
| 575 | FLI1 | 3355750 | GTCGGTCACTCCCAGTTGCAGTTCG |
| 576 | FLI1 | 3355750 | ACTCGCTGCTGGTCAGGGAGAAACT |
| 577 | FLI1 | 3355785 | CAAAGAACAGTTATGTGCCCCAAGT |
| 578 | FLI1 | 3355785 | GTGAATGACCTACGAAACCTGAGTT |
| 579 | FLI1 | 3355785 | CTTCGGGTAGGACGTGTGAATGACC |
| 580 | FLI1 | 3355785 | GGTACCCGGTCATACGGTCAAACTT |
| 581 | FLI1 | 3355784 | GTAGGTAGGAGGTACGGACAGTGAA |
| 582 | FLI1 | 3355784 | GGTCGAAGAAACCTCGGCGTAGTGT |
| 583 | FLI1 | 3355784 | CTCGGCGTAGTGTTATGACCTGGAG |
| 584 | FLI1 | 3355784 | AAGTGTGAATCCGTCGATGATGATC |
| 585 | FLI1 | 3355765 | ACAACAGTGTGGAGTCAATGGAGTC |
| 586 | FLI1 | 3355765 | ACCCGGTATTTCCTCATGTCGAACT |
| 587 | FLI1 | 3355765 | GGGAGATGTTGTGCCTTCACGACAA |
| 588 | FLI1 | 3355765 | GTCGAACTACCTCTAGCTGTGTAGG |
| 589 | FLI1 | 3355761 | GTTCTGCCCACTTAGTGAACAGTCC |
| 590 | FLI1 | 3355761 | CATTAAGCTCTTGGTCCGACGGACC |
| 591 | FLI1 | 3355761 | CCGTCCCTCGTAGATTTGGAAATAG |
| 592 | FLI1 | 3355761 | ATAGACTATGAGATAAGGGACACCT |
| 593 | FLI1 | 3355775 | GGACGAGTTACAGAGTTACCCTGAG |
| 594 | FLI1 | 3355775 | CACAACGAAAAGTACGGTCAACGAT |
| 595 | FLI1 | 3355775 | TTACACGAGGGTGGCGACACTACAG |
| 596 | FLI1 | 3355775 | CCCCACTCATGTGAAGGTCTTAAAT |
| 597 | FLI1 | 3355778 | CAGAGAGGGTAACCTTACGCTCAAG |
| 598 | FLI1 | 3355778 | ACGCTCAAGATGGTCCTTGACGAAC |
| 599 | FLI1 | 3355778 | TGTCGGAGTCATGGTAGTCACTACG |
| 600 | FLI1 | 3355778 | TCGACCCAGGATGAGTGACGTAAAG |
| 601 | FLI1 | 3355779 | CCTCCCCGTGTTTGCTAGTCATTCT |
| 602 | FLI1 | 3355779 | GGAACCTCCCCGTGTTTGCTAGTCA |
| 603 | FLI1 | 3355779 | CATTCTTATGTCTCGTTGCCGGGGT |
| 604 | FLI1 | 3355779 | CCGTGTTTGCTAGTCATTCTTATGT |
| 605 | FLI1 | 3355777 | TCCAATAGAAGACAGGGACTACTCC |
| 606 | FLI1 | 3355777 | AGTCTGTACGTGACCAGGGTATTCG |
| 607 | FLI1 | 3355777 | AAGGGACGAAAAACTCATCTGTAGT |
| 608 | FLI1 | 3355777 | ACTCCGATTCAGCATGGTTAAAGGG |
| 609 | FLI1 | 3355786 | TCCACCCTTCGAATATTAGATTAAA |
| 610 | FLI1 | 3355786 | GTAACTAACATTCCGGTCACTTCAA |
| 611 | FLI1 | 3355786 | AGTGGGTTGACCTTAAACTACCTTT |
| 612 | FLI1 | 3355786 | TGACCTTGTAACTAACATTCCGGTC |
| 613 | FLI1 | 3355787 | CACACAAATTCTGCGGTTCCCGTAA |
| 614 | FLI1 | 3355787 | CTGGAGCCAGTGTTTTCGTCAAAAT |
| 615 | FLI1 | 3355787 | CGTCTTAGGGAGAGTCACCTGTCAT |
| 616 | FLI1 | 3355787 | CACGACACGCGAACAGTCTGGTAGT |
| 617 | FLI1 | 3355783 | TAAACTGAAGGTGCCGTAACGGGTC |
| 618 | FLI1 | 3355783 | ACTGGTTTCACGTGCCGTTTTCTAT |
| 619 | FLI1 | 3355783 | GCCCGGGAGGCAATAATGATACTAT |
| 620 | FLI1 | 3355783 | GCTGGCTCAGCAGGTACATGTTCAT |
| 621 | FLI1 | 3355776 | CTTCTCCTCGAACCCCGTTATTGTA |
| 622 | FLI1 | 3355776 | CGAACCCCGTTATTGTACTTAAGAC |
| 623 | FLI1 | 3355776 | TCTTCTCCTCGAACCCCGTTATTGT |
| 624 | FLI1 | 3355776 | TCCTCGAACCCCGTTATTGTACTTA |
| 625 | FLI1 | 3355760 | CGATATACCTGCTCTTCTTACCGGG |
| 626 | FLI1 | 3355760 | GGTTGCTCTCCTCTCAGTAGCAGGG |
| 627 | FLI1 | 3355760 | AGGCCACCTGACGTCGCAATCGTTT |
| 628 | FLI1 | 3355760 | GGGTACTTGATGTTGTCGATATACC |
| 629 | FLI1 | 3355791 | CTGAGTGTCGTAACCATTGGGATCT |
| 630 | FLI1 | 3355791 | GATTCATGGAAGATCTGTTGTACAG |
| 631 | FLI1 | 3355791 | TCAAGGAAGTGACAATCCATCGAAT |
| 632 | FLI1 | 3355791 | TAAACGTTCCTTAATCTGAGTGTCG |
| 633 | FLI1 | 3355790 | TGAAAGGATAAATGAAGAACGTGAT |
| 634 | FLI1 | 3355790 | TAAAAAGCTTACATGGATGACGTCA |
| 635 | FLI1 | 3355790 | AGTTCTTAAAAAGCTTACATGGATG |
| 636 | FLI1 | 3355790 | AAATGAAGAACGTGATAGTTCTTAA |
| 637 | GPR116 | 2955921 | TTGTACTTGGTCGACCACTTCTCCG |
| 638 | GPR116 | 2955921 | ACTTGTACTTGGTCGACCACTTCTC |
| 639 | GPR116 | 2955921 | TGTACTTGGTCGACCACTTCTCCGT |
| 640 | GPR116 | 2955921 | GTACTTGTACTTGGTCGACCACTTC |
| 641 | GPR116 | 2955916 | GGATGAACTTGTCGGAGTCAAAAGG |
| 642 | GPR116 | 2955916 | TAAAACTCGTATTTACACTGTTGTC |
| 643 | GPR116 | 2955916 | ACCCTTATTGTGACTGGTTTAATGG |
| 644 | GPR116 | 2955916 | GGTTTAATGGCTGTAAAACTCGTAT |
| 645 | GPR116 | 2955910 | ACGTCAACGGAATTTCTTGACGGAG |
| 646 | GPR116 | 2955910 | GTAACGTCAACGGAATTTCTTGACG |
| 647 | GPR116 | 2955910 | TGACGGAGGGTTACCTGGAAAAACG |
| 648 | GPR116 | 2955910 | TTTCTTGACGGAGGGTTACCTGGAA |
| 649 | GPR116 | 2955923 | ACACGGAGTACAAATAACACTAAAT |
| 650 | GPR116 | 2955923 | AATGCTCAGATGATAAGTAGGAAAC |
| 651 | GPR116 | 2955923 | ACGTGACTTGACCTTAATGCTCAGA |
| 652 | GPR116 | 2955923 | GGGTTCCTCTTGGTGAAACACGGAG |
| 653 | GPR116 | 2955912 | GTTACTTCCAATCCGGATGACTGGT |
| 654 | GPR116 | 2955912 | TGGTACCGTGATTACAGATTCGTAG |
| 655 | GPR116 | 2955912 | TCTGACCAGTAAGTGTGTATCTCCC |
| 656 | GPR116 | 2955912 | TTTCGGAGTCCGAGTACCTGGAGTC |
| 657 | GPR116 | 2955906 | GTCCGAAGTTCCCGCACTGACACTG |
| 658 | GPR116 | 2955906 | ATGGTCCGAAGTTCCCGCACTGACA |
| 659 | GPR116 | 2955906 | CCGAAGTTCCCGCACTGACACTGTC |
| 660 | GPR116 | 2955906 | TCCCGCACTGACACTGTCCCAAGTT |
| 661 | GPR116 | 2955892 | TCTGTATCTAAGATCGACGTCGTCT |
| 662 | GPR116 | 2955892 | GACTACCTTGGGTCACGGGTTCGCC |
| 663 | GPR116 | 2955892 | CAAGTAGTCACGGATACCTCGGTCT |
| 664 | GPR116 | 2955892 | CCAGCAGACCTTGTTGTCAGTAGAT |
| 665 | GPR116 | 2955881 | TGTACGGTCCTAGGGCATTATCCAC |
| 666 | GPR116 | 2955881 | GGGTAACCGCCCTGGTAGTGAATGT |
| 667 | GPR116 | 2955881 | TCAGTAGGTCTTCGATACGGCCAAG |
| 668 | GPR116 | 2955881 | TACGGCCAAGAGTTTGCAAGGGTCG |
| 669 | GPR116 | 2955884 | AAGTTACGTTCGAGTCAAAGGACCA |
| 670 | GPR116 | 2955884 | CTCGGGTAGATACTTCGACTTAGAC |
| 671 | GPR116 | 2955884 | GTTAAAGTTACGTTCGAGTCAAAGG |
| 672 | GPR116 | 2955884 | ACGATTATTAAGTCAGACCTCGGGT |
| 673 | GPR116 | 2955883 | TCTTCAGTCCAAGTACACCTTTCGT |
| 674 | GPR116 | 2955883 | TAACCCATCAAGACTTTTCCACTGC |
| 675 | GPR116 | 2955883 | TTAGTTCCCGTTTTAATTCTTTAAC |
| 676 | GPR116 | 2955883 | CGTCTCTCCCCGAAACCTTTTAGTT |
| 677 | GPR116 | 2955877 | AAGTCCGCCGCTTTGCTTCACACAG |
| 678 | GPR116 | 2955877 | CAGCCTAAGCAGATAACAGTGGTAC |
| 679 | GPR116 | 2955877 | AGATCTTTTGAACGTCAGCCTAAGC |
| 680 | GPR116 | 2955877 | GTGACAGTCGGTGTTATGTTGATAC |
| 681 | GPR116 | 2955867 | CTTGTCCTATTAGGTTGGATGCACT |
| 682 | GPR116 | 2955917 | CGGTGTTTTTCAGGATGCCGACTTC |
| 683 | GPR116 | 2955887 | GTGCTCCTCCATAGAACTACCTCGT |
| 684 | GPR116 | 2955887 | TTGATACTACTCCAAATAACCTTGT |
| 685 | GPR116 | 2955887 | GGTTTCTAAAATATGGTGCTCCTCC |
| 686 | GPR116 | 2955887 | ACTGTCAGTTCTGGAGCTGGTCCCT |
| 687 | GPR116 | 2955866 | TACCCCTTGCACAAGAGCCCCGTCC |
| 688 | GPR116 | 2955866 | CGTCCAAAGGCCCTCGTCTACGGTT |
| 689 | GPR116 | 2955866 | ACGAACGTTTCGTTACCCCTTGCAC |
| 690 | GPR116 | 2955866 | GTCCAAAGGCCCTCGTCTACGGTTT |
| 691 | GPR116 | 2955878 | AGGGTTCGTAATGTCAGCCCTCTAT |
| 692 | GPR116 | 2955878 | TAATGTCAGCCCTCTATCGGGAGGA |
| 693 | GPR116 | 2955878 | TCTAAAAGGGTTCGTAATGTCAGCC |
| 694 | GPR116 | 2955878 | ACCTTTCTAAAAGGGTTCGTAATGT |
| 695 | GPR116 | 2955914 | GGAACCATATAGTTACCTGTGTTGT |
| 696 | GPR116 | 2955914 | ACCATATAGTTACCTGTGTTGTTCC |
| 697 | GPR116 | 2955914 | GAGGGAACCATATAGTTACCTGTGT |
| 698 | GPR116 | 2955914 | GGAGGAGGGAACCATATAGTTACCT |
| 699 | GPR116 | 2955885 | ACAAGTGGGCGACGGAGATTTCGAC |
| 700 | GPR116 | 2955885 | AATGAAAGGTATGCCCAAGGAGTAG |
| 701 | GPR116 | 2955885 | GTCATAACGTTGGTTTCTGCAGTAA |
| 702 | GPR116 | 2955885 | GATTTCGACTTGTAGTACCAACTAG |
| 703 | GPR116 | 2955898 | TGTTAGGACATAGAAACTTGACGAC |
| 704 | GPR116 | 2955898 | ACTACACGCTGTTGTTAGGACATAG |
| 705 | GPR116 | 2955898 | CGCTGTTGTTAGGACATAGAAACTT |
| 706 | GPR116 | 2955898 | ACTTCCACTACACGCTGTTGTTAGG |
| 707 | GPR116 | 2955879 | GGAACTAGACGAGAGTTGTCAAGGT |
| 708 | GPR116 | 2955879 | CCTACTCTACGAGGGATGTATGGAC |
| 709 | GPR116 | 2955879 | AATCGTATCTGTTTCGCCTTGTACT |
| 710 | GPR116 | 2955879 | GGACTTCCTAGAAAGATAATCGTAT |
| 711 | GPR116 | 2955924 | AAGAGGGAGGTGACCCGCACTCTCG |
| 712 | GPR116 | 2955924 | GTAGACTAGTCTCGCCCTCGGTCGG |
| 713 | GPR116 | 2955924 | CGGACTTTTGCGCTTTACTCAGAAC |
| 714 | GPR116 | 2955924 | TACTCAGAACGAACCAAGAGGGAGG |
| 715 | GPR116 | 2955904 | GACCTTCACACCAACACTGTATACT |
| 716 | GPR116 | 2955904 | ACCTGATGTTGAGGAAAGTTCGTCA |
| 717 | GPR116 | 2955904 | GGTGGTAGTGAACTCAATTATGTAT |
| 718 | GPR116 | 2955904 | AACATGTCTCGGAGTTAGTCTGGAT |
| 719 | GPR116 | 2955899 | GTAAAAACTTATACTCACGTTCTTC |
| 720 | GPR116 | 2955899 | ATAATCTGTAAAAACTTATACTCAC |
| 721 | GPR116 | 2955900 | TGTCGTCAACCTTTAGGTCTTGTCG |
| 722 | GPR116 | 2955900 | TTGTTGTACTGAAGCCACAGGTTCG |
| 723 | GPR116 | 2955900 | AACCGCGATACTTCTTGTCGTCAAC |
| 724 | GPR116 | 2955900 | GTCGTCTAAGAGCTAAATGTGGCGT |
| 725 | GPR116 | 2955911 | ACCACGAGGACGCTCTGTCCAATAC |
| 726 | GPR116 | 2955911 | AGAGTAAACAGTTCTCGCACTGCAG |
| 727 | GPR116 | 2955911 | ACAGTTCTCGCACTGCAGAAGGAGG |
| 728 | GPR116 | 2955911 | GTCCAATACCCACCGGAGCCCTTTC |
| 729 | GPR116 | 2955874 | TAAGTAAAATGAGAAACCTACGGAG |
| 730 | GPR116 | 2955874 | AGAAACCTACGGAGACCCTAGACTT |
| 731 | GPR116 | 2955874 | AAATGAGAAACCTACGGAGACCCTA |
| 732 | GPR116 | 2955874 | TAATAAGTAAAATGAGAAACCTACG |
| 733 | GPR116 | 2955908 | AGGCGGGAGATATCCAGGATGTTCT |
| 734 | GPR116 | 2955908 | CTTCTGGAGTACTTGTGAAGGAGGC |
| 735 | GPR116 | 2955908 | TTCTTCTGGAGTACTTGTGAAGGAG |
| 736 | GPR116 | 2955908 | TATCCAGGATGTTCTGGCTGAACCT |
| 737 | GPR116 | 2955864 | GTCGTTGCGATGTAACGTTTATTTT |
| 738 | GPR116 | 2955864 | ACGTGCGTATAATCTCAATTGGTAC |
| 739 | GPR116 | 2955864 | ACGTTTATTTTCAGGCTAGGGTTTT |
| 740 | GPR116 | 2955864 | CAATTGGTACATGATAACTATGTCG |
| 741 | GPR116 | 2955865 | ACAACGTGACTTCTGTCTGGGACAG |
| 742 | GPR116 | 2955865 | GAAACCCGTCATAGAAGGACTACAG |
| 743 | GPR116 | 2955865 | CCGAAGTTCGTCCATGAAGAGACAC |
| 744 | GPR116 | 2955865 | TTCCGGGTTGAAGAGACAGATATAA |
| 745 | GPR116 | 2955872 | CAAGAGGTTATAGTTCCTCTAAATT |
| 746 | GPR116 | 2955872 | AGAGGTTATAGTTCCTCTAAATTGT |
| 747 | GPR116 | 2955872 | TACTCAAGAGGTTATAGTTCCTCTA |
| 748 | GPR116 | 2955872 | CTCAAGAGGTTATAGTTCCTCTAAA |
| 749 | GPR116 | 2955873 | CGACTTATTCAAAAGTAACAGCTCT |
| 750 | GPR116 | 2955873 | AAAAGTAACAGCTCTACCAGAAGTG |
| 751 | GPR116 | 2955873 | TATTCAAAAGTAACAGCTCTACCAG |
| 752 | GPR116 | 2955873 | TCGAAACGACTTATTCAAAAGTAAC |
| 753 | GPR116 | 2955875 | ACACAAGGGTCCCTGGTTGGAACAC |
| 754 | GPR116 | 2955875 | TAACCCCAGGAGTGTGGTGAGAACC |
| 755 | GPR116 | 2955875 | AACGGTAGAGCCAGTAGTGCGACCC |
| 756 | GPR116 | 2955875 | CCTGAAGAATATACGCGGTGTGGAC |
| 757 | GPR116 | 2955876 | ACTACCCCTGTTACAGTGGACATAG |
| 758 | GPR116 | 2955876 | TACCCCTGTTACAGTGGACATAGAC |
| 759 | GPR116 | 2955876 | CCTGTTACAGTGGACATAGACACTG |
| 760 | GPR116 | 2955876 | CCCCTGTTACAGTGGACATAGACAC |
| 761 | GPR116 | 3172054 | TCGAAACGACTTATTCAAAAGTAAC |
| 762 | GPR116 | 3172054 | TATTCAAAAGTAACAGCTCTACCAG |
| 763 | GPR116 | 3172054 | CGACTTATTCAAAAGTAACAGCTCT |
| 764 | GPR116 | 3172054 | AAAAGTAACAGCTCTACCAGAAGTG |
| 765 | GPR116 | 3172055 | AGAGGTTATAGTTCCTCTAAATTGT |
| 766 | GPR116 | 3172055 | CAAGAGGTTATAGTTCCTCTAAATT |
| 767 | GPR116 | 3172055 | TACTCAAGAGGTTATAGTTCCTCTA |
| 768 | GPR116 | 3172055 | CTCAAGAGGTTATAGTTCCTCTAAA |
| 769 | GPR116 | 3207265 | TGGACCAAGTAACACCAGCGACGGT |
| 770 | GPR116 | 3207265 | GGAAGACCAGCGGTTGTGGACCAAG |
| 771 | GPR116 | 3207265 | AGAATACAACCCCACCCGAAAAGGT |
| 772 | GPR116 | 3207265 | AGATAGCGGACCAAAAGTAAGACGT |
| 773 | GPR116 | 3207266 | ACTACCCCTGTTACAGTGGACATAG |
| 774 | GPR116 | 3207266 | CCACTACCCCTGTTACAGTGGACAT |
| 775 | GPR116 | 3207266 | ACCCCTGTTACAGTGGACATAGACA |
| 776 | GPR116 | 3207266 | CCTGTTACAGTGGACATAGACACTG |
| 777 | GPR116 | 3207387 | ACCGGGAACCACACAACGTATCGAG |
| 778 | GPR116 | 3207387 | ACTGGAACAACTTGTTTACCGTCTC |
| 779 | GPR116 | 3207387 | AGACACGAGGATTACTGTGAACTGG |
| 780 | GPR116 | 3207387 | GGAACCACACAACGTATCGAGGGAT |
| 781 | GPR116 | 3207390 | TACTCAAGAGGTTATAGTTCCTCTA |
| 782 | GPR116 | 3207390 | AGAGGTTATAGTTCCTCTAAATTGT |
| 783 | GPR116 | 3207390 | CTCAAGAGGTTATAGTTCCTCTAAA |
| 784 | GPR116 | 3207390 | CAAGAGGTTATAGTTCCTCTAAATT |
| 785 | GPR116 | 3207391 | AAAAGTAACAGCTCTACCAGAAGTG |
| 786 | GPR116 | 3207391 | TCGAAACGACTTATTCAAAAGTAAC |
| 787 | GPR116 | 3207391 | CGACTTATTCAAAAGTAACAGCTCT |
| 788 | GPR116 | 3207391 | TATTCAAAAGTAACAGCTCTACCAG |
| 789 | GPR116 | 2955929 | CAAGTGGAGTGGACTCTCCCAAAAC |
| 790 | GPR116 | 2955929 | GTGGACTCTCCCAAAACCCGTCTAG |
| 791 | GPR116 | 2955929 | CTTACCCTACGGGAGCTCCAAGTGG |
| 792 | GPR116 | 2955929 | GTCTAGTCGTCATTCCACAATTTAA |
| 793 | GPR116 | 2955986 | CTTCAGACCAGAACACTTTGGGGTG |
| 794 | GPR116 | 2955986 | CTTCCGTCAAGTGGAGACGAGGGCT |
| 795 | GPR116 | 2955986 | GCTGTCGGACCCTTGGGCGTTCTCG |
| 796 | GPR116 | 2955986 | GGGGTCGTAAACTTCAGACCAGAAC |
| 797 | GRM7 | 2609074 | GAGCCGAACCTCCTGCTAAGGGCCT |
| 798 | GRM7 | 2609074 | TAGGAGCCGAACCTCCTGCTAAGGG |
| 799 | GRM7 | 2609074 | GAACCTCCTGCTAAGGGCCTCGCTC |
| 800 | GRM7 | 2609074 | CCTGCTAAGGGCCTCGCTCCGTACT |
| 801 | GRM7 | 2609075 | ACCCAAGACGGCGTCACAAGAGAGC |
| 802 | GRM7 | 2609075 | CGTCACAAGAGAGCGGAGGACGAGG |
| 803 | GRM7 | 2609075 | GGCGACTCGCCACCCAAGACGGCGT |
| 804 | GRM7 | 2609075 | TCCTTCCGCCTAGGCCCCGGCGACT |
| 805 | GRM7 | 2609071 | CGAATGAAAGCAGGTCCGCGAGTAG |
| 806 | GRM7 | 2609071 | GCGCGAGCTTGTCAGCGAATGAAAG |
| 807 | GRM7 | 2609071 | ACAAGGTCCCTGTGAATGCGCGAGC |
| 808 | GRM7 | 2609071 | GACGAGGCGCAGGACTGAAACTACT |
| 809 | GRM7 | 2609056 | CGTCGTTAAACCTTTCTGGTAGAAC |
| 810 | GRM7 | 2609056 | CCTTATGTTCCATAATGTGTCTTCG |
| 811 | GRM7 | 2609056 | GTCGTTAAACCTTTCTGGTAGAACT |
| 812 | GRM7 | 2609094 | CTACGGGAAGATAGGACAGAGTCAA |
| 813 | GRM7 | 2609094 | AGAGTCAAAGGGTTAGGATTTGCAG |
| 814 | GRM7 | 2609094 | ACGGGAAGATAGGACAGAGTCAAAG |
| 815 | GRM7 | 2609094 | CCTCTACGGGAAGATAGGACAGAGT |
| 816 | GRM7 | 2609070 | ACTTCCCGGGCCTGGAGCCGCTCGG |
| 817 | GRM7 | 2609070 | GGACTTCCCGGGCCTGGAGCCGCTC |
| 818 | GRM7 | 2609070 | GCTCGGGTGGTGGCAAGGGAGGTCG |
| 819 | GRM7 | 2609070 | TCGGGTGGTGGCAAGGGAGGTCGCG |
| 820 | GRM7 | 2609082 | CATCTTGATCTACTGTAACGTCCGT |
| 821 | GRM7 | 2609120 | ACTACTGGCCGCGATACTGAAGAAG |
| 822 | GRM7 | 2609120 | ACACAGATGGGAGCGTAGCCTTCCT |
| 823 | GRM7 | 2609120 | TCCGGGATCCGACCTTAATACACAG |
| 824 | GRM7 | 2609120 | CACACCTCAGGAAGTGCGTCTAAAG |
| 825 | GRM7 | 2609135 | CTAAAAACGGTTGCTACTCCTATAT |
| 826 | GRM7 | 2609135 | GGTCCTTGCGTTTCTGTCCTGGTAA |
| 827 | GRM7 | 2609135 | TTGAGGTCCCGGCAGCACTAAAAAC |
| 828 | GRM7 | 2609135 | GTCCTGGTAACTGAAACTATCTTAA |
| 829 | GRM7 | 2609210 | TTTGTACTGTACCTAGTCGTTGAGG |
| 830 | GRM7 | 2609210 | GGTTTCGAACAAGAAGAACTCGACT |
| 831 | GRM7 | 2609210 | ATCCTTTTGTACTGTACCTAGTCGT |
| 832 | GRM7 | 2609210 | ACTCGACTGGGTTGTGAATCCTTTT |
| 833 | GRM7 | 2609197 | TAGCAAGAACAGAATACCCGACCCA |
| 834 | GRM7 | 2609197 | CATTGAGAGGCAAAGAAAGTAGAAG |
| 835 | GRM7 | 2609197 | CTCTTTATGACACTAGCAAGAACAG |
| 836 | GRM7 | 2609197 | ACTCAAACTGAGTAAGGACGGTGGT |
| 837 | GRM7 | 2609183 | GACGTTTTCTGTACTGAGTACGAAA |
| 838 | GRM7 | 2609183 | TCTGTACTGAGTACGAAAAAATACC |
| 839 | GRM7 | 2609183 | TTTTCTGTACTGAGTACGAAAAAAT |
| 840 | GRM7 | 2609183 | GTACTGAGTACGAAAAAATACCGAC |
| 841 | GRM7 | 2609198 | TCGAAAACCTCCGAGACGAAGTCTT |
| 842 | GRM7 | 2609198 | ACCTCCGAGACGAAGTCTTCACACT |
| 843 | GRM7 | 2609198 | AAAACCTCCGAGACGAAGTCTTCAC |
| 844 | GRM7 | 2609198 | AACCTCCGAGACGAAGTCTTCACAC |
| 845 | GRM7 | 2609201 | ACAACAAATTCCGTCTTCCCTAAAG |
| 846 | GRM7 | 2609201 | TACCCGTATATCGATAGTGAAACAT |
| 847 | GRM7 | 2609201 | GATCCTCGGAAATCAGTACGTACGT |
| 848 | GRM7 | 2609201 | AAGGTCCTTATAAAACCGATCAACG |
| 849 | GRM7 | 2609194 | TGTTTGATGGTGCGAATGTTAGAGG |
| 850 | GRM7 | 2609194 | GTATGTTTGATGGTGCGAATGTTAG |
| 851 | GRM7 | 2609194 | ATGTTTGATGGTGCGAATGTTAGAG |
| 852 | GRM7 | 2609194 | TGTATGTTTGATGGTGCGAATGTTA |
| 853 | GRM7 | 2609160 | CCGTGAGGTCACTACAAATTGTTCT |
| 854 | GRM7 | 2609160 | CGTGGACCCGCAATACTGTAGAAAG |
| 855 | GRM7 | 2609160 | CGTCACCTGTCTGCTTGAAGTCGAG |
| 856 | GRM7 | 2609160 | AATGGCAGACTAGCCCGTCACCTGT |
| 857 | GRM7 | 2609149 | ACTACGGATGAAATGCAGGGCATGT |
| 858 | GRM7 | 2609149 | CAGGGCATGTGAACTTTTGTTGTCT |
| 859 | GRM7 | 2609149 | TCTTCTGTGTCTAGCGTTTACGTGT |
| 860 | GRM7 | 2609149 | TGACGTTCAACTGCTAATCACCCAG |
| 861 | GRM7 | 2609196 | GGACTTGAGTTACAGGTCTTTGCCT |
| 862 | GRM7 | 2609196 | TCGGTGGTACAGTAGCTCCGACAGT |
| 863 | GRM7 | 2609196 | GTTGCCACTCCGTTTCTGGCTCGAG |
| 864 | GRM7 | 2609196 | TTCGCTTCGAAGTTCCGCCATCAGT |
| 865 | GRM7 | 2609174 | TATCTCTGGTAAACACGGACGATGT |
| 866 | GRM7 | 2609174 | GAAGGTCCTGTAGCATCATGGGAAT |
| 867 | GRM7 | 2609174 | TGTTCTACTACAATCTTCCTACCCT |
| 868 | GRM7 | 2609174 | GGAAACTAATACTCTATCCTCTTAA |
| 869 | GRM7 | 2609200 | CTTCTCAAAATAAGATACGTTCTGT |
| 870 | GRM7 | 2609200 | TTTGTATAGATGGTATGGTGACCTC |
| 871 | GRM7 | 2609200 | CTATTGCCGTAGTATGACACCCCTT |
| 872 | GRM7 | 2609200 | GTGTCCCGGTCTGTGATGGTACTAT |
| 873 | GRM7 | 2609158 | ACTAACTGCGTCAGATACGATACCG |
| 874 | GRM7 | 2609158 | AACGACTTCATATATGCGTTACAAT |
| 875 | GRM7 | 2609158 | TAGAGACACGACTGATGGCCCCACA |
| 876 | GRM7 | 2609158 | GTCCTCCCATTTCAGGTCAAGCACT |
| 877 | GRM7 | 2609178 | GGACACTCGGAACGCTACCAATGGT |
| 878 | GRM7 | 2609178 | CTATGGGCGGAGTCACACGTGTGAT |
| 879 | GRM7 | 2609178 | ACGGTAGTTCTGAGCCCCACATGGG |
| 880 | GRM7 | 2609178 | GTCGATACAAGAAAACTGCCCGTAG |
| 881 | GRM7 | 2609209 | TGGAGTCGAACGTGTTTCTCCTAAC |
| 882 | GRM7 | 2609209 | CTGGAGTCGAACGTGTTTCTCCTAA |
| 883 | GRM7 | 2609209 | GGAGTCGAACGTGTTTCTCCTAACT |
| 884 | GRM7 | 2609209 | CCTGGAGTCGAACGTGTTTCTCCTA |
| 885 | GRM7 | 2609203 | GACAATGAACCATGTGATAGGGTGG |
| 886 | GRM7 | 2609203 | ATGAACCATGTGATAGGGTGGTTGT |
| 887 | GRM7 | 2609203 | TGACATATGGTGGTCATTCTTTCTC |
| 888 | GRM7 | 2609203 | TTTCAGACAATGAACCATGTGATAG |
| 889 | GRM7 | 2609136 | ACTTCTATAGCGTCTTCCCCGGTAG |
| 890 | GRM7 | 2609136 | AGTCGGGTTCGCTCGGTGCCACCTT |
| 891 | GRM7 | 2609136 | TCCCCGGTAGTGGTAAGTCGGGTTC |
| 892 | GRM7 | 2609136 | CGGTAAAAGAAACCCACCCTAGTCT |
| 893 | GRM7 | 2609173 | AAGGGTAGGTTCTATTTCAGTCTAC |
| 894 | GRM7 | 2609173 | ATAGTGGACAGTCCAGACCTCTTCG |
| 895 | GRM7 | 2609173 | TTTACACACGTACAACTCCTGACTT |
| 896 | GRM7 | 2609173 | TCAGTCTACGAGATCTTTACACACG |
| 897 | GRM7 | 2609202 | CGGTTGACCCAAACTAGGACTGTTT |
| 898 | GRM7 | 2609202 | GACTGTTTGTGATGTGTTTCTTGAC |
| 899 | GRM7 | 2609202 | ACTCACTTACAGCACTTTATGAAAC |
| 900 | GRM7 | 2609202 | AACGAGACGTTTATTCTCTCTATAG |
| 901 | GRM7 | 2609199 | TGACTGTTTACGTGGATAGTCCAAC |
| 902 | GRM7 | 2609199 | AAGGACGATTCCGTTGTTGAAAAAC |
| 903 | GRM7 | 2609199 | CCATGACTTAGGAGGTTGACTGTTT |
| 904 | GRM7 | 2609199 | AAAAGGAGATCGTACACTTACAACT |
| 905 | GRM7 | 2609207 | CATGTCACCTTGAACCTAATGTGAA |
| 906 | GRM7 | 2609207 | CCGACATGTCACCTTGAACCTAATG |
| 907 | GRM7 | 2609207 | AAAAAACCGACATGTCACCTTGAAC |
| 908 | GRM7 | 2609207 | AACCGACATGTCACCTTGAACCTAA |
| 909 | GRM7 | 2609195 | TAGTCACCGCGACCCCTACGATATG |
| 910 | GRM7 | 2609195 | ATTCACGTAGTCACCGCGACCCCTA |
| 911 | GRM7 | 2609195 | GTAGTCACCGCGACCCCTACGATAT |
| 912 | GRM7 | 2609195 | TGGATTCACGTAGTCACCGCGACCC |
| 913 | GRM7 | 2609190 | GACTCGTAGATTACCACGGACCTGT |
| 914 | GRM7 | 2609190 | CGGACCTGTGTGTGACAGTAACAAA |
| 915 | GRM7 | 2609190 | GTCCGAAAGGGACTTAGTAACATTC |
| 916 | GRM7 | 2609190 | TCTGCCCCGGAGACAGGATAAATAA |
| 917 | GRM7 | 2609213 | TGACCGTAGATCAGTTCGCTAACAG |
| 918 | GRM7 | 2609213 | GTAGACGTGACCGTAGATCAGTTCG |
| 919 | GRM7 | 2609213 | ACGTGACCGTAGATCAGTTCGCTAA |
| 920 | GRM7 | 2609213 | CGTAGATCAGTTCGCTAACAGACTC |
| 921 | GRM7 | 2609223 | GCCGACGTTAACACCTGGAAGGGAT |
| 922 | GRM7 | 2609223 | GGTGACTCTCGGTGTCCTGGCAAAA |
| 923 | GRM7 | 2609223 | CTCGGGATAAGAGAGTCTGCCACCT |
| 924 | GRM7 | 2609223 | ACAACTTTGAGTTCAGGGCGGGACC |
| 925 | GRM7 | 2609222 | TACAGTCAATATTATTGGACCAATA |
| 926 | GRM7 | 2609222 | CAGTCAATATTATTGGACCAATAGA |
| 927 | GRM7 | 2609222 | GTCAATATTATTGGACCAATAGATT |
| 928 | GRM7 | 2609222 | ACAGTCAATATTATTGGACCAATAG |
| 929 | GRM7 | 2609215 | ACATGGTGTGTATTATTTCAAATTC |
| 930 | GRM7 | 2609215 | GTAGGACATGGTGTGTATTATTTCA |
| 931 | GRM7 | 2609215 | AACAGTAGGACATGGTGTGTATTAT |
| 932 | GRM7 | 2609215 | AAGATAAACCAGAGAACATGGGTAA |
| 933 | MME | 2648729 | CCCGTAGCCGTACCAGTATCCTGTG |
| 934 | MME | 2648729 | ATCACGGGTCGTCAGGTTGAGTAAC |
| 935 | MME | 2648729 | TTGAGTAACTTGATACCCCCGTAGC |
| 936 | MME | 2648729 | TCCTGTGCTTTAGTGGGTACCGAAG |
| 937 | MME | 2648680 | TTCCAGGTTTCCCGCGCTCGCGGGT |
| 938 | MME | 2648680 | AGGTTTCCCGCGCTCGCGGGTCCCG |
| 939 | MME | 2648680 | GGTTTCCCGCGCTCGCGGGTCCCGC |
| 940 | MME | 2648680 | TCTTTCCAGGTTTCCCGCGCTCGCG |
| 941 | MME | 2648678 | TCTACACGTTCACCGCTTCGAACTG |
| 942 | MME | 2648678 | CCTCGTCGGCGGGTTGAGGACCGCG |
| 943 | MME | 2648678 | CTTCGAACTGGCTCTCGTCCGACCT |
| 944 | MME | 2648678 | GTTGAGGACCGCGCCCTAGACGACT |
| 945 | MME | 2648725 | TCTACTTATGAAGCTCTTGTATTAA |
| 946 | MME | 2648725 | TACTTATGAAGCTCTTGTATTAAGT |
| 947 | MME | 2648725 | CTACTTATGAAGCTCTTGTATTAAG |
| 948 | MME | 2648725 | ACTTATGAAGCTCTTGTATTAAGTT |
| 949 | MME | 2648707 | TATATCATCGTCACGTCTTTCGTTT |
| 950 | MME | 2648707 | GTAACATGTCCAGAACATATTTACT |
| 951 | MME | 2648707 | TTTGACTTCTATATCATCGTCACGT |
| 952 | MME | 2648707 | GTTTTCGTAACATGTCCAGAACATA |
| 953 | MME | 2648682 | TCCTCCCGAGACCTTCAGTGCAGTC |
| 954 | MME | 2648682 | GGACCTCCTCCCGAGACCTTCAGTG |
| 955 | MME | 2648682 | CCTCCCGAGACCTTCAGTGCAGTCC |
| 956 | MME | 2648682 | ACCTCCTCCCGAGACCTTCAGTGCA |
| 957 | MME | 2648721 | GTGAACCTACCTACGGCTCTGTTTT |
| 958 | MME | 2648721 | AACGTGTCTAGGCTCTTCAAAAATA |
| 959 | MME | 2648721 | AAGTCTGAAATCTACTGGAGTGAAC |
| 960 | MME | 2648721 | CAGCTCCTAAACTAACGTGTCTAGG |
| 961 | MME | 2648681 | GTCCCAGGCGTCGATTCCAGGTCGC |
| 962 | MME | 2648681 | CTTCCTCGGCGATGACCCTGGACTT |
| 963 | MME | 2648681 | TCCTCGGCGATGACCCTGGACTTCT |
| 964 | MME | 2648681 | TCGCGCTAGGCTCGCGGGTCCTGGG |
| 965 | MME | 2648724 | AATTTCTTTCCTAGCCGATAGGACT |
| 966 | MME | 2648724 | GTTAATTTCTTTCCTAGCCGATAGG |
| 967 | MME | 2648724 | CCTAGCCGATAGGACTACTGTAACA |
| 968 | MME | 2648724 | TCTTTCCTAGCCGATAGGACTACTG |
| 969 | MME | 2648710 | TACTTACGTGACCTTAGATATTTCT |
| 970 | MME | 2648710 | AATGATACTTACGTGACCTTAGATA |
| 971 | MME | 2648710 | TAATGATACTTACGTGACCTTAGAT |
| 972 | MME | 2648743 | GGTCAAACGACTACAGGGATCTTTT |
| 973 | MME | 2648743 | TCTTCGTAACGTCGGGAACCGATCT |
| 974 | MME | 2648743 | CCCAGATTCCAGATAGTTCAGTTAG |
| 975 | MME | 2648743 | GGGATCCCCAGTGACATGACTGAAC |
| 976 | MME | 2648717 | CAACAAATACGAGGTCTTATAAATT |
| 977 | MME | 2648717 | CCAACAAATACGAGGTCTTATAAAT |
| 978 | MME | 2648717 | TTACTTTAGTACAGTTGACACTTAT |
| 979 | MME | 2648717 | CAGTTGACACTTATAATCATAATGT |
| 980 | MME | 2648742 | TAGGTCTTTTCTTCACGGCCCAAAC |
| 981 | MME | 2648742 | ACGTCTTGAGACGTCTCAAAAGTCT |
| 982 | MME | 2648742 | CGTTCTTAAGTATGTACTTAGGTCT |
| 983 | MME | 2648742 | GAAAGTGACGGCGTTCTTAAGTATG |
| 984 | MME | 2648679 | TCGGAGGTTGAAGAGGGCTTAGGGT |
| 985 | MME | 2648679 | GGGCACGCGAGTAACCAGCCCTACA |
| 986 | MME | 2648679 | GCCTACGTGCCTGACTCTCCGCGAA |
| 987 | MME | 2648679 | TCTCCGCGAACCGACCCGAGAGTCG |
| 988 | MME | 2648702 | AAACTCTTATACTTAGACGGACACC |
| 989 | MME | 2648702 | AACTCTTATACTTAGACGGACACCT |
| 990 | MME | 2648686 | GGTCAATTATCTATGACCATAGTCA |
| 991 | MME | 2648686 | CTTCGGTGACCTCGTTCTATATTTT |
| 992 | MME | 2648686 | TAACGAATCAGTTTTGGAGTTCCAT |
| 993 | MME | 2648686 | TGGTAACTACAATCAGGTCTCAAGT |
| 994 | MME | 2648733 | GGTAAAATCACTAAATCTCACAAAG |
| 995 | MME | 2648733 | TCACTAAATCTCACAAAGAGTATAA |
| 996 | MME | 2648733 | CTAAATCTCACAAAGAGTATAAATT |
| 997 | MME | 2648733 | ATCTCACAAAGAGTATAAATTATTA |
| 998 | MME | 2648684 | ACCAACCGTGAAGCACAGGTCTTGA |
| 999 | MME | 2648684 | ACTAAATAGGACTCATGGAGACAGG |
| 1000 | MME | 2648684 | CAGGTCTTGAGGCATAAAGTCCGAC |
| 1001 | MME | 2648684 | TGTGGATTAATTACAGACCCTTTGT |
| 1002 | MME | 2648720 | GCAACACGTTTGATACAGTTACCCT |
| 1003 | MME | 2648720 | TGTCGTTGAACCTCTGCAACACGTT |
| 1004 | MME | 2648720 | GACACCCCTCCGAAATACACCTTCG |
| 1005 | MME | 2648720 | GTTTGATACAGTTACCCTTATACCT |
| 1006 | MME | 2648687 | ATCGACACTGTTACTAGCGTGAGAT |
| 1007 | MME | 2648687 | TAGCGTGAGATACGTTGGATGCTAC |
| 1008 | MME | 2648687 | GACGAGGAGTGGTAGTATCGACACT |
| 1009 | MME | 2648687 | TCTTTGTCGCTACCTGAGGTGACCT |
| 1010 | MME | 2648685 | GACAACCATTATAACAGCTCGACTT |
| 1011 | MME | 2648685 | GGAATGAGAAGAGATACCCTAACCC |
| 1012 | MME | 2648685 | CCCAAATCAGTATACAAGGTGTCAC |
| 1013 | MME | 2648685 | CATACAAAAACGCATGGGACGAAAT |
| 1014 | MME | 2648716 | TACTAGGTTACGAAGACATATTGTT |
| 1015 | MME | 2648716 | CGATGCCGATTTGGACTTCTAGCTT |
| 1016 | MME | 2648716 | GGACTTCTAGCTTTACTAGGTTACG |
| 1017 | MME | 2648716 | ATTGTTCTACTGTAACCGGGTCTAG |
| 1018 | MME | 2648683 | AGTAAAGGTATCAAGGGACGCCGGA |
| 1019 | MME | 2648683 | AGACGGAACCCCTCAATACAAAACA |
| 1020 | MME | 2648683 | GATGGTCTAACGTGGCCCCGACTAA |
| 1021 | MME | 2648683 | ATACAAAACAATGGCTCTAGGCGCG |
| 1022 | MME | 2648709 | TTAAACAAACAACCGTGACTACTAT |
| 1023 | MME | 2648709 | AATTAAACAAACAACCGTGACTACT |
| 1024 | MME | 2648709 | TGTCGACTTTTTCGATAACGTGTTG |
| 1025 | MME | 2648709 | TTTTTCGATAACGTGTTGACTTAAG |
| 1026 | MME | 2648727 | CCTCGTCGACATCAGTTACGTAAAA |
| 1027 | MME | 2648727 | ACCTCGTCGACATCAGTTACGTAAA |
| 1028 | MME | 2648727 | CTCGTCGACATCAGTTACGTAAAAT |
| 1029 | MME | 2648727 | CGTCGACATCAGTTACGTAAAATGA |
| 1030 | MME | 2648688 | TACCATAAACGTTCAGTAGTCTGAC |
| 1031 | MME | 2648688 | CCATAAACGTTCAGTAGTCTGACGT |
| 1032 | MME | 2648688 | AAACGTTCAGTAGTCTGACGTATTT |
| 1033 | MME | 2648688 | TTCAGTAGTCTGACGTATTTTAGTC |
| 1034 | MME | 2648732 | TTACCTTAATTATGTGACCCTCTTT |
| 1035 | MME | 2648732 | ATTACCTTAATTATGTGACCCTCTT |
| 1036 | MME | 2648732 | AATTACCTTAATTATGTGACCCTCT |
| 1037 | MME | 2648715 | AAAGACACCGGTCTAACTAAGCAGT |
| 1038 | MME | 2648715 | TACACCTAAAATACTAAAGACACCG |
| 1039 | MME | 2648715 | TCCTTCTTTCTAACGGGTAGCTACT |
| 1040 | MME | 2648715 | GGGTAGCTACTTTTGGTCGAACGAA |
| 1041 | MME | 2648718 | TACAGGACCTCTAAGTATTACCTAG |
| 1042 | MME | 2648718 | TTGGATGTTCCTCAGGTCTTTACGA |
| 1043 | MME | 2648718 | CGTCGGAGTCGGCTTGGATGTTCCT |
| 1044 | MME | 2648718 | GTATTACCTAGAACATTCGTCGGAG |
| 1045 | MME | 2648731 | TTCTACCTCTGGAGCAACTGACCAC |
| 1046 | MME | 2648731 | AGGGTCACGTACCACATAGTCATAC |
| 1047 | MME | 2648731 | ACTGACCACCTGAGTTGTCAGACGT |
| 1048 | MME | 2648731 | ACGTTCATTGAAATTCCTCGTTAGG |
| 1049 | MME | 2648705 | GACGAGCTGACTAGGTTTTGTACCT |
| 1050 | MME | 2648705 | AAAAGTTTATACGAACGCCTCCGAC |
| 1051 | MME | 2648705 | TGCATTACAGTAAGGGCTCTGGTCG |
| 1052 | MME | 2648705 | GCCTCCGACCAACTTTGCATTACAG |
| 1053 | MME | 2648736 | TATCCGGTCTCATACGCCAATTGAG |
| 1054 | MME | 2648736 | CCGGTCTCATACGCCAATTGAGGTA |
| 1055 | MME | 2648736 | CACCACACCTTGGATATCCGGTCTC |
| 1056 | MME | 2648736 | ACACCACACCTTGGATATCCGGTCT |
| 1057 | MME | 2648734 | GATTTAGTGTTTGTTGATAAAAAGA |
| 1058 | MME | 2648734 | CCTGAACTGGATTTAGTGTTTGTTG |
| 1059 | MME | 2648734 | TGAACTGGATTTAGTGTTTGTTGAT |
| 1060 | MME | 2648734 | ACCTGAACTGGATTTAGTGTTTGTT |
| 1061 | MME | 2648701 | GTCTACCGAGTACGCTCTTTCATCG |
| 1062 | MME | 2648701 | TGGTATTCCACAGAGTGTCTTTGAC |
| 1063 | MME | 2648701 | ATCACAATCTTACACGATACCTTAG |
| 1064 | MME | 2648701 | TCCTCTTCGAAGTAAGCAAGATAAT |
| 1065 | MME | 2648708 | ACCTCTTGGAGATGAGTTTGACAAT |
| 1066 | MME | 2648708 | TCTGTATATACCCACCGGTCATCGT |
| 1067 | MME | 2648708 | GTTTGACAATGGTCTGTATATACCC |
| 1068 | MME | 2648708 | ATACCCACCGGTCATCGTTGTCTTT |
| 1069 | MME | 2648703 | GTTTTCCCGTGTTTATCTCAGACTC |
| 1070 | MME | 2648703 | CCTCCGTTACTTGACTCTTGCCTAG |
| 1071 | MME | 2648703 | TACCTTACCTGAGACCTCCGTTACT |
| 1072 | MME | 2648703 | TTGCCTAGACTGGAGACACCGTTCC |
| 1073 | MME | 2648745 | GAGACATAACACGACTAACACCTAG |
| 1074 | MME | 2648745 | CGAGGGATTCTGACACTGTTGACAG |
| 1075 | MME | 2648745 | CTCCGGAGACTACCTGGAAGATCTT |
| 1076 | MME | 2648745 | TCTTCGAGAGCTGTTATGGGCAACC |
| 1077 | MME | 2648744 | CAGGGACTCTCACAGAGACTATTTT |
| 1078 | MME | 2648744 | CGGTAACCTGTCAACAGATCTCTAT |
| 1079 | MME | 2648744 | CCCGTTTAGACGTGGATACATCGAG |
| 1080 | MME | 2648744 | ATACATCGAGACGTAGAGGACAGAA |
| 1081 | NPR3 | 2805680 | ACATCTCGTATGTTTGTCGAGAGGG |
| 1082 | NPR3 | 2805680 | TCTTCCAGCAAAACTTTACGCCGGC |
| 1083 | NPR3 | 2805680 | ACAGTTTATAGGAACCCCGGGAAAT |
| 1084 | NPR3 | 2805680 | GGCTTAACATCTCGTATGTTTGTCG |
| 1085 | NPR3 | 2805664 | GTAGTACGACCACCGCGTGTCCGTA |
| 1086 | NPR3 | 2805664 | TACACACGCTCGTCACTGTGGTAGG |
| 1087 | NPR3 | 2805664 | CTCTGATGCGGAAGAAGTTGTAACT |
| 1088 | NPR3 | 2805664 | GTACTGGTCACCTCTGATGCGGAAG |
| 1089 | NPR3 | 2805646 | GGGCACGCATGATGAGCCGACCCGC |
| 1090 | NPR3 | 2805646 | GCACGCATGATGAGCCGACCCGCAA |
| 1091 | NPR3 | 2805646 | CACGAGTGAAAGAGGGGCACGCATG |
| 1092 | NPR3 | 2805646 | ATGATGAGCCGACCCGCAACGACCG |
| 1093 | NPR3 | 2805676 | CCTCCCTTTTAATATGTCGTCTGAA |
| 1094 | NPR3 | 2805676 | TTTAATATGTCGTCTGAACCTTGTC |
| 1095 | NPR3 | 2805676 | CTACGGTAGGAGGAGATGCAGAACC |
| 1096 | NPR3 | 2805676 | GAGATGTACTTCATGAGTCTCGACC |
| 1097 | NPR3 | 2805643 | AAGGACGAGAGTCACGCGACTGTCT |
| 1098 | NPR3 | 2805643 | TCGACCCTGTGACACTGGAGCCGTG |
| 1099 | NPR3 | 2805643 | CGCTAAGGTCGCGTTTGGACGCACC |
| 1100 | NPR3 | 2805643 | CTCGTTGTTCAAAGTGAAAGGACGA |
| 1101 | NPR3 | 2805647 | GGGTCCTACTGAGCATGAACAAAAG |
| 1102 | NPR3 | 2805647 | CTCCCAAACGTGTGCAGGTAGATGT |
| 1103 | NPR3 | 2805647 | AGCACGCGTTATAGGTCCGGTCACT |
| 1104 | NPR3 | 2805647 | GGTCCACCGAATGCTCCTAAGTCTG |
| 1105 | NPR3 | 2805678 | ATAGCGGCCCGTCCACAGGTATCTA |
| 1106 | NPR3 | 2805678 | GTTGCCTCTGGCTATACCCCTAAAG |
| 1107 | NPR3 | 2805678 | CCCCTAAAGAGACACTAACGGTACT |
| 1108 | NPR3 | 2805678 | GGTATCTACGGTTGCCTCTGGCTAT |
| 1109 | NPR3 | 2805636 | CTACACGACACATGCCTTGGACCGG |
| 1110 | NPR3 | 2805636 | CACGACACATGCCTTGGACCGGGAC |
| 1111 | NPR3 | 2805636 | TACACGACACATGCCTTGGACCGGG |
| 1112 | NPR3 | 2805636 | CCCTACACGACACATGCCTTGGACC |
| 1113 | NPR3 | 2805645 | TCTGCGTGTCCAAATGTGGGCCACT |
| 1114 | NPR3 | 2805645 | GCCTCCCGCTTATATATGTTCATAT |
| 1115 | NPR3 | 2805645 | TAACGTCTCTTCCTGCGAAGGAGAG |
| 1116 | NPR3 | 2805645 | AGGAGAGATAGAAAACCGCGTAATC |
| 1117 | NPR3 | 2805642 | GTCCTGGAAAGAACCTCAGCGCCTT |
| 1118 | NPR3 | 2805642 | CGCTACGAGGAGACCAGTGCCTGAA |
| 1119 | NPR3 | 2805642 | TCGCCAACCTAGAAACTCGTGAGTC |
| 1120 | NPR3 | 2805642 | CACGGAGCTCGCGAGGTCTCTTCCT |
| 1121 | NPR3 | 2805662 | ACTAGTAAGATGACGGACAATTGGT |
| 1122 | NPR3 | 2805662 | ATAGACAACCTTAGGGTCACCTTCT |
| 1123 | NPR3 | 2805662 | AGGTCAGTAGAAAGGTTATACCAAC |
| 1124 | NPR3 | 2805662 | GACAATTGGTGTGTACGTTCGAAAG |
| 1125 | NPR3 | 2805681 | ACCGGATCTTCTTAGCCGTCACTGT |
| 1126 | NPR3 | 2805681 | CCGAAATGATCCTCGACCGAACGAT |
| 1127 | NPR3 | 2805681 | GTCCTTAACAGCACCCCCGAAATGA |
| 1128 | NPR3 | 2805681 | GACCGAACGATTACCGGAAGATGAA |
| 1129 | NPR3 | 2805684 | AAACCCTCGTAAAGTGTGTTCCTAT |
| 1130 | NPR3 | 2805684 | ACTAATTAGTGGTAGACGGAGGTCC |
| 1131 | NPR3 | 2805684 | GTCTTCCCCGCAAGAACTTCTTAAG |
| 1132 | NPR3 | 2805684 | GCACAGTGAGACAATTTACAAGTAT |
| 1133 | NPR3 | 2805683 | AGTCTAGGGTAAAAAGTCATCGAAT |
| 1134 | NPR3 | 2805683 | TTTATGTCTTATTGGTAACTCTCCG |
| 1135 | NPR3 | 2805683 | AACTCTCCGCTTGGGTCGTTCTTCT |
| 1136 | NPR3 | 2805683 | GTAGCCCTTAATGCCCTTCTAAGGT |
| 1137 | NPR3 | 2805696 | GACGTATACCCAATGTTTTTGAGAC |
| 1138 | NPR3 | 2805696 | CCAAAGACCTACGATATGGTTAAAT |
| 1139 | NPR3 | 2805696 | TGTCCAGTTTTACGCATCTACGAAA |
| 1140 | NPR3 | 2805696 | TCACAGACAGAATACCAGTAGTAAC |
| 1141 | NPR3 | 2805685 | CAGAGTATTTGCGATGAGACCTAAC |
| 1142 | NPR3 | 2805685 | CTGTCCAAACACCAACTCCTGAAGA |
| 1143 | NPR3 | 2805685 | TCGGGATAAAGCGTGATTGTAAAAT |
| 1144 | NPR3 | 2805685 | CAGGCTACAGATGTAAGTCCAAGAC |
| 1145 | NPR3 | 2805688 | ACCCTTCGACAGTACTCTCACGTGG |
| 1146 | NPR3 | 2805688 | GAGTCCAATGATGAAAGTGAATATG |
| 1147 | NPR3 | 2805688 | CAGAACCTTAATGTATTGACCCCAG |
| 1148 | NPR3 | 2805688 | CCCAGAAAGGAGTTATTGTAAAACT |
| 1149 | NPR3 | 2805691 | GACTCTCCCGCACTATTTTCTTAAT |
| 1150 | NPR3 | 2805691 | CTCTTCAGGCGAAGACAACGAGGGT |
| 1151 | NPR3 | 2805691 | CGAAGACAACGAGGGTGGACTCAGT |
| 1152 | NPR3 | 2805691 | TCACACTAGTTGACTTTGTTGATAC |
| 1153 | NPR3 | 2805692 | AGGTCCGTTCGTTTCGCAACATGGT |
| 1154 | NPR3 | 2805692 | TAGGTCCGTTCGTTTCGCAACATGG |
| 1155 | NPR3 | 2805692 | GTCCGTTCGTTTCGCAACATGGTGA |
| 1156 | NPR3 | 2805692 | CGTTCGTTTCGCAACATGGTGAACC |
| 1157 | NPR3 | 2805686 | TCAAGACCGGTCTAGTACTCAAAGT |
| 1158 | NPR3 | 2805686 | CTTGTTGAAACAACTCTCAATGATG |
| 1159 | NPR3 | 2805686 | GATGAACTGTCGTTCGTGTCTTTAC |
| 1160 | NPR3 | 2805686 | CGTTCAATAAAATCCCACTGTGAGG |
| 1161 | NPR3 | 2805689 | TCAAGTGTGTTCATTGTCACCTCCG |
| 1162 | NPR3 | 2805699 | ACTGGTGTGAACGAGAGCCCCTTCA |
| 1163 | NPR3 | 2805699 | TCACCGCAGTGATGGAAGAACATTC |
| 1164 | NPR3 | 2805699 | TAGACTCGAAACAAGGGACACGTAC |
| 1165 | NPR3 | 2805699 | AACGGGTACGTCCATCTTTAAACAC |
| 1166 | NPR3 | 2805697 | AAAAACATATCTCAGGTAGAGAGGG |
| 1167 | NPR3 | 2805697 | AACATATCTCAGGTAGAGAGGGAGT |
| 1168 | NPR3 | 2805697 | CTTAAAAAACATATCTCAGGTAGAG |
| 1169 | NPR3 | 2805697 | TTCTTAAAAAACATATCTCAGGTAG |
| 1170 | NPR3 | 2805690 | CGTTCGACGGACAGGGTCTACGACC |
| 1171 | NPR3 | 2805690 | CGCGTTCGACGGACAGGGTCTACGA |
| 1172 | NPR3 | 2805690 | TCGACGGACAGGGTCTACGACCGGG |
| 1173 | NPR3 | 2805690 | CGGACAGGGTCTACGACCGGGTACA |
| 1174 | NPR3 | 2805698 | AGTTGTTACTCTACTTCCGGTAACG |
| 1175 | NPR3 | 2805698 | ACCTTACGGGAGTGAAGAGGGATAA |
| 1176 | NPR3 | 2805698 | ACACATTCTGTACGTCAGTTGTTAC |
| 1177 | NPR3 | 2805698 | GTGTCCTTACCAAGATGTCTGGGAT |
| 1178 | OR4K6P | 3527237 | TCGGTTAATCGAAAATGGGAGATAA |
| 1179 | OR4K6P | 3527237 | GTTAATCGAAAATGGGAGATAAACG |
| 1180 | OR4K6P | 3527237 | CTCGGTTAATCGAAAATGGGAGATA |
| 1181 | OR4K6P | 3527237 | GGTTAATCGAAAATGGGAGATAAAC |
| 1182 | OR4K6P | 3527236 | AAAAACGTGGAGAAGTGGCCCTGAC |
| 1183 | OR4K6P | 3527236 | ACGTGGAGAAGTGGCCCTGACTCTA |
| 1184 | OR4K6P | 3527236 | AACGTGGAGAAGTGGCCCTGACTCT |
| 1185 | OR4K6P | 3527236 | CTAGAAAAAAACGTGGAGAAGTGGC |
| 1186 | OR4K6P | 3527233 | CTTAAGTACAATGAACCTGAATGAC |
| 1187 | OR4K6P | 3527234 | CGAGTCGTTGGACAGAGAGTAACTG |
| 1188 | OR4K6P | 3527234 | AGTCGTTGGACAGAGAGTAACTGTA |
| 1189 | OR4K6P | 3527234 | CATGAAGGACGAGTCGTTGGACAGA |
| 1190 | OR4K6P | 3527234 | GACGAGTCGTTGGACAGAGAGTAAC |
| 1191 | OR4K6P | 3527235 | TACCTGAAAAAACGAGACGCATTCT |
| 1192 | OR4K6P | 3527235 | AGGAAACGGTGTGGTTTCTACTAAT |
| 1193 | OR4K6P | 3527235 | ACGAGACGCATTCTGGTAGAGAAAA |
| 1194 | OR4K6P | 3527235 | GGAAACGGTGTGGTTTCTACTAATA |
| 1195 | OR4K6P | 3527238 | GTGTTTGAAGGACCATCTGTTTTAA |
| 1196 | OR4K6P | 3527238 | AGACACTAGAAGGAAACCAGTAGGT |
| 1197 | OR4K6P | 3527238 | TGTCCTAGGAGGTTCCGAGAAAGAT |
| 1198 | OR4K6P | 3527238 | AGTCCCTGATGAGGAGGTGTCCTAG |
| 1199 | OR4K6P | 3780615 | CTTAAGTACAATGAACCTGAATGAC |
| 1200 | OR4K6P | 3780617 | ACGAGACGCATTCTGGTAGAGAAAA |
| 1201 | OR4K6P | 3780617 | CCTGAAAAAACGAGACGCATTCTGG |
| 1202 | OR4K6P | 3780617 | TGAAAAAACGAGACGCATTCTGGTA |
| 1203 | OR4K6P | 3780617 | TACCTGAAAAAACGAGACGCATTCT |
| 1204 | OR4K6P | 3780621 | GTGTTTGAAGGACCATCTGTTTTAA |
| 1205 | OR4K6P | 3780621 | AGTCCCTGATGAGGAGGTGTCCTAG |
| 1206 | OR4K6P | 3780621 | AGACACTAGAAGGAAACCAGTAGGT |
| 1207 | OR4K6P | 3780621 | TGTCCTAGGAGGTTCCGAGAAAGAT |
| 1208 | OR4K6P | 3925119 | AGTCCCTGATGAGGAGGTGTCCTAG |
| 1209 | OR4K6P | 3925119 | AGACACTAGAAGGAAACCAGTAGGT |
| 1210 | OR4K6P | 3925119 | TGTCCTAGGAGGTTCCGAGAAAGAT |
| 1211 | OR4K6P | 3925119 | GTGTTTGAAGGACCATCTGTTTTAA |
| 1212 | OR4K6P | 3925121 | ATAAGTTGTTAATACTCGGTTTCTC |
| 1213 | OR4K6P | 3925121 | TTGTTAATACTCGGTTTCTCACACA |
| 1214 | OR4K6P | 3925121 | GTTGTTAATACTCGGTTTCTCACAC |
| 1215 | OR4K6P | 3925121 | AAGTTGTTAATACTCGGTTTCTCAC |
| 1216 | OR4K6P | 3925122 | ACGAGACGCATTCTGGTAGAGAAAA |
| 1217 | OR4K6P | 3925122 | TACCTGAAAAAACGAGACGCATTCT |
| 1218 | OR4K6P | 3925122 | TGAAAAAACGAGACGCATTCTGGTA |
| 1219 | OR4K6P | 3925122 | CCTGAAAAAACGAGACGCATTCTGG |
| 1220 | OR4K6P | 3925124 | CTTAAGTACAATGAACCTGAATGAC |
| 1221 | OR4K7P | 3780618 | ATAACGGTATACATTTGGAGAGGTG |
| 1222 | OR4K7P | 3780618 | GATAAGTTGTTAATACTCGGTTTCT |
| 1223 | OR4K7P | 3780618 | TAACGGTATACATTTGGAGAGGTGA |
| 1224 | OR4K7P | 3780618 | TTTGGAGAGGTGATAAGTTGTTAAT |
| 1225 | OR4K7P | 3780619 | CACACACAACTCGAACACCGTCAAA |
| 1226 | OR4K7P | 3780619 | CACACAACTCGAACACCGTCAAAGA |
| 1227 | OR4K7P | 3780619 | TCGAACACCGTCAAAGAACAACCTG |
| 1228 | OR4K7P | 3780619 | GAACACCGTCAAAGAACAACCTGTC |
| 1229 | OR4K7P | 3780620 | ACTCGGTTAATCAAAAAGGGAGATA |
| 1230 | OR4K7P | 3780620 | ACCCGAAAGATGTATGTTACTCGGT |
| 1231 | OR4K7P | 3780620 | GGGAAGACACAAGGGTTACAACATC |
| 1232 | OR4K7P | 3780620 | AGATGTATGTTACTCGGTTAATCAA |
| 1233 | OR4K7P | 3925120 | ACTCGGTTAATCAAAAAGGGAGATA |
| 1234 | OR4K7P | 3925120 | AGATGTATGTTACTCGGTTAATCAA |
| 1235 | OR4K7P | 3925120 | GGGAAGACACAAGGGTTACAACATC |
| 1236 | OR4K7P | 3925120 | ACCCGAAAGATGTATGTTACTCGGT |
| 1237 | P3H2 | 2710502 | TTTCGATAGTGGGTTCTATCTAGCT |
| 1238 | P3H2 | 2710502 | CCAGGGAAGTCCTCACTTGCATCTC |
| 1239 | P3H2 | 2710502 | GGGAAGTCCTCACTTGCATCTCCCT |
| 1240 | P3H2 | 2710502 | TTCTATCTAGCTCTGGATTCTCTTC |
| 1241 | P3H2 | 2710503 | ATACCTCCTGCTGTCCTACTCTTAG |
| 1242 | P3H2 | 2710503 | TACCTCCTGCTGTCCTACTCTTAGC |
| 1243 | P3H2 | 2710483 | CGAGACACCAAGTGGAACCTGGGTG |
| 1244 | P3H2 | 2710483 | ACCGAGACACCAAGTGGAACCTGGG |
| 1245 | P3H2 | 2710483 | TGGAACCTGGGTGAAATATCTCTTA |
| 1246 | P3H2 | 2710483 | GTGGAACCTGGGTGAAATATCTCTT |
| 1247 | P3H2 | 2710492 | CTCTTAAGTATAAGTGTCTCTACCT |
| 1248 | P3H2 | 2710492 | CCTCCTCTTAAGTATAAGTGTCTCT |
| 1249 | P3H2 | 2710492 | TTCCTCCTCTTAAGTATAAGTGTCT |
| 1250 | P3H2 | 2710492 | TCCTCTTAAGTATAAGTGTCTCTAC |
| 1251 | P3H2 | 2710496 | CTTTTCAAACTTCCACGTTGACAGG |
| 1252 | P3H2 | 2710496 | GGGTATGTGGGTTACTTTTCAAACT |
| 1253 | P3H2 | 2710496 | ACGTTGACAGGACTTTCGTGAGTTT |
| 1254 | P3H2 | 2710496 | ACTTCCACGTTGACAGGACTTTCGT |
| 1255 | P3H2 | 2710542 | GATGTCGCCTCTGATGCTCGCTCGC |
| 1256 | P3H2 | 2710542 | GCCGCGCCGGCGGATGATGTCGCCT |
| 1257 | P3H2 | 2710542 | GCTGGACGAGATGCGGTCGCCGCGC |
| 1258 | P3H2 | 2710542 | TCGGGAAGCTGGACGAGATGCGGTC |
| 1259 | P3H2 | 2710493 | GCAGCACAAGTATAGAGGTTCCGTC |
| 1260 | P3H2 | 2710493 | TAGAAGACTTCATCGACGGTATTGG |
| 1261 | P3H2 | 2710493 | CGTGTCAAACGTAGGGTAAAGTGTT |
| 1262 | P3H2 | 2710493 | GAGACAAAACGAACTGCAGCACAAG |
| 1263 | P3H2 | 2710506 | AAGGAGACGTGATACTAATGGATGT |
| 1264 | P3H2 | 2710506 | GTGATGTACGTCCACGAACAAACAG |
| 1265 | P3H2 | 2710506 | ACACTCCCTTGAACGGTGGGCGGGA |
| 1266 | P3H2 | 2710506 | ATGGATGTCAAACGGATGATAGCTC |
| 1267 | P3H2 | 2710487 | TCGATCCTGAGACCACTGTAAGTTT |
| 1268 | P3H2 | 2710487 | CGGTATAAGGTTACCCCCGGTTCGG |
| 1269 | P3H2 | 2710487 | TCCCTGGAGCAAAATAGAGAACTCG |
| 1270 | P3H2 | 2710487 | GTTCGGGGGTTTGCTCTCTGCAAGA |
| 1271 | P3H2 | 2710543 | ATGACGGCGGCGGTGACACCCCGCC |
| 1272 | P3H2 | 2710543 | CCCTCGCGTAGACCCGCGGCGGCGA |
| 1273 | P3H2 | 2710543 | ACGGCGGCGGTGACACCCCGCCGGG |
| 1274 | P3H2 | 2710543 | TACGCCCTCGCGTAGACCCGCGGCG |
| 1275 | P3H2 | 2710544 | CTCGCATTGGCAGGGCGCGGAGAGA |
| 1276 | P3H2 | 2710544 | GGGCTTCGGGAGAGCTCGCATTGGC |
| 1277 | P3H2 | 2710544 | GGCAGGGCGCGGAGAGACTCCGCCT |
| 1278 | P3H2 | 2710544 | GCCTGCGCTCCACGGGGCTTCGGGA |
| 1279 | P3H2 | 2710539 | GTGGCGCAGTCGCTCCTACACGCGT |
| 1280 | P3H2 | 2710539 | GGCGTAGGGCGGTGGCGCAGTCGCT |
| 1281 | P3H2 | 2710539 | TCGCGTCTCACGGGATGTTGATGGA |
| 1282 | P3H2 | 2710539 | ACACGCGTCGCTGAAGGTCGCGTCT |
| 1283 | P3H2 | 2710482 | ACTATCAGTGATTAGACAGAACTCG |
| 1284 | P3H2 | 2710482 | TCGGTTAATCCGAACGAAGTACTTG |
| 1285 | P3H2 | 2710482 | TCAGGAAATTTAACGAAGCAGACTC |
| 1286 | P3H2 | 2710482 | CACTCAAAGAGTGGGCGGGTCTTTC |
| 1287 | P3H2 | 2710509 | TCTACCGATAGTCCGTGAAGCTTGT |
| 1288 | P3H2 | 2710509 | TGTCTTACGGCCTGGGATACACTCC |
| 1289 | P3H2 | 2710509 | AAAGCAACTTCTATGTCTTACGGCC |
| 1290 | P3H2 | 2710509 | CCGACCAGACATACTTCGATAACGT |
| 1291 | P3H2 | 2710494 | GGGTAGGTACGACTGTTGACAAACA |
| 1292 | P3H2 | 2710494 | ACAAACAACCTAGGTCTCCGGTTGC |
| 1293 | P3H2 | 2710494 | GGACGAATGTGTAAAGCTCTGATAT |
| 1294 | P3H2 | 2710494 | GTCTCCGGTTGCTTACGACCTTCCT |
| 1295 | P3H2 | 2710540 | ACAACCCCGCCCGCGCGACAATAGC |
| 1296 | P3H2 | 2710540 | GAACAACCCCGCCCGCGCGACAATA |
| 1297 | P3H2 | 2710540 | GACGGGGAAAAGGCGAGGAACAACC |
| 1298 | P3H2 | 2710540 | ACCCCGCCCGCGCGACAATAGCGTC |
| 1299 | P3H2 | 2710504 | TTGCAGTATTCGACCTCAGACTCGA |
| 1300 | P3H2 | 2710504 | TTTGCAGTATTCGACCTCAGACTCG |
| 1301 | P3H2 | 2710504 | GACCTCAGACTCGACTATTTTAGTC |
| 1302 | P3H2 | 2710495 | ACTGTAGTCGCTTTTCCGAGCTTCC |
| 1303 | P3H2 | 2710495 | AGACCAATACTTCCAGCTCAGGGTG |
| 1304 | P3H2 | 2710495 | TATGTGTGTACCAGACGGCTTGTCG |
| 1305 | P3H2 | 2710495 | TCAGGGTGACTTCTCGCGAGCAGAC |
| 1306 | P3H2 | 2710497 | ACCCCTACCAAGAATCGTAACTGAT |
| 1307 | P3H2 | 2710497 | ACTACTGCATAGAAGAACATAAACG |
| 1308 | P3H2 | 2710497 | AGAATCGTAACTGATCTCAAGAGTC |
| 1309 | P3H2 | 2710497 | TCTGGACTACTTAAGTATCAATAGT |
| 1310 | P3H2 | 2710546 | CGGTCCGTGCCGGAGGCGGAGAGTC |
| 1311 | P3H2 | 2710488 | CATACTCTGGAGTCAGGGAGATACT |
| 1312 | P3H2 | 2710488 | TACTCTGGAGTCAGGGAGATACTAC |
| 1313 | P3H2 | 2710488 | ACTCTGGAGTCAGGGAGATACTACC |
| 1314 | P3H2 | 2710488 | TCTGGAGTCAGGGAGATACTACCTT |
| 1315 | P3H2 | 2710489 | GGACCGAAGGTTCCAGTTGATGGTC |
| 1316 | P3H2 | 2710489 | GGTAGGACCGAAGGTTCCAGTTGAT |
| 1317 | P3H2 | 2710489 | ACCCTCCGTCGTGTGGTAGGACCGA |
| 1318 | P3H2 | 2710489 | GTACCCTCCGTCGTGTGGTAGGACC |
| 1319 | P3H2 | 2710505 | CGGGACCTCACACGGTTTCGGATAG |
| 1320 | P3H2 | 2710505 | ACACCTAATGATACTCTCAGACGAC |
| 1321 | P3H2 | 2710505 | AACTGGGCCGTAGGTAACTCCGGTC |
| 1322 | P3H2 | 2710505 | AACCACTCATACACTTTCGGGACCT |
| 1323 | P3H2 | 2710545 | CTCGCCGGTCTAGCGCCGCCTCAGC |
| 1324 | P3H2 | 2710545 | GACCCCGAGCGCCTCGCCGGTCTAG |
| 1325 | P3H2 | 2710545 | TCAGCCGCGCGAAGGGGCTCCCTTC |
| 1326 | P3H2 | 2710545 | TCGCGGCCGCCAGTGGACCCCGAGC |
| 1327 | P3H2 | 2710476 | CCGCATTACTAGTGGGTCCGAGGCC |
| 1328 | P3H2 | 2710476 | GCGACGGAGTCCATAGTACCCGCAT |
| 1329 | P3H2 | 2710476 | GTGCACACGAGGTCAAGATTTTAAT |
| 1330 | P3H2 | 2710476 | GAATCCACGATTGCCGGTACTCGAG |
| 1331 | P3H2 | 2710486 | AGGTCTCCACCAAGAGTGATTTCTC |
| 1332 | P3H2 | 2710486 | AATGTCTGGGGCTTTCTTTTACGAT |
| 1333 | P3H2 | 2710486 | CAATACGCTCATAACGGTACCACTG |
| 1334 | P3H2 | 2710486 | CCGACCTTAAACAATACGCTCATAA |
| 1335 | P3H2 | 2710485 | GTACCGGACTTCTTATATCGGGTCA |
| 1336 | P3H2 | 2710485 | ATGGAGTCTATCCTAGGTACCTTAC |
| 1337 | P3H2 | 2710485 | TAGGTACCTTACCAGTTTTTGGGAG |
| 1338 | P3H2 | 2710485 | ACCAGTTTTTGGGAGGTACCGGACT |
| 1339 | P3H2 | 2710541 | GACGCCCTTTAGGCGTGCGCGACAC |
| 1340 | P3H2 | 2710541 | CGCCCTTTAGGCGTGCGCGACACGG |
| 1341 | P3H2 | 2710541 | CGGACGCCCTTTAGGCGTGCGCGAC |
| 1342 | P3H2 | 2710541 | CCGCGGACGCCCTTTAGGCGTGCGC |
| 1343 | P3H2 | 2710484 | CCACTTCCGTCAGTGGTTCCCTTTC |
| 1344 | P3H2 | 2710484 | TTTACACCCGCGTACTAGTCGAAGA |
| 1345 | P3H2 | 2710484 | CTCCTCTCTTGGGAGTACCCCACTT |
| 1346 | P3H2 | 2710484 | TTTTACACCCGCGTACTAGTCGAAG |
| 1347 | P3H2 | 2710510 | GTAACTCTTAATGTCCCGCTGTCGA |
| 1348 | P3H2 | 2710510 | CACAACTTCGTAACGTCAACCATCT |
| 1349 | P3H2 | 2710510 | TAATGTCCCGCTGTCGACCACAACT |
| 1350 | P3H2 | 2710510 | CGTAACGTCAACCATCTGTCTCTTC |
| 1351 | P3H2 | 2710475 | AAACACGAACACAGACTAAACAAAT |
| 1352 | P3H2 | 2710475 | TCTAAACACGAACACAGACTAAACA |
| 1353 | P3H2 | 2710475 | GACTAAACAAATTATTTCCCTCCGA |
| 1354 | P3H2 | 2710475 | GAGTAACGACGATAGGTCGTGTGTC |
| 1355 | P3H2 | 3164926 | TTTTATACACCTATGAGGTAAACCG |
| 1356 | P3H2 | 3164926 | TATACACCTATGAGGTAAACCGTTC |
| 1357 | P3H2 | 3164926 | TAACCTTATTAACCACCTTGTCCGG |
| 1358 | P3H2 | 3164926 | ACCTACTAGGTCTTTAAAATCTTCC |
| 1359 | P3H2 | 2710549 | GTAGGTGTGGTTCAACCTGCTTTTC |
| 1360 | P3H2 | 2710549 | GGGACTAGGTTACGGGCCTGTGTGT |
| 1361 | P3H2 | 2710549 | TGTAGGTGTGGTTCAACCTGCTTTT |
| 1362 | P3H2 | 2710549 | ACTAGGTTACGGGCCTGTGTGTAGG |
| 1363 | POTEB2 | 3612777 | TCTTTCTTCTTCTAGAGAACGCACT |
| 1364 | POTEB2 | 3612777 | CTTACTTCTTCGTAATTGCTTTTGG |
| 1365 | POTEB2 | 3612777 | TCTTCTTCTAGAGAACGCACTTTTG |
| 1366 | POTEB2 | 3612777 | TCTTCTAGAGAACGCACTTTTGTCG |
| 1367 | POTEB2 | 3612778 | CTAAGACTGATTATTTGTTTTCGTC |
| 1368 | POTEB2 | 3612778 | TTACTATGGGTCTTTGTTGAAAGAC |
| 1369 | POTEB2 | 3612778 | TCTACTCTAAGACTGATTATTTGTT |
| 1370 | POTEB2 | 3612778 | CGTCTATCTTCACCGACTTTTCCTT |
| 1371 | POTEB2 | 3612779 | TACCTTCATTAGGACACCCTAATGG |
| 1372 | POTEB2 | 3612779 | CCTTCATTAGGACACCCTAATGGTC |
| 1373 | POTEB2 | 3612779 | TAGGACACCCTAATGGTCTTTTGGA |
| 1374 | POTEB2 | 3612779 | TTCGTACCTTCATTAGGACACCCTA |
| 1375 | POTEB2 | 3612781 | TTGGTCTTTATTTATTCCTGACACT |
| 1376 | POTEB2 | 3612781 | GGTCTTTATTTATTCCTGACACTAT |
| 1377 | POTEB2 | 3612781 | TCTTTATTTATTCCTGACACTATCT |
| 1378 | POTEB2 | 3612781 | TTATTTATTCCTGACACTATCTCTC |
| 1379 | POTEB2 | 3612782 | TCAGTCACTTTTATCGGTCGGTCTC |
| 1380 | POTEB2 | 3612782 | AGTGTTTCCGAATTTCAGTCACTTT |
| 1381 | POTEB2 | 3612782 | CCTTCTCAGTGTTTCCGAATTTCAG |
| 1382 | POTEB2 | 3612782 | TTTCAGTCACTTTTATCGGTCGGTC |
| 1383 | POTEB2 | 3612786 | CTTAATGAAAGACTGATATTTCTTT |
| 1384 | POTEB2 | 3612786 | CTACGATTTTTAGAGAAGACTTTTG |
| 1385 | POTEB2 | 3612786 | TAGAGAAGACTTTTGTCGTTAGGTC |
| 1386 | POTEB2 | 3612786 | ACACTTAATGAAAGACTGATATTTC |
| 1387 | POTEB2 | 3612787 | GTCTCTTTCAATCGTGCCGAAGACG |
| 1388 | POTEB2 | 3612787 | TATGTCTCTTTCAATCGTGCCGAAG |
| 1389 | POTEB2 | 3612787 | TGCCGAAGACGTATTCCTCCGTCGT |
| 1390 | POTEB2 | 3612787 | CTCTTTCAATCGTGCCGAAGACGTA |
| 1391 | POTEB2 | 3612790 | AAACGAACCGCATGTACTTGTTTTT |
| 1392 | POTEB2 | 3612790 | AAAACGAACCGCATGTACTTGTTTT |
| 1393 | POTEB2 | 3612790 | GAAAACGAACCGCATGTACTTGTTT |
| 1394 | POTEB2 | 3612791 | TGGCGAGATGTGATACGATAGATGT |
| 1395 | POTEB2 | 3612791 | ACCTTGTACCGCGACTACCTTTATA |
| 1396 | POTEB2 | 3612791 | ACCGCGACTACCTTTATAAGTTCTA |
| 1397 | POTEB2 | 3612791 | CTACCTTTATAAGTTCTACTCATAC |
| 1398 | POTEB2 | 3612795 | GTTCTCGTTGCACCCGTGAACCCCT |
| 1399 | POTEB2 | 3612795 | GACTGTACTTGTTCTCCCTGTTCGT |
| 1400 | POTEB2 | 3612795 | GAAGTACCTCGGCTCCATGGTGCAG |
| 1401 | POTEB2 | 3612795 | GTGAACCCCTCTGATGCTGCTGTCG |
| 1402 | POTEB2 | 3612796 | CGTTCACCACGACAGTGACGAAGGG |
| 1403 | POTEB2 | 3612796 | TGAGTCCTCGTTCTACCCGTTCACC |
| 1404 | POTEB2 | 3612796 | CCCGTTCACCACGACAGTGACGAAG |
| 1405 | POTEB2 | 3612796 | TCACCACGACAGTGACGAAGGGGAC |
| 1406 | POTEB2 | 3612798 | CACCGACTCCAAACAAGTTACGGGC |
| 1407 | POTEB2 | 3612798 | CCAAACAAGTTACGGGCGACGGAGA |
| 1408 | POTEB2 | 3612798 | ACTCCAAACAAGTTACGGGCGACGG |
| 1409 | POTEB2 | 3612798 | CAAGTTACGGGCGACGGAGACGACA |
| 1410 | POTEB2 | 3800384 | TCTTTCTTCTTCTAGAGAACGCACT |
| 1411 | POTEB2 | 3800384 | CTTTCTTCTTCTAGAGAACGCACTT |
| 1412 | POTEB2 | 3800384 | TCTTCTTCTAGAGAACGCACTTTTG |
| 1413 | POTEB2 | 3800384 | TCTTCTAGAGAACGCACTTTTGTCG |
| 1414 | POTEB2 | 3800387 | TTCGTACCTTCATTAGGACACCCTA |
| 1415 | POTEB2 | 3800387 | CCTTCATTAGGACACCCTAATGGTC |
| 1416 | POTEB2 | 3800387 | TAGGACACCCTAATGGTCTTTTGGA |
| 1417 | POTEB2 | 3800387 | TACCTTCATTAGGACACCCTAATGG |
| 1418 | POTEB2 | 3800392 | TAGAGAAGACTTTTGTCGTTAGGTC |
| 1419 | POTEB2 | 3800392 | ACACTTAATGAAAGACTGATATTTC |
| 1420 | POTEB2 | 3800392 | CTTAATGAAAGACTGATATTTCTTT |
| 1421 | POTEB2 | 3800392 | CTACGATTTTTAGAGAAGACTTTTG |
| 1422 | POTEB2 | 3800395 | GAAAACGAACCGCATGTACTTGTTT |
| 1423 | POTEB2 | 3800395 | AAAACGAACCGCATGTACTTGTTTT |
| 1424 | POTEB2 | 3800395 | AAACGAACCGCATGTACTTGTTTTT |
| 1425 | POTEB2 | 3800396 | GAATATACCACGACTATAACTTAGT |
| 1426 | POTEB2 | 3800396 | TGACGAGAATATACCACGACTATAA |
| 1427 | POTEB2 | 3800396 | CGGTTTCGTGACGAGAATATACCAC |
| 1428 | POTEB2 | 3800396 | CGAGAATATACCACGACTATAACTT |
| 1429 | POTEB2 | 3914572 | AAACGAACCGCATGTACTTGTTTTT |
| 1430 | POTEB2 | 3914572 | GAAAACGAACCGCATGTACTTGTTT |
| 1431 | POTEB2 | 3914572 | AAAACGAACCGCATGTACTTGTTTT |
| 1432 | POTEB2 | 3914576 | ACACTTAATGAAAGACTGATATTTC |
| 1433 | POTEB2 | 3914576 | TAGAGAAGACTTTTGTCGTTAGGTC |
| 1434 | POTEB2 | 3914576 | CTACGATTTTTAGAGAAGACTTTTG |
| 1435 | POTEB2 | 3914576 | CTTAATGAAAGACTGATATTTCTTT |
| 1436 | POTEB2 | 3914579 | AGTGTTTCCGAATTTCAGTCACTTT |
| 1437 | POTEB2 | 3914579 | CCTTCTCAGTGTTTCCGAATTTCAG |
| 1438 | POTEB2 | 3914579 | TTTCAGTCACTTTTATCGGTCGGTC |
| 1439 | POTEB2 | 3914579 | TCAGTCACTTTTATCGGTCGGTCTC |
| 1440 | POTEB2 | 3914582 | TTCGTACCTTCATTAGGACACCCTA |
| 1441 | POTEB2 | 3914582 | CCTTCATTAGGACACCCTAATGGTC |
| 1442 | POTEB2 | 3914582 | TACCTTCATTAGGACACCCTAATGG |
| 1443 | POTEB2 | 3914582 | TAGGACACCCTAATGGTCTTTTGGA |
| 1444 | RP11- | 3612740 | TATACTTTTGGATAAGACCAGATTT |
| | 403B2.10 | | |
| 1445 | RP11- | 3612740 | ATACTTTTGGATAAGACCAGATTTA |
| | 403B2.10 | | |
| 1446 | RP11- | 3612741 | ACCCGAGACTAGTCACGGACAACGC |
| | 403B2.10 | | |
| 1447 | RP11- | 3612741 | CCCGAGACTAGTCACGGACAACGCA |
| | 403B2.10 | | |
| 1448 | RP11- | 3612741 | CACCCGAGACTAGTCACGGACAACG |
| | 403B2.10 | | |
| 1449 | RP11- | 3612743 | AAGAGATGGAACTCACGCGGTCCGC |
| | 403B2.10 | | |
| 1450 | RP11- | 3612743 | ACCTCAGAAATTCTAAAAGAGATGG |
| | 403B2.10 | | |
| 1451 | RP11- | 3612743 | TGGACCTCGCTCTCGGCGGATGGAC |
| | 403B2.10 | | |
| 1452 | RP11- | 3612743 | AGATGGAACTCACGCGGTCCGCGCC |
| | 403B2.10 | | |
| 1453 | RP11- | 3612746 | TTTACGTCAACTTTCTATAAAGTAT |
| | 403B2.10 | | |
| 1454 | RP11- | 3612746 | CGTCAACTTTCTATAAAGTATTTCC |
| | 403B2.10 | | |
| 1455 | RP11- | 3612746 | TACGTCAACTTTCTATAAAGTATTT |
| | 403B2.10 | | |
| 1456 | RP11- | 3612746 | TCAACTTTCTATAAAGTATTTCCTT |
| | 403B2.10 | | |
| 1457 | RP11- | 3612750 | CTTCTGAAACTTGCGTGAGGAGTCT |
| | 403B2.10 | | |
| 1458 | RP11- | 3612750 | CGACAGCATTTTTCGCGTCTCTTCT |
| | 403B2.10 | | |
| 1459 | RP11- | 3612750 | GAGGGTGGACGATTCCATCGACGGG |
| | 403B2.10 | | |
| 1460 | RP11- | 3612750 | CGACGGGGATTAGATTCAGCTTACC |
| | 403B2.10 | | |
| 1461 | RP11- | 3612752 | CCTGATTAACGTCCTCGGTAATATC |
| | 403B2.10 | | |
| 1462 | RP11- | 3612752 | GACCTGATTAACGTCCTCGGTAATA |
| | 403B2.10 | | |
| 1463 | RP11- | 3612752 | ACCTGATTAACGTCCTCGGTAATAT |
| | 403B2.10 | | |
| 1464 | RP11- | 3612754 | TGTATGTCGACATGTAGTCTTTGTC |
| | 403B2.10 | | |
| 1465 | RP11- | 3612754 | ATGAGACCCTGTATGTCGACATGTA |
| | 403B2.10 | | |
| 1466 | RP11- | 3612754 | GTATGTCGACATGTAGTCTTTGTCT |
| | 403B2.10 | | |
| 1467 | RP11- | 3612754 | AGACCCTGTATGTCGACATGTAGTC |
| | 403B2.10 | | |
| 1468 | RP11- | 3612758 | CCAAGTGAAGGTAATGTCATACTCA |
| | 403B2.10 | | |
| 1469 | RP11- | 3612758 | ACCGTTTTTAACAGACTGAGTGTCT |
| | 403B2.10 | | |
| 1470 | RP11- | 3612758 | ATGTCATACTCACCGTTTTTAACAG |
| | 403B2.10 | | |
| 1471 | RP11- | 3612758 | GTTTTCTTCCAAGTGAAGGTAATGT |
| | 403B2.10 | | |
| 1472 | RP11- | 3612760 | AGACGGTACGTTTAAATGCGAATCA |
| | 403B2.10 | | |
| 1473 | RP11- | 3612762 | CAGCGCCTAAAGTAGTCTCCAACCT |
| | 403B2.10 | | |
| 1474 | RP11- | 3612762 | AGCGCCTAAAGTAGTCTCCAACCTC |
| | 403B2.10 | | |
| 1475 | RP11- | 3612762 | TCAGCGCCTAAAGTAGTCTCCAACC |
| | 403B2.10 | | |
| 1476 | RP11- | 3800341 | TATACTTTTGGATAAGACCAGATTT |
| | 403B2.10 | | |
| 1477 | RP11- | 3800341 | ATACTTTTGGATAAGACCAGATTTA |
| | 403B2.10 | | |
| 1478 | RP11- | 3800343 | CGACGGGGATTAGATTCAGCTTACC |
| | 403B2.10 | | |
| 1479 | RP11- | 3800343 | CATCGACGGGGATTAGATTCAGCTT |
| | 403B2.10 | | |
| 1480 | RP11- | 3800343 | GACGGGGATTAGATTCAGCTTACCC |
| | 403B2.10 | | |
| 1481 | RP11- | 3800343 | TCGACGGGGATTAGATTCAGCTTAC |
| | 403B2.10 | | |
| 1482 | RP11- | 3800345 | CTGAAACTTGCGTGAGGAGTCTCAG |
| | 403B2.10 | | |
| 1483 | RP11- | 3800345 | CTTCTGAAACTTGCGTGAGGAGTCT |
| | 403B2.10 | | |
| 1484 | RP11- | 3800345 | TGAAACTTGCGTGAGGAGTCTCAGG |
| | 403B2.10 | | |
| 1485 | RP11- | 3800345 | TCTGAAACTTGCGTGAGGAGTCTCA |
| | 403B2.10 | | |
| 1486 | RP11- | 3800349 | AATAGTAGAAAACGACAGCATTTTT |
| | 403B2.10 | | |
| 1487 | RP11- | 3800349 | ACGACAGCATTTTTCGCGTCTCTTC |
| | 403B2.10 | | |
| 1488 | RP11- | 3800349 | ACAGCATTTTTCGCGTCTCTTCTTT |
| | 403B2.10 | | |
| 1489 | RP11- | 3800349 | GAATAGTAGAAAACGACAGCATTTT |
| | 403B2.10 | | |
| 1490 | RP11- | 3800351 | CCCGAGACTAGTCACGGACAACGCA |
| | 403B2.10 | | |
| 1491 | RP11- | 3800351 | AAGTTTATTTCGACCTGATTAACGT |
| | 403B2.10 | | |
| 1492 | RP11- | 3800351 | GAAGTTTATTTCGACCTGATTAACG |
| | 403B2.10 | | |
| 1493 | RP11- | 3800351 | CTCACCCGAGACTAGTCACGGACAA |
| | 403B2.10 | | |
| 1494 | RP11- | 3800355 | ACCGTTTTTAACAGACTGAGTGTCT |
| | 403B2.10 | | |
| 1495 | RP11- | 3800355 | GTTTTCTTCCAAGTGAAGGTAATGT |
| | 403B2.10 | | |
| 1496 | RP11- | 3800355 | ATGTCATACTCACCGTTTTTAACAG |
| | 403B2.10 | | |
| 1497 | RP11- | 3800355 | CCAAGTGAAGGTAATGTCATACTCA |
| | 403B2.10 | | |
| 1498 | RP11- | 3800363 | AGACGGTACGTTTAAATGCGAATCA |
| | 403B2.10 | | |
| 1499 | RP11- | 3800365 | GTCGGCGGCGGGTGCCGTGCCGTCG |
| | 403B2.10 | | |
| 1500 | RP11- | 3800365 | TCGGCGGCGGGTGCCGTGCCGTCGG |
| | 403B2.10 | | |
| 1501 | RP11- | 3914598 | CGACGGGGATTAGATTCAGCTTACC |
| | 403B2.10 | | |
| 1502 | RP11- | 3914598 | CTTCTGAAACTTGCGTGAGGAGTCT |
| | 403B2.10 | | |
| 1503 | RP11- | 3914598 | GAGGGTGGACGATTCCATCGACGGG |
| | 403B2.10 | | |
| 1504 | RP11- | 3914598 | TGAAACTTGCGTGAGGAGTCTCAGG |
| | 403B2.10 | | |
| 1505 | RP11- | 3914599 | ACTACTAAGGGCGTGTCTCGTTCCT |
| | 403B2.10 | | |
| 1506 | RP11- | 3914599 | AAGGGCGTGTCTCGTTCCTACCCAG |
| | 403B2.10 | | |
| 1507 | RP11- | 3914599 | GCGTGTCTCGTTCCTACCCAGATAT |
| | 403B2.10 | | |
| 1508 | RP11- | 3914599 | AGGACACTACTAAGGGCGTGTCTCG |
| | 403B2.10 | | |
| 1509 | RP11- | 3914604 | TCGTCTTTACTGAAGTAGACAATAT |
| | 403B2.10 | | |
| 1510 | RP11- | 3914604 | TCTTTACTGAAGTAGACAATATAGA |
| | 403B2.10 | | |
| 1511 | RP11- | 3914604 | CAATCGTCTTTACTGAAGTAGACAA |
| | 403B2.10 | | |
| 1512 | RP11- | 3914604 | GTCTTTACTGAAGTAGACAATATAG |
| | 403B2.10 | | |
| 1513 | RP11- | 3914605 | GACTCATGAGACCCTGTATGTCGAC |
| | 403B2.10 | | |
| 1514 | RP11- | 3914605 | TCATGAGACCCTGTATGTCGACATG |
| | 403B2.10 | | |
| 1515 | RP11- | 3914605 | ACTCATGAGACCCTGTATGTCGACA |
| | 403B2.10 | | |
| 1516 | RP11- | 3914605 | AGACTCATGAGACCCTGTATGTCGA |
| | 403B2.10 | | |
| 1517 | RP11- | 3914609 | CCAAGTGAAGGTAATGTCATACTCA |
| | 403B2.10 | | |
| 1518 | RP11- | 3914609 | ATGTCATACTCACCGTTTTTAACAG |
| | 403B2.10 | | |
| 1519 | RP11- | 3914609 | ACCGTTTTTAACAGACTGAGTGTCT |
| | 403B2.10 | | |
| 1520 | RP11- | 3914609 | GTTTTCTTCCAAGTGAAGGTAATGT |
| | 403B2.10 | | |
| 1521 | RP11- | 3914611 | AATAGTAGAAAACGACAGCATTTTT |
| | 403B2.10 | | |
| 1522 | RP11- | 3914611 | ACAGCATTTTTCGCGTCTCTTCTTT |
| | 403B2.10 | | |
| 1523 | RP11- | 3914611 | GAATAGTAGAAAACGACAGCATTTT |
| | 403B2.10 | | |
| 1524 | RP11- | 3914611 | ACGACAGCATTTTTCGCGTCTCTTC |
| | 403B2.10 | | |
| 1525 | RP11- | 3915491 | AGACGGTACGTTTAAATGCGAATCA |
| | 403B2.10 | | |
| 1526 | RP11- | 3915497 | GTTTTCTTCCAAGTGAAGGTAATGT |
| | 403B2.10 | | |
| 1527 | RP11- | 3915497 | ACCGTTTTTAACAGACTGAGTGTCT |
| | 403B2.10 | | |
| 1528 | RP11- | 3915497 | CCAAGTGAAGGTAATGTCATACTCA |
| | 403B2.10 | | |
| 1529 | RP11- | 3915497 | ATGTCATACTCACCGTTTTTAACAG |
| | 403B2.10 | | |
| 1530 | RP11- | 3915500 | GAAGTTTATTTCGACCTGATTAACG |
| | 403B2.10 | | |
| 1531 | RP11- | 3915500 | CTCGGTAATATCCTTGAAACGAACG |
| | 403B2.10 | | |
| 1532 | RP11- | 3915500 | AAGTTTATTTCGACCTGATTAACGT |
| | 403B2.10 | | |
| 1533 | RP11- | 3915500 | CGGTAATATCCTTGAAACGAACGAG |
| | 403B2.10 | | |
| 1534 | RP11- | 3915503 | TGAAACTTGCGTGAGGAGTCTCAGG |
| | 403B2.10 | | |
| 1535 | RP11- | 3915503 | TCTGAAACTTGCGTGAGGAGTCTCA |
| | 403B2.10 | | |
| 1536 | RP11- | 3915503 | CTGAAACTTGCGTGAGGAGTCTCAG |
| | 403B2.10 | | |
| 1537 | RP11- | 3915503 | CTTCTGAAACTTGCGTGAGGAGTCT |
| | 403B2.10 | | |
| 1538 | SPINK1 | 2880434 | GTTACTTGAATTACCTACGTGGTTC |
| 1539 | SPINK1 | 2880434 | GAATAGGGTTACTTACGCACAATAC |
| 1540 | SPINK1 | 2880434 | AGACACCCTGACTACCTTTATGAAT |
| 1541 | SPINK1 | 2880434 | TCTATATACTGGGACAGACACCCTG |
| 1542 | SPINK1 | 2880452 | CAAAAGTTGACTGGAGACCTGCGTC |
| 1543 | SPINK1 | 2880452 | ACCTCCGGTCCGATACTGTGTCTCA |
| 1544 | SPINK1 | 2880452 | CCCTCTAGACACTATATCGGGTCAT |
| 1545 | SPINK1 | 2880452 | GAAGACTTCTCTGCACCATTCACGC |
| 1546 | SPINK1 | 2880439 | AAGAAGAGTCACGGAACCGGGACAA |
| 1547 | SPINK1 | 2880439 | TACTTCCATTGTCCGTAGAAAGAAG |
| 1548 | SPINK1 | 2880439 | CGTAGAAAGAAGAGTCACGGAACCG |
| 1549 | SPINK1 | 2880439 | CGGAACCGGGACAACTCAGATAGAC |
| 1550 | SPINK1 | 2880430 | TCCAAAACTTTAGGGTAGTCCAGTG |
| 1551 | SPINK1 | 2880430 | CCAAAACTTTAGGGTAGTCCAGTGG |
| 1552 | SPINK1 | 2880430 | GTTCCAAAACTTTAGGGTAGTCCAG |
| 1553 | SPINK1 | 2880430 | TTGGTTCCAAAACTTTAGGGTAGTC |
| 1554 | SPINK1 | 2880433 | CGATTATAAGGGACAGAATGAACAC |
| 1555 | SPINK1 | 2880433 | AGTACTCGTACATATCCTACCGAAG |
| 1556 | SPINK1 | 2880433 | TTTCTTATCTTACGGTCGGCCCACG |
| 1557 | SPINK1 | 2880433 | TCGTTGACTTTGGAATCGTACAGAG |
| 1558 | SPINK1 | 2880429 | TAACAACTTATTTACATAGACTTAT |
| 1559 | SPINK1 | 2880429 | CCGGAATAACAACTTATTTACATAG |
| 1560 | SPINK1 | 2880429 | AATAACAACTTATTTACATAGACTT |
| 1561 | SPINK1 | 2880429 | GGAATAACAACTTATTTACATAGAC |
| 1562 | SPINK1 | 2880435 | GTGACCTCGACTGAGGGACCCTTCT |
| 1563 | SPINK1 | 2880435 | GACCTCGACTGAGGGACCCTTCTCT |
| 1564 | SPINK1 | 2880435 | TTGTGACCTCGACTGAGGGACCCTT |
| 1565 | SPINK1 | 2880435 | ATTGTGACCTCGACTGAGGGACCCT |
| 1566 | TTN | 2589513 | GGACGCCTTGGTTTACTATTCTGAC |
| 1567 | TTN | 2589513 | GGATATGCTCTGGTCGTACACTTTG |
| 1568 | TTN | 2589513 | ACACTTTGGGTTCCCCTGTCGATAA |
| 1569 | TTN | 2589513 | CGGACACTATATCGTTTTCTATGAG |
| 1570 | TTN | 2589527 | TAGATATTGGTAACCATCTTTTCTC |
| 1571 | TTN | 2589527 | TTTTTCGTAGATATTGGTAACCATC |
| 1572 | TTN | 2589527 | TCGTAGATATTGGTAACCATCTTTT |
| 1573 | TTN | 2589527 | TCTTTTCTCTGAGGGGGACAACTTC |
| 1574 | TTN | 2589398 | GGTCACTGATAGGAGCGTCTTTTAC |
| 1575 | TTN | 2589398 | TCTCGATAACGGTCCTGGCGCCATT |
| 1576 | TTN | 2589398 | CGCCATTTGTAATCGGGTGGAAGAC |
| 1577 | TTN | 2589398 | AGACCCGAGCAGGTTTCGTACTACC |
| 1578 | TTN | 2589323 | GGCACACAATCGGTTCTTACGTCGT |
| 1579 | TTN | 2589323 | CCAGCGACCGACTTCACGTTGATGT |
| 1580 | TTN | 2589323 | GTGGCACTGACGAGAGTCACTTCCT |
| 1581 | TTN | 2589323 | GTCGTTCCAACACCCGATGTAGTAT |
| 1582 | TTN | 2589361 | AGAGAGACTCAACCTGTTTTGGACT |
| 1583 | TTN | 2589361 | GCGGTTCAGGTGGACAATTGGACTT |
| 1584 | TTN | 2589361 | TCACACGAGCTCATTTCAGAGAAGT |
| 1585 | TTN | 2589361 | CTCTTCACTAGGAGCCAGGGAACGT |
| 1586 | TTN | 2589371 | GTAACAACTCTTTGCCCTGTGAAGG |
| 1587 | TTN | 2589371 | CTCGGATGACATCGGGTTATAGGTA |
| 1588 | TTN | 2589371 | TGGTGGACCGTTTAACATAGTCGAT |
| 1589 | TTN | 2589371 | TGGATGGAGTTAAGTCTCGGATGAC |
| 1590 | TTN | 2589355 | CACAGTTCCGAATATTACTCTTTCC |
| 1591 | TTN | 2589355 | GCTAGGTTCTCACAACCCACAAGGA |
| 1592 | TTN | 2589355 | CAAGGCACAGTTCCGAATATTACTC |
| 1593 | TTN | 2589355 | TTTTCGCTAGGTTCTCACAACCCAC |
| 1594 | TTN | 2589748 | AAGGAAACCGGTTTGACTTTCTAAG |
| 1595 | TTN | 2589748 | GTGGAAGTGGATTGACCTCCTAAAG |
| 1596 | TTN | 2589748 | CTCGGACACGTCAGGCGATAGTTAT |
| 1597 | TTN | 2589748 | ATCGTTGTGAACTCTAAGGAAACCG |
| 1598 | TTN | 2589789 | TTTACGGACAAATAGGTGGACGGTA |
| 1599 | TTN | 2589789 | AAAGTTACGGCCCAAAGACCTTGTC |
| 1600 | TTN | 2589789 | CACAGAGGACTAGTCCTTTACGGAC |
| 1601 | TTN | 2589789 | TCCTTTACGGACAAATAGGTGGACG |
| 1602 | TTN | 2589500 | GACACCCTGTCTATTCTGGAGTCCT |
| 1603 | TTN | 2589500 | ACCGTTCTTTGCGTACGATTAGGAT |
| 1604 | TTN | 2589500 | AGTCTATAACCTGTCATGTGGACAC |
| 1605 | TTN | 2589500 | CCTTTTGAAGTTCTAATATGACCAC |
| 1606 | TTN | 2589459 | CACTTCCTAGAGTATGGTTTACCAC |
| 1607 | TTN | 2589459 | CACTTATGAAGAAGGCACAATTTCG |
| 1608 | TTN | 2589459 | ACCCTCCTCTTGTATATATGGTCAG |
| 1609 | TTN | 2589459 | GATGGACCTGGTACGTTTCTATAAT |
| 1610 | TTN | 2589482 | CTCAAATGACAGTGACCAGATGTCT |
| 1611 | TTN | 2589482 | TGGCACGTCTCTCAACACTCAAATG |
| 1612 | TTN | 2589482 | CATGGATAAGGCACACTCACGTTCT |
| 1613 | TTN | 2589482 | GGACCCGGTCATGCATTGAATCTTC |
| 1614 | TTN | 2589524 | AGTCCAAGGTCTTTTTCACCTCGAA |
| 1615 | TTN | 2589524 | TTTCACCTCGAATGTGGAGACTTTC |
| 1616 | TTN | 2589524 | GGTCTTTTTCACCTCGAATGTGGAG |
| 1617 | TTN | 2589524 | CCAAGGTCTTTTTCACCTCGAATGT |
| 1618 | TTN | 2589315 | CAGCTACAGTGGTTTAGGTGACAAT |
| 1619 | TTN | 2589315 | CCATCGGCTGAGTGACCTATACAAG |
| 1620 | TTN | 2589315 | GAGTCTTCACGGAAACCACGGACGT |
| 1621 | TTN | 2589315 | TCGGCACTGACATGTTCTGGAGTCT |
| 1622 | TTN | 2589486 | GAGTAATATTTTCTAACGTCCGACC |
| 1623 | TTN | 2589486 | AGAGCACGATCTGAAAAACACCTTC |
| 1624 | TTN | 2589486 | CTACCTTCATATGTGTCTGAGTAAT |
| 1625 | TTN | 2589486 | CTTATACGAACGCCCCATCTTCTGT |
| 1626 | TTN | 2589512 | TTCTTTCACGTTCGAAACTACGTCT |
| 1627 | TTN | 2589512 | CGCACTTCAACTTGACGAATTTGGT |
| 1628 | TTN | 2589512 | CTGTAAGGACCTGTTACCTTTGACT |
| 1629 | TTN | 2589512 | CCTGCAATGGTAAATACTCTTTCTT |
| 1630 | TTN | 2589475 | CGTATACAACTACTTGGACATTTAT |
| 1631 | TTN | 2589475 | AATACCTGAAACACTGACTAGATCT |
| 1632 | TTN | 2589475 | GACTAGATCTTAAGTGTCAAGGACT |
| 1633 | TTN | 2589475 | ACACGAGCATTGTTTACACCGGGAC |
| 1634 | TTN | 2589791 | TGTCTCGCTACGTCCTCTTATGTGG |
| 1635 | TTN | 2589791 | CCTTGTCCTCAAGACAGTGAGAGAT |
| 1636 | TTN | 2589791 | AAATAGAGACTCTGGTCTGTCTCGC |
| 1637 | TTN | 2589791 | CTGCTACAACTACGGGTGACCATAT |
| 1638 | TTN | 2589650 | CTTTTCTGAAGAGCTTCTTACCTCC |
| 1639 | TTN | 2589650 | TTTAACTTTTCTGAAGAGCTTCTTA |
| 1640 | TTN | 2589650 | TTTCTGAAGAGCTTCTTACCTCCTT |
| 1641 | TTN | 2589650 | AACTTTTCTGAAGAGCTTCTTACCT |
| 1642 | TTN | 2589616 | GTTAATGGTTTGCACTTTTTCTCGT |
| 1643 | TTN | 2589616 | AGTTAATGGTTTGCACTTTTTCTCG |
| 1644 | TTN | 2589616 | TTAATGGTTTGCACTTTTTCTCGTC |
| 1645 | TTN | 2589381 | CGGAAATGACATTGACTGGAACAAC |
| 1646 | TTN | 2589381 | CCACGATAGTCACGAGGTAGTCTTT |
| 1647 | TTN | 2589381 | TCGCTCTAGATGGAAGCTTCAGAAC |
| 1648 | TTN | 2589381 | GGTACAATTACAGGGTCTTACACGG |
| 1649 | TTN | 2589318 | TGACTAGTCACCATGGCTCACGTAT |
| 1650 | TTN | 2589318 | ACTCGCTTATGTCGCTTAGTTAACG |
| 1651 | TTN | 2589318 | ACCTGGTTTGGGTACATGCTACCAC |
| 1652 | TTN | 2589318 | AAGTCTCAACGACGGCACTTGCACT |
| 1653 | TTN | 2589615 | CTTTTCTTCATCGTGGTGGACAATC |
| 1654 | TTN | 2589615 | ATGGACAGGGATCTTTTCTTCATCG |
| 1655 | TTN | 2589615 | CTCCCAACAGCGTCTTCTTTTTCAT |
| 1656 | TTN | 2589615 | TTTCTTCATCGTGGTGGACAATCTC |
| 1657 | TTN | 2589418 | AGACATACCTTATTCACTAGGAGAC |
| 1658 | TTN | 2589418 | TTCTAATATAAAAGGCCTATGTACG |
| 1659 | TTN | 2589418 | GGTCACTGTAGACGTTCACGATTTT |
| 1660 | TTN | 2589418 | GACCACAGACTAAGTTACTTTCGGT |
| 1661 | TTN | 2589458 | AGGATGTCGCCTCCGTTACTGATAA |
| 1662 | TTN | 2589458 | GGTGGACATCTACATCTCCAAGTAT |
| 1663 | TTN | 2589458 | TCATGGTTAAGGCACACGCTCGTCT |
| 1664 | TTN | 2589458 | GCGGCGCCCATAATCACTTGGAAGA |
| 1665 | TTN | 2589490 | CCAGTAAGTCTTGCGAGTGGAACTC |
| 1666 | TTN | 2589490 | CTGAGGGTTCGAGAGCTTGGCTACC |
| 1667 | TTN | 2589490 | GGACCGCGTCTTCACTAAACCATAT |
| 1668 | TTN | 2589490 | GACTGACTTCCTGCCTTCTCTTAGG |
| 1669 | TTN | 2589784 | CACTCCGCGGGTTCTAAAAGGACGT |
| 1670 | TTN | 2589784 | TAAAAGGACGTAGAAGTCCTGCAGT |
| 1671 | TTN | 2589784 | GAAGTCCTGCAGTGACATTTCACGC |
| 1672 | TTN | 2589784 | TCACGCCACTGTGCCGAGTTAAGGA |
| 1673 | TTN | 2589687 | ACTGTTGTATACCTAAAGAATAAGT |
| 1674 | TTN | 2589687 | CACTGTTGTATACCTAAAGAATAAG |
| 1675 | TTN | 2589460 | GTGACCTCTTATTCGAGCCGAGTCG |
| 1676 | TTN | 2589460 | CTGGACCTCTGAGTACTGTAATAAC |
| 1677 | TTN | 2589460 | CACAGGCACGTCTACGGCCTTAAAT |
| 1678 | TTN | 2589460 | CTCGACAGGGTCAAGGTTGACAATC |
| 1679 | TTN | 2589759 | GTCACCGGAGATATAGACATTTCGT |
| 1680 | TTN | 2589759 | CGACGAACACGAAGACCTTCTGTGT |
| 1681 | TTN | 2589759 | CGAGTTCCCGAAGGACGGTAGAAAC |
| 1682 | TTN | 2589759 | TTTGGTTAAGCGACACGAGTTCCCG |
| 1683 | TTN | 2589535 | CTTTCATCGACAAGGGTTTTTCGGT |
| 1684 | TTN | 2589535 | TTTCATCGACAAGGGTTTTTCGGTC |
| 1685 | TTN | 2589535 | TTCATCGACAAGGGTTTTTCGGTCT |
| 1686 | TTN | 2589819 | TGGACGGCGCGGAATGAAATAATGT |
| 1687 | TTN | 2589819 | AACCTACGGTTCAACCGCCGTTGGG |
| 1688 | TTN | 2589819 | TCTACACCAATACTGACTATGATCG |
| 1689 | TTN | 2589819 | GACCACAAGGAGATTGGTGACCTAT |
| 1690 | TTN | 2589647 | TTCTTCTACCAATAAAGTCTTCTTT |
| 1691 | TTN | 2589647 | CGTATGTTTCTTCTACCAATAAAGT |
| 1692 | TTN | 2589647 | GTGTGTCTCCTCCTCCACAGTCAGT |
| 1693 | TTN | 2589647 | TTTTCTACAAGAAACGAAGAGTGTG |
| 1694 | TTN | 2589642 | GACTCGATGGACTCTTTGGTCGAGG |
| 1695 | TTN | 2589642 | TTTGGTCGAGGTCTTCTTCACCGGG |
| 1696 | TTN | 2589642 | GGGACTCGATGGACTCTTTGGTCGA |
| 1697 | TTN | 2589642 | CTCGATGGACTCTTTGGTCGAGGTC |
| 1698 | TTN | 2589684 | GAACCTCACATGTCATCGACCGTGG |
| 1699 | TTN | 2589684 | GCCCTCTGTGGACATGGAACCTCAC |
| 1700 | TTN | 2589684 | CATAGGCCGGAATTCTAGTAGTTAC |
| 1701 | TTN | 2589684 | AAACTCCACGTCTTGGGACAACCGT |
| 1702 | TTN | 2589812 | TCAGACGCATATGGACAACAAGAAG |
| 1703 | TTN | 2589812 | GAACCTCGAGGCTGAATGTAAGGGT |
| 1704 | TTN | 2589812 | CCTCGGACGACGTGGTGAACCTCGA |
| 1705 | TTN | 2589812 | TACCGTTCGCGTAGTTTGTACCTCT |
| 1706 | TTN | 2589532 | AACGAGGACTTCTCCTTTAACGAGG |
| 1707 | TTN | 2589532 | CCTCCTTGGTCTCCAAGGTGGAGGT |
| 1708 | TTN | 2589532 | CTTGGACTCCTTTAACGAGGACTTC |
| 1709 | TTN | 2589532 | GGACTTCTCCTTTAACGAGGACTTC |
| 1710 | TTN | 2589330 | GTCGTCTGAACGGACCCGTGATTAA |
| 1711 | TTN | 2589330 | TGGTTAAGGCACAAAGACGTCAATT |
| 1712 | TTN | 2589330 | TGAGTTACTGCCAACACGAGGGTAT |
| 1713 | TTN | 2589330 | GACTAGGTCACCAACGAGTTTATGT |
| 1714 | TTN | 2589481 | GGTACTGACACTCTCGACTTCTGGA |
| 1715 | TTN | 2589481 | TCAAAGGCTCACTCTCGGGTTTTAG |
| 1716 | TTN | 2589481 | CGTCGGTGTGGGAAGCAGTTTCAAC |
| 1717 | TTN | 2589481 | CTTCTGGACAGACGTTGACAATGAC |
| 1718 | TTN | 2589821 | AACGAGCAGAATACTAAGCGCTTCG |
| 1719 | TTN | 2589821 | GCCCGCTAAATGAACGTCACGACAT |
| 1720 | TTN | 2589821 | ACGACATTTACTCCGACCTTGGCAG |
| 1721 | TTN | 2589821 | AGCGCTTCGTAAACGCCTTCTGTCG |
| 1722 | TTN | 2589421 | ACCTGGTGGTCATCCTGGTCAATCA |
| 1723 | TTN | 2589421 | GGACCTGGTGGTCATCCTGGTCAAT |
| 1724 | TTN | 2589421 | GACCTGGTGGTCATCCTGGTCAATC |
| 1725 | TTN | 2589707 | AACCGTTTCTGTACACGAGTCGAGT |
| 1726 | TTN | 2589707 | CACGAGTTGGCTACGCTTAGTGAAC |
| 1727 | TTN | 2589707 | TCACAGTACAATGACCACGAGTTGG |
| 1728 | TTN | 2589707 | ACACACCCTTTGTGAGGAGTAAACT |
| 1729 | TTN | 2589810 | TAGTGATTAATAACAGGGACGGTGT |
| 1730 | TTN | 2589810 | GACGGTGTGGGTCACTAAGACCCCT |
| 1731 | TTN | 2589810 | CCCTTACCTGACACCAACGGGTTTT |
| 1732 | TTN | 2589810 | CATCAGTAATTTCTTCTACCATGAG |
| 1733 | TTN | 2589417 | CTACAGTCATACTTAAGGCCCAAAG |
| 1734 | TTN | 2589417 | GGACTACGTGGACTAGTCGGTTAAC |
| 1735 | TTN | 2589417 | CTGAGACGTAATCATTGGACCTTAT |
| 1736 | TTN | 2589417 | ACCCGATCTCAATGGTTTCTAGGAT |
| 1737 | TTN | 2589308 | GTGTCCGATGAACTAACTTTACGTT |
| 1738 | TTN | 2589308 | GACGACTCCATGGTCCTTGTCAGTT |
| 1739 | TTN | 2589308 | ACGTCTTATGTCCAAGGCGCAGGAT |
| 1740 | TTN | 2589308 | CACATTGTGGTGAGGTTGGTTCTAA |
| 1741 | TTN | 2589345 | CACATCGATATTTCCGTGATCTAGG |
| 1742 | TTN | 2589345 | GATCTAGGTAAATGTCAAGGTTCAG |
| 1743 | TTN | 2589345 | GAAACGACCAGTTCTGGGCTCTCAC |
| 1744 | TTN | 2589345 | TCGGATTCTACCCACGCACATTTGT |
| 1745 | TTN | 2589422 | GGTTTGCAAGTCTAAGGCCGTTTAT |
| 1746 | TTN | 2589422 | TGCGAAGTGGAGCTGAAGTCTCTAT |
| 1747 | TTN | 2589422 | GACCGGCAATGAGTCCGTTTGGTTT |
| 1748 | TTN | 2589422 | GCTCAACCACTTCGAAAACGGGAGT |
| 1749 | TTN | 2589699 | CATTGACGTGACATAGGCAGGTACA |
| 1750 | TTN | 2589699 | TGACGTGACATAGGCAGGTACAAAG |
| 1751 | TTN | 2589699 | TCATTGACGTGACATAGGCAGGTAC |
| 1752 | TTN | 2589699 | TTTCATTGACGTGACATAGGCAGGT |
| 1753 | TTN | 2589538 | GACGACTTCAACACCTTCTCGGTCT |
| 1754 | TTN | 2589538 | GAGGACGACTTCAACACCTTCTCGG |
| 1755 | TTN | 2589538 | CCTTCAACGGGATCTTCTCGGAGGA |
| 1756 | TTN | 2589538 | CTCGGACTCCTTCAACGGGATCTTC |
| 1757 | TTN | 2589497 | CAAAACGTGTTGACAGCGGACCTGG |
| 1758 | TTN | 2589497 | TTGACAGCGGACCTGGTCTGCCCAC |
| 1759 | TTN | 2589497 | TCTTCCGTTTTATGTGAGGGAACAA |
| 1760 | TTN | 2589497 | AGATCACGGGTGGAGGCTCAATTCG |
| 1761 | TTN | 2589483 | CCATATTTTAACACGAAGTCTTGTT |
| 1762 | TTN | 2589483 | CCAACTACAACCGTTCGGAGACTGT |
| 1763 | TTN | 2589483 | ATGATGCCGACTTGTTTGAAGAAAG |
| 1764 | TTN | 2589483 | TCGTCTTCGACTTACCAAATTTCTT |
| 1765 | TTN | 2589778 | GGGTTGTCCAGAAGCAGCCTAATAT |
| 1766 | TTN | 2589778 | CCAAAGTATTGTTAGCCGGGTAGGG |
| 1767 | TTN | 2589778 | CCCTATGTGCACTGCGGAAAAGTCT |
| 1768 | TTN | 2589778 | GGACTCCAAATGACCGAGAGGGAAA |
| 1769 | TTN | 2589449 | GTCACAAGGCTTTACAAGTGCAACT |
| 1770 | TTN | 2589449 | GAATCGTAAGGGTTTCGCCAGGCCC |
| 1771 | TTN | 2589449 | CAAGCTCCTCTGTGATAGTTTCAAT |
| 1772 | TTN | 2589449 | AAGGTTAGCGGAACCGAGTCACAAG |
| 1773 | TTN | 2589304 | CCGGAGAAGGACTGTAATTTACCAT |
| 1774 | TTN | 2589304 | ACGGTCTAACAACCTTCCGGAGAAG |
| 1775 | TTN | 2589304 | TAACCAGCAGGTCATGGACGGTACT |
| 1776 | TTN | 2589304 | CCAAGGTGTGAAGCCGAAGTACAAT |
| 1777 | TTN | 2589344 | AATGGTACTGACACGGAAAGGCTCC |
| 1778 | TTN | 2589344 | CGGTCTGTGACTGGAGGCATGATCT |
| 1779 | TTN | 2589344 | ACCCAGAGACACTGGTTGTTGACAT |
| 1780 | TTN | 2589344 | AGGACTTTTGCTACCACCTCGTGGT |
| 1781 | TTN | 2589796 | CTTCAATCACAAAGGGTACTGTGAC |
| 1782 | TTN | 2589796 | TAATTCGGTTCACTGTTTGTGTCTG |
| 1783 | TTN | 2589796 | TGACAAGGTCATTTTACCAAGGTAT |
| 1784 | TTN | 2589796 | AGGTATTCTCACACCTTTAATTCGG |
| 1785 | TTN | 2589545 | TTACGGGAACCGAGGAGGATTTTTC |
| 1786 | TTN | 2589545 | TCCGAGGGTATCTCCAACAAGGACT |
| 1787 | TTN | 2589545 | CGGTCTCCGAGGGTATCTCCAACAA |
| 1788 | TTN | 2589545 | GGATTTTTCGGACTTCAGGGAGGAC |
| 1789 | TTN | 2589384 | CTTTTGACCACCAAGAGGTTAATGT |
| 1790 | TTN | 2589384 | GGGTGGAGGGTTATAACACCTACAG |
| 1791 | TTN | 2589384 | ACCTACAGTCTGTGCTAAGTCATAG |
| 1792 | TTN | 2589384 | GTCATAGAGATTGAACCTGACTGGG |
| 1793 | TTN | 2589510 | CTTTTGCGAAGAATTGAAACGTGTT |
| 1794 | TTN | 2589510 | GGAATTACGTTAATGTTGACGGTAA |
| 1795 | TTN | 2589510 | CGACCACTTCAGGAGATGGTCCGGG |
| 1796 | TTN | 2589510 | CACCTTTTGCGAAGAATTGAAACGT |
| 1797 | TTN | 2589502 | CCTACCGTGATTCGTAAGTTACCAC |
| 1798 | TTN | 2589502 | CACACTTCATAGGTCCCTCGGGTTT |
| 1799 | TTN | 2589502 | CCTCGGGTTTTGTAAGGCAACCGAT |
| 1800 | TTN | 2589502 | GTGTGTTCACCGTTTGACTAGTAAC |
| 1801 | TTN | 2589405 | TAAGGAATTGTGAACACTTGGGTCG |
| 1802 | TTN | 2589405 | CCGACATAAGCATTTACAGTCTCAT |
| 1803 | TTN | 2589405 | AGACCAACTGTGATACCGGAAGGAA |
| 1804 | TTN | 2589405 | ACCAGTCTTTTCCTGTTCAACTAGA |
| 1805 | TTN | 2589329 | GTAATGGGACTGTACCCGTTCCGGT |
| 1806 | TTN | 2589329 | CTTTTCGTGTTCTACCCATTTTCAC |
| 1807 | TTN | 2589329 | CCGTAAGGTCTTGGATCGTTGTATT |
| 1808 | TTN | 2589329 | GTTGTCATATAGGAACTTTCTTCTC |
| 1809 | TTN | 2589380 | CGATGGTTAGGAAAACCGTGCTTCC |
| 1810 | TTN | 2589380 | GGTGATACCGTTTCCTCTTGGACAT |
| 1811 | TTN | 2589380 | CCGCCGAGTGGTTAATTCAGGATAT |
| 1812 | TTN | 2589380 | CGCCCACTTATATGGTAGTGACGAT |
| 1813 | TTN | 2589413 | ACACCAATGACCTGATGTTGTTCCT |
| 1814 | TTN | 2589413 | GGATATCTAAGGCACATTTTCGACT |
| 1815 | TTN | 2589413 | GGGATCAGACCTTATTCGGCCTAGC |
| 1816 | TTN | 2589413 | GTTCCTGGACGTGGTACATCTACAA |
| 1817 | TTN | 2589845 | TCGCTGATCATGACGACTCGAAGAG |
| 1818 | TTN | 2589845 | GTCACCTGCTATAAGGGACTTTCGG |
| 1819 | TTN | 2589845 | GCTACCGGCGCGATTTGACTGCTAG |
| 1820 | TTN | 2589845 | GGCCGCACGTCTAGAGGAAATCGCT |
| 1821 | TTN | 2589700 | AAGTAGTTATTTCACCGAAGGGAAT |
| 1822 | TTN | 2589700 | AGGAAAACGTAATCTCACACATCAC |
| 1823 | TTN | 2589700 | GGTCTTGAGAGTCGGTTCACCAAGT |
| 1824 | TTN | 2589700 | GGGTACTGACAGTGATGACCTTTAG |
| 1825 | TTN | 2589445 | AGGAGCAAACGAACTTCCGCAATTT |
| 1826 | TTN | 2589445 | TCAGGAATAACCAGTGCACAATCGG |
| 1827 | TTN | 2589445 | GAGGAGGAAACCTGTTACCACCGAG |
| 1828 | TTN | 2589445 | TACCACCGAGAGGTTAATGACCGAT |
| 1829 | TTN | 2589787 | GTTTGAGGGTTACCGTACAGAGAAA |
| 1830 | TTN | 2589787 | AGGGTTACCGTACAGAGAAATAGTC |
| 1831 | TTN | 2589787 | TTATGTAGTTTGAGGGTTACCGTAC |
| 1832 | TTN | 2589787 | GTAGTTTGAGGGTTACCGTACAGAG |
| 1833 | TTN | 2589592 | CTTCATCTTCTTAAGTAGTTTAATC |
| 1834 | TTN | 2589592 | AACTTTTTCAAGTATCCCATTATCT |
| 1835 | TTN | 2589592 | CCGCTCAAAGTACTTCATCTTCTTA |
| 1836 | TTN | 2589592 | CTTATAAAACTTCTTCCGCTCAAAG |
| 1837 | TTN | 2589802 | GTAAAATGCGCCCTCTTTTATACTG |
| 1838 | TTN | 2589802 | TCTCCAGAAGCACTGGAATGGACAT |
| 1839 | TTN | 2589802 | ACAAACTCCAACTCGACAGGGTGAG |
| 1840 | TTN | 2589802 | CAGGGTGAGACCTTAACTACAGGAC |
| 1841 | TTN | 2589471 | GAACCTGTTTCCGACTATACTAAGA |
| 1842 | TTN | 2589471 | AGGTGTCACTGATAACAACTATCAT |
| 1843 | TTN | 2589471 | CAACTATCATTCTCTTCACTGTGAC |
| 1844 | TTN | 2589471 | ACTTGAAGGACGGTGGCATTGGCCT |
| 1845 | TTN | 2589416 | TCAGGTGGACAATTAGGACTTCGTT |
| 1846 | TTN | 2589416 | TCAGCTAGATTGAACCGTCGGTGGT |
| 1847 | TTN | 2589416 | CCACTTAGTCTAGGTCGAGTACAAG |
| 1848 | TTN | 2589416 | AGTACAAGGCCTCGGTCAGGATCAT |
| 1849 | TTN | 2589515 | GGGTTCGTGTCCAAATAACGTCTAC |
| 1850 | TTN | 2589515 | CTGGAGGTGCCGATTTGAACAACAT |
| 1851 | TTN | 2589515 | TGAAAGGCTACGACCACTTATGTGG |
| 1852 | TTN | 2589515 | CATCGTTATAGGCACTCTCAGGGTT |
| 1853 | TTN | 2589472 | GTCTCCTTTAGGTCCGACACCTGTG |
| 1854 | TTN | 2589472 | GTCTGGGTGTCTCCTTTAGGTCCGA |
| 1855 | TTN | 2589472 | CGTCCCACGATCGTTTGGTTCGTCT |
| 1856 | TTN | 2589472 | GATCGTTTGGTTCGTCTGGGTGTCT |
| 1857 | TTN | 2589305 | ACCCGACTCGTCTGGAGCGTCTTGA |
| 1858 | TTN | 2589305 | GCGTCTTGACGATACAGATATTTCT |
| 1859 | TTN | 2589305 | AGTGTTATCCTCCAAACGACCTTCG |
| 1860 | TTN | 2589305 | CGACCTTCGATGACTCATACTTAAG |
| 1861 | TTN | 2589424 | GGATGAAAAAGGCTTAACGCCGACT |
| 1862 | TTN | 2589424 | AGGTCCAGGGCAACCTTGTGGTAAG |
| 1863 | TTN | 2589424 | ACAACTCTGTAGTCTCCGTGAACAA |
| 1864 | TTN | 2589424 | GGGTCACTGTATATGTCAATGGGCT |
| 1865 | TTN | 2589609 | CGGTCAAGGACGAGGATTCTTTCAC |
| 1866 | TTN | 2589609 | GTCAAGGACGAGGATTCTTTCACCT |
| 1867 | TTN | 2589609 | GGTCAAGGACGAGGATTCTTTCACC |
| 1868 | TTN | 2589609 | TCAAGGACGAGGATTCTTTCACCTC |
| 1869 | TTN | 2589402 | GTAGGGTGGACATTTACCTTTTTTC |
| 1870 | TTN | 2589402 | AAGTTAAGACTAGTATTTTCTACAC |
| 1871 | TTN | 2589402 | GGTTTCTAGGAATACGGTCTCGTTT |
| 1872 | TTN | 2589402 | GAGGCTTAACTTCGGGTACACATAC |
| 1873 | TTN | 2589514 | CGCCCGAGACTGGTAGTTGCTACGT |
| 1874 | TTN | 2589514 | GGACCTCACAAGTAGAACGCATTTT |
| 1875 | TTN | 2589514 | ACCTTTTGCGGTTGTTGGACCTCAC |
| 1876 | TTN | 2589514 | ACCGGCTTAACACGGTCCGCAGTAA |
| 1877 | TTN | 2589807 | GGCGACACCTCGAGTTCAGAGCTTT |
| 1878 | TTN | 2589807 | CGGACCATATGACGCTGACGATAAT |
| 1879 | TTN | 2589807 | CCATGACCGGGCTTCTGTTACAAAC |
| 1880 | TTN | 2589807 | GGACCTGTAGCACCTGACGTTTAGT |
| 1881 | TTN | 2589375 | TGGAAGGATTACCTGCCACCGACTT |
| 1882 | TTN | 2589375 | CCTAGGGTAACTGGGTGGACCTTTT |
| 1883 | TTN | 2589375 | CTTAAGGCACACTAGCGGTTTTTAC |
| 1884 | TTN | 2589375 | ACCCGATTCGGACTTATATGACCCC |
| 1885 | TTN | 2589525 | GGTGGACGAGGTGGATTCCTTCTAC |
| 1886 | TTN | 2589525 | GACGAGGTGGATTCCTTCTACACTT |
| 1887 | TTN | 2589525 | CGAGGTGGATTCCTTCTACACTTCC |
| 1888 | TTN | 2589525 | TGGACGAGGTGGATTCCTTCTACAC |
| 1889 | TTN | 2589682 | GGGTACCTACAAAATTGACCCTGGT |
| 1890 | TTN | 2589682 | GTGTTCATAGCATTTTCCTTGTGGA |
| 1891 | TTN | 2589682 | ATTGACCCTGGTTACATTGAAAGTG |
| 1892 | TTN | 2589682 | GTTACATTGAAAGTGTTCATAGCAT |
| 1893 | TTN | 2589758 | ACGCGAGTAGAACTCAGTCTCGAAT |
| 1894 | TTN | 2589758 | TCCCAGGGCGTCAACTTCGTGAACT |
| 1895 | TTN | 2589758 | AGAGGGTTGGATGTCGACGTCTAAC |
| 1896 | TTN | 2589758 | ACGTCTAACATGTCAGGGTCTTTTG |
| 1897 | TTN | 2589820 | TTCCTTTGGTGTCGGCACTGACTCT |
| 1898 | TTN | 2589820 | TTGGTGTCGGCACTGACTCTTTAAA |
| 1899 | TTN | 2589820 | CTTTGGTGTCGGCACTGACTCTTTA |
| 1900 | TTN | 2589820 | GTGTCGGCACTGACTCTTTAAATGA |
| 1901 | TTN | 2589672 | GACCGTCCCTTTATTTCGGAAGTCT |
| 1902 | TTN | 2589672 | GTCGAAGTCGAAACGATCACCCTGT |
| 1903 | TTN | 2589672 | ACCGATTTCGTCTAAGCCCTCTAAT |
| 1904 | TTN | 2589672 | ACCGACCTTCACTGTGATGGTTTAG |
| 1905 | TTN | 2589429 | TCCTTCCTCGTAAGATGTTTAAATC |
| 1906 | TTN | 2589429 | TCGTAAGATGTTTAAATCTCAATCT |
| 1907 | TTN | 2589429 | CCTCGTAAGATGTTTAAATCTCAAT |
| 1908 | TTN | 2589429 | TTCCTCGTAAGATGTTTAAATCTCA |
| 1909 | TTN | 2589320 | CACTCGGGTCGCTTCGAAGGTTGAA |
| 1910 | TTN | 2589320 | TCGGACGCGACCTGTGCACAGTTGT |
| 1911 | TTN | 2589320 | GGTGGGCCGTATGGACTTCTTCAAC |
| 1912 | TTN | 2589320 | ACTAATGGTCAAGGCCCACTGGCGT |
| 1913 | TTN | 2589829 | CGTCAACGATGACGATTTCGGTTTC |
| 1914 | TTN | 2589829 | TCCTTCGGCTCTTTTGACGGAACAG |
| 1915 | TTN | 2589829 | ACTCTTGATCTCTTTGATACCGATG |
| 1916 | TTN | 2589829 | GACGGAACAGATGTTATCGTCAACG |
| 1917 | TTN | 2589754 | GTCAATAAAATTGCACGTTTCATCC |
| 1918 | TTN | 2589754 | CGGTGTGGACAGTGATTTCCCCAAC |
| 1919 | TTN | 2589754 | TTCACCATGACTCCTCCAACGATGT |
| 1920 | TTN | 2589754 | GTCGACAACAGAGACTGCTTTTTGT |
| 1921 | TTN | 2589769 | CCATAGCTTTATTTAGAAATGATCT |
| 1922 | TTN | 2589769 | TTATTTAGAAATGATCTCGGTCTAC |
| 1923 | TTN | 2589769 | TAGAAATGATCTCGGTCTACTTTCT |
| 1924 | TTN | 2589769 | AGCTTTATTTAGAAATGATCTCGGT |
| 1925 | TTN | 2589435 | GGTCACTTGGATTCCTCACGTGCAT |
| 1926 | TTN | 2589435 | CTCTTGGAGAACTGTCACTTGGACT |
| 1927 | TTN | 2589435 | CACGTGCATGTGCTAAGGGTTTAAC |
| 1928 | TTN | 2589435 | GACCGGGAGGTCACCCTGGGTATTT |
| 1929 | TTN | 2589367 | TCGACGTTCCAATGATTCGAAGAAC |
| 1930 | TTN | 2589367 | TTGGTCGGCGAATCAAACCTGACAC |
| 1931 | TTN | 2589367 | TGAAAGGCATATTACCGTCATTTGT |
| 1932 | TTN | 2589367 | ACTACGGTTACACGTCTGAGAGTCG |
| 1933 | TTN | 2589365 | CATTGACCTGAGTATCTTTTAGTGC |
| 1934 | TTN | 2589365 | TCTTACGACGACCTGAATCACTTGG |
| 1935 | TTN | 2589365 | AGACTAACCTCTCAACGCGGACTCT |
| 1936 | TTN | 2589365 | TTTAGTGCTAATACTCAAGTCTCAA |
| 1937 | TTN | 2589613 | GGGTACATCTTCTTATAGAACATCT |
| 1938 | TTN | 2589613 | TTCTCCTCAAGTATTGACTCCTTCT |
| 1939 | TTN | 2589613 | TCCTTCTTCACCACGGTCACTATGG |
| 1940 | TTN | 2589613 | TTCTCCTTCAACATTGGGTACATCT |
| 1941 | TTN | 2589705 | ACGTCGTGTCGCAACTGTCACTTTC |
| 1942 | TTN | 2589705 | ACCAAGGCTTTACTGTCGCTTGAAG |
| 1943 | TTN | 2589705 | GAATGCTAGTTACTTCGATCACGAC |
| 1944 | TTN | 2589705 | CCCCTGATGTAAACACTCCGAGTAT |
| 1945 | TTN | 2589740 | GATCGGACGTTTCATTGACCATGGG |
| 1946 | TTN | 2589740 | GTAAACAACTTTTCAATCTCGGTAG |
| 1947 | TTN | 2589740 | GGGTCGATCGGACGTTTCATTGACC |
| 1948 | TTN | 2589740 | ACGTTTCATTGACCATGGGGAGGTT |
| 1949 | TTN | 2589724 | TTCGTCATCGACACTACGGATGAAT |
| 1950 | TTN | 2589724 | TGAAGCCTTCGTCATCGACACTACG |
| 1951 | TTN | 2589724 | TTCCACCTTTTACTGAAGCCTTCGT |
| 1952 | TTN | 2589724 | ATCGACACTACGGATGAATTCTCAC |
| 1953 | TTN | 2589362 | AGGATATTTTCCAGCAGGATGTGGT |
| 1954 | TTN | 2589362 | ACAAGGATATTTTCCAGCAGGATGT |
| 1955 | TTN | 2589307 | CTTCACGGATTTCGGCGGACCATAT |
| 1956 | TTN | 2589307 | TCCACCGCAGTAGTCTGAATGGTAT |
| 1957 | TTN | 2589307 | AGACCGTGAATACTGGACCAAGACC |
| 1958 | TTN | 2589307 | ACTACTGTAGGTTCAGGCGAGACAC |
| 1959 | TTN | 2589773 | TCAACGACTTTCTATAAGTTGTGGA |
| 1960 | TTN | 2589773 | CAACGACTTTCTATAAGTTGTGGAA |
| 1961 | TTN | 2589773 | TTCAACGACTTTCTATAAGTTGTGG |
| 1962 | TTN | 2589423 | GCACAGTCACGACAGTTGTAACAAC |
| 1963 | TTN | 2589423 | ACATGGTCTCGCAGGACTTCTGGAC |
| 1964 | TTN | 2589423 | GACATTGAAACTGAACCTTAGGAGG |
| 1965 | TTN | 2589423 | AGCCGAGTAACCCTGACTCTTCAAG |
| 1966 | TTN | 2589752 | CATTTATGGCTTCTGGTAGTTCCTC |
| 1967 | TTN | 2589752 | GATCAGTAGCTGTTTCATTTATGGC |
| 1968 | TTN | 2589752 | AGTTTTATGTATATGCGATCAGTAG |
| 1969 | TTN | 2589752 | CTCATACAGACACTCCGGAACTTAC |
| 1970 | TTN | 2589736 | GAGTCACCGTAACTTCAGTTCGTAC |
| 1971 | TTN | 2589736 | TAGCTCTCATGGTCGAGGGAGGCCC |
| 1972 | TTN | 2589736 | CGAGGAACGGTTAATGCCACTGAAC |
| 1973 | TTN | 2589736 | ACTGGAAACTCTTGTTACACCGGTC |
| 1974 | TTN | 2589801 | AGTCTGGCATCGACTTAGGGTCCTT |
| 1975 | TTN | 2589801 | CGACACAAACTTACACTTCAACGGT |
| 1976 | TTN | 2589801 | GGTCTAAGGTTTCCGCTTACCAACT |
| 1977 | TTN | 2589801 | TCCCTACCGTTTGTGGATGGTGACT |
| 1978 | TTN | 2589335 | AGACTTAAGATGTTTCGACGACTAG |
| 1979 | TTN | 2589335 | GGTTAACGTCTACCGCCATCACTAT |
| 1980 | TTN | 2589335 | GACCTGTCCCAGGTAAAAGACTTGG |
| 1981 | TTN | 2589335 | CCGTATGGTCTTCACTGTTTCTAAT |
| 1982 | TTN | 2589689 | TAGGACTTACAGCTACGACGTCTGT |
| 1983 | TTN | 2589689 | TTCGGAGTAGGATATCTCTGTGACT |
| 1984 | TTN | 2589689 | GGAACTTACACTCGAAGTCCCGTGA |
| 1985 | TTN | 2589689 | ACGCAACCAAGGTAGCGAGAGTTTC |
| 1986 | TTN | 2589837 | CTCTTGGTCACTAGTCGCGACATCT |
| 1987 | TTN | 2589837 | CGCTGACAACAACGACGGCAACTAT |
| 1988 | TTN | 2589837 | ACGAGTCTCCTGTTGGTGCTGACGA |
| 1989 | TTN | 2589837 | CTACGACTGTTTTCACGTCGACAAC |
| 1990 | TTN | 2589640 | TCTCCACGGTTTCTTTGGACAGGGA |
| 1991 | TTN | 2589640 | GTCTCCACGGTTTCTTTGGACAGGG |
| 1992 | TTN | 2589640 | CTTTTCTAAGGGCAAGGACAACGTT |
| 1993 | TTN | 2589640 | CTAAGGGCAAGGACAACGTTTCTTT |
| 1994 | TTN | 2589584 | ATAAACTCCTACATGGACTTCTCGG |
| 1995 | TTN | 2589584 | TTATAAACTCCTACATGGACTTCTC |
| 1996 | TTN | 2589584 | TAAACTCCTACATGGACTTCTCGGT |
| 1997 | TTN | 2589584 | TATAAACTCCTACATGGACTTCTCG |
| 1998 | TTN | 2589725 | GGTCGAGTTTAGCATCTCTTTCGAT |
| 1999 | TTN | 2589725 | ACTTTACCGAATTTCTGCCCTTTGT |
| 2000 | TTN | 2589725 | GTCAACTACAATGCCTCTTTCTAGG |
| 2001 | TTN | 2589725 | TGGAACCTTACACAACACCGACCTT |
| 2002 | TTN | 2589669 | CCTTAGTGACTGAAGGCTCCAGAAG |
| 2003 | TTN | 2589669 | TTATACTTTTTATACGGGCGTACAT |
| 2004 | TTN | 2589669 | GAGGTTAGAATTTCTTTCCTCGACC |
| 2005 | TTN | 2589669 | TTCTCCTTTAACTATAGTACCTTGA |
| 2006 | TTN | 2589626 | GGAGGTTTTCAGTAATTCTACCTTC |
| 2007 | TTN | 2589626 | GAGGTTTTCAGTAATTCTACCTTCT |
| 2008 | TTN | 2589626 | AAGGAGGTTTTCAGTAATTCTACCT |
| 2009 | TTN | 2589626 | CCAAGGAGGTTTTCAGTAATTCTAC |
| 2010 | TTN | 2589531 | CAGTAGTTTTTTGGTCTTCGTGGCG |
| 2011 | TTN | 2589531 | TCTTCTAGTAGGGTCTCTTCTTTCA |
| 2012 | TTN | 2589531 | TTTGGTCTTCGTGGCGGAGGATTTC |
| 2013 | TTN | 2589531 | CTTCTTTCAAGGACAGTAGTTTTTT |
| 2014 | TTN | 2589404 | TGGTCACGAACGTAGTCTAGGAGAT |
| 2015 | TTN | 2589404 | TGCCACCCTCGGTTCACTGTGTAAT |
| 2016 | TTN | 2589404 | GTCTTTCTGTACCAGCTGGCAATGG |
| 2017 | TTN | 2589404 | TGACGACAGTTGCTTATACCGGGAC |
| 2018 | TTN | 2589670 | CGCACCGTAAATTGCTTGTACCACT |
| 2019 | TTN | 2589670 | GACCCAATATGGCTACGCACCGTAA |
| 2020 | TTN | 2589670 | CCTCCAGCACAAAAGTAGGTGGTTT |
| 2021 | TTN | 2589670 | ACGTTTTCAACCTCCACTGGGTTAG |
| 2022 | TTN | 2589364 | CCGACACTTTAAGTTCCTATGTAAC |
| 2023 | TTN | 2589364 | AACCACTTACCTGTTACACGTGAGG |
| 2024 | TTN | 2589364 | GTCATAAGGAAAGAACCTCGTTTGG |
| 2025 | TTN | 2589364 | CTGTATTGGTCTAGAAGTCATAAGG |
| 2026 | TTN | 2589766 | CGTAACTAGACTCACGAAAGTATAA |
| 2027 | TTN | 2589766 | AAACTTAAGTAATGGAACCTCTGGG |
| 2028 | TTN | 2589766 | AGTGAACACAGTTAGTACGACACAT |
| 2029 | TTN | 2589766 | TCACGGAGTCATTAATAATCGTAAC |
| 2030 | TTN | 4079079 | ATAAATGTAACAACCACTAACTTTG |
| 2031 | TTN | 2589408 | GAACATGCGTTCCTTAAGTGACAAT |
| 2032 | TTN | 2589408 | GGTTCAACCATAACCCGCGGGACGT |
| 2033 | TTN | 2589408 | GGTCAGATACTACCACCACGTGGCT |
| 2034 | TTN | 2589408 | TCAAGTCCCACACACGGGTTTTGGT |
| 2035 | TTN | 2589774 | CACTCTGTCAACTTTCTATAAGGTG |
| 2036 | TTN | 2589347 | GTCAAGGCACAAACACGTCTTTTGG |
| 2037 | TTN | 2589347 | GGCCGGAACTACTTCCTGACTACAT |
| 2038 | TTN | 2589347 | GGACCATGAGGATTTCAACACGTAC |
| 2039 | TTN | 2589347 | AGGCTCTGCAATAACAACAGTTTCG |
| 2040 | TTN | 2589776 | GTCTTTGTCCTTAGTGATCCAGCAG |
| 2041 | TTN | 2589776 | CCAGCAGGTGAAAGAGGACTCAGAC |
| 2042 | TTN | 2589776 | TCCGTTTCGAACAGGTCTCACTTAG |
| 2043 | TTN | 2589776 | CCTTTCGCACTCCAAGGACTTTGAT |
| 2044 | TTN | 2589843 | AACAAAGCTGACGAGTCTAGAGTCT |
| 2045 | TTN | 2589843 | GTTTCTGTTAACAAAGCTGACGAGT |
| 2046 | TTN | 2589843 | TCTTCTTCATGGACGATTTTTCTGT |
| 2047 | TTN | 2589843 | AGTCTTAGTTCTGTTTGGGCTTAAC |
| 2048 | TTN | 2589739 | AACCGTAGCTTCTGTCACCACTTAT |
| 2049 | TTN | 2589739 | GTCACTGGTGACGTCATCGTAACAT |
| 2050 | TTN | 2589739 | TCCGGGTTTTACTCCGACCGTCACT |
| 2051 | TTN | 2589739 | CTTACAGAAAACACCTCAGATGACG |
| 2052 | TTN | 2589389 | CGGTGGACAGTTACACTGACAATTC |
| 2053 | TTN | 2589389 | CCTCGGAGGGTAATAACTACCGCCT |
| 2054 | TTN | 2589389 | AGGGCCACTTTGAGCACTACGACAG |
| 2055 | TTN | 2589389 | GGATGAAGAAGGCTCACAAACGACT |
| 2056 | TTN | 2589709 | TAATGCCGGTTGAGGACTTATGTCC |
| 2057 | TTN | 2589709 | AACAATGCCTTGACCTTGGAGACCT |
| 2058 | TTN | 2589709 | CCTCTAAGCCAAAGAAATGTTACGG |
| 2059 | TTN | 2589709 | ATGTTACGGTTCAACGACCCTGTGG |
| 2060 | TTN | 2589674 | CTGAGAACGTGTCGAGTCGACTTAT |
| 2061 | TTN | 2589674 | ATAGACCTTTTGTCGCGGGTGGACT |
| 2062 | TTN | 2589674 | GTTATATGGACGATACGACACTTAC |
| 2063 | TTN | 2589674 | CCGCCTTTCATGGTCTAATCAATAG |
| 2064 | TTN | 2589803 | GTCAGGGACTACACTGAAGACAATT |
| 2065 | TTN | 2589803 | ACTACTGGCACATGTCCGGTAACAC |
| 2066 | TTN | 2589803 | CCTGGAATGTTCGACTATCAACCGT |
| 2067 | TTN | 2589803 | GAGTACGAAGTCTGCTTCCTGGAAT |
| 2068 | TTN | 2589443 | GAGTGTCTAATCCTAAGGACGACAG |
| 2069 | TTN | 2589443 | AGGACGACAGTAGTTCCCTGCGGGT |
| 2070 | TTN | 2589443 | CCCTGCGGGTTGTGGTTTTAGTAGA |
| 2071 | TTN | 2589443 | ACCAAACATTTCGACCGAGTGTCTA |
| 2072 | TTN | 2589735 | AAGTCCCTGATTCCTCTAATGTCGG |
| 2073 | TTN | 2589735 | GACGTTGTTAGTGGCTCCTTCGACA |
| 2074 | TTN | 2589735 | TGAAAGCTCCAGGTTTTACTACAAC |
| 2075 | TTN | 2589735 | TTCGACACAGATATCTACAGTGGGT |
| 2076 | TTN | 2589661 | TTAGGGAGGACACCAACGAGGAGGA |
| 2077 | TTN | 2589661 | ACGATGGTTGTGGGCTTCTTTTCTT |
| 2078 | TTN | 2589661 | AACGATGGTTGTGGGCTTCTTTTCT |
| 2079 | TTN | 2589661 | AGGAGGATAGGGGGAAAACGATGGT |
| 2080 | TTN | 2589332 | CAGTGGGCCCTTTGGTAGTGTGAAT |
| 2081 | TTN | 2589332 | AGGACCCCTAGCGATACTCAAGTCT |
| 2082 | TTN | 2589332 | ACAACCTTGATATTCGGGCGGGAGT |
| 2083 | TTN | 2589332 | GGTGGTAACGCACTACCTCCGTCAT |
| 2084 | TTN | 2589349 | CACTCACCTGAACATCTTCTGGTTT |
| 2085 | TTN | 2589349 | CGGTCGAAATGGTTGCATAATCTTT |
| 2086 | TTN | 2589349 | GGACGGATACTACCACCATCGTTTT |
| 2087 | TTN | 2589349 | CTTTCTAGATGGACTACCGGCGACC |
| 2088 | TTN | 2589528 | GGTTTCACGGACTCTTTTAGTAGGG |
| 2089 | TTN | 2589710 | CCCTTCAGGTATTAAGACCTCTCGT |
| 2090 | TTN | 2589710 | GTCATAAGGACGCTCTAACTTTTAC |
| 2091 | TTN | 2589710 | ACACCTCGAGACCATAGATGTAATC |
| 2092 | TTN | 2589710 | TCTCGTGGATGTGACCTTGTGAAGG |
| 2093 | TTN | 2589479 | GGAGAGTGGCGACTGCTACGTAAAC |
| 2094 | TTN | 2589479 | TATTTCGACCACAAAGTCTAGGTAG |
| 2095 | TTN | 2589479 | CGACCACAAAGTCTAGGTAGACTTT |
| 2096 | TTN | 2589479 | GAACCAGGAGAGTGGCGACTGCTAC |
| 2097 | TTN | 2589677 | CGTAGTTGAAAGGAATGGCAAGTTC |
| 2098 | TTN | 2589677 | CTCTGGGTGATGCTGAATGTTTTAC |
| 2099 | TTN | 2589677 | GGACTTTAACCCCATAGGACCATAT |
| 2100 | TTN | 2589677 | AGTTCGTCAACCTCGGCCAATTCCA |
| 2101 | TTN | 2589683 | CTGTTTACCAGATAGGCCGAGGAGT |
| 2102 | TTN | 2589683 | CCTTTATGGTCTGGTGGGACTGTCT |
| 2103 | TTN | 2589683 | GATGATTATACCGACCAAGACTACT |
| 2104 | TTN | 2589683 | CACATTTCAGATACCCAGTGGAGGT |
| 2105 | TTN | 2589386 | AGCCTCGGTTAACAACGCTCTGTAG |
| 2106 | TTN | 2589386 | TGGAAGTCCCAGTCACGGCTTTTAT |
| 2107 | TTN | 2589386 | CCCAGATGGACTAAACACGATGAAC |
| 2108 | TTN | 2589386 | CCCCTTCTAGGAGATCGTTGACTGT |
| 2109 | TTN | 2589825 | GAGTCGTCTGGTGAAACCTCATGCC |
| 2110 | TTN | 2589825 | GTGCAACAGGGATTTCGTCAGTTCG |
| 2111 | TTN | 2589825 | AGGCGGCGTTTCCATCGACTCGGAG |
| 2112 | TTN | 2589825 | CGTCAGTTCGGATCTCATTAGGTCC |
| 2113 | TTN | 2589767 | GAAACGTCGTGAGTGGAATTGAATT |
| 2114 | TTN | 2589767 | CATCGATGTTTGTTTAGTCCTTACC |
| 2115 | TTN | 2589767 | CTCGAAACGTCGTGAGTGGAATTGA |
| 2116 | TTN | 2589767 | TTAGTCCTTACCGACTCTCGAAACG |
| 2117 | TTN | 2589493 | ACCGTCCCAGTCTTTTGAACAATAT |
| 2118 | TTN | 2589493 | ACGTCACGACCTCACACTTCAGAGG |
| 2119 | TTN | 2589493 | TGAAGGACATTGGACTTACAGCACG |
| 2120 | TTN | 2589493 | AACTTAAGTGATTCGGAGAACTCCT |
| 2121 | TTN | 2589794 | AGTCCTTACAAGTGAAACCCCTACG |
| 2122 | TTN | 2589794 | ACCCCTACGACGACTGATGTGGAAA |
| 2123 | TTN | 2589794 | CGACTTTCTGTAGTTGCGACTTCTT |
| 2124 | TTN | 2589794 | GTCACTTGATACTTCCGTAGAGAAT |
| 2125 | TTN | 2589295 | CGTCGTCACGATCGTCGTACTGACT |
| 2126 | TTN | 2589295 | TCCTTCGAAGAGCAGAGTCAGTCAG |
| 2127 | TTN | 2589295 | ACGTTCGTACAGACGGGTTTCGTAC |
| 2128 | TTN | 2589295 | AGGTACGTTCTCAGGAAACATCTTT |
| 2129 | TTN | 2589517 | GTCACGGTCACCAACCTTTCTTTCG |
| 2130 | TTN | 2589517 | TGGTTGGGGGTAGCGACGGGGTCAT |
| 2131 | TTN | 2589517 | CACGGTCACCAACCTTTCTTTCGTC |
| 2132 | TTN | 2589517 | GGTAGCGACGGGGTCATTGTCACGG |
| 2133 | TTN | 2589336 | CTTAGACTAAGGCAACATCGGTTCT |
| 2134 | TTN | 2589336 | CTGTTCGGCGCAACACACCAGATAC |
| 2135 | TTN | 2589336 | ACTTATGTAGAAGGCCCAGGCTCGG |
| 2136 | TTN | 2589336 | GGCTCGGCACTTGTTTATACCTTAA |
| 2137 | TTN | 2589722 | GAACCTTCCCAGCTGATCGAAGAAT |
| 2138 | TTN | 2589722 | AAGGATCACTTTGGACCCGCTGTCG |
| 2139 | TTN | 2589722 | CCCGCTGTCGTTCGGTATGGACTAA |
| 2140 | TTN | 2589722 | ACCTTGAACAGAGTCCAGGATTTAC |
| 2141 | TTN | 2589619 | TACTTGCTATACTTCTCGTACTTCT |
| 2142 | TTN | 2589619 | TCGCCCTCATACTTGCTATACTTCT |
| 2143 | TTN | 2589619 | CTTCTTATGCTCCTCGCCCTCATAC |
| 2144 | TTN | 2589619 | CTCATACTTGCTATACTTCTCGTAC |
| 2145 | TTN | 2589805 | AAGTCCTGTATCTTCAAGGTCTTAG |
| 2146 | TTN | 2589805 | TATGTCGAAACAGTAGCTGCCCTTT |
| 2147 | TTN | 2589805 | GAGCAGCACCTGCAGTCTTGGAGTG |
| 2148 | TTN | 2589805 | TTGGAGTGCCAGTTCCTACATTGGT |
| 2149 | TTN | 2589300 | CCGCATGTGGAAGAGGACTAATACT |
| 2150 | TTN | 2589300 | AGGTCCGAAGGCTACTCCCGTTAAT |
| 2151 | TTN | 2589300 | GAAACGGACTTCTGTGCCCAATAAT |
| 2152 | TTN | 2589300 | GGCCGACATTCGTGGTTCTGACATT |
| 2153 | TTN | 2589306 | CAGTGTCCGATGATGTAGCTTGCGT |
| 2154 | TTN | 2589306 | TCAAGACAATCGAACAGGACCAGGG |
| 2155 | TTN | 2589306 | CCCGAACAAGGGCTACGACTCATAG |
| 2156 | TTN | 2589306 | GACCAGGGCCGGGTTTCTACTACCA |
| 2157 | TTN | 2589772 | GATCTCTCTATGAGGTGTGGAGGTC |
| 2158 | TTN | 2589772 | CCATCCTCTTTCTATGAGGTGTGGG |
| 2159 | TTN | 2589772 | CCTCTTTCTATGAGGTGTGGGGGTC |
| 2160 | TTN | 2589772 | GGTCCCCTCTGTGATCTCTCTATGA |
| 2161 | TTN | 2589415 | TAACTACACTGAGGTCAACCATCGT |
| 2162 | TTN | 2589415 | CTCACGGTAAGGGTTTTCATTGAAC |
| 2163 | TTN | 2589415 | GAACTTTAAGCATTACGACGGGTAC |
| 2164 | TTN | 2589415 | AATCACGGTAGTAGTTTCCTCACGG |
| 2165 | TTN | 2589534 | TCTTTCTCGCCTCAGAGGAGGGGGT |
| 2166 | TTN | 2589534 | AGGTCTTTCTCGCCTCAGAGGAGGG |
| 2167 | TTN | 2589534 | GGACAAGGTCTTTCTCGCCTCAGAG |
| 2168 | TTN | 2589534 | CAAGGTCTTTCTCGCCTCAGAGGAG |
| 2169 | TTN | 2589297 | GAGTTTCTCCTTCGAAGTTTCCAAG |
| 2170 | TTN | 2589297 | GGTTCCTTCCTTAGCAGTTCACAGT |
| 2171 | TTN | 2589297 | CCGTCGCTAGTCTGGGATTGGTAGT |
| 2172 | TTN | 2589297 | GAACACTTTAGTCACCGCTCGGTAG |
| 2173 | TTN | 2589817 | TCAGGGTCGTTCTTTACGAAATAGT |
| 2174 | TTN | 2589817 | CTGTGTTCACTGACGTAAACAAGTT |
| 2175 | TTN | 2589817 | TGTGTTCACTGACGTAAACAAGTTC |
| 2176 | TTN | 2589806 | TCGTTAACTAGTGAAAGTGTGTCCT |
| 2177 | TTN | 2589806 | CGTGAGACTGTCTTTCCAAGTGAAG |
| 2178 | TTN | 2589806 | GACTGGTAACTATGCAGACTACGAC |
| 2179 | TTN | 2589806 | GTCGACACATGAACACCTTCTACTT |
| 2180 | TTN | 2589741 | AAGCCCGTGCATGTGTACATTTCAG |
| 2181 | TTN | 2589741 | GGGCCGAGTTTCCTACAAGACGGAC |
| 2182 | TTN | 2589741 | TGAGGAGAGTGTTAGTCTACCAAAT |
| 2183 | TTN | 2589741 | TCGTCAGACGGACTTCTCGTGAAAG |
| 2184 | TTN | 2589815 | CTATGACATCACCAGTCTTGAATAC |
| 2185 | TTN | 2589815 | GACATCACCAGTCTTGAATACATCT |
| 2186 | TTN | 2589815 | CCAGTCTTGAATACATCTTCTAGTC |
| 2187 | TTN | 2589815 | ATCACCAGTCTTGAATACATCTTCT |
| 2188 | TTN | 2589723 | GGTAATCACGAGTCACCAAATTCCT |
| 2189 | TTN | 2589723 | CAAGACCCGAGAAGATAAGTATACC |
| 2190 | TTN | 2589723 | AATGTGTACGTTTCACAGTTTACAT |
| 2191 | TTN | 2589723 | GTCCTCTACTACGTACGTCACCGTA |
| 2192 | TTN | 2589366 | CACTTGGAGAACTTAGACTCGGTCA |
| 2193 | TTN | 2589366 | AGGTAAACATCATGGTCTACGTGGT |
| 2194 | TTN | 2589366 | GTTACTAACAACATACCCTTTCTGG |
| 2195 | TTN | 2589366 | GACTCGGTCATCAACGGTTCTTAGG |
| 2196 | TTN | 2589695 | CAAGAGCTCCGGTGTATGTGACCGT |
| 2197 | TTN | 2589695 | GACACTTCGAGACCACAGACAGAAT |
| 2198 | TTN | 2589695 | ACCGTGTGGAGGTTAGAGTCACTCG |
| 2199 | TTN | 2589695 | ATCTCCTAATACGTGTCATGTCGAC |
| 2200 | TTN | 2589693 | GCAAGTACTCTGAGATCCGAAGGGT |
| 2201 | TTN | 2589693 | ATGTTTGACTGGTCTCGGTGTATCC |
| 2202 | TTN | 2589693 | CGGTGTATCCCGTCATATTAACGAG |
| 2203 | TTN | 2589693 | AACGTAAACTTACGGCGTAGTTACC |
| 2204 | TTN | 2589359 | GTCACCGTACTTGGTCAGTTGTTAC |
| 2205 | TTN | 2589359 | AACACCTGTTTCCAGTTGTTTTGAT |
| 2206 | TTN | 2589359 | GGTTTCTGAGGTACCAGTATGTCAC |
| 2207 | TTN | 2589359 | GTTCTTAAGACTTACGATACATCGG |
| 2208 | TTN | 2589452 | ATATTCAAGGCACAATAACGGTTCT |
| 2209 | TTN | 2589452 | AGTGTCCAGACGAACTCCCTGTTCT |
| 2210 | TTN | 2589452 | CTGACTCTCTAGATTTACACTGTAG |
| 2211 | TTN | 2589452 | CACAGATGAATTGACCAGACTAGGT |
| 2212 | TTN | 2589641 | GTTCTTTGGGCACGGTCTCCTCTTT |
| 2213 | TTN | 2589641 | AGGACACGGATTCTTCCTTGGACGA |
| 2214 | TTN | 2589641 | TTCGGTCAAGGACACGGATTCTTCC |
| 2215 | TTN | 2589641 | ACTCCAAGGGTTCTTTGGGCACGGT |
| 2216 | TTN | 2589714 | ACCTTACGTTTCATCGACCTAGTAG |
| 2217 | TTN | 2589714 | ACAGTCACCTCGTTTCATGGTTTGG |
| 2218 | TTN | 2589714 | ACAATCCACGAAGAACGTAGAACCT |
| 2219 | TTN | 2589714 | TAGTGGATAAAGTCAACGGACCAAA |
| 2220 | TTN | 2589822 | TGTCGTTCTCATGGTCGTGGACAAC |
| 2221 | TTN | 2589822 | CAACTTTAAGGACAATGAGGTGGTT |
| 2222 | TTN | 2589822 | ACAATGAGGTGGTTGAAACCAGAGC |
| 2223 | TTN | 2589822 | CTCATGGTCGTGGACAACTTTAAGG |
| 2224 | TTN | 2589464 | GGGTGACTGACTCCCGAGAAATATA |
| 2225 | TTN | 2589464 | ATACACAAGGCTCAACGACGTCTTT |
| 2226 | TTN | 2589464 | TTTCTCTCTGCAGTCCGCTTTTCCG |
| 2227 | TTN | 2589464 | CCTGTTTCGCTGATGTGGCTTTAAC |
| 2228 | TTN | 2589790 | AAGGGTCTCCTACGCCGTCAGATAT |
| 2229 | TTN | 2589790 | ACTACCCGTTCTTATGTGCGAAAAC |
| 2230 | TTN | 2589790 | GCGAAGACACGGCACTATAGGCCTT |
| 2231 | TTN | 2589790 | TAGGCCTTCTGGTGTCGGGTTTTAA |
| 2232 | TTN | 2589589 | CTTCTTTTTCAAGCACTTCGACAAG |
| 2233 | TTN | 2589589 | TTTCGGACTTCAAGGTGGTCGATTT |
| 2234 | TTN | 2589589 | TTCGGACTTCAAGGTGGTCGATTTC |
| 2235 | TTN | 2589589 | CTTTTTCAAGCACTTCGACAAGACT |
| 2236 | TTN | 4086830 | AAACTTCTACCACAACAAGGACCAC |
| 2237 | TTN | 2589488 | CTGGATCAGATGTGGGAGTCTTAAT |
| 2238 | TTN | 2589488 | TCAGTTATGACTTCCACGGTTTCGG |
| 2239 | TTN | 2589488 | CACAGAAACTGGTTAGTGTCTCCAC |
| 2240 | TTN | 2589488 | GAGTCCTAGCATCAAGGAGAATTCC |
| 2241 | TTN | 2589309 | ACACTGTTACTGTGCCATGGCGAGG |
| 2242 | TTN | 2589309 | GGGCACTACCGTACTGTGAATGAAC |
| 2243 | TTN | 2589309 | GGCACAGTGTCGGTAATTACGTTCT |
| 2244 | TTN | 2589309 | CGTCTAGCTACCCAGGCACATTTAT |
| 2245 | TTN | 2589680 | TTCACCTCTTATATGAACGTATCAA |
| 2246 | TTN | 2589680 | GCTACATCTATGTAGTGTTTCACCT |
| 2247 | TTN | 2589680 | TGAACGTATCAATCGTTACTTCGAC |
| 2248 | TTN | 2589680 | ACGACTTGACCTCAACAAGCTACAT |
| 2249 | TTN | 2589824 | CAGGTACACCTATTTGCGGGGGCGT |
| 2250 | TTN | 2589824 | TCGATCGGGAGTGAAATGACAAAGT |
| 2251 | TTN | 2589824 | TCTTTTGATGTCTAGATTGTTGCCT |
| 2252 | TTN | 2589824 | GATTGTTGCCTTTCTAATCAGGTAC |
| 2253 | TTN | 2589494 | AATGAGACTCCCTACATTTCAATCT |
| 2254 | TTN | 2589494 | GACCCCTTCAGGTTGATTGTCGTTT |
| 2255 | TTN | 2589494 | CCTTTTCAAGTATGTGAATGAGACT |
| 2256 | TTN | 2589494 | TGAGTGCGGTTGGAGAAACACTTTC |
| 2257 | TTN | 2589338 | TCTCGACCGTAAAGACCGTTTGGAC |
| 2258 | TTN | 2589338 | TGCCTGGAGCGTAGATATGAGTAGT |
| 2259 | TTN | 2589338 | GGCTAGCGGAATTATCACCTACGAT |
| 2260 | TTN | 2589338 | GGACGCGGATGATAACTCACCATAT |
| 2261 | TTN | 2589638 | GGTAACATTGAGTTTCTCTCCTTAG |
| 2262 | TTN | 2589638 | TATTGGTAACATTGAGTTTCTCTCC |
| 2263 | TTN | 2589638 | TGAGTTTCTCTCCTTAGAGGTGGTG |
| 2264 | TTN | 2589638 | AACATTGAGTTTCTCTCCTTAGAGG |
| 2265 | TTN | 2589582 | TTCCCGACTTCAACATGGACAGTTT |
| 2266 | TTN | 2589582 | TCCGAGAGTCTCTTCAACAGGGCCT |
| 2267 | TTN | 2589582 | CAACAGGGCCTTTTCTTTCACGTAG |
| 2268 | TTN | 2589582 | TCCCGACTTCAACATGGACAGTTTC |
| 2269 | TTN | 2589444 | GGATTAGACTTTCTCGAGTCCTTCA |
| 2270 | TTN | 2589444 | TCTTCCATGATGACTGACCTTTTCT |
| 2271 | TTN | 2589444 | CCGTCGGGTTAGTTTCCTATGTAAC |
| 2272 | TTN | 2589444 | GCTTCAGTTCGTAGAGTGATCCTAC |
| 2273 | TTN | 2589326 | AGAAAGGCACAGTCAAGTCTCTTGT |
| 2274 | TTN | 2589326 | TCCTTCGGGTAAATGGTAACTGCAT |
| 2275 | TTN | 2589326 | TAGACAGAGTCGACTTAGCACACAG |
| 2276 | TTN | 2589326 | GCACTTAGCCCATGTTGTCGAACCG |
| 2277 | TTN | 2589652 | GGACTTTTCTTTCGTGGTGGTGGAG |
| 2278 | TTN | 2589652 | CTTTTCTTTCGTGGTGGTGGAGGAT |
| 2279 | TTN | 2589652 | TTCTTTCGTGGTGGTGGAGGATTTC |
| 2280 | TTN | 2589652 | GTTTCATGGACTTTTCTTTCGTGGT |
| 2281 | TTN | 2589399 | GACACCCACTTTCGGTTACTATGAG |
| 2282 | TTN | 2589399 | TACTATGAGAACAGGCCAGTTGACT |
| 2283 | TTN | 2589399 | CGTGCTCAGTGGTTTCAGTTGTTCC |
| 2284 | TTN | 2589399 | CCAGTTGACTTATAGGAACACGACC |
| 2285 | TTN | 2589636 | CTCCTTCTTCAAGGTGGTGGTGGTT |
| 2286 | TTN | 2589636 | GTTTCTTCTTTCAAGGACTTCTTTC |
| 2287 | TTN | 2589636 | GACTTCTTTCCTTTGGACAAGGAGC |
| 2288 | TTN | 2589636 | GGACAAGGAGCCTTCCTCCTTCTTC |
| 2289 | TTN | 2589622 | ACTTTGAGTTTGGATTTTCTCTCCT |
| 2290 | TTN | 2589622 | TTTCTCTCCTCCTTGGTGGTCGATT |
| 2291 | TTN | 2589622 | GTCTCCTTGGCTTCTCTCGACAGGG |
| 2292 | TTN | 2589622 | TGGCTTCTCTCGACAGGGTCTTCTT |
| 2293 | TTN | 2589792 | TAGGTGGGCCTACAGACTACGACCC |
| 2294 | TTN | 2589792 | CTCTTTATACAGGTGGCGGAAGACT |
| 2295 | TTN | 2589792 | CCGTTGCACAGTTGACGTTTTGAGA |
| 2296 | TTN | 2589792 | CTCTACAAGCGTAGGCTTCATAATT |
| 2297 | TTN | 2589351 | TTATAACAACCGTAACCGTTCGGAT |
| 2298 | TTN | 2589691 | GTGGGTCCAAGTAATTCTTCGATCT |
| 2299 | TTN | 2589691 | TACAGTAAGCAACTGAGCCACCGAC |
| 2300 | TTN | 2589691 | TTACGTTTTAGCCACCCAGAGGTCT |
| 2301 | TTN | 2589691 | ACGACCTTTACGTGTTAGAGTCACA |
| 2302 | TTN | 2589503 | GAGAATAGAAGTAGTGTGGAGAGTC |
| 2303 | TTN | 2589503 | CGGAGAATAGAAGTAGTGTGGAGAG |
| 2304 | TTN | 2589503 | GGAGAATAGAAGTAGTGTGGAGAGT |
| 2305 | TTN | 2589692 | ACGGCCCGTCATGTGGACGATACGT |
| 2306 | TTN | 2589692 | TTTCTGAGAACAAGACGAGTCGACC |
| 2307 | TTN | 2589692 | GCGGGACCTCCGTTGATGTTCTACT |
| 2308 | TTN | 2589692 | CTTCACGGAGGAAAGAAACTAGATT |
| 2309 | TTN | 2589536 | GAGACCACCAAGGGTTTTTCGGTCT |
| 2310 | TTN | 2589536 | TCCACGGGTTCCTCCAACAAGGACT |
| 2311 | TTN | 2589536 | CACGGAGACCACCAAGGGTTTTTCG |
| 2312 | TTN | 2589536 | ACGGGTTCCTCCAACAAGGACTTTT |
| 2313 | TTN | 2589456 | GAGAAGGCACATTCTCGACTTTTGT |
| 2314 | TTN | 2589456 | GGTCCAGGTGGTACACAAAGTTTCG |
| 2315 | TTN | 2589456 | TCTGGAGTCAGGAAACATTTACCCT |
| 2316 | TTN | 2589456 | GAGTCTTACCTAACAACAGTGAAGT |
| 2317 | TTN | 2589844 | GAAGCAAGTTGCTGACGTCTCGTAC |
| 2318 | TTN | 2589844 | ACTGACCTTAGGGATGTGGACACCA |
| 2319 | TTN | 2589844 | AAGATGGCCCTACCTCGGCTTTAGG |
| 2320 | TTN | 2589844 | CCGCTGGAGATGTCGAATGACTAAC |
| 2321 | TTN | 2589603 | CACCAGTAAGGGTTCTTTCTCCTCC |
| 2322 | TTN | 2589603 | GTAAGGGTTCTTTCTCCTCCGAGGG |
| 2323 | TTN | 2589603 | CAGTAAGGGTTCTTTCTCCTCCGAG |
| 2324 | TTN | 2589603 | TCTTTCACCAGTAAGGGTTCTTTCT |
| 2325 | TTN | 2589394 | CATCCGCTAGGTCAGAAGTGACTTG |
| 2326 | TTN | 2589394 | GGTCAGAAGTGACTTGGTCGTTAAC |
| 2327 | TTN | 2589394 | CGCTAGGTCAGAAGTGACTTGGTCG |
| 2328 | TTN | 2589394 | GAAGTGACTTGGTCGTTAACGGTTT |
| 2329 | TTN | 2589727 | CTTATGGTCACGTAACATAGGTTAC |
| 2330 | TTN | 2589727 | ACGAGTACGTCATGATCTCAACGGG |
| 2331 | TTN | 2589727 | CTTGTGGAGGCAAACTTCAGTGAAC |
| 2332 | TTN | 2589727 | AGGTATATTGGTTCACACTGGGAAG |
| 2333 | TTN | 2589737 | CCGTGAGGAGGGAAACTCTAGTGAA |
| 2334 | TTN | 2589737 | TCTAGGAGTTCAAACATCGACGTCT |
| 2335 | TTN | 2589737 | GTCCTAGTAGACCAATCGGACGTCT |
| 2336 | TTN | 2589737 | GACCGCTTATGGTCACAGCCCACTG |
| 2337 | TTN | 2589775 | TTTGTTAGGAGTTATGTAAGTCCTC |
| 2338 | TTN | 2589775 | TTAGGAAGTCTATATTGATTTGTTA |
| 2339 | TTN | 2589526 | AACTTTTCATATAATTTGGACTTCT |
| 2340 | TTN | 2589526 | CATATAATTTGGACTTCTCGGGCTT |
| 2341 | TTN | 2589526 | TTCATATAATTTGGACTTCTCGGGC |
| 2342 | TTN | 2589526 | CTTTTCATATAATTTGGACTTCTCG |
| 2343 | TTN | 2589847 | GACCTCCCATCATGGCGTTGGAAAC |
| 2344 | TTN | 2589847 | TCGTGGCTGCAAATGCGTCGGCAAT |
| 2345 | TTN | 2589847 | TACTGTTGAGTTCGTGGCTGCAAAT |
| 2346 | TTN | 2589847 | CGTCGGCAATGTTTCGCAACACCAT |
| 2347 | TTN | 2589293 | GGTGTCAGTTCTTGTCCCCTCCAAG |
| 2348 | TTN | 2589293 | CTTAAACCTAGACTGAGACGGTGAC |
| 2349 | TTN | 2589293 | CCCCTCCAAGGTGTAACTTTTGTGT |
| 2350 | TTN | 2589293 | ACCACCTGAAATATGGGACTCAAAT |
| 2351 | TTN | 2589327 | AGGAGGCGTCAGAATCGAACCGAAT |
| 2352 | TTN | 2589327 | TGCCACCACGGGCTTAGGTAGTAAT |
| 2353 | TTN | 2589327 | GGAAGTTTCTACACTGGGCCCCTAG |
| 2354 | TTN | 2589327 | GGGCCCCTAGACGATGTAACTACAC |
| 2355 | TTN | 2589487 | CTGGAATCCTAACAACTCGGAGAAT |
| 2356 | TTN | 2589487 | GTGACTCAAGCTACTGCGACAGAAG |
| 2357 | TTN | 2589487 | AAGACCACGTTCCACTTAGCAGAGT |
| 2358 | TTN | 2589487 | TTCACGGAAAACTGTTGGCACAGAG |
| 2359 | TTN | 2589823 | TCGGCCATCACGATAACGGTGTAAT |
| 2360 | TTN | 2589823 | GTGGTTTAGCCACTTCCGAGGATGA |
| 2361 | TTN | 2589823 | TGGTGGCAGGCACTTCTCGCGAAAC |
| 2362 | TTN | 2589823 | TCGCGAAACTTCATGACGTGCCTGC |
| 2363 | TTN | 2589508 | CACTGTAACCTACTATATAGAGTCT |
| 2364 | TTN | 2589508 | ACTACGGTTTGAGGTATGTTCGTCT |
| 2365 | TTN | 2589508 | CTGTAACCTACTATATAGAGTCTAT |
| 2366 | TTN | 2589508 | GGTATGTTCGTCTTGTCATGAGTAG |
| 2367 | TTN | 2589696 | GTAGAGAGACCTTCTAAAACAGTGT |
| 2368 | TTN | 2589696 | CCTTACTCGGAGTGTAAGTGGTCAC |
| 2369 | TTN | 2589696 | CAACAAACTCCACGTCGGTAATCTT |
| 2370 | TTN | 2589696 | CCTCTGATAAGAACGGAGCAATGTT |
| 2371 | TTN | 2589428 | CTCCCCACTAGGCTTAGTAACGGAT |
| 2372 | TTN | 2589428 | GGAATGGAGTTCTTCGGTGGTAACT |
| 2373 | TTN | 2589428 | ATCGAGTAGGTACCAGTAGTTCTTG |
| 2374 | TTN | 2589428 | TCTGGTGTCGGTAATCGAGTAGGTA |
| 2375 | TTN | 2589348 | ATGGCACGCACACTACGGAACATAG |
| 2376 | TTN | 2589348 | AGGCACCGTTTGGATATGGACTACA |
| 2377 | TTN | 2589348 | GCAAGACGCACGTTCACGATGAAAT |
| 2378 | TTN | 2589348 | GGACTTCCCGGAGACTTTCAATGAC |
| 2379 | TTN | 2589673 | CACCGACCAAGGGTTGGATATTGAC |
| 2380 | TTN | 2589673 | TTCACAGGTTACTACGTCCGAGACG |
| 2381 | TTN | 2589673 | CTTGCTGCGACCAAACATGTGTACG |
| 2382 | TTN | 2589673 | CCGAGACGAGACACGTGCAGAAGTT |
| 2383 | TTN | 2589406 | CACTCTCCTGCTGGTCGGGGATTTC |
| 2384 | TTN | 2589760 | AAAACTGCCACTACTAGTATCGGAC |
| 2385 | TTN | 2589760 | TCTTCCTGAAGTATCGCGGCTTGAA |
| 2386 | TTN | 2589760 | CCGTGACCGGGTTAAAAGTAGTTTC |
| 2387 | TTN | 2589760 | TCGACCCTCCAGGAACAGGAGGAGT |
| 2388 | TTN | 2589694 | GTGGCATGAAATAACTTGGAGACCT |
| 2389 | TTN | 2589694 | ACCCTCTCATAAGTACGTTCCGTCT |
| 2390 | TTN | 2589694 | TGGAGACCTTGTACACCTTCGTCAG |
| 2391 | TTN | 2589694 | GTTTCGATGCTAGTCGAGGACGTAT |
| 2392 | TTN | 2589678 | AAGGTTCATCGTGTCATCTCCTACG |
| 2393 | TTN | 2589678 | GCTGACTTTTTAGCCGTTAGGACCT |
| 2394 | TTN | 2589678 | GGAAGAGTCTCCACATTATATTGAT |
| 2395 | TTN | 2589678 | ACTAGGATCTCCCATGTAAGTGACC |
| 2396 | TTN | 2589363 | TGCAGGGACCTGGATAATATCAACT |
| 2397 | TTN | 2589363 | CGTGGTCTGTAACTAGAACTGGATC |
| 2398 | TTN | 2589363 | ATGTTGAAGGCATAGACACGATAAT |
| 2399 | TTN | 2589363 | ACCTCAACCTCTTGTACGACTGCAG |
| 2400 | TTN | 2589396 | ATAAGTCGCACTTGGCCTTCCTGAG |
| 2401 | TTN | 2589396 | GGGTCCTATCTGGACTCACACCGAT |
| 2402 | TTN | 2589396 | GACCTTGATTACAGACGAACCTACG |
| 2403 | TTN | 2589396 | CCTGAGTCCTCTGATATGGTAATGA |
| 2404 | TTN | 2589352 | GTAAGTCCTGTGGTTTAAGTTTTGT |
| 2405 | TTN | 2589352 | GGGTAAGTCCTGTGGTTTAAGTTTT |
| 2406 | TTN | 2589352 | GTTGACCCGAACTACTCCCGGAACT |
| 2407 | TTN | 2589352 | TTTTGTTGACCCGAACTACTCCCGG |
| 2408 | TTN | 2589703 | AGGTCATCCTCGAGAATTTCCAAGA |
| 2409 | TTN | 2589703 | CAGCCCCTTATAGTGACGTTTCGAT |
| 2410 | TTN | 2589703 | CCAAGACTACACTAAGAGGTTACAC |
| 2411 | TTN | 2589703 | ACCCTTCACTGTGCACAAGAACGAG |
| 2412 | TTN | 2589392 | CGGCGAATACGAGCCCTGGGAGTCA |
| 2413 | TTN | 2589392 | TCACTGCGTAGGTTCCGGCGAATAC |
| 2414 | TTN | 2589392 | GTCCGACAATTGACCTGATTCCAGT |
| 2415 | TTN | 2589392 | CCTGCGACACTAGGAGGACAATAAT |
| 2416 | TTN | 2589743 | GCTGTCACCGGTTATGTGTAAACTC |
| 2417 | TTN | 2589743 | CACTGTCGTCCTCTAGGGCGGTGTG |
| 2418 | TTN | 2589743 | TCGTCTTGACTAGGTCCACTGTCGT |
| 2419 | TTN | 2589743 | AAGTCGACTCGACGTGCTGTCACCG |
| 2420 | TTN | 2589708 | CCGGTCATGAGAACGAGTCGAAGGT |
| 2421 | TTN | 2589708 | GGGTGGAAGAAAACGTTCTGTTAAT |
| 2422 | TTN | 2589708 | TGACACCCCAATGGACAATGTGAGT |
| 2423 | TTN | 2589708 | GAGTGAACAGCTAATTTACCGAGAC |
| 2424 | TTN | 2589733 | GTTGGTTACGTCACCCGTAGACTGT |
| 2425 | TTN | 2589733 | ATTCCGTCCCGTGTTGGTTACGTCA |
| 2426 | TTN | 2589733 | CCCTGTATGTGAACAAGACGGTGTT |
| 2427 | TTN | 2589733 | ACGTCACCCGTAGACTGTCAGTTTC |
| 2428 | TTN | 2589322 | GACCAATGGTAGTCTCGTCCAAGAC |
| 2429 | TTN | 2589322 | TTCGGCTTGACCTACGGGCTAATGT |
| 2430 | TTN | 2589322 | GTTCCGGCACAGACAGTACCAGTTT |
| 2431 | TTN | 2589322 | ATGACCGTTTGCTCGTTGACGACAC |
| 2432 | TTN | 2589378 | GTCTAAGACAAGACTACTTTCTACG |
| 2433 | TTN | 2589378 | AGACAAGACTACTTTCTACGTCGTA |
| 2434 | TTN | 2589378 | TAAGACAAGACTACTTTCTACGTCG |
| 2435 | TTN | 2589378 | ACTCCGAAGTCTAAGACAAGACTAC |
| 2436 | TTN | 2589755 | GGGTAATGACTTGGTCTTCAACTTA |
| 2437 | TTN | 2589755 | AGTGTCTTCCAACTTGGGTAATGAC |
| 2438 | TTN | 2589755 | TCAACTTAGATTTATAGACTAGAGT |
| 2439 | TTN | 2589755 | GATTTATAGACTAGAGTTGACTTCT |
| 2440 | TTN | 2589501 | CCAAGTTCTTACTGGTCGCGGATGT |
| 2441 | TTN | 2589501 | GGTCCAGCCAGAGTTACGTTCTGCT |
| 2442 | TTN | 2589501 | GAGGGTTTAATCTCATCTTCGATAC |
| 2443 | TTN | 2589501 | TCCCTTTTGAGTAAGCTAGTGTAAG |
| 2444 | TTN | 2589454 | CAGGCAGTCATGTGGCAGTTTCTTT |
| 2445 | TTN | 2589454 | GTGTTTACTTACCAGTGCGACGTGT |
| 2446 | TTN | 2589454 | ACCTCTTTGTGTTGGACAATGACAC |
| 2447 | TTN | 2589454 | TTGAAGCCCACTCACGACAGTTACG |
| 2448 | TTN | 2589746 | CTTAGTCGTGCGGAGGTAACGTTCG |
| 2449 | TTN | 2589746 | GTCACCCTCAATGAGTACACTTCGT |
| 2450 | TTN | 2589746 | CGTCACTTACTGCAGCCGTCACTAT |
| 2451 | TTN | 2589746 | GACTCCGATATGAACTATAATGCCT |
| 2452 | TTN | 2589343 | TGGGTAGTGCAACAAAGGCCAGACT |
| 2453 | TTN | 2589343 | TCACTGGGCTCTCTCGAAGACGAAG |
| 2454 | TTN | 2589343 | ACATGTGATACAGCAACTTCGTGAT |
| 2455 | TTN | 2589343 | AGGACAAGAATAATTCCTCGTTGAT |
| 2456 | TTN | 2589374 | CACACATGATAGTACCAAGGGAGGT |
| 2457 | TTN | 2589374 | AGTCTCGGACACGAACGTCACTTAG |
| 2458 | TTN | 2589374 | TTCGACTACAAAGTCCGGCGGGTGG |
| 2459 | TTN | 2589374 | CCACGGATATGGGAATGTCGCTGAT |
| 2460 | TTN | 2589649 | CAAAGTTTCTTCTAACAAGGTGTTT |
| 2461 | TTN | 2589649 | AGGGCCTGAGGTCATGTCCTTCTTC |
| 2462 | TTN | 2589649 | AGTTTCTTCTAACAAGGTGTTTTTG |
| 2463 | TTN | 2589649 | GTCCTTCTTCAATAACTTCACTTTC |
| 2464 | TTN | 2589387 | TGAGGACCTGGTCAACACCTGGACT |
| 2465 | TTN | 2589387 | CGGATGTCATGAGACGTTTTCTAAA |
| 2466 | TTN | 2589387 | GGGTTCGCTTTAGTGACAACACCGT |
| 2467 | TTN | 2589387 | AGGACATCGTAACCGACCTTTTTCG |
| 2468 | TTN | 2589489 | GTGAATGCAACAGTACCATCCCCGG |
| 2469 | TTN | 2589489 | CGGTGTAATGTTCTATACCCGTGAA |
| 2470 | TTN | 2589489 | GTCAGGTCACCTTCTCCCTACTATT |
| 2471 | TTN | 2589489 | CCTACTATTCTGTGAACTTAGACCT |
| 2472 | TTN | 2589333 | GCACCAGGGTGCGACCGTATTCATT |
| 2473 | TTN | 2589333 | CTAGGACAGTGATAACCCGGTTAAT |
| 2474 | TTN | 2589333 | GACCCTAGGAGGAGAGTAACTACCT |
| 2475 | TTN | 2589333 | TCTTGTACCAGACAGCACAGTGTGT |
| 2476 | TTN | 2589360 | CAATGTCACATGTCCAACCGGTTCT |
| 2477 | TTN | 2589360 | GTTTCGAAACGGAATCTCAGACTAG |
| 2478 | TTN | 2589360 | CTTCACGGTTAAAGACCTGCAGGAT |
| 2479 | TTN | 2589360 | CTGCAGGATTCGGTTGGTAATGGAC |
| 2480 | TTN | 2589542 | GGATTTTTCGGACTTCAGTGTGGAC |
| 2481 | TTN | 2589542 | TCCGAGGCTTTCTTCAACAAGGACT |
| 2482 | TTN | 2589542 | CGAGGCTTTCTTCAACAAGGACTTT |
| 2483 | TTN | 2589542 | GACTTTTCTTTCACGGTCGCCGAGG |
| 2484 | TTN | 2589299 | TATTTGAGAGACTTCTGTTCCCTCC |
| 2485 | TTN | 2589299 | GTCACCTGAAATATGAACATGTCAT |
| 2486 | TTN | 2589299 | CACAGGAGATCGACGTTTAATTGTT |
| 2487 | TTN | 2589299 | TATTCTGACTATGAAGACTGTCACC |
| 2488 | TTN | 2589688 | AGATGACAGCAACCATTTCTTCAAG |
| 2489 | TTN | 2589688 | GTTTCTATTCCCTCTTTAGCAATCT |
| 2490 | TTN | 2589688 | CTTCGAGTCACTGTATAGATGACAG |
| 2491 | TTN | 2589688 | GGTAACTTCCGCGACTTGGGTAAAG |
| 2492 | TTN | 2589836 | TTCGTTCTCGTCTACGTGCATTGAG |
| 2493 | TTN | 2589836 | CGTCTACGTGCATTGAGTACTCGTC |
| 2494 | TTN | 2589836 | GACGACATTGATTCCATCATCACCG |
| 2495 | TTN | 2589836 | TCACCGGCGGCTATTTCGGTTCCTT |
| 2496 | TTN | 2589468 | CCAATCTACGTTATTCTACGGTCAT |
| 2497 | TTN | 2589468 | ATTTCTGCGTCACTGAGAGTGTACC |
| 2498 | TTN | 2589468 | ACGGTCATTTCCTGTGTTGTATGTC |
| 2499 | TTN | 2589468 | CACCGTCGGGTTAGTGTCCTATGAC |
| 2500 | TTN | 2589665 | ACACTTCACAGGAAACTACTACGGT |
| 2501 | TTN | 2589665 | ACCGGCGACGGTAATATACTGGTAG |
| 2502 | TTN | 2589665 | GTTGAAGTCCTTACTACCGGCGACG |
| 2503 | TTN | 2589665 | CACTCGTAGTCAGACGGTGGAAACT |
| 2504 | TTN | 2589590 | CCTCCACGGATTCTTTTAACACCAT |
| 2505 | TTN | 2589590 | TTTCATGCACAAGGACTTCTCGGGT |
| 2506 | TTN | 2589590 | ACGGATTCTTTTAACACCATCTTCT |
| 2507 | TTN | 2589590 | TGGCCTCCACGGATTCTTTTAACAC |
| 2508 | TTN | 2589715 | AGAAAGTGTGCTTCTGACTTTTTAT |
| 2509 | TTN | 2589715 | GGAGAAAGTGTGCTTCTGACTTTTT |
| 2510 | TTN | 2589715 | AGGGAGAAAGTGTGCTTCTGACTTT |
| 2511 | TTN | 2589715 | GAAAGTGTGCTTCTGACTTTTTATG |
| 2512 | TTN | 2589328 | GCGGAGAAGTAACGGATGGTTCCAT |
| 2513 | TTN | 2589328 | ACTGGCCAATTGTCGAGGACTCAAT |
| 2514 | TTN | 2589328 | TCCAGGAGGGTGTCACCAGTTTCAT |
| 2515 | TTN | 2589328 | ATCTCTCGGGCAGTTGGGTGGTCCA |
| 2516 | TTN | 2589438 | AGTCGTTCGGAAGTCGGTGACAACC |
| 2517 | TTN | 2589438 | GGACCTCCGTCACGTTATTCGCACT |
| 2518 | TTN | 2589438 | GGGACAATGGGTGATATAACAACTC |
| 2519 | TTN | 2589438 | ACTCACGGAACGTACCCTGGGATGA |
| 2520 | TTN | 2589436 | CCACCATCTACTATGAACGTGAAAT |
| 2521 | TTN | 2589436 | ACACGGTAAGGGCTGTGATTGGACC |
| 2522 | TTN | 2589436 | CTCACTGTGACCGAATATATGGTAG |
| 2523 | TTN | 2589436 | AGGCGTCAGAGCGTCCTCACTGTGA |
| 2524 | TTN | 2589410 | CACCGTCAGGTTATTGACCGATGAT |
| 2525 | TTN | 2589410 | GACCGATGATATACCTTGCAGCTCT |
| 2526 | TTN | 2589410 | TGGATAGCGACTGGACTTCAAGTCT |
| 2527 | TTN | 2589410 | TGACCGTTTACCCACTCCCAGTTGT |
| 2528 | TTN | 2589473 | GGATGGCGCACTTCCGGAATTTGTC |
| 2529 | TTN | 2589473 | CTCCTTCCGTTTACCATACGGATGG |
| 2530 | TTN | 2589473 | TCACTGTCCAGAACTCCTTCCGTTT |
| 2531 | TTN | 2589473 | TTACCATACGGATGGCGCACTTCCG |
| 2532 | TTN | 2589369 | GTCTTACAATACACCGAGCACTGGG |
| 2533 | TTN | 2589369 | AGCACTGGGTACACTAGGTGGTCCT |
| 2534 | TTN | 2589369 | CCAATATCGGGCTTTACGGCGTCCT |
| 2535 | TTN | 2589369 | GACCGGATCAACTTCTAGTGTCTAT |
| 2536 | TTN | 2589519 | CTTCTCCAACTTCATGGATGACAAT |
| 2537 | TTN | 2589519 | TCTCCAACTTCATGGATGACAATGT |
| 2538 | TTN | 2589519 | CTCCAACTTCATGGATGACAATGTT |
| 2539 | TTN | 2589480 | GAGTCAGTAGTCAAGTCGAGTTTAC |
| 2540 | TTN | 2589480 | CTCTGGTTCAGGAGGACATTTGGAT |
| 2541 | TTN | 2589480 | TACCCTTGGAGGAGACTTTCTACCT |
| 2542 | TTN | 2589480 | GAAGGAGTCTAGTCTGAGTCAGTAG |
| 2543 | TTN | 2589319 | ACCTATGGTGGTTTGTGTCGTAATC |
| 2544 | TTN | 2589611 | GGACACGAATAAGGATTTTTCCTCT |
| 2545 | TTN | 2589611 | GGTCTCCTCTTTCAAGGACACGAAT |
| 2546 | TTN | 2589611 | TTTTCCTCTTCGGAGGCGGTCGTTT |
| 2547 | TTN | 2589611 | TTCCTCTTCGGAGGCGGTCGTTTTC |
| 2548 | TTN | 2589662 | TGGTACGGATAGTCTCGTCACGGTG |
| 2549 | TTN | 2589662 | TTAGGGTTGGTACGGATAGTCTCGT |
| 2550 | TTN | 2589662 | CTCAAGGTTAGGGTTGGTACGGATA |
| 2551 | TTN | 2589662 | GTTGGTACGGATAGTCTCGTCACGG |
| 2552 | TTN | 2589412 | CCCGGTGGTTGTCCAGGATAATTAT |
| 2553 | TTN | 2589412 | CGGACAAGGATGACGTTTCACCTGT |
| 2554 | TTN | 2589412 | ACGTCGGCCATCGTTTTGTCATCGG |
| 2555 | TTN | 2589412 | AGTACCGTCGGTGGATTCCTACTAC |
| 2556 | TTN | 2589718 | CACCTTTTACAACGTTGAGATGTCA |
| 2557 | TTN | 2589718 | GTTCGTAGGTATCTTCCGCGGGTCG |
| 2558 | TTN | 2589718 | GGTGGTTCTAAACAGAGGTTTGACT |
| 2559 | TTN | 2589718 | TGTCGGAGTGACAACATCGGCCTCT |
| 2560 | TTN | 2589655 | GATTTTTTCTTCAAGTGCTCCTTAC |
| 2561 | TTN | 2589655 | TCACCCTTCTCCGAATGGTTCTTTC |
| 2562 | TTN | 2589655 | TCTTCCGATACTGCTTCCCCTCCTT |
| 2563 | TTN | 2589655 | TCTTCCCGTTCTTATGATACTTTCC |
| 2564 | TTN | 2589610 | TTTTCCACCTTCGAGGTGGACGGTT |
| 2565 | TTN | 2589610 | ATTTTTCCACCTTCGAGGTGGACGG |
| 2566 | TTN | 2589610 | GGATTTTTCCACCTTCGAGGTGGAC |
| 2567 | TTN | 2589610 | TTCCACCTTCGAGGTGGACGGTTTC |
| 2568 | TTN | 2589461 | TCTCAATTAAGAGCGGGTTATTTCC |
| 2569 | TTN | 2589461 | GTTACGATAACCACAGTCGCTCGGT |
| 2570 | TTN | 2589461 | GGGAACTGGATGTACACTGACTACG |
| 2571 | TTN | 2589461 | GTCGCTCGGTAGACTTTAGAGACTT |
| 2572 | TTN | 2589478 | TCCGGTTGACGTTGGACCACAAAAC |
| 2573 | TTN | 2589478 | CTTGCAAGTCTGTTCCCGTAAATAT |
| 2574 | TTN | 2589478 | ACCAAGGTTCATGTCCGATAGGTTC |
| 2575 | TTN | 2589478 | GAACAGACGGATACGGCTTGAACAG |
| 2576 | TTN | 2589756 | GACTCCCAGGATCTTAAGTCGTTCC |
| 2577 | TTN | 2589756 | CCTTTATCTGTAGGATTGTCGACTC |
| 2578 | TTN | 2589756 | TTCCTCGGTTTTGTTCAAACGTTCT |
| 2579 | TTN | 2589756 | TTATCTGTAGGATTGTCGACTCCCA |
| 2580 | TTN | 2589656 | TCGGTAAACTTGTTGGAATAATACT |
| 2581 | TTN | 2589656 | TGTGTCTCGGTAAACTTGTTGGAAT |
| 2582 | TTN | 2589656 | TGGTATGTGTCTCGGTAAACTTGTT |
| 2583 | TTN | 2589656 | CATCTTGGTATGTGTCTCGGTAAAC |
| 2584 | TTN | 2589676 | AAAATTTTGACTGGCCTCGGAACGT |
| 2585 | TTN | 2589676 | ACACGGTATTCACCTAGTCTTGGAT |
| 2586 | TTN | 2589676 | CCGGTCATAAGGACGTGTCGATGTT |
| 2587 | TTN | 2589676 | CTTGGATAGAGGCACAGAACCATAT |
| 2588 | TTN | 2589376 | TCGACCTAGAGGCACCGTAGATAGT |
| 2589 | TTN | 2589376 | CTTTAAGGTCACGAGCCAGCTGGCT |
| 2590 | TTN | 2589376 | ACCGGATTCGGGTTTGTGCTACCAC |
| 2591 | TTN | 2589376 | GGGCGGAATGATGACCTAATCTCAT |
| 2592 | TTN | 2589605 | CTCCTTTAAGGTGGACTCCTTCTCC |
| 2593 | TTN | 2589605 | TCAAGGAGGGCTTCTTCTTATACAT |
| 2594 | TTN | 2589605 | GGAGGGCTTCTTCTTATACATGGAC |
| 2595 | TTN | 2589605 | TTCTTCTTATACATGGACTCCTTCT |
| 2596 | TTN | 2589799 | GTGGAGGTTTTGTAGACGGTTTGAG |
| 2597 | TTN | 2589799 | CGGTGGAGGTTTTGTAGACGGTTTG |
| 2598 | TTN | 2589799 | CCGGTGGAGGTTTTGTAGACGGTTT |
| 2599 | TTN | 2589799 | ACCGGTGGAGGTTTTGTAGACGGTT |
| 2600 | TTN | 2589498 | CTGTCGTGCGAAACTTTGGCTTTAG |
| 2601 | TTN | 2589498 | TAGAGACTTCTACTATAGGTGCGGT |
| 2602 | TTN | 2589498 | CCTCTGACTCTGTCGTGCGAAACTT |
| 2603 | TTN | 2589498 | TGCGGTTGACCTTTGAGTTCCCTCT |
| 2604 | TTN | 2589401 | GAGACCTCCTGCCTCCGTCATTGTA |
| 2605 | TTN | 2589401 | AAGACTATATCTGCGACTACGAACG |
| 2606 | TTN | 2589401 | GTGCCGAGATCGAAGTCAGTGTTTT |
| 2607 | TTN | 2589401 | GTCGGAGGTAAACTGTAAAGACTAT |
| 2608 | TTN | 2589321 | AGGATACAGCAATGGTCCGAGTAGT |
| 2609 | TTN | 2589321 | GTCTCGGTTAACATCGGTCTTTGAG |
| 2610 | TTN | 2589321 | ATCGTCCGAGTTGACCCACTAACAA |
| 2611 | TTN | 2589321 | TGAGTGATGTAGCACCTTTCTGCGC |
| 2612 | TTN | 2589667 | GCGATCGCGATTTGATTGACATTAA |
| 2613 | TTN | 2589667 | ACAGTTGAATTTCTGGTCCCGTTAA |
| 2614 | TTN | 2589667 | ATAGCTAACCAAACACCAGGTGTGT |
| 2615 | TTN | 2589667 | ACCACTGGCTGTATGTGAGTCTCAG |
| 2616 | TTN | 2589604 | GGTTTCTTTGGACATGGTCTCTTCT |
| 2617 | TTN | 2589618 | TAGGGACATTTCGGACAGGGTCTTC |
| 2618 | TTN | 2589618 | GGATAGGGACATTTCGGACAGGGTC |
| 2619 | TTN | 2589618 | GATAGGGACATTTCGGACAGGGTCT |
| 2620 | TTN | 2589618 | ATAGGGACATTTCGGACAGGGTCTT |
| 2621 | TTN | 2589354 | ACCGTGTACCATAGTCGTTGTCAAC |
| 2622 | TTN | 2589354 | GACCACCGACGGTTTATTCGTTGAT |
| 2623 | TTN | 2589354 | CCGGGTCAAGCCAAACTACTTCAAT |
| 2624 | TTN | 2589354 | ACCCTTGGAGGTCGGATATGACCAC |
| 2625 | TTN | 2589753 | GAGTGTTGTAGGTATTGTTTACGAT |
| 2626 | TTN | 2589753 | TCCACTATAACATGTGGAGTGTTGT |
| 2627 | TTN | 2589753 | ATAACATGTGGAGTGTTGTAGGTAT |
| 2628 | TTN | 2589753 | CATGTGGAGTGTTGTAGGTATTGTT |
| 2629 | TTN | 2589301 | CACTGACCAGTAGGTTTTGGATAGC |
| 2630 | TTN | 2589301 | CGGTCACTACCTCCACGTTTCTAAT |
| 2631 | TTN | 2589301 | ACTACTACGGTGTCAAATGGTTCAG |
| 2632 | TTN | 2589301 | CTCGTTACGATGGAACCAGACGTTT |
| 2633 | TTN | 2589439 | GGCAAAACCATAACCGGGTGGACAC |
| 2634 | TTN | 2589439 | GCACACGCTCGTCTTTTGGCAAAAC |
| 2635 | TTN | 2589439 | GTTCCCCATCAACGTCTGAAAGTAC |
| 2636 | TTN | 2589439 | GTCCGGGTGGGTTTCTAGACTTTCA |
| 2637 | TTN | 2589296 | AGTCGATGTCGAAGGAATTACCAGG |
| 2638 | TTN | 2589296 | AAAGGCACCGGTCACAAGTCGATGT |
| 2639 | TTN | 2589296 | CGTATTCGGCGAGGTCTTTACATAT |
| 2640 | TTN | 2589296 | AGTCAGTCGCTGTCACCTTTCATGT |
| 2641 | TTN | 2589467 | TTTTTATGTCTAAGGCACACAACCG |
| 2642 | TTN | 2589467 | TAGTTGACTTGGTTAGAATTATTTC |
| 2643 | TTN | 2589467 | CGACCTGGACCTTTTGGTTCGTTTA |
| 2644 | TTN | 2589467 | TGTCTAAGGCACACAACCGACTTTT |
| 2645 | TTN | 2589835 | TAAAGGCGTCGATTTCGGTTTCTTG |
| 2646 | TTN | 2589835 | ATCATTAAAGGCGTCGATTTCGGTT |
| 2647 | TTN | 2589835 | CATTAAAGGCGTCGATTTCGGTTTC |
| 2648 | TTN | 2589835 | TTCCATCATTAAAGGCGTCGATTTC |
| 2649 | TTN | 2589797 | AAGTGACAGCTCGAATGTGTGGGAT |
| 2650 | TTN | 2589797 | CGGTAGCACCTACTCAGACAAATAC |
| 2651 | TTN | 2589797 | ACCTCGGTCACGGTCTGACGTGCAC |
| 2652 | TTN | 2589797 | AGACTCCTAATTTTTGACACGGTAG |
| 2653 | TTN | 2589450 | TGCCGCAGTACTGTTTGGACTGAAA |
| 2654 | TTN | 2589450 | GAGGTCTCTCCGAGTGTATGTGACT |
| 2655 | TTN | 2589450 | ACCGTCAGGGTAGGTTCCTATATAG |
| 2656 | TTN | 2589450 | CATACTCAAGGCACAGTTTCGACAG |
| 2657 | TTN | 2589745 | CCTCTAGAAAAGCAAATTATCACGT |
| 2658 | TTN | 2589745 | CCTTGTTTACGAGATGAAGTCACAC |
| 2659 | TTN | 2589745 | ACCGACGTACCGTTGGGTGAATGAG |
| 2660 | TTN | 2589745 | TGAGAACTCGGACGTCTGTATCACT |
| 2661 | TTN | 2589719 | GAAGTCTGTAGCCACTTATGGTGAC |
| 2662 | TTN | 2589719 | CGTCGAAGGGAGGATATCATCTTTG |
| 2663 | TTN | 2589719 | GGTGGCAAACTCCACCATACCATGT |
| 2664 | TTN | 2589719 | TACCATGTTTCTGTTCGCCGTTGAG |
| 2665 | TTN | 2589477 | TCAGTCGGCCTTTTGTACCTGATTT |
| 2666 | TTN | 2589477 | ACCACTATATTGGTTCCTGAGTCAT |
| 2667 | TTN | 2589477 | ACCTCCTTCAGGCAATTGACCTATG |
| 2668 | TTN | 2589477 | AACTGAACCCTTGGTGGACTACTAC |
| 2669 | TTN | 2589717 | GAAGGAATTCCTAAGAGAGTCAACT |
| 2670 | TTN | 2589717 | GACCGTGAGGCCTTGAGAGACAACT |
| 2671 | TTN | 2589717 | GGGCGTCAGTAACAACTCTTCCGTC |
| 2672 | TTN | 2589717 | CACTGACATCCTCTTTGCACGTGAG |
| 2673 | TTN | 2589518 | GACTTTAGTTCGGTCGTTATGGAGA |
| 2674 | TTN | 2589518 | ACTTGGCTTTGGTTTCGGGCTTCGT |
| 2675 | TTN | 2589518 | TTAGTTCGGTCGTTATGGAGAGGGA |
| 2676 | TTN | 2589518 | AGAGGGACGTGGACTTGGCTTTGGT |
| 2677 | TTN | 2589314 | AGGGTCGACCGTCTGGTCATCTCGA |
| 2678 | TTN | 2589314 | ACCAAGAAACGACCAAGATTTGACT |
| 2679 | TTN | 2589314 | CCACTATGGATAACGACCGGCAGGT |
| 2680 | TTN | 2589314 | TCTTCTGGTAGGTACAGGGTCGACC |
| 2681 | TTN | 2589800 | GTGTTTTCCTCTGAATAGTAACGAC |
| 2682 | TTN | 2589800 | CTACCGGTGTTTTCCTCTGAATAGT |
| 2683 | TTN | 2589800 | GGTGGTTTAATCTACTGTAACCTCT |
| 2684 | TTN | 2589800 | GACGGTGGTTTAATCTACTGTAACC |
| 2685 | TTN | 2589346 | CAGACACCGGCAATTAACGTTTCAT |
| 2686 | TTN | 2589346 | TACCACCACGTCTATAGCTGATAAT |
| 2687 | TTN | 2589346 | TCATGAACTATTCGGACCAGGTGGT |
| 2688 | TTN | 2589346 | CGGTGGAACGTACCTGTTATACACT |
| 2689 | TTN | 2589530 | GGTCACTTCTTCCAGGGTTGACAAT |
| 2690 | TTN | 2589530 | AAGGTCACTTCTTCCAGGGTTGACA |
| 2691 | TTN | 2589530 | TCACTTCTTCCAGGGTTGACAATTC |
| 2692 | TTN | 2589530 | CACTTCTTCCAGGGTTGACAATTCT |
| 2693 | TTN | 2589701 | TCACTGTGGAGTTGGGAATAACCCC |
| 2694 | TTN | 2589701 | TGCTACGACCTTACTCTCTTACGAG |
| 2695 | TTN | 2589701 | TACGACAACTCAATGCCCGGTATCA |
| 2696 | TTN | 2589701 | CCCCTCAGGTCTTCGTAGATTAAGA |
| 2697 | TTN | 2589466 | GTACCGGGGGACCTTTTGGTTGACA |
| 2698 | TTN | 2589466 | AGGAGGTACCGGGGGACCTTTTGGT |
| 2699 | TTN | 2589466 | GGACCTTTTGGTTGACATTTTCTAC |
| 2700 | TTN | 2589466 | ACCTTTTGGTTGACATTTTCTACAT |
| 2701 | TTN | 2589523 | TTCCTTCTTCAACAAGACTTTTCGC |
| 2702 | TTN | 2589523 | ACCTCCACTTTTCTTTCAAGCGTTT |
| 2703 | TTN | 2589523 | CTTGGTTTCCTTCTTCAACAAGACT |
| 2704 | TTN | 2589523 | GACTTGGTTTCCTTCTTCAACAAGA |
| 2705 | TTN | 2589777 | TCACGAAGGGTGAGAACTACTGACT |
| 2706 | TTN | 2589777 | CGTCCACGCAACGTATGGAAGATCT |
| 2707 | TTN | 2589777 | GGTGTACGGTCGCTTAGAAAACCAT |
| 2708 | TTN | 2589777 | AAACTGGTTCGTTTTCCGCGGGTAG |
| 2709 | TTN | 2589391 | GGCTATTGACCCACTCCACGTTGAA |
| 2710 | TTN | 2589391 | GTTAAGGCACACATACGGCAATTAT |
| 2711 | TTN | 2589391 | CCGCCGTCGGGATAGTAACCAATAG |
| 2712 | TTN | 2589391 | AGACACTCAGATAGAACCCCGTTCG |
| 2713 | TTN | 2589447 | GGTTCTTTAGAACGACAATGACTGT |
| 2714 | TTN | 2589447 | CCGTCACTTTAGTGGGTAATACAAT |
| 2715 | TTN | 2589447 | ATGACTGTAATTTCGACTTAGAACG |
| 2716 | TTN | 2589447 | AGAACGATGAACTGTACCCTACGGG |
| 2717 | TTN | 2589342 | TCGACTGCGACCCTCTATACTTTAG |
| 2718 | TTN | 2589342 | GAGAGTTTACCCTCGGTGGATTCAT |
| 2719 | TTN | 2589342 | GTCTTTTGGCGAAACCGTAGTCACT |
| 2720 | TTN | 2589342 | AACAACACGATCTGTCCGGACCAGG |
| 2721 | TTN | 2589331 | TCAGGCCTCTCTCGGAATCTTAATT |
| 2722 | TTN | 2589331 | GGTCACGGAGCTCATTGAACCAAGT |
| 2723 | TTN | 2589331 | GCTGCATGAACCTAGGTGGTCGGAT |
| 2724 | TTN | 2589331 | AGCCCTGGTAGCACCACATATGTGT |
| 2725 | TTN | 2589734 | TCTTTTACCTGTCGTAATTTCCAAG |
| 2726 | TTN | 2589734 | TACATATCACCGACCCAGTGTAGGG |
| 2727 | TTN | 2589734 | TTATAGTCGATCACTTTTCATGTTT |
| 2728 | TTN | 2589734 | TCACCGACCCAGTGTAGGGTATTCG |
| 2729 | TTN | 2589659 | ACAACGTTTCGGGTTTCTCTACTGT |
| 2730 | TTN | 2589659 | AACGTTTCGGGTTTCTCTACTGTGG |
| 2731 | TTN | 2589659 | CAACAACGTTTCGGGTTTCTCTACT |
| 2732 | TTN | 2589659 | CAACGTTTCGGGTTTCTCTACTGTG |
| 2733 | TTN | 2589713 | CCGACGATTACAGCGACCCAGACTA |
| 2734 | TTN | 2589713 | GCGACCCAGACTACTTACAGCACGT |
| 2735 | TTN | 2589713 | ACAGCACGTCACGATTGACATGTTC |
| 2736 | TTN | 2589713 | GTGTACGCACCGACGATTACAGCGA |
| 2737 | TTN | 2589465 | TCACTTGGGTCACTTGGGTCACTGG |
| 2738 | TTN | 2589465 | CCGAGTACCTTCCTGTCCTTATGAG |
| 2739 | TTN | 2589465 | ACCGCCTTCCCCAAGGGTGGTGAGT |
| 2740 | TTN | 2589465 | TGAGTAAGGCTCAATCTCGACACTT |
| 2741 | TTN | 2589400 | GGCACGACTTTTGGCTAAACCGTAA |
| 2742 | TTN | 2589400 | TTCTACCAACGCGTCAAGGGTAAAC |
| 2743 | TTN | 2589400 | CAGGCACGACTTTTGGCTAAACCGT |
| 2744 | TTN | 2589400 | AGTGTAGAGGTTTCTACCAACGCGT |
| 2745 | TTN | 2589430 | AACCTGATTTAGTAGACGTCTAGAC |
| 2746 | TTN | 2589430 | GGGTGATTTTCTACCACCTAGGTTT |
| 2747 | TTN | 2589430 | GACCTATGTAGCAACTTATATTTCT |
| 2748 | TTN | 2589430 | CTAGACCTCACCAGAGGGGGTGATT |
| 2749 | TTN | 2589804 | GGGTAACGATAGGATGTTCCTGAAT |
| 2750 | TTN | 2589804 | CCCACTGTAACAAGTCGAACTTCAA |
| 2751 | TTN | 2589804 | GGTTTTTCAGACACTCCCACTGTAA |
| 2752 | TTN | 2589804 | GTGGTCACCCGCACAGAGACAGATA |
| 2753 | TTN | 2589633 | TTGATAGGAAGCGTCAAGGAGTTTC |
| 2754 | TTN | 2589633 | TTTTCTCTAAACAACGACTTCTTTT |
| 2755 | TTN | 2589633 | CTTCAGAGATTCTTTTGACAACATC |
| 2756 | TTN | 2589633 | GGAGTTTCTCACCTTCAGTGCGCCG |
| 2757 | TTN | 2589811 | ACCATCTGGATACGGTCTCTGCAAG |
| 2758 | TTN | 2589811 | TACAGTGGACGTTCCTACAGAGGAC |
| 2759 | TTN | 2589811 | ATGCGTACAGAGGACGTGCCTACAG |
| 2760 | TTN | 2589811 | ACTCGTCCAGAGGATATGCGTACAG |
| 2761 | TTN | 2589720 | GAACTTACGTTCTATCGACCTAGGG |
| 2762 | TTN | 2589720 | AAACACTCCGAGTCTTAGGGCGACC |
| 2763 | TTN | 2589720 | GTCCACTGAGAAGTGCTGAACTTAC |
| 2764 | TTN | 2589720 | GAGTCACCGGCAGTATGTCTACTTG |
| 2765 | TTN | 2589368 | CCATCTAGGTGGTGCTTATTCATAC |
| 2766 | TTN | 2589368 | GTTAGCACCAAGTACGACCACTTAG |
| 2767 | TTN | 2589368 | GTCATGCACAGCTGTCACCTTTAAT |
| 2768 | TTN | 2589368 | TTCTCGTGGCTGAAACGGTGGTCAG |
| 2769 | TTN | 2589628 | AGGACTTCGTGGATTCTTTTAACAC |
| 2770 | TTN | 2589628 | ACGGTCTTTTCTTTCAAGGACGAGG |
| 2771 | TTN | 2589628 | ACTTCGTGGATTCTTTTAACACGGT |
| 2772 | TTN | 2589628 | AAGGACGAGGTCAAGGATTTTTCCT |
| 2773 | TTN | 2589729 | AGACCACGGTCCCATCTTTTATCAC |
| 2774 | TTN | 2589729 | TTCTTCGGGTCAGGTCAGAATCACG |
| 2775 | TTN | 2589729 | TAGGCTCACAGAACCATAGATCTGC |
| 2776 | TTN | 2589729 | ACTTCGAGCTTTACTGCGTCCGTGC |
| 2777 | TTN | 2589358 | ACGTCCAAGATTCAGTAAGGGTCAT |
| 2778 | TTN | 2589358 | GGACTACCAGCAACCTACTTTCGAT |
| 2779 | TTN | 2589358 | AGGTACTGGCAGACAACCTTGGCAG |
| 2780 | TTN | 2589358 | GACGGTTCCTACTCCAACTTGAGGG |
| 2781 | TTN | 2589757 | CAGATTTTCTCTCGGGCACCGTTAT |
| 2782 | TTN | 2589757 | TCCAGCTCCGACATTTGTAGTGGGT |
| 2783 | TTN | 2589757 | CCGTTCAGGAATTCTAAAGGTGATC |
| 2784 | TTN | 2589757 | ACATGGAACAATGAAGCCGTTTCAG |
| 2785 | TTN | 2589317 | TTCGTCCCGTAACTGGAACGTTCGG |
| 2786 | TTN | 2589317 | GGTGGAGGACAGTATTGCACCTCGT |
| 2787 | TTN | 2589317 | GAGGAACGCGAACTACCACAGACAT |
| 2788 | TTN | 2589317 | CGGCCCGTTAATAACTGTGGTGACT |
| 2789 | TTN | 2589506 | CTTCGACTTTCCTGTCGGAAACTGT |
| 2790 | TTN | 2589506 | TACAAGTGCCGGTCACCTTCGACTT |
| 2791 | TTN | 2589506 | ACCTTTTCGGAGACATGCCTCATCT |
| 2792 | TTN | 2589506 | CTTGAAAGACTTGGACTACAAGTGC |
| 2793 | TTN | 2589726 | AGATATTGGACCGATTTCCTACTAG |
| 2794 | TTN | 2589726 | CTATCTTTTATGGTGATGACAAAAC |
| 2795 | TTN | 2589726 | GGAAAGTCTCATGGCACCGTCCAAG |
| 2796 | TTN | 2589726 | TCCTCAACTCTCCACAATACGAAAG |
| 2797 | TTN | 2589357 | CCCTTGGTTCAGGTCGATGTCAAAT |
| 2798 | TTN | 2589357 | ACCCCTTGGTTCAGGTCGATGTCAA |
| 2799 | TTN | 2589357 | CTTGGTTCAGGTCGATGTCAAATAA |
| 2800 | TTN | 2589357 | TGGTTCAGGTCGATGTCAAATAATA |
| 2801 | TTN | 2589356 | CACAGGGTCTGGTCACGTAGTGAAT |
| 2802 | TTN | 2589356 | GACAACTACGGCACTTTCGACGACT |
| 2803 | TTN | 2589356 | AGGACCCCATGCAACAACTTTACGT |
| 2804 | TTN | 2589356 | GACCGGACTCTCAGTGAGCTAAACT |
| 2805 | TTN | 2589671 | GGGTCGTTGGTTCTTTCGACGCCAT |
| 2806 | TTN | 2589671 | ACAGTCTTGGAGTCTCATAGTCTCA |
| 2807 | TTN | 2589671 | TCGACGCCATCTACCTTCTGAGAAA |
| 2808 | TTN | 2589671 | CCTTCTGAGAAAAAACACAGTCTTG |
| 2809 | TTN | 2589632 | TCCTTCCACACAGGTAAAGTCAAAT |
| 2810 | TTN | 2589632 | CCTTCCACACAGGTAAAGTCAAATA |
| 2811 | TTN | 2589712 | CGTAGAACGTGATGGGCAGAGAAAC |
| 2812 | TTN | 2589712 | CCTCTGGCCACGTTGTAGATAAAAC |
| 2813 | TTN | 2589712 | AAGTGTTCGCAATAATCTCCTTGGG |
| 2814 | TTN | 2589712 | CCAAAGATTGTTGCGACCGGTTCGT |
| 2815 | TTN | 2589721 | CGCTGAGAACATGATGCTACAACGA |
| 2816 | TTN | 2589721 | ACCATCGCTGAGAACATGATGCTAC |
| 2817 | TTN | 2589721 | AATGGTTACTACAACCATCGCTGAG |
| 2818 | TTN | 2589721 | CTACAACCATCGCTGAGAACATGAT |
| 2819 | TTN | 2589786 | GACCCGTCATGAGAACGTTTCGTCG |
| 2820 | TTN | 2589786 | ACACGTCGATGTGAGTGTCACTGAG |
| 2821 | TTN | 2589786 | CGACCCCTTCGGTGAACACGTCGAT |
| 2822 | TTN | 2589786 | ACCAAAGTATTGGTCGTCGATTAAG |
| 2823 | TTN | 2589831 | CTCTTGTTCACGTTTATTGAGTCCT |
| 2824 | TTN | 2589831 | CTCTTCCGTAATGATGGTTTTCTCT |
| 2825 | TTN | 2589831 | TTTCAGTATCAACGGTGTGGGTTTC |
| 2826 | TTN | 2589831 | GTCAACATGGATTTCAGTATCAACG |
| 2827 | TTN | 2589617 | AGGATTCTTTGAGTTTGGAGGTGGT |
| 2828 | TTN | 2589617 | ACTTCTTTTTCATGGTCACGGGTAA |
| 2829 | TTN | 2589617 | ACGGGTAAGGATTCTTTGAGTTTGG |
| 2830 | TTN | 2589617 | TTTTCATGGTCACGGGTAAGGATTC |
| 2831 | TTN | 2589505 | GACCCATACTGTCCTCTCCAAAGGA |
| 2832 | TTN | 2589505 | GGAAGTATTGACAGTCGACCCATAC |
| 2833 | TTN | 2589505 | GGAAGGTCCGACGATTACGGTTTAG |
| 2834 | TTN | 2589505 | CCTTTCTTCGTATAAGACTAGGAAG |
| 2835 | TTN | 2589697 | ACCTCGGGAGGCTGTGTCCGTATAT |
| 2836 | TTN | 2589697 | ACTTAGACTCGAACAACCTCGGGAG |
| 2837 | TTN | 2589697 | ACAGTCAGGTCGAAAAGCCTTTTGC |
| 2838 | TTN | 2589697 | GACGGTTACATCGACCAAGGCTACT |
| 2839 | TTN | 2589808 | CCAAGGGCTGAACTTTACTTTCAGT |
| 2840 | TTN | 2589808 | ACAGTTATATTTCCTTCCAAGGGCT |
| 2841 | TTN | 2589808 | ACTTTCAGTCTCGATGCCCATTGGG |
| 2842 | TTN | 2589808 | GGGACTGTAACATACCAACTTTTTG |
| 2843 | TTN | 2589433 | GGTCGCTGACGATCTCTAGGTTAAC |
| 2844 | TTN | 2589433 | ATCGTCGCACTGAGCATTGAGGTAC |
| 2845 | TTN | 2589433 | ACCCAGGTCGTTCAGACGGTAGTCT |
| 2846 | TTN | 2589433 | AGGCTGATAGTTAAGGCCCATATAC |
| 2847 | TTN | 2589668 | GTCTCTCCCTGCTTTTCCTTAAACT |
| 2848 | TTN | 2589668 | AATAAGTCGTTTCTGACAGTGTCTG |
| 2849 | TTN | 2589668 | AAGTCGTTTCTGACAGTGTCTGTCT |
| 2850 | TTN | 2589668 | AGTCTCTCCCTGCTTTTCCTTAAAC |
| 2851 | TTN | 2589744 | CCGTTTATGTGCACGGACCGACTTT |
| 2852 | TTN | 2589744 | GTAGACAACCCCTTAATTATCAATT |
| 2853 | TTN | 2589744 | CACAACGTCAGAACTATTAGGGACT |
| 2854 | TTN | 2589744 | CCTGTTTGGCAATGGGACGTTCGAC |
| 2855 | TTN | 2589507 | GTTCTGGTTCCGTTTACAATGACAA |
| 2856 | TTN | 2589507 | GGCTAGGGATGAAGTGACACTTTAA |
| 2857 | TTN | 2589507 | CCGGACGCGGCGTAGAATTTTTAGT |
| 2858 | TTN | 2589507 | TTCTCTAACAGGGAAGTGGGTTTAT |
| 2859 | TTN | 2589620 | CGACAAAGTCATGTTGCCCTTCTTC |
| 2860 | TTN | 2589620 | ACAAAGTCATGTTGCCCTTCTTCTT |
| 2861 | TTN | 2589620 | GTCATGTTGCCCTTCTTCTTATACT |
| 2862 | TTN | 2589620 | AAAGTCATGTTGCCCTTCTTCTTAT |
| 2863 | TTN | 2589353 | AGATTCCGTCAATAACATGTTATAG |
| 2864 | TTN | 2589353 | ACCTTGAGGAAAACACTGTAGTTAG |
| 2865 | TTN | 2589353 | TTCTAGTCTACGAACACGTTACCGT |
| 2866 | TTN | 2589353 | CCGTGGTTTTAATAACCGATGGTAG |
| 2867 | TTN | 2589675 | ACGACTGAAACTCACGGTGCAGTGC |
| 2868 | TTN | 2589675 | GTGCAGTGCCCGTGTGTTGGCTATT |
| 2869 | TTN | 2589675 | GTTGGCTATTTCCAGTCGACCCGGT |
| 2870 | TTN | 2589675 | GGTGGGAAGAAACTGTAGGCAGAAC |
| 2871 | TTN | 2589732 | CTTCCTACTGTGGAGATGGTCAGAT |
| 2872 | TTN | 2589732 | CGGTGTCAGTGTTCTACAGTTAGGG |
| 2873 | TTN | 2589732 | AATTTCCGAGAACACCCACCGTGAC |
| 2874 | TTN | 2589732 | GTGAGTCCTCGTCGGGCGAGTCAAA |
| 2875 | TTN | 2589441 | CGACTTTTGAGGAGTCATTAATAAT |
| 2876 | TTN | 2589441 | AGTCATTAATAATAAGGCCTCACAT |
| 2877 | TTN | 2589441 | TTTGCTAGAGTATGTCCGTTTATGT |
| 2878 | TTN | 2589441 | AATAATAAGGCCTCACATTTGCTAG |
| 2879 | TTN | 2589379 | AAGGCTAGAGTCCACGTTTCATTGT |
| 2880 | TTN | 2589379 | GGACGCTACCCACTCTCGTTATTTT |
| 2881 | TTN | 2589379 | CCACCGTCGCTTTAATGTCCTATAG |
| 2882 | TTN | 2589379 | ATGCTTAAGGCACAGTCACGTCTTT |
| 2883 | TTN | 2589341 | CCGTCACGTCAGCATCCGATAGTGG |
| 2884 | TTN | 2589341 | GTGGTACCCAGTGATTACAATGAGC |
| 2885 | TTN | 2589341 | ACCAGTAGGCGTGTTGAGTGAAGTT |
| 2886 | TTN | 2589341 | CAGTGTTGTTAGTCACGACCTGAAT |
| 2887 | TTN | 2589420 | ACTGGTTTCTAATGTACCAATAGAG |
| 2888 | TTN | 2589420 | TTTCTAATGTACCAATAGAGAACCT |
| 2889 | TTN | 2589420 | GGTTTCTAATGTACCAATAGAGAAC |
| 2890 | TTN | 2589420 | TCTAATGTACCAATAGAGAACCTTC |
| 2891 | TTN | 2589455 | GGAAGTCTAGGGTTTTGTCGTGTAC |
| 2892 | TTN | 2589455 | TACCGAATAATCTTCCTGAGTGGAT |
| 2893 | TTN | 2589455 | ACTTACGGAACTTTCGGTTACATCT |
| 2894 | TTN | 2589455 | ACCAGAGCACAGTTGTTTTCGGAAG |
| 2895 | TTN | 2589474 | TAGAAGAATTGTACCCTAGGTGGAT |
| 2896 | TTN | 2589474 | TCCTATATATCAACTTTCTACAGGT |
| 2897 | TTN | 2589474 | CTCTCTAGCTTGTCGGTTATCGTAG |
| 2898 | TTN | 2589474 | ACCACCAAGTGCGTAGTTTCCTATA |
| 2899 | TTN | 2589403 | CGGTCTTTACGACAACCTCAGTCAA |
| 2900 | TTN | 2589403 | GGATGCACTACCTCCACGATTTTAG |
| 2901 | TTN | 2589403 | GGACGTCTAGCGACCTGTCTCATGA |
| 2902 | TTN | 2589403 | CGGAACAACAGTGACCGGATTTCCT |
| 2903 | TTN | 2589634 | GACGGATTCTTTGGGCAGGGTCTCC |
| 2904 | TTN | 2589634 | ACAGCGATTCTTTCGAGGAGGAGGG |
| 2905 | TTN | 2589634 | CAAGGACAGCGATTCTTTCGAGGAG |
| 2906 | TTN | 2589634 | CGGTCAAGGACAGCGATTCTTTCGA |
| 2907 | TTN | 2589768 | CACCTTCCCGGAGGGTCCAAATAGT |
| 2908 | TTN | 2589768 | GTCACGAATCAAGCACCGAGAGGTT |
| 2909 | TTN | 2589768 | AGACTGACAATACGTGGTTATCCAT |
| 2910 | TTN | 2589768 | CAAGGGCTTAAATGAGGACTGGTAT |
| 2911 | TTN | 2589431 | GGAGGACCAGGAGGTAAAGGGTTTC |
| 2912 | TTN | 2589529 | TCTTTCTTGGACACGGACAATGGTT |
| 2913 | TTN | 2589529 | ACGGGTTCTTTGAACAAGGTCATTT |
| 2914 | TTN | 2589529 | CGGACTCCACGGGTTCTTTGAACAA |
| 2915 | TTN | 2589529 | GACTCCACGGGTTCTTTGAACAAGG |
| 2916 | TTN | 2589350 | TCTTACGAAACAACGAGCACTAGGT |
| 2917 | TTN | 2589350 | TTCTTTGTTACAGTGTGACTTTACC |
| 2918 | TTN | 2589350 | TACACTGGGTGGACCAGCGGGACTT |
| 2919 | TTN | 2589350 | ATAATGTTCTTTGTTACAGTGTGAC |
| 2920 | TTN | 2589583 | CAAGGAGAGTCTTTCGGACTTCAGG |
| 2921 | TTN | 2589583 | ACACCAAGGAGAGTCTTTCGGACTT |
| 2922 | TTN | 2589583 | ACCAAGGAGAGTCTTTCGGACTTCA |
| 2923 | TTN | 2589583 | AACACCAAGGAGAGTCTTTCGGACT |
| 2924 | TTN | 2589509 | CAGGAGTGGGCTCTCCGTTTACAAT |
| 2925 | TTN | 2589509 | GGGACTTCCTACAGTGACAAGGTCT |
| 2926 | TTN | 2589509 | CCGAGCTAAGCTTACACAGGAGTGG |
| 2927 | TTN | 2589509 | AGGTCTTTCCGCTGTCCGAGCTAAG |
| 2928 | TTN | 2589409 | GGACAGTTGTTCTCACGTTAGGGAC |
| 2929 | TTN | 2589409 | TGCAGGAACTGTCTGGACCCGGAAC |
| 2930 | TTN | 2589409 | AGGCGTAAGATCGAGCTCAGTTTCC |
| 2931 | TTN | 2589409 | ACGGGTTCCAAGTCGGTAGCAATTG |
| 2932 | TTN | 2589631 | TCGGACTCCTTATACAACACCTTCT |
| 2933 | TTN | 2589631 | CTTTTCGACGTGTAATAAAGATTCT |
| 2934 | TTN | 2589631 | TTCTCGGACTCCTTATACAACACCT |
| 2935 | TTN | 2589631 | GGACTCCTTATACAACACCTTCTTT |
| 2936 | TTN | 2589462 | GTCTCCAGGTCAAAGTCCAAGCCCG |
| 2937 | TTN | 2589462 | GACTACAGTAGCTTCCTTGTCTCCA |
| 2938 | TTN | 2589462 | ACGTCCTGGACTGACATTGAAGTCT |
| 2939 | TTN | 2589462 | GGGTGTCTTTAGGATAGGTAACTTC |
| 2940 | TTN | 2589742 | TGGGAACGCGTTGCACCTATCACAA |
| 2941 | TTN | 2589742 | ACAATTACCATGGACGTCTGACCTG |
| 2942 | TTN | 2589742 | GGTACTCCCACAGGACCAAATTCCT |
| 2943 | TTN | 2589742 | CCCTTAAAGTGAACAGCTCGGTGTT |
| 2944 | TTN | 2589597 | TTTCACCTCCGTGGTGGTCGATTTC |
| 2945 | TTN | 2589597 | CTAATTCTTTCGTCATGGACTTCGT |
| 2946 | TTN | 2589597 | TTTTTCACCTCCGTGGTGGTCGATT |
| 2947 | TTN | 2589597 | AGGACAAGGATTTTTTCACCTCCGT |
| 2948 | TTN | 2589630 | AAGGACGATTTTATCTCCTCGGAGG |
| 2949 | TTN | 2589630 | TATCTCCTCGGAGGTGGCCGATTTC |
| 2950 | TTN | 2589630 | ACGATTTTATCTCCTCGGAGGTGGC |
| 2951 | TTN | 2589630 | GGTTTTTAAGGACGATTTTATCTCC |
| 2952 | TTN | 2589339 | CCGGGTAATGAACGTAGCTAAGAAT |
| 2953 | TTN | 2589339 | GGTCGAAAGCTACCTCCATCGTTCT |
| 2954 | TTN | 2589339 | ATGTAACAACTCTCTGCACTGGAAG |
| 2955 | TTN | 2589339 | GGTAGACTCCAACATCCCGGGTAAT |
| 2956 | TTN | 2589834 | CTATTGACGACGTAGGTACCACCAT |
| 2957 | TTN | 2589834 | GTGTTGTTCTAGTTTACGTGGATTC |
| 2958 | TTN | 2589834 | CCATCAACGGTGACGTTTCAGGTGT |
| 2959 | TTN | 2589834 | TGTTCTAGTTTACGTGGATTCAATA |
| 2960 | TTN | 2589537 | CGTGGACGACAGCAACGGTTTTTTG |
| 2961 | TTN | 2589537 | TTTCTTTCGTGGACGACAGCAACGG |
| 2962 | TTN | 2589537 | ATGGTGGATTCTTTGGACAGGGTCT |
| 2963 | TTN | 2589537 | ACGGTTTTTTGGACTTGATGGTGGT |
| 2964 | TTN | 2589654 | TAAGTTCAAGTTTTCCTCCAGATAC |
| 2965 | TTN | 2589654 | GTTCAAGTTTTCCTCCAGATACTTC |
| 2966 | TTN | 2589654 | ATTAAGTTCAAGTTTTCCTCCAGAT |
| 2967 | TTN | 2589654 | AAGTTCAAGTTTTCCTCCAGATACT |
| 2968 | TTN | 2589849 | AGTGGCTAAGTACAGCCTCTACCAG |
| 2969 | TTN | 2589849 | AAATCCTAATCTCCGAGTGGCTAAG |
| 2970 | TTN | 2589849 | CCTGCAGCAAAGTCTTCGTTGGAAC |
| 2971 | TTN | 2589849 | CAGTCTTTTTGGTTGAGAGGTATCC |
| 2972 | TTN | 2589591 | GTGATTTCAACAAGGAGCTTTTCTC |
| 2973 | TTN | 2589484 | TTCGGTCTCGATTCGAACTTGACCG |
| 2974 | TTN | 2589484 | CGGTCTCGATTCGAACTTGACCGTC |
| 2975 | TTN | 2589484 | GTTTCTTCGGTCTCGATTCGAACTT |
| 2976 | TTN | 2589484 | GTTTCTGTTTCTTCGGTCTCGATTC |
| 2977 | TTN | 2589750 | CCGGTGGATCGGTTTAAGTGGACAC |
| 2978 | TTN | 2589750 | AAGGTTACTCATACCGTCACAGTCG |
| 2979 | TTN | 2589750 | GGGACCTTCATCGTGACCCGGTGGA |
| 2980 | TTN | 2589750 | GGTTTCACGAGGGTTACAGGCCAAG |
| 2981 | TTN | 2589390 | ACGCCTGAATTCCTTCTGTGAGTAT |
| 2982 | TTN | 2589390 | TGCACGACCTCAATGATACTCTGAT |
| 2983 | TTN | 2589390 | TGAAACTGTGAAAGAACGCGACACT |
| 2984 | TTN | 2589390 | GGACTCCCTCTTGAACTACGCCTGA |
| 2985 | TTN | 2589783 | TCCTTAGGCTCTCTGACTTTGTTAG |
| 2986 | TTN | 2589783 | GGTTCCTGATATGTCGACGTAACAT |
| 2987 | TTN | 2589783 | TCCTGTTGCAGTCGTCATGATTCAC |
| 2988 | TTN | 2589783 | GACCTGAGTGCTTCCACGTTTCTAT |
| 2989 | TTN | 2589463 | ACACTGTTTTGCTGTACAACTGGAT |
| 2990 | TTN | 2589463 | TACAACTGGATTTCACCCTCGGTGG |
| 2991 | TTN | 2589463 | TGGGATGCGGGACCGTCACCAACTA |
| 2992 | TTN | 2589463 | GTCACCAACTACACTGTTTTGCTGT |
| 2993 | TTN | 2589395 | AACCCTTGGCGGAGAACTTCTACCT |
| 2994 | TTN | 2589395 | GACCCGAGTTCAGAGACGTTGACAC |
| 2995 | TTN | 2589395 | TAGCACGATACGAAAGAACCCTTGG |
| 2996 | TTN | 2589395 | CGACGTCGCACCTCTTTGAATATCT |
| 2997 | TTN | 2589373 | TACCGTCTAACAGTGAGGTGGTCGT |
| 2998 | TTN | 2589373 | CCTGAGTTCCGTTGAAGCATATGAT |
| 2999 | TTN | 2589373 | TATACCAACTCTAACGGGACGGTCT |
| 3000 | TTN | 2589373 | ATTGGGTGCACAAGACCTATGTTCG |
| 3001 | TTN | 2589470 | GCGCTACTACCACCTAGATTCTAGT |
| 3002 | TTN | 2589470 | TGTTCGAGAGTAGGTGGCAGTTCCT |
| 3003 | TTN | 2589470 | GTTTGATACAACACCTCTCTGCTCG |
| 3004 | TTN | 2589470 | TGTACCTTGGGTGGTGCGCTACTAC |
| 3005 | TTN | 2589770 | AAACCATGTGGGTTACTTCGGTAAC |
| 3006 | TTN | 2589770 | AAGTCGAATACTTTATACCGCAAGG |
| 3007 | TTN | 2589770 | ACCGCAAGGCTAAGTAAACCATGTG |
| 3008 | TTN | 2589770 | TCTGCCATGTGGTTTACTCGTAAAG |
| 3009 | TTN | 2589370 | TTCTGTTGACCGGAACTTCTTCCAC |
| 3010 | TTN | 2589370 | CCGATAGTAGATCTTGCGTTCCTTT |
| 3011 | TTN | 2589370 | TTGTAGCACCCGTAACCGTTCGGCT |
| 3012 | TTN | 2589370 | TCGTAGGAGACCCAATTCAACTTAT |
| 3013 | TTN | 2589621 | TTTTCATAGTTAACTTCGAGGTTTT |
| 3014 | TTN | 2589621 | TCATAGTTAACTTCGAGGTTTTTCT |
| 3015 | TTN | 2589621 | TTTTCTCTTGGAGTTGGGTAGTTTC |
| 3016 | TTN | 2589621 | GTTTTTCTCTTGGAGTTGGGTAGTT |
| 3017 | TTN | 2589377 | GGCCGAACAGACTTCCCACACTTAT |
| 3018 | TTN | 2589377 | ACGGCTCGAAGCTTCTTGTGAACAA |
| 3019 | TTN | 2589377 | AACACGGTTAATTTCCAGCAGGACG |
| 3020 | TTN | 2589377 | GACCCTGGAGGGAGACTATCTACCT |
| 3021 | TTN | 2589788 | GTAGAGCCAAGAAATCTTACTGAGT |
| 3022 | TTN | 2589788 | CTTCCTTGAATGTGCAAACAACGAT |
| 3023 | TTN | 2589788 | GATCATTACGACATCCGGTTCATAG |
| 3024 | TTN | 2589788 | AACGGCTTCGAATAGGTCTTCTACT |
| 3025 | TTN | 2589340 | GTAGGAAGACTTGGTCAGAACCGTT |
| 3026 | TTN | 2589340 | GAAGACTTGGTCAGAACCGTTAACT |
| 3027 | TTN | 2589340 | ATCGGTAGGAAGACTTGGTCAGAAC |
| 3028 | TTN | 2589340 | TTTGGATCGGTAGGAAGACTTGGTC |
| 3029 | TTN | 2589681 | CACTGTCTACGTTGCACAGAAACCT |
| 3030 | TTN | 2589681 | CTACGTTGCACAGAAACCTCCTAAG |
| 3031 | TTN | 2589681 | TGTCTACGTTGCACAGAAACCTCCT |
| 3032 | TTN | 2589681 | GTCTACGTTGCACAGAAACCTCCTA |
| 3033 | TTN | 2589599 | GTGGATCGTATCTCCTTCAACTTCT |
| 3034 | TTN | 2589599 | CTTGGTGGATCGTATCTCCTTCAAC |
| 3035 | TTN | 2589599 | GACTTCTTCTCGGATAAAGTCTTCT |
| 3036 | TTN | 2589599 | AGGGTCTTCTTGGTGGATCGTATCT |
| 3037 | TTN | 2589816 | CTCTTTGTCGTGGACCTAAACATAT |
| 3038 | TTN | 2589816 | TGTCGTGGACCTAAACATATGAGAC |
| 3039 | TTN | 2589816 | GACCTAAACATATGAGACTCATACT |
| 3040 | TTN | 2589816 | GTGGACCTAAACATATGAGACTCAT |
| 3041 | TTN | 2589334 | TGAAGGCCCATAGACGACATTTGAC |
| 3042 | TTN | 2589334 | CCAGGTGGACGATTCTATTCTTAGC |
| 3043 | TTN | 2589334 | AGGTAGTGGGAACCGACCTCATTCG |
| 3044 | TTN | 2589334 | ACACCATAGGTTGGACTTTGGACCT |
| 3045 | TTN | 2589771 | CTGAGACCTCTCTATGAGGTGTGGG |
| 3046 | TTN | 2589771 | CCTCTCTGAGATCTCTCTATAAGGT |
| 3047 | TTN | 2589771 | GGGGTCCTCTCTGAGATCTCGCTAT |
| 3048 | TTN | 2589771 | TCTCTCTATAAGGTGTGGGGGTCCT |
| 3049 | TTN | 2589838 | TAGTCCAGACAATCCAGAGGTAACG |
| 3050 | TTN | 2589838 | CAGCCGGTCACGATCGATGCGTCGT |
| 3051 | TTN | 2589838 | GAGGTAACGAGTACGCATTCTGAGT |
| 3052 | TTN | 2589838 | CATTCTGAGTCCGTAGGTGGCACCG |
| 3053 | TTN | 2589411 | AGGTGTGGATGACAACGATTCGTAT |
| 3054 | TTN | 2589411 | GGATGACAACGATTCGTATTTAAAT |
| 3055 | TTN | 2589411 | ACTGAGGTGTGGATGACAACGATTC |
| 3056 | TTN | 2589411 | GAACTGAGGTGTGGATGACAACGAT |
| 3057 | TTN | 2589818 | CTACTACGACCTCTTATGTGATAAC |
| 3058 | TTN | 2589818 | GGCCACTTACGTTCGACCACTAAAG |
| 3059 | TTN | 2589818 | ACAACAAGCGTTATTCGTACCTCTT |
| 3060 | TTN | 2589818 | ACTCAATGTTGTTTGTTTGGCCACT |
| 3061 | TTN | 2589312 | CACCGACGTTGTTTGGCGAAGCCCT |
| 3062 | TTN | 2589312 | AGCCCTAACCGAGAATGAACGTCAG |
| 3063 | TTN | 2589312 | ATACACCATCTTGTTGCACTGCGAG |
| 3064 | TTN | 2589312 | CTCCAGTATCTCACAGCGTCGTCGT |
| 3065 | TTN | 2589841 | AACTCTACCAGTATCTACCACGGCG |
| 3066 | TTN | 2589841 | ACTTCGGGTGAAACTACGGTCTAGT |
| 3067 | TTN | 2589841 | TGTGGTGGCAGATAACGACGGTTTC |
| 3068 | TTN | 2589841 | AAGGAGGCTTCGGTTTCAGTTCTAG |
| 3069 | TTN | 2589457 | TGTGTCGCAAACTCATAACTGTTGT |
| 3070 | TTN | 2589457 | TTCGCACGTCGTGGGAACCAATCCT |
| 3071 | TTN | 2589457 | CTCTAAGAGAGGCTGGACTGGTACC |
| 3072 | TTN | 2589457 | TACTGGTGCCATAACGTTTTCGAGG |
| 3073 | TTN | 2589325 | CACGGTACGAATCTCACGTTGATGT |
| 3074 | TTN | 2589325 | GACGGTTACGGTACAGATAAGCAAC |
| 3075 | TTN | 2589325 | CCTTCTTGGGATGGTGCTACCACCG |
| 3076 | TTN | 2589325 | CCGGTCGCTTCGAAGTTCTGGATAT |
| 3077 | TTN | 2589813 | TACCGTCAACTGTAAAGACTTAGAC |
| 3078 | TTN | 2589813 | GTGAAAAGTAACGTTCTACAGACCT |
| 3079 | TTN | 2589813 | AGTTCTTAGTTCTTAATATCTTAAG |
| 3080 | TTN | 2589813 | TCAACTGTAAAGACTTAGACTTCGA |
| 3081 | TTN | 2589594 | TCAGGAACACGGATTTTTCCTTCGA |
| 3082 | TTN | 2589485 | GAGTTATTTCTATTCCACCTTCAGG |
| 3083 | TTN | 2589485 | TAGTCCGGAGGTGTTCTATAAGAAC |
| 3084 | TTN | 2589485 | TCTATGTAGCTGATGTCTAAACACT |
| 3085 | TTN | 2589485 | CACCTTCAGGTTACCGATTCTTTAT |
| 3086 | TTN | 2589453 | TGGTACCGTCTATACACTAATGTCG |
| 3087 | TTN | 2589453 | ACTGACCAGCGGGACATGGATGTTT |
| 3088 | TTN | 2589453 | CCTACGTGACGCTTTTCTGGTACCG |
| 3089 | TTN | 2589453 | AAACGTCGTCGGTCCCATCTTCAAA |
| 3090 | TTN | 2589419 | GTTAATATAATAACTCTTCTTCCTT |
| 3091 | TTN | 2589419 | GGTTAATATAATAACTCTTCTTCCT |
| 3092 | TTN | 2589303 | TACGACCGATAATGAAGGCCCAAAG |
| 3093 | TTN | 2589303 | CGAGATAACTTCTTGAGGCGTCACT |
| 3094 | TTN | 2589303 | AGGCCCAAAGTCGAGTCTTGTGAAA |
| 3095 | TTN | 2589303 | AGATCTTCACAGGAGTCAACACTAG |
| 3096 | TTN | 2589793 | GGTACAAACTTACACTTCAAAGACT |
| 3097 | TTN | 2589793 | GTGTAATTCCTGTAATTCCATGACC |
| 3098 | TTN | 2589793 | TCAAAGACTTGGACTGTAGTGACAT |
| 3099 | TTN | 2589793 | ATGACCTCTTCTTCGCTCGGTACAA |
| 3100 | TTN | 2589686 | CCTTTTATGTGAACAGTCTAGTTTT |
| 3101 | TTN | 2589686 | GCTACGACCCTACGTTCTCACGAAG |
| 3102 | TTN | 2589686 | TCACGAAGCGGTGCGATAGGCAAGA |
| 3103 | TTN | 2589686 | GTCTAGTTTTTGCTACGACCCTACG |
| 3104 | TTN | 2589292 | GACAGAGACTGTATTCCTCACGGAC |
| 3105 | TTN | 2589292 | CAAGCGAGTAAAATGCGACAGAGAC |
| 3106 | TTN | 2589292 | TCCCGGACACGGGAATATGAGATGT |
| 3107 | TTN | 2589292 | AGTTCATAAGGATACGGTCTCGTCA |
| 3108 | TTN | 2589593 | TAGTCAACGGAAGGCGGTTCTTCAT |
| 3109 | TTN | 2589593 | TCTTTTTCATAGTCAACGGAAGGCG |
| 3110 | TTN | 2589593 | TTTCATAGTCAACGGAAGGCGGTTC |
| 3111 | TTN | 2589593 | CGGAAGGCGGTTCTTCATCATCATT |
| 3112 | TTN | 2589795 | CACGTCCCTTTTGAGGTAGTCGACT |
| 3113 | TTN | 2589795 | GACACCGTTACTGGTTCAGTCACGG |
| 3114 | TTN | 2589795 | GGTCGTGTCTCCTGAGCCGTCTTAT |
| 3115 | TTN | 2589795 | ACACTCCACAGGGTGAAGTTACAGG |
| 3116 | TTN | 2589645 | ACTTCTCTTTTAAGTGCAACGGTAA |
| 3117 | TTN | 2589645 | GACTTCTCTTTTAAGTGCAACGGTA |
| 3118 | TTN | 2589645 | CTTTTAAGTGCAACGGTAAAGGTTT |
| 3119 | TTN | 2589645 | TCTCTTTTAAGTGCAACGGTAAAGG |
| 3120 | TTN | 2589491 | CACTTCATTCCTGTCGCTCTTGAAG |
| 3121 | TTN | 2589491 | GTTGTTTACAGATGACCTACTACTT |
| 3122 | TTN | 2589491 | ACTGTAGTATAGGTTCCCTCGTCAC |
| 3123 | TTN | 2589491 | TCGTCACGCGTAAGAACAGTAGTTG |
| 3124 | TTN | 2589414 | TCATGGAGAAGGCTCATCGACGCCT |
| 3125 | TTN | 2589414 | AGACTTACTTCCGTTGGTCATGGAG |
| 3126 | TTN | 2589414 | TGGACCCGGTGGATCTCTAGACCTT |
| 3127 | TTN | 2589414 | ACCTGCACCAGGAAAACAACTTTGT |
| 3128 | TTN | 2589372 | ACGACGTGGGACTCTGAAAAACAAG |
| 3129 | TTN | 2589372 | CGATGATAAAGTCCCAAGAGCGTCT |
| 3130 | TTN | 2589372 | CGGGCCCTGGTACCTCTTAGAAATC |
| 3131 | TTN | 2589372 | CGACTGGACGCGTTTCAACAATGAT |
| 3132 | TTN | 2589657 | TAGTAACTACATAGGAGATTTCGAC |
| 3133 | TTN | 2589657 | AAGCTTCTTGGAATACTGCTTGACC |
| 3134 | TTN | 2589657 | AGTAACTTAGAAAGCTTCTTGGAAT |
| 3135 | TTN | 2589657 | CTTAGAAAGCTTCTTGGAATACTGC |
| 3136 | TTN | 2589765 | CCGTCCCAAGTGGATCTGTAAACGT |
| 3137 | TTN | 2589765 | CACCTGGGTTCAGAAGAGTATATTC |
| 3138 | TTN | 2589765 | CCTCTGGTATAGTCTACTGGTGCGT |
| 3139 | TTN | 2589765 | ACCGAAGTTCCCTGGTTAACACGAG |
| 3140 | TTN | 2589539 | GTCTCCACGGTTTCCATCGACAGGG |
| 3141 | TTN | 2589539 | GGTTTCCATCGACAGGGTCTTTTCT |
| 3142 | TTN | 2589539 | TTTTCTTCCACGGACTTCGATAAGG |
| 3143 | TTN | 2589539 | ACTTCGATAAGGAGGGTTTGGCCTT |
| 3144 | TTN | 2589541 | CACGGTCACGGAGGAGGATTTTTCG |
| 3145 | TTN | 2589541 | TTTCGGACTTCACGGTGGGTGTTTT |
| 3146 | TTN | 2589541 | GGATTTTTCGGACTTCACGGTGGGT |
| 3147 | TTN | 2589541 | CCGAGGGTTTCTTCAACAAGGACTT |
| 3148 | TTN | 2589543 | GATTTTTCGGACTTCAGGGTGGACA |
| 3149 | TTN | 2589543 | GGGAGCCGAGGAGGATTTTTCGGAC |
| 3150 | TTN | 2589543 | GGATTTTTCGGACTTCAGGGTGGAC |
| 3151 | TTN | 2589547 | CCGAGGGTTTCTTCAACAAGGACTT |
| 3152 | TTN | 2589547 | CTTTCACGGTCACTGAGGAGGATTT |
| 3153 | TTN | 2589547 | CACGGTCACTGAGGAGGATTTTTTG |
| 3154 | TTN | 2589547 | TTCACGGTCACTGAGGAGGATTTTT |
| 3155 | TTN | 2589548 | CCGAGGGTTTCTTCAACAGGAACTT |
| 3156 | TTN | 2589548 | GGGAACCGAGGAGGATTTTTCGGAC |
| 3157 | TTN | 2589548 | GATTTTTCGGACTTCAGGGTGGACA |
| 3158 | TTN | 2589548 | ACGGGAACCGAGGAGGATTTTTCGG |
| 3159 | TTN | 2589549 | CAGGGTGTTCTTTAACACGGTCTTT |
| 3160 | TTN | 2589549 | TTCGGTCTTCAAGGTGGACAATGTC |
| 3161 | TTN | 2589549 | TTCAGGGTGTTCTTTAACACGGTCT |
| 3162 | TTN | 2589549 | TTTCGGTCTTCAAGGTGGACAATGT |
| 3163 | TTN | 2589550 | TGTTTTGGTCTTCGGGGTGGACGGT |
| 3164 | TTN | 2589550 | CTTTCAAGGATTCCGAGGAGGGTGT |
| 3165 | TTN | 2589550 | TCGAGTTCTTCAACAGGGTCTTTTC |
| 3166 | TTN | 2589550 | ACTTCGAGTTCTTCAACAGGGTCTT |
| 3167 | TTN | 2589551 | AGTTCTTCGGCGTCTTTTTCTTTAA |
| 3168 | TTN | 2589551 | GGTTTTTTGGTCTTCGAGGTTAACA |
| 3169 | TTN | 2589551 | TTTGGTCTTCGAGGTTAACAGGGTC |
| 3170 | TTN | 2589551 | GTCTTCAAGGAGTTCTTCGGCGTCT |
| 3171 | TTN | 2589553 | AGGGTTCCGTGGTTAGTTTTTTGGT |
| 3172 | TTN | 2589553 | TAGTTTTTTGGTCTTCGGGGGCGTC |
| 3173 | TTN | 2589555 | TAGACACCGACACGGGTTTTTTGGC |
| 3174 | TTN | 2589555 | ACCGACACGGGTTTTTTGGCCTTCG |
| 3175 | TTN | 2589555 | GACACCGACACGGGTTTTTTGGCCT |
| 3176 | TTN | 2589555 | ACACCGACACGGGTTTTTTGGCCTT |
| 3177 | TTN | 2589558 | CACGGTCACGGAGGAGGATTTTTCG |
| 3178 | TTN | 2589558 | TTTCGGACTTCACGGTGGGTGTTTT |
| 3179 | TTN | 2589558 | GGATTTTTCGGACTTCACGGTGGGT |
| 3180 | TTN | 2589558 | CCGAGGGTTTCTTCAACAAGGACTT |
| 3181 | TTN | 2589559 | AGGGTTCCGTGGTTAGTTTTTTGGT |
| 3182 | TTN | 2589559 | TAGTTTTTTGGTCTTCGGGGGCGTC |
| 3183 | TTN | 2589561 | TAGACACCGACACGGGTTTTTTGGC |
| 3184 | TTN | 2589561 | CTTTCGTAGACACCGACACGGGTTT |
| 3185 | TTN | 2589561 | TCGTAGACACCGACACGGGTTTTTT |
| 3186 | TTN | 2589561 | ACCGACACGGGTTTTTTGGCCTTCG |
| 3187 | TTN | 2589565 | TTTCGGACTTCACGGTGGGTGTTTT |
| 3188 | TTN | 2589565 | GGATTTTTCGGACTTCACGGTGGGT |
| 3189 | TTN | 2589565 | CCGAGGGTTTCTTCAACAAGGACTT |
| 3190 | TTN | 2589565 | CACGGTCACGGAGGAGGATTTTTCG |
| 3191 | TTN | 2589566 | GGATTTTTCGGACTTCAGGGTGGAC |
| 3192 | TTN | 2589566 | CCGAGGGTTTCTTCAACAGGAACTT |
| 3193 | TTN | 2589566 | GATTTTTCGGACTTCAGGGTGGACA |
| 3194 | TTN | 2589566 | GGGAGCCGAGGAGGATTTTTCGGAC |
| 3195 | TTN | 2589567 | TTTCGGTCTTCAAGGTGGACAATGT |
| 3196 | TTN | 2589567 | CAGGGTGTTCTTTAACACGGTCTTT |
| 3197 | TTN | 2589567 | TTCAGGGTGTTCTTTAACACGGTCT |
| 3198 | TTN | 2589567 | TTCGGTCTTCAAGGTGGACAATGTC |
| 3199 | TTN | 2589568 | ACTTCGAGTTCTTCAACAGGGTCTT |
| 3200 | TTN | 2589568 | TCGAGTTCTTCAACAGGGTCTTTTC |
| 3201 | TTN | 2589568 | CTTTCAAGGATTCCGAGGAGGGTGT |
| 3202 | TTN | 2589568 | TGTTTTGGTCTTCGGGGTGGACGGT |
| 3203 | TTN | 2589569 | TCTTCAAGGAGTTCTTCGGTGTCTT |
| 3204 | TTN | 2589569 | TTTGGTCTTCGAGGTTAACAGGGTC |
| 3205 | TTN | 2589569 | GTCTTCAAGGAGTTCTTCGGTGTCT |
| 3206 | TTN | 2589569 | GGTTTTTTGGTCTTCGAGGTTAACA |
| 3207 | TTN | 2589571 | TAGTTTTTTGGTCTTCGGGGGCGTC |
| 3208 | TTN | 2589571 | AGGGTTCCGTGGTTAGTTTTTTGGT |
| 3209 | TTN | 2589573 | TCGTAGACACCGACACGGGTTTTTT |
| 3210 | TTN | 2589573 | TAGACACCGACACGGGTTTTTTGGC |
| 3211 | TTN | 2589573 | ACCGACACGGGTTTTTTGGCCTTCG |
| 3212 | TTN | 2589573 | CTTTCGTAGACACCGACACGGGTTT |
| 3213 | TTN | 2589577 | CCGAGGGTTTCTTCAACAAGGACTT |
| 3214 | TTN | 2589577 | CACGGTCACGGAGGAGGATTTTTCG |
| 3215 | TTN | 2589577 | GGATTTTTCGGACTTCACGGTGGGT |
| 3216 | TTN | 2589577 | TTTCGGACTTCACGGTGGGTGTTTT |
| 3217 | TTN | 2589578 | TCTCGGACTTCAGGGTGGACAATTT |
| 3218 | TTN | 2589606 | CTCCTTCAAGATGGACTTCTTCTCC |
| 3219 | TTN | 2589606 | TCTCCTTCAAGATGGACTCCTTCTC |
| 3220 | TTN | 2589606 | AGGACTTCTCCTTCAAGATGGACTC |
| 3221 | TTN | 2589606 | GGACATCGAGATGGAGTCCTTCTCC |
| 3222 | TTN | 2589840 | AGCGATCCCTGGTGCGTGAAGAGAA |
| 3223 | TTN | 2589840 | ACCTAGTGAAGGAGATAGTCTAACC |
| 3224 | TTN | 2589840 | GACGGATGATCCCAGAGTCCCCAAA |
| 3225 | TTN | 2589840 | TCTCACCAAGCGAAGAAGTCACAGG |
| 3226 | TTN | 2589857 | GATTTTTCGGACTTCAGGGTGGACA |
| 3227 | TTN | 2589857 | GGATTTTTCGGACTTCAGGGTGGAC |
| 3228 | TTN | 2589857 | CCGAGGGTTTCTTCAACAGGAACTT |
| 3229 | TTN | 2589858 | TTCAGGGTGTTCTTTAACACGGTCT |
| 3230 | TTN | 2589858 | TTCGGTCTTCAAGGTGGACAATGTC |
| 3231 | TTN | 2589858 | TTTCGGTCTTCAAGGTGGACAATGT |
| 3232 | TTN | 2589858 | CAGGGTGTTCTTTAACACGGTCTTT |
| 3233 | TTN | 2589859 | CTTTCAAGGATTCCGAGGAGGGTGT |
| 3234 | TTN | 2589859 | TCGAGTTCTTCAACAGGGTCTTTTC |
| 3235 | TTN | 2589859 | TGTTTTGGTCTTCGGGGTGGACGGT |
| 3236 | TTN | 2589859 | ACTTCGAGTTCTTCAACAGGGTCTT |
| 3237 | TTN | 2589860 | GTCTTCAAGGAGTTCTTCGGCGTCT |
| 3238 | TTN | 2589860 | TTTGGTCTTCGAGGCTAACAGGGTC |
| 3239 | TTN | 2589860 | AGTTCTTCGGCGTCTTTTTCTTTAA |
| 3240 | TTN | 2589860 | TTTTGGTCTTCGAGGCTAACAGGGT |
| 3241 | TTN | 2589870 | GTCGGGACAACAGTAACATAACGGT |
| 3242 | TTN | 2589870 | CGACCGAGGTGCCTGATTTTATTAT |
| 3243 | TTN | 2589870 | ATAACGGTCATGGATTCTGTGTCAG |
| 3244 | TTN | 2589870 | GAGGAGACCGATCCGACATTCTTGG |
| 3245 | VGLL3 | 2684856 | CATGTCATTGTATAAGTTACCAAGA |
| 3246 | VGLL3 | 2684856 | ATGTCATTGTATAAGTTACCAAGAC |
| 3247 | VGLL3 | 2684856 | ACATGTCATTGTATAAGTTACCAAG |
| 3248 | VGLL3 | 2684877 | GTAACCCAGTCATCACCTACTTGTG |
| 3249 | VGLL3 | 2684877 | CTTGTGAAGAGTTCTCGAAACCCGG |
| 3250 | VGLL3 | 2684877 | AGTTTTTCGTTCTACCCCGATTGGG |
| 3251 | VGLL3 | 2684877 | GAGTTCTCGAAACCCGGTTCGGTAG |
| 3252 | VGLL3 | 2684865 | TCTTGGTTGATGTCAGTGGAGACGA |
| 3253 | VGLL3 | 2684865 | TGTATCACGGGTCGCACCCTAAGCT |
| 3254 | VGLL3 | 2684865 | TATCACGGGTCGCACCCTAAGCTAT |
| 3255 | VGLL3 | 2684865 | CACGGGTCGCACCCTAAGCTATGTC |
| 3256 | VGLL3 | 2684854 | GTGTAACTGCACCATTTCGAAATTG |
| 3257 | VGLL3 | 2684854 | AGGTATGAGACCTTACGACGACTAG |
| 3258 | VGLL3 | 2684854 | CACGTCTAAGAAGGATCGACTTCAC |
| 3259 | VGLL3 | 2684854 | GTAGAACGCTACAGGATTCAGAGGT |
| 3260 | VGLL3 | 2684887 | TCGGCGATATATTCGCGCCGTCCCT |
| 3261 | VGLL3 | 2684887 | TATTCGCGCCGTCCCTTGTAGGCCT |
| 3262 | VGLL3 | 2684887 | CAGGGACTCGGCGATATATTCGCGC |
| 3263 | VGLL3 | 2684887 | CGACGCAGGGACTCGGCGATATATT |
| 3264 | VGLL3 | 2684861 | ATCGGTTGGTGGAACAGTCCTTTCC |
| 3265 | VGLL3 | 2684861 | GTAGACTCGGAAACGGTTGACACGT |
| 3266 | VGLL3 | 2684861 | CGTCAACTGACCAAAAGCCGGAAAG |
| 3267 | VGLL3 | 2684861 | AGTCGTTATCCTGTGCTTTCCGTAT |
| 3268 | VGLL3 | 2684829 | AGTTTACGTCCAGAGTATTATACAC |
| 3269 | VGLL3 | 2684829 | CAGTAATAGAAGTTAAACAAGTTAT |
| 3270 | VGLL3 | 2684829 | GTTACTATATTCTACTACCTTCTGA |
| 3271 | VGLL3 | 2684829 | TTGTATACAGTAATAGAAGTTAAAC |
| 3272 | VGLL3 | 2684873 | CCTCAAGTAGGACTGAAGGTCCAGT |
| 3273 | VGLL3 | 2684873 | CAGGAACCGGCCCTGTGTTGGACGT |
| 3274 | VGLL3 | 2684873 | AGAGAGTTCGGTCGCCTTATCAAAG |
| 3275 | VGLL3 | 2684873 | GACCGGAATAGGAAACTGTAGAGTC |
| 3276 | VGLL3 | 2684853 | ATGTTCTCTGATAAACGTCTCTCGG |
| 3277 | VGLL3 | 2684853 | CTCGACGTTCTGAAACAAGCTTTGT |
| 3278 | VGLL3 | 2684853 | GGGATAAGGAAGACAACTTTCGAAT |
| 3279 | VGLL3 | 2684853 | CGTACTGTGAGATAGGAAAGAACAC |
| 3280 | VGLL3 | 2684834 | TCAGGCCTCCTTGCAAACTCGGACC |
| 3281 | VGLL3 | 2684834 | GACTCAGGCCTCCTTGCAAACTCGG |
| 3282 | VGLL3 | 2684834 | ATTAGGATCATAATATCCTCCGTCT |
| 3283 | VGLL3 | 2684834 | GGATCATAATATCCTCCGTCTCCGA |
| 3284 | VGLL3 | 2684859 | ACTGTACAAGTCGATCCGTCTCAAG |
| 3285 | VGLL3 | 2684859 | ACGGAACACAGGAAGACTCAAAAGT |
| 3286 | VGLL3 | 2684859 | ACATGAGAGTAGTGAGGCGTGAAAC |
| 3287 | VGLL3 | 2684859 | GACAGACACGAAAGATCCAATGGAG |
| 3288 | VGLL3 | 2684835 | ACTCCTTGACTCTTTACAACCCTTG |
| 3289 | VGLL3 | 2684835 | CCTTGACTCTTTACAACCCTTGGAC |
| 3290 | VGLL3 | 2684835 | GACCAAAGACGACATGTGTCCTTTC |
| 3291 | VGLL3 | 2684835 | TTGACTCTTTACAACCCTTGGACCA |
| 3292 | VGLL3 | 2684869 | GAGACGGGACCTAGGTAGGATACCC |
| 3293 | VGLL3 | 2684869 | GGACCTAGGTAGGATACCCGGAGAC |
| 3294 | VGLL3 | 2684869 | CCTAGGTAGGATACCCGGAGACGAC |
| 3295 | VGLL3 | 2684869 | ACCGAGACGGGACCTAGGTAGGATA |
| 3296 | VGLL3 | 2684833 | CCGTACATCCAGGTTAAGTCAAAAG |
| 3297 | VGLL3 | 2684833 | CCCGATGATAGACGGAGGTGTTAAA |
| 3298 | VGLL3 | 2684833 | GCTTGACAAAATAACTCCCGATGAT |
| 3299 | VGLL3 | 2684833 | TGAGCCGACAATCCGGTAAGAGATT |
| 3300 | VGLL3 | 2684855 | AAAGTACATCAATAATATCACGAAG |
| 3301 | VGLL3 | 2684855 | GTTGTTAATCATAACCTGAAGGTAG |
| 3302 | VGLL3 | 2684855 | TCAGTATTACAAACGCAACCGTAAA |
| 3303 | VGLL3 | 2684855 | AGTGAGAACATTAGCTCTTCCTGAT |
| 3304 | VGLL3 | 2684831 | ACGGTCAAATTACCTCTCCGAGGAT |
| 3305 | VGLL3 | 2684831 | CCCTTAACGTGGTACATGTGAAAAT |
| 3306 | VGLL3 | 2684831 | CGGCACCGATCTCGTTTTCAATTAT |
| 3307 | VGLL3 | 2684831 | AGAACATCACGAGAGACCCTTAACG |
| 3308 | VGLL3 | 2684852 | TCAGACCCTTTTATAGCAATTCAGT |
| 3309 | VGLL3 | 2684852 | GAAGTCCTGATTAGTTCCTAGTTAC |
| 3310 | VGLL3 | 2684852 | TGTACTATAGTACGATACACGGTAA |
| 3311 | VGLL3 | 2684852 | ACACAGTTAATATTGAGTCATTCAG |
| 3312 | VGLL3 | 2684832 | CAATGTTTCCCCATAACTACCGTCA |
| 3313 | VGLL3 | 2684832 | GTCAATAACTTCTGCCTTCCTCAAG |
| 3314 | VGLL3 | 2684832 | TTGTGTTGGTAAATGCTAGAGTCAG |
| 3315 | VGLL3 | 2684832 | CTTCCTCAAGTGAACTCGGTAACGT |
| 3316 | VGLL3 | 2684889 | CACCCGCGGCGTCGGGAGCGCCCTC |
| 3317 | VGLL3 | 2684830 | AATCAATACGACAGTAAAAATTGAT |
| 3318 | VGLL3 | 2684830 | CAATACGACAGTAAAAATTGATTAT |
| 3319 | VGLL3 | 2684830 | ATACGACAGTAAAAATTGATTATTT |
| 3320 | VGLL3 | 2684830 | TCAATACGACAGTAAAAATTGATTA |
| 3321 | VGLL3 | 2684883 | CGGAATACCTCGCAGGGTCATAGAC |
| 3322 | VGLL3 | 2684883 | GGGTCGGAATACCTCGCAGGGTCAT |
| 3323 | VGLL3 | 2684883 | ATACCTCGCAGGGTCATAGACGGGT |
| 3324 | VGLL3 | 2684883 | ACCTCGCAGGGTCATAGACGGGTTG |
| 3325 | VGLL3 | 2684871 | GTACTGCACATGTACGCCGTGGTGG |
| 3326 | VGLL3 | 2684871 | GTCGGTATACGTACTGCACATGTAC |
| 3327 | VGLL3 | 2684871 | CACTCGGGTAGGATGTCGGTATACG |
| 3328 | VGLL3 | 2684871 | TCGGGTAGGATGTCGGTATACGTAC |
| 3329 | VGLL3 | 2684867 | TACGCCGGTCCTAAGGACGAGGGGT |
| 3330 | VGLL3 | 2684867 | ACGTACGCCGGTCCTAAGGACGAGG |
| 3331 | VGLL3 | 2684867 | ACGCCGGTCCTAAGGACGAGGGGTC |
| 3332 | VGLL3 | 2684867 | CGTACGCCGGTCCTAAGGACGAGGG |
| 3333 | VGLL3 | 2684857 | ACGCACGATGGTGTGTTCCGATTAT |
| 3334 | VGLL3 | 2684857 | TCTACGCACGATGGTGTGTTCCGAT |
| 3335 | VGLL3 | 2684857 | TGTCTACGCACGATGGTGTGTTCCG |
| 3336 | VGLL3 | 2684857 | CACGATGGTGTGTTCCGATTATAAA |
| 3337 | VGLL3 | 2684885 | GGCTCCTGGGCGGAAGCGGCGTCAT |
| 3338 | VGLL3 | 2684885 | TACTTCCACGGGCGCGTACCCGGGG |
| 3339 | VGLL3 | 2684885 | CCGGGGGCGACTAACGGTCAGGGAG |
| 3340 | VGLL3 | 2684885 | GCGGAAGCGGCGTCATCGTCGACCT |
| 3341 | VGLL3 | 2684863 | TGTTCTCATTCCTTAGTGGCACCAT |
| 3342 | VGLL3 | 2684863 | CTCATTCCTTAGTGGCACCATGACT |
| 3343 | VGLL3 | 2684863 | TCTGTTCTCATTCCTTAGTGGCACC |
| 3344 | VGLL3 | 2684863 | GTTCTCATTCCTTAGTGGCACCATG |
| 3345 | VGLL3 | 2684879 | TACCTCATGGAATTGAGAGCGACAC |
| 3346 | VGLL3 | 2684879 | CTCATGGAATTGAGAGCGACACAGG |
| 3347 | VGLL3 | 2684879 | GAGAGCGACACAGGAAAAGTGAATA |
| 3348 | VGLL3 | 2684879 | CCTCATGGAATTGAGAGCGACACAG |

What is claimed is:

1. A method comprising: providing a biological sample from a prostate cancer subject; detecting the presence or expression level of at least one or more targets selected from Table 1, Table 2, Table 6, Table 7, Table 15 or SEQ ID NOs: 1-3348; and administering a treatment to the subject, wherein the treatment is selected from the group consisting of surgery, chemotherapy, radiation therapy, immunotherapy/biological therapy, hormonal therapy, and photodynamic therapy.

2. The method of claim 1, wherein the alteration in the expression level of said target is reduced expression of said target.

3. The method of claim 1, wherein the alteration in the expression level of said target is increased expression of said target.

4. The method of claim 1, wherein the level of expression of said target is determined by using a method selected from the group consisting of in situ hybridization, a PCR-based method, an array-based method, an immunohistochemical method, an RNA assay method and an immunoassay method.

5. The method of claim 1, wherein said reagent is selected from the group consisting of a nucleic acid probe, one or more nucleic acid primers, and an antibody.

6. The method of claim 1, wherein the target comprises a nucleic acid sequence.

7. A method comprising:
(a) providing a biological sample from a subject with prostate cancer;
(b) detecting the presence or expression level in the biological sample for a plurality of targets, wherein the plurality of targets comprises one or more targets selected from Table 1, Table 2, Table 6, Table 7, Table 15 or SEQ ID NOs: 1-3348;
(c) subtyping the prostate cancer in the subject based on the presence or expression levels of the plurality of targets; and
(d) administering a treatment to the subject, wherein the treatment is selected from the group consisting of surgery, chemotherapy, radiation therapy, immunotherapy/biological therapy, hormonal therapy, and photodynamic therapy.

8. The method of claim 7, wherein the expression level of said target is reduced expression of said target.

9. The method of claim 7, wherein the expression level of said target is increased expression of said target.

10. The method of claim 7, wherein the level of expression of said target is determined by using a method selected from the group consisting of in situ hybridization, a PCR-based method, an array-based method, an immunohistochemical method, an RNA assay method and an immunoassay method.

11. The method of claim 7, wherein said reagent is selected from the group consisting of a nucleic acid probe, one or more nucleic acid primers, and an antibody.

12. The method of claim 7, wherein the target comprises a nucleic acid sequence.

13. The method of claim 7, wherein the prostate cancer subtype is selected from the group consisting of ERG+. ETS+, SPINK1+, and Triple-Negative.

14. A system for analyzing a cancer, comprising:
(a) A probe set comprising a plurality of target sequences, wherein
(i) the plurality of target sequences hybridizes to one or more targets selected from Table 1, Table 2, Table 6, Table 7, Table 15 or SEQ ID NOs: 1-3348; or
(ii) the plurality of target sequences comprises one or more targets selected from Table 1, Table 2, Table 6, Table 7, Table 15 or SEQ ID NOs: 1-3348; and
(b) a computer model or algorithm for analyzing an expression level and/or expression profile of the target hybridized to the probe in a sample from a subject suffering from prostate cancer.

15. The system of claim 14, further comprising a label that specifically binds to the target, the probe, or a combination thereof.

16. A method of treating a subject with prostate cancer, comprising: providing a biological sample comprising prostate cancer cells from the subject; determining the level of expression or amplification of at least one or more targets selected from Table 1, Table 2, Table 6, Table 7, or Table 15 using at least one reagent that specifically binds to said targets; subtyping the prostate cancer based on the level of expression or amplification of the at least one or more targets; and prescribing a treatment regimen based on the prostate cancer subtype.

17. The method of claim 16, wherein the prostate cancer subtype is selected from the group consisting of ERG+. ETS+, SPINK1+, and Triple-Negative.

18. A kit for analyzing a prostate cancer, comprising:
(a) a probe set comprising a plurality of target sequences, wherein the plurality of target sequences comprises at least one target sequence listed in Table 1, Table 2, Table 6, Table 7, Table 15 or SEQ ID NOs: 1-3348; and
(b) a computer model or algorithm for analyzing an expression level and/or expression profile of the target sequences in a sample.

19. The kit of claim 18, further comprising a computer model or algorithm for correlating the expression level or expression profile with disease state or outcome.

20. The kit of claim 18, further comprising a computer model or algorithm for designating a treatment modality for the individual.

21. The kit of claim 18, further comprising a computer model or algorithm for normalizing expression level or expression profile of the target sequences.
